(19) United States
(12) Patent Application Publication
(10) Pub. No.: US 2025/0311734 A1
(43) Pub. Date: Oct. 9, 2025

(54) Compositions and Methods for Preventing, Treating, Suppressing and/or Eliminatting Phytopathogenic Infestations and Infections

(71) Applicants: NOVOZYMES A/S, Bagsvaerd (DK); NOVONESIS PLANT BIOSOLUTIONS A/S, Lyngby (DK)

(72) Inventors: Kate Sarah BRANDON SUTTON, Durham, NC (US); Svend Gunnar KAASGAARD, Skovlunde (DK); Ines Marques NUNES, Malmo (SE); Gregory Stephen MALONEY, Durham, NC (US); Casey RUARK-SEWARD, Christianburg, VA (US); Jason QUINLAN, Woodland, CA (US); Mary Ann STRINGER, Soeborg (DK); Sharon INCH, Durham, NC (US); Calum RUSSELL, Durham, NC (US); Louisa LIBERMAN, Durham, NC (US); Jennifer PETITTE, Raleigh, NC (US); Birgitte Gjerde BENNEDSEN, Skovlunde (DK); Adam GINN, Roanoke, VA (US); Cari MCGREGOR, Durham, NC (US); Meaghan THOMPSON, Troutville, VA (US)

(73) Assignees: NOVOZYMES A/S, Bagsvaerd (DK); NOVONESIS PLANT BIOSOLUTIONS A/S, Lyngby (DK)

(21) Appl. No.: 18/864,972

(22) PCT Filed: May 12, 2023
(86) PCT No.: PCT/US2023/066918

§ 371 Date: Nov. 12, 2024

Related U.S. Application Data
(63) Continuation-in-part of application No. PCT/US2022/073761, filed on Jul. 15, 2022.
(60) Provisional application No. 63/342,064, filed on May 14, 2022.
(60) Provisional application No. 63/476,590, filed on Dec. 21, 2022.

(51) Int. Cl. A01N 63/50; A01P 3/00; A01P 7/04; C12N 9/04; C12N 9/08; C12N 9/24; C12N 9/50; C12N 15/82

(52) U.S. Cl. CPC A01N 63/50; A01P 3/00; A01P 7/04; C12N 9/0006; C12N 9/0065; C12N 9/2402; C12N 9/2482; C12N 9/50; C12N 15/8279; C12Y 101/03004; C12Y 111/01006; C12Y 302/01008; C12Y 302/01015; C12Y 304/21

(57) ABSTRACT

The present disclosure provides proteins useful for preventing, treating, suppressing and/or eliminating infestations and infections of plants by various phytopathogenic pests, as well as formulations comprising such proteins, polynucleotides encoding such proteins, organisms expressing such proteins, and methods of using such proteins, formulations, polynucleotides and organisms in agriculture and other fields of endeavor.

### RELATED APPLICATIONS

The claims priority to International Patent Application No. PCT/US2022/073761, filed Jul. 15, 2022, and published as WO 2023/288,294 on Jan. 19, 2023, and to U.S. Provisional Application Nos. 63/342,064, filed May 14, 2022, and 63/476,590, filed Dec. 21, 2022, the disclosure of each of which is incorporated herein by reference in its entirety.

### REFERENCE TO A SEQUENCE LISTING

The application contains a Sequence Listing in computer readable form. The name of the file containing the Sequence Listing is SQ.XML, which was created on May 12, 2023, and contains 89,744,839 bytes. The computer readable form is incorporated herein by reference.

### BRIEF DESCRIPTION OF THE DRAWINGS

FIG. 1 is a table showing significant results of efficacy testing of various enzyme treatments against Blumeria graminis. Results are presented as percent disease control relative to an untreated, uninoculated control (none—uninoculated control) and an untreated, inoculated control (none—inoculated control) at two timepoints/disease pressures. 100% control means no disease was detected in the treatment and 0% control means disease was at the same level as the untreated, inoculated control comparator.

FIG. 2 is a table showing significant results of efficacy testing of various enzyme treatments against Botrytis cinerea. Results are presented as percentage increase of the spectral parameters (Fv/Fm, ChlIdx and mARI) and the percentage of water-soaked lesions (% WSL) as compared to the untreated, inoculated control (none—inoculated control).

FIG. 3 is a table showing significant results of efficacy testing of various enzyme treatments against Fusarium graminearum. Results are presented as percentage increase of the spectral parameters (Fv/Fm, ChlIdx and cGFP) as compared to the untreated, inoculated control (none—inoculated control) after 72 hours.

FIG. 4 is a table showing significant results of efficacy testing of various enzyme treatments against Magnaporthe grisea. Results are presented as the half maximal effective concentration for absorbance at 612 nm (Abs EC₅₀; indicator of mycelia growth inhibition) and half maximal effective concentration for fluorescence after reaction with Resazurin (ex 570 nm-em 585 nm) (Fluorescence EC₅₀; indicator of cell viability).

FIG. 5 is a table showing significant results of efficacy testing of various enzyme treatments against Phakopsora pachyrhizi. Results are presented as the mean number of combined Phakopsora pachyrhizi lesions and pustules.

FIG. 6A, FIG. 6B, FIG. 6C and FIG. 6D are tables showing significant results of efficacy testing of various enzyme treatments against Phytophthora infestans. Results for pipette-treated tomato leaf discs treated are presented in FIG. 6A as mean percent disease. Results for spray-treated tomato leaf discs are presented in FIG. 6B as (−) no disease reduction, (+) mild disease reduction, (++) moderate disease reduction or (+++) extreme disease reduction and in FIG. 6C as mean percent disease. Results for spray-treated potato leaf discs are presented in FIG. 6D as mean percent healthy.

FIG. 7 is a table showing significant results of efficacy testing of various enzyme treatments against Pseudoperonospora cubensis. Results are presented as (−) no disease reduction, (+) mild disease reduction, (++) moderate disease reduction or (+++) extreme disease reduction.

FIG. 8A and FIG. 8B are tables showing significant results of efficacy testing of various enzyme treatments against Zymoseptoria tritici. Results are presented as percent disease control relative to untreated, inoculated control at various timepoints/disease pressures. 100% control means no disease was detected in the treatment and 0% control means disease was at the same level as the untreated, inoculated control comparator (none—inoculated control).

FIG. 9 is a table showing significant results of efficacy testing of various enzyme treatments against Botrytis cinerea, Fusarium graminearum, Fusarium virgulforme, and Zymoseptoria tritici. Results are presented as the minimum inhibitory concentration (MIC₅₀) for average hyphal branch length and mycelial area (i.e., the minimum enzyme concentration required to inhibit average hyphal branch length/total mycelium area to 50% or less of the corresponding untreated control).

FIG. 10A and FIG. 10B are tables showing significant results of efficacy testing of various enzyme treatments against Botrytis cinerea, Fusarium graminerum, Magnaporthe grisea, Penicillium digitatum, Penicillium expansum, Penicillium italicum, Phytophthora infestans and Zymoseptoria tritici. Results are presented as (−) no growth inhibition, (+) visible stress reaction and/or mild growth inhibition, (++) moderate growth inhibition or (+++) extreme growth inhibition.

FIG. 11 is a table showing significant results of efficacy testing of various enzyme treatments against leaf-eating insect larvae. Results are presented as relative growth rates of the larvae after two days of feeding on treated leaf discs.

FIG. 12 is a table showing significant results of efficacy testing of various enzyme treatments against leaf-eating insect larvae. Results are presented as relative growth rates of the larvae after two days of feeding on treated leaf discs.

FIG. 13 is a table showing significant results of efficacy testing of various enzyme treatments against gray mold (Botrvtis cinerea). Results are presented as percent disease.

FIG. 14A and FIG. 14B are tables showing significant results of efficacy testing of various enzyme treatments against Fusarium graminearum. Results are presented as percentage increase of the spectral parameters (Fv/Fm, ChlIdx and cGFP) as compared to corresponding untreated, inoculated controls (none—inoculated control; none—inoculated Silwet™ control; none—inoculated pH 6 buffer control; none—inoculated pH 7 buffer control; none—inoculated pH 8 buffer control).

FIG. 15A and FIG. 15B are tables showing significant results of efficacy testing of various enzyme treatments against Puccinia striiformis. Results are presented as area under the disease progress curve.

FIG. 16 is a table showing results of significant results of efficacy testing of various enzyme treatments against Botrytis cinerea, Fusarium graminerum, Penicillium digitatum, Penicillium expansum, Penicillium italicum, Phytophthora infestans, Pyricularia grisea and Zymoseptoria tritici. Results are presented as the amount of oligosaccharides solubilized after hydrolysis of crude cell wall preparations (mg glucose equivalents per gram of crude cell wall material (mg Glc/g CW)) FIG. 17 is a table showing significant results of efficacy testing of various enzyme treatments against Botrytis cinerea and Penicillium expansum. Results are presented as minimum enzyme concentrations necessary to prevent spore germination in the presence of enzyme, 24 hours after enzyme washout, or 48 hours after enzyme washout.

FIG. 18 is a table showing significant results of efficacy testing of various enzyme treatments against Botrytis cinerea. Results are presented as the minimum inhibitory concentration (MIC₅₀) for average total mycelial area (i.e., the minimum enzyme concentration required to inhibit average total mycelium area to 50% or less of the corresponding untreated control).

FIG. 19 is a table showing significant results of efficacy testing of various enzyme treatments against Botrytis cinerea and Penicillium expansum. Results are presented as percent inhibition of germ tube elongation as compared to the corresponding buffer control solution.

### DETAILED DESCRIPTION

This description is not intended to be a detailed catalog of all the different ways in which the inventive concepts disclosed herein may be implemented or of all the features that may be added thereto. For example, features illustrated with respect to one embodiment may be incorporated into other embodiments and features illustrated with respect to a particular embodiment may be deleted from that embodiment. In addition, numerous variations and additions to the various embodiments suggested herein, which do not depart from the instant inventions, will be apparent to those skilled in the art in light of the instant disclosure. Hence, the following description is intended to illustrate some embodiments of the instant inventions and not to exhaustively specify all permutations, combinations and variations thereof.

The terminology used herein is for the purpose of describing particular embodiments only and is not intended to be limiting of the instant inventions.

Unless otherwise defined, all terms (including technical and scientific terms) used herein have the same meaning as commonly understood by one of ordinary skill in the art to which the inventions belong. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the singular forms “a,” “an,” and “the” are intended to include the plural forms as well, unless the context clearly indicates otherwise.

As used herein, “additive,” when referring to effects of combinations within a composition means that the effects of the combinations are generally about the same as the sum of effects of the individual components of the combination alone. The combination of individual components producing this effect may be called an additive combination.

As used herein, the terms “agricultural, floricultural, horticultural and/or silvicultural apparatus/facility” and “agricultural/floricultural/horticultural/silvicultural apparatus/facility” refer to an apparatus or facility utilized in one or more aspects of the plant propagation, cultivation and/or harvesting, including, but not limited to, breeding, planting, irrigating, fertilizing, growing, monitoring, testing, pruning, harvesting, processing, packaging and/or storing plants and plant parts. Exemplary apparatuses and facilities include cultivators, seed containers, seeders, planting pots, hydroponic growth systems, growth chambers, greenhouses, broadcasters, fertilization drills, fertilizer spreaders, irrigation systems, harvesting apparatuses, postharvest storage containers, postharvest treatment chambers, and postharvest shipping containers.

As used herein, the term “agriculturally acceptable carrier” refers to a substance or composition that can be used to deliver a beneficial agent to a plant, plant part or plant growth medium (e.g., soil) without causing/having an unduly adverse effect on plant growth, development and/or yield. As used herein, the term “foliar-compatible carrier” refers to a material that can be foliarly applied to a plant or plant part without causing/having an unduly adverse effect on the plant, plant part, plant growth, plant health, or the like. As used herein, the term “seed-compatible carrier” refers to a material that can be applied to a seed without causing/having an unduly adverse effect on the seed, the plant that grows from the seed, seed germination, or the like. As used herein, the term “soil-compatible carrier” refers to a material that can be added to a soil without causing/having an unduly adverse effect on plant growth, soil structure, soil drainage, or the like.

As used herein, the term “and/or” is intended to include any and all combinations of one or more of the associated listed items, as well as the lack of combinations when interpreted in the alternative (“or”). Thus, the phrase “A, B and/or C” is to be interpreted as “A, A and B, A and B and C, A and C, B, B and C, or C.”

As used herein, “antagonistic,” when referring to effects of combinations within a composition means that the effects of the combinations are generally less than the sum of effects of the individual components of the combination alone. These compositions may be called antagonistic combinations.

As used herein, the term “aqueous” refers to a composition that contains more than a trace amount of water (i.e., more than 0.5% water by weight, based upon the total weight of the composition).

As used herein, the terms “associated with,” in association with” and “associated therewith,” when used in reference to a relationship between a composition of the present disclosure and a plant or plant part, refer to at least a juxtaposition or close proximity of the composition and the plant or plant part. Such a juxtaposition or close proximity may be achieved by contacting or applying the composition directly to the plant or plant part and/or by applying the composition to the plant growth medium (e.g., soil) in which the plant or plant part will be grown (or is currently being grown). According to some embodiments, the composition is applied as a coating to the outer surface of the plant or plant part. According to some embodiments, the composition is applied to soil at, near or surrounding the site in which the plant or plant part will be grown (or is currently being grown).

As used herein, the term “beneficial agent” may refer to any agent having at least one agriculturally/floriculturally/horticulturally/silviculturally beneficial property (e.g., an ability to fix atmospheric nitrogen, an ability to solubilize phosphate, an ability to produce one or more agriculturally/floriculturally/horticulturally/silviculturally beneficial small molecules, such as plant signal molecules, an ability to stimulate one or more plant defense systems, and ability to produce one or more photoprotective agents, such as pesticidal toxins).

As used herein, the term “biostimulant” refers to an agent or combination of agents the application of which enhances one or more metabolic and/or physiological processes of a plant or plant part (e.g., carbohydrate biosynthesis, ion uptake, nucleic acid uptake, nutrient delivery, photosynthesis and/or respiration).

As used herein, the term “binding module” refers to the region of an enzyme that mediates binding to the enzyme to a substrate.

As used herein, the term “catalytic domain” refers to the region of an enzyme containing the catalytic machinery of the enzyme.

As used herein, the term “cDNA” refers to a DNA molecule that can be prepared by reverse transcription from a mature, spliced, mRNA molecule obtained from a eukaryotic or prokaryotic cell. cDNAs lack intron sequences that may be present in the corresponding genomic DNA. The initial, primary RNA transcript is a precursor to mRNA that is processed through a series of steps, including splicing, before appearing as mature spliced mRNA.

As used herein, the term “coding sequence” refers to a polynucleotide that directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

As used herein, the terms “colony forming unit” and “cfu” refer to a microbial cell/spore capable of propagating on or in a suitable growth medium or substrate (e.g., a soil) when conditions (e.g., temperature, moisture, nutrient availability, pH, etc.) are favorable for germination and/or microbial growth.

As used herein, the term “consists essentially of,” when used in reference to compositions and methods of the present disclosure, means that the compositions/methods may contain additional components/steps so long as the additional components/steps do not materially alter the composition/method. The term “materially alter,” as applied to a composition/method of the present disclosure, refers to an increase or decrease in the effectiveness of the composition/method of at least 20%. For example, a component added to a composition of the present disclosure may be deemed to “materially alter” the composition if it increases or decreases the composition's ability to inhibit the growth of a target phytopathogen by at least 20%.

As used herein, the term “control sequences” refers to nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptidic.

As used herein, the term “derived from,” when used in reference to a relationship between an organism and a protein and/or polynucleotide, means that the protein and/or polynucleotide is naturally occurring in said organism.

As used herein, the term “diazotroph” refers to an organism capable of converting atmospheric nitrogen (N₂) into a form that may be utilized by a plant or plant part (e.g., ammonia (NH₃), ammonium (NH₄+), etc.).

As used herein, the term “dispersant” refers to an agent or combination of agents the application of which reduces the cohesiveness of like particles, the surface tension of a liquid, the interfacial tension between two liquids and/or the interfacial tension between or a liquid and a solid.

As used herein, the terms “effective amount,” “effective concentration” and “effective amount/concentration” refer to an amount or concentration that is sufficient to cause a desired effect (e.g., inhibiting plant disease, enhancing plant yield). The absolute value of the amount/concentration that is sufficient to cause the desired effect may be affected by factors such as the type and magnitude of effect desired, the type, size and volume of material to which the composition will be applied, the type(s) of enzymes in the composition, the amount(s) of enzyme(s) in the composition, the stability of the enzyme(s) in the composition and the storage conditions (e.g., temperature, relative humidity, duration). Those skilled in the art will understand how to select an effective amount/concentration using routine dose-response experiments. In some examples, an effective amount of a substance when used alone may be different than an effective amount of the same substance when used as part of a combination.

As used herein, the term “endogenous gene” refers to a gene consisting of an endogenous polynucleotide.

As used herein, the term “endogenous polynucleotide” refers to a polynucleotide that is native to the referenced host cell.

As used herein, the terms “enhanced growth” and “enhanced plant growth” refer to an improvement in one or more characteristics of plant growth and/or development as compared to one or more control plants (e.g., a plant germinated from an untreated seed or an untreated plant). Exemplary plant growth/development characteristics include, but are not limited to, biomass, carbohydrate biosynthesis, chlorophyll content, cold tolerance, drought tolerance, height, leaf length, leaf mass, leaf number, leaf surface area, leaf volume, nutrient uptake (e.g., calcium, magnesium, nitrogen, phosphorous and/or potassium uptake), rate(s) of photosynthesis, root area, root diameter, root length, root mass, root nodulation (e.g., nodule mass, nodule number, nodule volume), root number, root surface area, root volume, salt tolerance, seed germination, seedling emergence, shoot diameter, shoot length, shoot mass, shoot number, shoot surface area, shoot volume, spread, stomatal conductance and survival rate.

As used herein, the terms “enhanced stability” and “enhanced enzyme stability” refer to an improvement in one or more characteristics of enzyme stability as compared to one or more controls (e.g., a control composition that is identical to a composition of the present disclosure except that it lacks one or more of the components found in the composition of the present disclosure). Exemplary enzyme stability characteristics include, but are not limited to, maintenance of enzymatic activity after being applied to a plant or plant part and/or stored for a defined period of time and the ability to cause a desired effect (e.g., reduced phytopathogenicity of a target pest) after being applied to a plant or plant part and/or stored for a defined period of time.

As used herein, the terms “enhanced yield” and “enhanced plant yield” refer to an improvement in one or more characteristics of plant yield as compared to one or more control plants (e.g., a control plant germinated from an untreated seed). Exemplary plant yield characteristics include, but are not limited to, biomass; bushels per acre; grain weight per plot (GWTPP); nutritional content; percentage of plants in a given area (e.g., plot) that fail to produce grain; yield at standard moisture percentage (YSMP), such as grain yield at standard moisture percentage (GYSMP); yield per plot (YPP), such as grain weight per plot (GWTPP); and yield reduction (YRED).

As used herein, the term “expression” refers to any step involved in the production of a polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

As used herein, the term “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the term “extension” refers to an addition of one or more amino acids to the amino and/or carboxyl terminus of a polypeptide.

As used herein, the term “foliage” refers to those portions of a plant that normally grow above the ground, including, but not limited to, leaves, stalks, stems, flowers, fruiting bodies and fruits.

As used herein, the terms “foliar application” and “foliarly applied” refer to the application of one or more active ingredients to the foliage of a plant (e.g., to the leaves of the plant). Application may be affected by any suitable means, including, but not limited to, spraying/fogging the plant with a composition comprising the active ingredient(s). In some embodiments, the active ingredient(s) is/are applied to the leaves, stems and/or stalk of the plant and not to the flowers, fruiting bodies or fruits of the plant.

As used herein, the term “fragment” refers to a polypeptide having one or more amino acids absent from the amino and/or carboxyl terminus of the mature polypeptide.

As used herein, the term “fusion protein” refers to a polypeptide in which one polypeptide is fused at the N-terminus and/or the C-terminus of a polypeptide of the present disclosure. A fusion protein is produced by fusing a polynucleotide encoding another polypeptide to a polynucleotide of the present disclosure, or by fusing two or more polynucleotides of the present disclosure together. Techniques for producing fusion proteins are known in the art and include ligating the coding sequences encoding the polypeptides so that they are in frame and that expression of the fusion protein is under control of the same promoter(s) and terminator. Fusion proteins may also be constructed using intein technology in which fusion proteins are created post-translationally (Cooper et al., 1993, EMBO J. 12: 2575-2583; Dawson et al., 1994, Science 266: 776-779). A fusion protein can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

As used herein, the term “heterologous,” when used to describe the relationship between a polynucleotide or polypeptide and a host cell, refers to a polynucleotide or polypeptide that does not naturally occur in the host cell. For the purposes of the present disclosure, extraneous copies of polynucleotides that are otherwise native to the referenced host cell are deemed heterologous polynucleotides.

As used herein, the term “heterologous,” when used to describe the relationship between a polynucleotide or polypeptide and a control sequence (e.g., a promoter sequence), refers to a polynucleotide or polypeptide is not naturally associated with the control sequence (i.e., the control sequence is from a gene other than the gene encoding the mature polypeptide).

As used herein, the terms “host strain” and “host cell” refer to an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) and plant cells capable of expressing a protein of interest. The term “host cell” includes protoplasts created from cells.

As used herein, the terms “inoculant composition” and “inoculum” refer to a composition comprising microbial cells and/or spores, said cells/spores being capable of propagating/germinating on or in a suitable growth medium or substrate (e.g., a soil) when conditions (e.g., temperature, moisture, nutrient availability, pH, etc.) are favorable for germination and/or microbial growth.

As used herein, the term “introduced,” when used to describe the insertion of a nucleic acid sequence into a cell, encompasses “transfection”, “transformation” or “transduction,” as known in the art.

As used herein, the term “isolated” refers to a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

As used herein, the term “isomer” includes all stereoisomers of the compounds and/or molecules to which it refers, including enantiomers and diastereomers, as well as all conformers, roatmers and tautomers, unless otherwise indicated. Compounds and/or molecules disclosed herein include all enantiomers in either substantially pure levorotatory or dextrorotatory form, or in a racemic mixture, or in any ratio of enantiomers.

Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose a (S)-enantiomer, that embodiment also includes the (R)-enantiomer; where embodiments disclose a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers and tautomers of compounds and/or molecules depicted.

As used herein, the term “mature polypeptide” refers to a polypeptide in its mature form following N-terminal and/or C-terminal processing (e.g., removal of signal peptide).

As used herein, the term “mature polypeptide coding sequence” refers to a polynucleotide that encodes a mature polypeptide.

As used herein, the term “modified microbial strain” refers to a microbial strain that is modified from a strain isolated from nature. Modified microbial strains may be produced by any suitable method(s), including, but not limited to, chemical or other form of induced mutation to a polynucleotide within any genome within the strain; the insertion or deletion of one or more nucleotides within any genome within the strain, or combinations thereof; an inversion of at least one segment of DNA within any genome within the strain; a rearrangement of any genome within the strain; generalized or specific transduction of homozygous or heterozygous polynucleotide segments into any genome within the strain; introduction of one or more phage into any genome of the strain; transformation of any strain resulting in the introduction into the strain of stably replicating autonomous extrachromosomal DNA; any change to any genome or to the total DNA composition within the strain isolated from nature as a result of conjugation with any different microbial strain; and any combination of the foregoing. The term modified microbial strains includes a strain with (a) one of more heterologous nucleotide sequences, (b) one or more non-naturally occurring copies of a nucleotide sequence isolated from nature (i.e., additional copies of a gene that naturally occurs in the microbial strain from which the modified microbial strain was derived), (c) a lack of one or more nucleotide sequences that would otherwise be present in the natural reference strain by for example deleting nucleotide sequence, and (d) added extrachromosomal DNA. In some embodiments, modified microbial strains comprise a combination of two or more nucleotide sequences (e.g., two or more naturally occurring genes that do not naturally occur in the same microbial strain) or comprise a nucleotide sequence isolated from nature at a locus that is different from the natural locus.

As used herein, the term “native” refers to a polynucleotide or polypeptide naturally occurring in a host cell.

As used herein, the term “naturally occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in a laboratory, modification of a wild-type sequence, formulations comprising one or more synthetic components, formulations comprising an artificial combination of otherwise naturally occurring components).

As used herein, the term “non-aqueous” refers to a composition that comprises no more than a trace amount of water (i.e., no more than 0.5% water by weight, based upon the total weight of the composition).

As used herein, the term “nutrient” refers to a compound or element useful for nourishing a plant (e.g., vitamins, macrominerals, micronutrients, trace minerals, organic acids, etc. that are necessary for plant growth and/or development).

As used herein, the term “obtained from,” when used in reference to a relationship between an organism and a protein, means that the protein is expressed in the organism, whether from a naturally occurring polynucleotide therein or from a heterologous polynucleotide that was introduced into the organism.

As used herein, the term “polynucleotide” encompasses DNA, RNA, heteroduplexes, and synthetic molecules capable of encoding a polypeptide. Polynucleotides may be single-stranded or double-stranded and may comprise chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

As used herein, the term “nucleic acid construct” refers to a polynucleotide, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

As used herein, the term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

As used herein, the term “phosphate-solubilizing microorganism” refers to a microorganism capable of converting insoluble phosphate into a soluble form of phosphate.

As used herein, the term “phytopathogenic pest” includes any organism or virus that negatively affects a plant, including, but not limited to, organisms and viruses that spread disease, damage host plants and/or compete for soil nutrients. The term “phytopathogenic pest” encompasses organisms and viruses that are known to associate with plants and to cause a detrimental effect on the plant's health and/or vigor. Phytopathogenic pests include, but are not limited to, arachnids (e.g., mites, ticks, spiders, etc.), bacteria, fungi, gastropods (e.g., slugs, snails, etc.), invasive plants (e.g., weeds), insects (e.g., white flies, thrips, weevils, etc.), nematodes (e.g., root-knot nematode, soybean cyst nematode, etc.), rodents and viruses (e.g., tobacco mosaic virus (TMV), tomato spotted wilt virus (TSWV), cauliflower mosaic virus (CaMV), etc.).

As used herein, the term “plant” includes all plant populations, including, but not limited to, agricultural, floricultural, horticultural and silvicultural plants. The term “plant” encompasses plants obtained by conventional plant breeding and optimization methods (e.g., marker-assisted selection) and plants obtained by genetic engineering, including cultivars protectable and not protectable by plant breeders' rights.

As used herein, the term “plant cell” refers to a cell of an intact plant, a cell taken from a plant, or a cell derived from a cell taken from a plant. Thus, the term “plant cell” includes cells within seeds, suspension cultures, embryos, meristematic regions, callus tissue, leaves, shoots, gametophytes, sporophytes, pollen and microspores.

As used herein, the term “plant growth regulator” refers to an agent or combination of agents the application of which accelerates or retards the growth/maturation rate of a plant through direct physiological action on the plant or which otherwise alters the behavior of a plant through direct physiological action on the plant. “Plant growth regulator” shall not be interpreted to include any agent or combination of agents excluded from the definition of “plant regulator” that is set forth section 2(v) of the Federal Insecticide, Fungicide, and Rodenticide Act (7 U.S.C. § 136(v)). Thus, “plant growth regulator” does not encompass microorganisms applied to a plant, plant part or plant growth medium for the purpose of enhancing the availability and/or uptake of nutrients, nutrients necessary to normal plant growth, soil amendments applied for the purpose of improving soil characteristics favorable for plant growth or vitamin hormone products as defined by 40 C.F.R. § 152.6(f).

As used herein, the term “plant part” refers to any part of a plant, including cells and tissues derived from plants. Thus, the term “plant part” may refer to any of plant components or organs (e.g., leaves, stems, roots, etc.), plant tissues, plant cells and seeds. Examples of plant parts, include, but are not limited to, anthers, embryos, flowers, fruits, fruiting bodies, leaves, ovules, pollen, rhizomes, roots, seeds, shoots, stems and tubers, as well as scions, rootstocks, protoplasts, calli and the like.

As used herein, the term “plant propagation material” refers to a plant part from which a whole plant can be generated. Examples of plant propagation materials include, but are not limited to, cuttings (e.g., leaves, stems), rhizomes, seeds, tubers and cells/tissues that can be cultured into a whole plant.

As used herein, the term “protein” is not meant to refer to a specific amino acid chain length and encompasses peptides, oligopeptides and polypeptides. It is to be understood that the term “protein” also encompasses two or more polypeptides combined to form an encoded product, as well as hybrid polypeptides and fusion proteins.

As used herein, the term “purified” refers to a polynucleotide, protein or cell that is substantially free from other components as determined by analytical techniques well known in the art (e.g., a purified polynucleotide or protein may form a discrete band in an electrophoretic gel, chromatographic eluate, and/or a media subjected to density gradient centrifugation). A purified polynucleotide or protein is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polynucleotide, protein, cell, nucleic acid, amino acid, or other specified material or component that is present in a composition at a relative or absolute concentration that is higher than a starting composition.

In one aspect, the term “purified” as used herein refers to the protein or cell being essentially free from components (especially insoluble components) from the production organism. In other aspects, the term “purified” refers to the protein being essentially free of insoluble components (especially insoluble components) from the native organism from which it is obtained. In one aspect, the protein is separated from some of the soluble components of the organism and culture medium from which it is recovered. The protein may be purified (i.e., separated) by one or more of the unit operations filtration, precipitation, or chromatography.

Accordingly, the protein may be purified such that only minor amounts of other proteins, in particular, other proteins, are present. The term “purified” as used herein may refer to removal of other components, particularly other proteins and most particularly other enzymes present in the cell of origin of the protein. The protein may be “substantially pure”, i.e., free from other components from the organism in which it is produced, e.g., a host organism for recombinantly produced protein. In one aspect, the protein is at least 40% pure by weight of the total protein material present in the preparation. In one aspect, the protein is at least 50%, 60%, 70%, 80% or 90% pure by weight of the total protein material present in the preparation. As used herein, a “substantially pure protein” may denote a protein preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other protein material with which the protein is natively or recombinantly associated.

It is, therefore, preferred that the substantially pure protein is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total protein material present in the preparation. Proteins of the present disclosure are preferably in a substantially pure form (i.e., the preparations are essentially free of other protein material). This can be accomplished, for example by preparing the protein by well-known recombinant methods or by classical purification methods.

As used herein, the term “recombinant” is used in its conventional meaning to refer to the manipulation, e.g., cutting and rejoining, of nucleic acid sequences to form constellations different from those found in nature. The term recombinant refers to a cell, nucleic acid, protein or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

As used herein, the terms “recover” and “recovery” refer to the removal of a protein from at least one fermentation broth component selected from the list of a cell, a nucleic acid, or other specified material, e.g., recovery of the protein from the whole fermentation broth, or from the cell-free fermentation broth, by protein crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the protein and using relevant solid-liquid separation methods to harvest the protein from the broth media by use of classification separation by particle sizes. Recovery encompasses isolation and/or purification of the protein.

As used herein, the relatedness between two amino acid sequences or between two nucleotide sequences is described by the parameter “sequence identity”.

For purposes of the present disclosure, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present disclosure, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment).

As used herein, the term “signal peptide” refers to a sequence of amino acids attached to the N-terminal portion of a protein, which facilitates the secretion of the protein outside the cell. The mature form of an extracellular protein lacks the signal peptide, which is cleaved off during the secretion process.

As used herein, the terms “stabilizing compound” and “stabilizer” refer to an agent or combination of agents the application of which enhances the stability of an enzyme.

As used herein, the term “subsequence” refers to a polynucleotide having one or more nucleotides absent from the 5′ and/or 3′ end of a mature protein coding sequence; wherein the subsequence encodes a fragment having enzymatic activity.

As used herein, the term “variant” refers to a protein comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

As used herein, the term “wild-type” in reference to an amino acid sequence or nucleic acid sequence means that the amino acid sequence or nucleic acid sequence is a native or naturally occurring sequence.

While certain aspects of the present disclosure will hereinafter be described with reference to embodiments thereof, it will be understood by those of ordinary skill in the art that various changes in form and details may be made therein without departing from the spirit and scope of the present disclosure as defined by the claims.

All publications, patent applications, patents and other references mentioned herein are incorporated by reference in their entirety, except insofar as they contradict any disclosure expressly set forth herein.

The present disclosure provides proteins useful for a) preventing, treating, suppressing and/or eliminating infestations/infections of/by myriad pests, including, but not limited to, phytopathogenic pests, such as arachnids, bacteria, fungi, gastropods, insects, nematodes, oomycetes, protozoa, viruses and weeds; b) treating surfaces/substances that are susceptible to infestation/infection; c) cleansing infested/infected surfaces/substances; d) reducing disease severity in plants and plant parts affected directly or indirectly by phytopathogenic pests; e) enhancing plant growth environments; f) improving nutrient availability in plant growth media; g) reducing the amount(s) of exogenous fertilizer needed to achieve a desired result; h) improving plant growth, development and yield characteristics; i) prolonging the shelf-life of harvested plants and plant parts; j) delaying the ripening of plants and plant parts; k) hastening the ripening of plants and plant parts; l) improving the efficacy of biological/chemical pesticides; m) preventing, treating, suppressing and/or eliminating pesticide-induced resistance/phytotoxicity, as well as polynucleotides encoding such proteins, organisms expressing such proteins, formulations comprising such proteins, polynucleotides and organisms, and methods of using such proteins, polynucleotides, organisms and formulations in agriculture and other fields of endeavor.

As those skilled in the art will appreciate, proteins, polynucleotides and organisms of the present disclosure may be used (and may be formulated for use) at any time(s) throughout the agricultural, floricultural, horticultural, and silvicultural processes, such as prior to planting, at the time of planting, after planting, prior to germination, after germination, prior to seedling emergence, at the time of seedling emergence, after seedling emergence, prior to the vegetative stage, during the vegetative stage, after the vegetative stage, prior to the reproductive stage, during the reproductive stage, after the reproductive stage, prior to flowering, at the time of flowering, after flowering, prior to fruiting, at the time of fruiting, after fruiting, prior to ripening, at the time of ripening, after ripening, prior to harvest, at the time of harvest, after harvesting, prior to transport/storage, at the time of transport/storage, and/or after transport/storage.

Accordingly, proteins of the present disclosure may be formulated for any suitable method of application, including, but not limited to, on-seed application, in-furrow application, foliar application, preharvest application, and postharvest application.

As those skilled in the art will further appreciate, proteins, polynucleotides, organisms and formulations of the present disclosure may affect the desired outcome(s)—including prevention, treatment, suppression and/or elimination of infestations/infections-without being toxic. As will be explained in further detail below, compositions of the present disclosure may exert their effects through various non-lethal means, such as reducing the attraction of a pest to a treated surface by degrading a food source, for example. Moreover, in many instances, otherwise toxic proteins of the present disclosure may be used in non-lethal doses to enhance the efficacy of and/or expand the target pest range of various chemical pesticides and biological pesticides.

Finally, those skilled in the art will appreciate that proteins, polynucleotides, organisms and formulations of the present disclosure may be used in combination to achieve the desire outcome(s). The present disclosure thus extends to formulations comprising two or more proteins of the present disclosure, to hybrid proteins comprising two or more distinct catalytic domains, to fusion proteins comprising two or more enzymatic polypeptides, etc. Although certain combinations will be described in detail below, it is to be understood that the present disclosure is not limited to those combinations but extends to all possible combinations of proteins, formulations, polynucleotides, and organisms described herein.

In some embodiments, proteins of the present disclosure exhibit one or more catalytic activities belonging to Enzyme Commission classification number 1 (EC 1). For example, in some embodiments, proteins of the present disclosure exhibit glucose oxidase, cellobiose dehydrogenase, amino acid oxidase, laccase, catalase, peroxidase and/or oxygenase activity useful for a) preventing/treating/suppressing/eliminating/reducing the detrimental effects of infestations/infections of/by various pests, including, but not limited to, phytopathogenic pests, such as acarids, bacteria, fungi, gastropods, insects, nematodes, oomycetes, protozoa, viruses and weeds; b) reducing one or more aspects of disease severity in plants affected by one or more phytopathogenic pests; c) pretreating surfaces/substances that are susceptible to infestation/infection by pests; d) cleaning surfaces/substances that are infested/infected by pests; e) enhancing the environments in which plants are grown by; f) improving nutrient availability in plant growth media; g) reducing the amounts of exogenous fertilizer needed to achieve a desired result; h) improving plant growth, development, and yield characteristics; i) prolonging the shelf-life of harvested plants and plant parts; j) delaying/hastening the ripening of a plant or plant part; k) improving the efficacy of chemical pesticides; and/or l) reducing chemical-pesticide-induced resistance/phytotoxicity.

In some embodiments, proteins of the present disclosure exhibit one or more oxidoreductase activities belonging to EC 1 and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 1-15 and 183-2755.

In some embodiments, proteins of the present disclosure exhibit one or more oxidoreductase activities belonging to EC 1.1, such as oxidase activities belonging to EC 1.1.3 (e.g., glucose oxidase activity belonging to EC 1.1.3.4, hexose oxidase activity belonging to EC 3.1.1.5, galactose oxidase activity belonging to EC 1.1.3.9) and/or EC 1.1.99 (e.g., cellobiose oxidase activity belonging to EC 1.1.99.18), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one, two, three, four, five or more of the amino acid sequences set forth herein as SEQ ID NO(s): 1-8.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 1-5;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 1-5;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 92-96 or the cDNA sequence thereof;
d) a polypeptide derived from any one of SEQ ID NO(s): 1-5 by substitution, deletion, or insertion of one or more amino acids;
e) a polypeptide derived from a mature polypeptide of any one of SEQ ID NO(s): 1-5 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucose oxidase activity belonging to EC 1.1.3.4. Examples of proteins that exhibit glucose oxidase activity and have an amino acid sequence that is at least 70% identical to one or more of SEQ ID NOs: 1-5 are set forth herein as SEQ ID NOs: 183-2205.

In some such embodiments, the protein is derived from Aspergillus (e.g., A. chevalieri, A. cristatus, A. flavus, A. niger, A. niveoglaucus, A. nomiae, A. oryzae., A. terreus, A. tubingensis), Beauveria (e.g., B. bassiana), Escherichia (e.g., E. coli), Komagataella (e.g., K. pastoris), Penicillium (e.g., P. adametzii. P. amagasakiense, P. chrysogenum, P. decumbens, P. expansum, P. polonicum, P. viridicatum), or Talaromyces (e.g., T. bacillisporus, T. flavus, T. stipitatus, T. variabilis). For example, in some embodiments, the protein is a native Aspergillus (e.g., A. chevalieri, A. cristatus, A. flavus, A. niger, A. niveoglaucus, A. nomiae, A. oryzae., A. terreus, A. tubingensis), Beauveria (e.g., B. bassiana), Escherichia (e.g., E. coli), Komagataella (e.g., K pastoris), Penicillium (e.g., P. adametz, P. amagasakiense, P. chrysogenum, P. decumbens, P. expansum, P. polonicum, P. viridicatum), or Talaromyces (e.g., T. bacillisporus, T. flavus, T. stipitatus, T. variabilis) glucose oxidase or is a functional fragment/mutant/variant of a native Aspergillus (e.g., A. chevalieri, A. cristatus, A. flavus, A. niger, A. niveoglaucus, A. nomiae, A. oryzae., A. terreus, A. tubingensis), Beauveria (e.g., B. bassiana), Escherichia (e.g., E. coli), Komagataella (e.g., K. pastoris), Penicillium (e.g., P. adametzii, P. amagasakiense, P. chrysogenum, P. decumbens, P. expansum, P. polonicum, P. viridicatum), or Talaromyces (e.g., T. bacillisporus, T. favus, T. stipitatus, T. variabilis) glucose oxidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 6-8;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 6-8;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 97-99 or the cDNA sequence thereof;
d) a polypeptide derived from any one of SEQ ID NO(s): 6-8 by substitution, deletion, or insertion of one or more amino acids;
e) a polypeptide derived from a mature polypeptide of any one of SEQ ID NO(s): 6-8 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has cellobiose oxidase activity belonging to EC 1.1.99.18. Examples of proteins that exhibit cellobiose oxidase activity and have an amino acid sequence that is at least 70% identical to one or more of SEQ ID NOs: 6-8 are set forth herein as SEQ ID NOs: 2206-2217.

In some such embodiments, the protein is derived from Chaetomium, Humicola, Microdochium, Myceliophthora, Myriococcum, Neurospora or Remersonia. For example, in some embodiments, the protein is a native Chaetomium, Humicola, Microdochium, Myceliophthora, Myriococcum, Neurospora or Remersoma cellobiose oxidase or is a functional fragment/mutant/variant of a native Chaetomium, Humicola, Microdochium, Myceliophthora, Myriococcum, Neurospora or Remersonia cellobiose oxidase.

Those skilled in the art will understand how to identify, isolate, characterize, produce, recover and formulate proteins having one or more oxidoreductase activities belonging to EC 1.1. See, e.g., CN101348794-A; CN101348795-A; CN102517304-A; CN103275942-A; CN103525778-A; CN103614350-A; CN103981159-A; CN104312989-A; CN104711273-A; CN104711274-A; CN105002147-A; CN105420252-A; CN105746635-A; CN105950577-A; CN105950578-A; CN106119219-A; CN107012130-A; CN107988177-A; CN108003244-A; CN108004256-A; CN108118036-A; CN108118037-A; CN108118038-A; CN108251389-A; CN108251390-A; CN108251391-A; CN108251392-A; CN108374001-A; CN108893453-A; CN109207446-A; CN109321586-A; CN109423483-A; CN109666657-A; CN110577939-A; CN110592034-A; CN110628738-A; CN110885801-A; CN111004786-A; CN112143717-A; CN112760299-A; CN112877306-A; CN113061189-A; CN113403290-A; CN113528476-A; CN113862233-A; CN114058637-A; CN114181916-A; CN114395540-A; CN114395541-A; CN114736879-A; CN114736880-A; CN114736881-A; CN115029327-A; CN115029328-A; CN115612628-A; CN1229139-A; EP1892529-A1; EP2415863-A1; EP2562250-A1; EP2796547-A1; EP3572503-A1; EP3984368-A1; FR2979918-A1; JP2011139677-A; JP2012157315-A; JP2015002686-A; JP2018198581-A; U.S. Ser. No. 10/233,430-B1; US2004053425-A1; US2022283151-A1; WO2010039840-A1; WO2010053161-A1; WO2010121933-A1; WO2010135499-A1; WO2011068050-A1; WO2012068236-A2; WO2013026575-A2; WO2013159005-A2; WO2013181760-A; WO2014000746-A1; WO2014013073-A1; WO2014081700-A1; WO2014114810-A1; WO2014173822-A2; WO2015109405-A1; WO2016026842-A1; WO2016031611-A1; WO2016090472-A1; WO2016090473-A1; WO2016050905-A1; WO2018039802-A1; WO2019110497-A1; WO2020125700-A1; WO2020239064-A1; WO2020254336-A1; WO2022138668-A1; WO2022256274-A1; WO2023004432-A2; WO2023288294-A1; WO8912675-A; WO9521924-A1.

In some embodiments, proteins of the present disclosure exhibit one or more oxidoreductase activities belonging to EC 1.4, such as oxidase activities belonging to EC 1.4.3 (e.g., D-aspartate oxidase activity belonging to EC 1.4.3.1, L-amino acid oxidase activity belonging to EC 1.4.3.2, D-amino acid oxidase activity belonging to EC 1.4.3.3, D-glutamate oxidase activity belonging to EC 1.4.3.7, L-glutamate oxidase activity belonging to EC 1.4.3.11, cyclohexylamine oxidase activity belonging to EC 1.4.3.12, protein-lysine 6-oxidase activity belonging to EC 1.4.3.13, L-lysine oxidase activity belonging to EC 1.4.3.14, D-glutamate(D-aspartate) oxidase activity belonging to EC 1.4.3.15, L-aspartate oxidase activity belonging to EC 1.4.3.16, glycine oxidase activity belonging to EC 1.4.3.19, L-lysine 6-oxidase activity belonging to EC 1.4.3.20, L-arginine oxidase activity belonging to EC 1.4.3.25).

In some embodiments, proteins of the present disclosure exhibit one or more oxidase activities belonging to EC 1.10, such as activities belonging to EC 1.10.3 (e.g., laccase activity belonging to EC 1.10.3.2), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to the amino acid sequence set forth herein as SEQ ID NO: 9.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 9;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 9;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 100 or the cDNA sequence thereof,
d) a polypeptide derived from any one of SEQ ID NO(s): 9 by substitution, deletion, or insertion of one or more amino acids;
e) a polypeptide derived from a mature polypeptide of any one of SEQ ID NO(s): 9 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has laccase activity belonging to EC 1.10.3.2. Examples of proteins that exhibit laccase activity and have an amino acid sequence that is at least 70% identical to SEQ ID NO: 9 are set forth herein as SEQ ID NOs: 2218-2251.

In some such embodiments, the protein is derived from Chaetomium, Chrysocorona, Melanocarpus, Myceliophthoora, Myriococcum, or Thermothelomyces. For example, in some embodiments, the protein is a native Chaetomium, Chrysocorona, Melanocarpus, Myceliophthoora, Myriococcum, or Thermothelomyces laccase or is a functional fragment/mutant/variant of a native Chaetomium, Chrysocorona, Melanocarpus, Myceliophthoora, Myriococcum, or Thermothelomyces laccase.

Those skilled in the art will understand how to identify, isolate, characterize, produce, recover and formulate proteins having one or more oxidase activities belonging to EC 1.10. See, e.g., EP3444323-A1; IN9601521-14; US2008148432-A1; US2009155415-A1; US2012227131-A1; US2022298533-A1; U.S. Pat. No. 6,060,442-A; WO2009075860-A2; WO2012068236-A2; WO2013181760-A1; WO2014081700-A1; WO2015109405-A1; WO2016029107-A1; WO2016090059-A1; WO2016090474-A1; WO2017089304-A1; WO2022029293-A1; WO2022270590-A1; WO2023288294-A1; WO9838286-A1.

In some embodiments, enzymes of the present disclosure exhibit peroxidase activity belonging to EC 1.11, such as peroxidase activity belonging to EC 1.11.1 (e.g., catalase activity belonging to EC 1.11.1.6, peroxidase activity belonging to EC 1.11.1.7, lignin peroxidase activity belonging to EC 1.11.1.14), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 10-13.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 10-12;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 10-12;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 101-103 or the cDNA sequence thereof;
d) a polypeptide derived from any one of SEQ ID NO(s): 10-12 by substitution, deletion, or insertion of one or more amino acids;
e) a polypeptide derived from a mature polypeptide of any one of SEQ ID NO(s): 10-12 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has catalase activity belonging to EC 1.11.1.6. Examples of proteins that exhibit catalase activity and have an amino acid sequence that is at least 70% identical to one or more of SEQ ID NOs: 10-12 are set forth herein as SEQ ID NOs: 2252-2296.

In some such embodiments, the protein is derived from Aspergillus, Myceliophthora, Penicillium, Rasamsonia, Talaromyces or Thermoascus. For example, in some embodiments, the protein is a native Aspergillus, Myceliophthora, Penicillium, Rasamsonia, Talaromyces or Thermoascus catalase or is a functional fragment/mutant/variant of a native Aspergillus, Mycehophthora, Penicillium, Rasamsonia, Talaromyces or Thermoascus catalase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 13;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 13;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 104 or the cDNA sequence thereof,
d) a polypeptide derived from any one of SEQ ID NO(s): 13 by substitution, deletion, or insertion of one or more amino acids;
e) a polypeptide derived from a mature polypeptide of any one of SEQ ID NO(s): 13 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has peroxidase activity belonging to EC 1.11.1.7. Examples of proteins that exhibit peroxidase activity and have an amino acid sequence that is at least 70% identical to SEQ ID NO: 13 are set forth herein as SEQ ID NOs: 2297-2381.

In some such embodiments, the protein is derived from Arthromyces or Coprinopsis. For example, in some embodiments, the protein is a native A Arthromyces or Coprinopsis peroxidase or is a functional fragment/mutant/variant of a native Arthromyces or Coprinopsis peroxidase.

Those skilled in the art will understand how to identify, isolate, characterize, produce, recover and formulate proteins having one or more peroxidase activities belonging to EC 1.11. See, e.g., CN105441400-A; CN108070574-A; CN112522227-A; EP2295582-A2; EP486067-A2; JP2007143405-A; JP2010044058-A; KR2011013726-A; US2005108791-A1; US2007118916-A1; US2008148432-A1; US2013074202-A1; US2020306342-A1; WO2004108765-A2; WO2006114616-A1; WO2007020428-A1; WO2007044043-A2; WO2009104622-A1; WO2010027755-A1; WO2011068297-A1; WO2012068236-A2; WO2012072777-A1; WO2012130120-A1; WO2013091547-A1; WO2014018368-A2; WO2014081700-A1; WO2014202616-A2; WO2015048332-A2; WO2015182941-A1; WO2017040907-A1; WO2018089391-A1; WO2020200321-A1; WO2020200322-A1; WO2022074170-A1; WO2022251056-A1; WO2023002065-A2; WO2023019266-A2; WO9317721-A1; WO9318166-A2; WO9510602-A1; WO9515391-A2; WO9810060-A1; WO9835026-A1.

In some embodiments, proteins of the present disclosure exhibit one or more oxygenase activities belonging to EC 1.14, such as oxygenase activities belonging to EC 1.14.16 (e.g., phenylalanine 4-monooxygenase activity belonging to EC 1.14.16.1, tyrosine 3-monooxygenase activity belonging to EC 1.14.16.2, tryptophan 5-monooxygenase activity belonging to EC 1.14.16.4, phenylalanine 3-monooxygenase activity belonging to EC 1.14.16.7), EC 1.14.18 (e.g., tyrosinase activity belonging to EC 1.14.18.1) and/or EC 1.14.99 (e.g., lytic chitin monooxygenase activity belonging to EC 1.14.99.53, lytic cellulose monooxygenase activity belonging to EC 1.14.99.54, lytic starch monooxygenase activity belonging to EC 1.14.99.55, lytic cellulose monooxygenase activity belonging to EC 1.14.99.56, lytic cellulose monooxygenase activity belonging to EC 1.14.99.56), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 14-15.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 14-15;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 14-15;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 105-106 or the cDNA sequence thereof,
d) a polypeptide derived from any one of SEQ ID NO(s): 14-15 by substitution, deletion, or insertion of one or more amino acids;
e) a polypeptide derived from a mature polypeptide of any one of SEQ ID NO(s): 14-15 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has lytic cellulose monooxygenase activity belonging to EC 1.14.99.56. Examples of proteins that exhibit lytic cellulose monooxygenase activity and have an amino acid sequence that is at least 70% identical to one or more of SEQ ID NOs: 14-15 are set forth herein as SEQ ID NOs: 2382-2755.

In some such embodiments, the protein is derived from Penicillium, Rasamsonia, or Thermoascus. For example, in some embodiments, the protein is a native Penicillium, Rasamsonia, or Thermoascus monooxygenase or is a functional fragment/mutant/variant of a native Penicillium, Rasamsonia, or Thermoascus monooxygenase.

Those skilled in the art will understand how to identify, isolate, characterize, produce, recover and formulate proteins having one or more oxygenase activities belonging to EC 1.14. See, e.g., CN103232949-A; CN103255072-A; CN106544329-A; CN109554355-A; CN110093326-A; JP2016039821-A; US2008299613-A1; US2011099671-A1; US2014075603-A1; US2018019412-A1; US2019153414-A1; WO2011080267-A2; WO2011121768-A1; WO2011153516-A2; WO2012000892-A1; WO2012021394-A1; WO2012021395-A1; WO2012044835-A1; WO2012044836-A1; WO2012068509-A1; WO2012089023-A1; WO2013028915-A2; WO2013036898-A2; WO2013110242-A1; WO2013119302-A2; WO2014085251-A1; WO2014093835-A1; WO2014138983-A1; WO2014140165-A1; WO2014202616-A2; WO2014202711-A1; WO2015105835-A1; WO2015187935-A1; WO2016045569-A1; WO2016090473-A1; WO2016145358-A1; WO2017070219-A1; WO2018019948-A1; WO2018106656-A1; WO2018142002-A1; WO2019005755-A1; WO2019083831-A1; WO2019229228-A1.

In some embodiments, proteins of the present disclosure exhibit one or more catalytic activities belonging to Enzyme Commission classification number 2 (EC 2). For example, in some embodiments, proteins of the present disclosure exhibit aminoacyltransferase activity useful for a) preventing/treating/suppressing/eliminating/reducing the detrimental effects of infestations/infections of/by various pests, including, but not limited to, phytopathogenic pests, such as acarids, bacteria, fungi, gastropods, insects, nematodes, oomycetes, protozoa, viruses and weeds; b) reducing one or more aspects of disease severity in plants affected by one or more phytopathogenic pests; c) pretreating surfaces/substances that are susceptible to infestation/infection by pests; d) cleaning surfaces/substances that are infested/infected by pests; e) enhancing the environments in which plants are grown by; f) improving nutrient availability in plant growth media; g) reducing the amounts of exogenous fertilizer needed to achieve a desired result; h) improving plant growth, development, and yield characteristics; i) prolonging the shelf-life of harvested plants and plant parts; j) delaying/hastening the ripening of a plant or plant part; k) improving the efficacy of chemical pesticides; and/or l) reducing chemical-pesticide-induced resistance/phytotoxicity.

In some embodiments, proteins of the present disclosure exhibit transferase activity belonging to EC 2 and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 16 and 2756-2769.

In some embodiments, proteins of the present disclosure exhibit one or more acyltransferase activities belonging to EC 2.3, such as aminoacyl transferase activities belonging to EC 2.3.2 (e.g., D-glutamyl transferase activity belonging to EC 2.3.2.1, gamma-glutamyl transferase activity belong to EC 2.3.2.2, aspartyl transferase activity belonging to EC 2.3.2.7, protein-glutamine gamma-glutamyl transferase activity belonging to EC 2.3.2.13, D-alanine gamma-glutamyl transferase activity belonging to EC 2.3.2.14), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequence set forth herein as SEQ ID NO: 16.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 16;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 16;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 107 or the cDNA sequence thereof;
d) a polypeptide derived from any one of SEQ ID NO(s): 16 by substitution, deletion, or insertion of one or more amino acids;
e) a polypeptide derived from a mature polypeptide of any one of SEQ ID NO(s): 16 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has gamma-glutamyl transferase activity belonging to EC 2.3.2.2. Examples of proteins that exhibit gamma-glutamyl transferase activity and have an amino acid sequence that is at least 70% identical to SEQ ID NO: 16 are set forth herein as SEQ ID NOs: 2756-2769.

In some such embodiments, the protein is derived from Bacillus. For example, in some embodiments, the protein is a native Bacillus gamma-glutamyl transferase or is a fragment/mutant/variant of a native Bacillus gamma-glutamyl transferase.

Those skilled in the art will understand how to identify, isolate, characterize, produce, recover and formulate proteins having one or more acyltransferase activities belonging to EC 2.3. See, e.g., CN107828754-A; CN108611333-A; CN108929866-A; CN112111468-A; CN114806988-A; EP441353-A; KR1814024-B1; WO2003087149-A2; WO2005075652-A1; WO2014081884-A1; WO2015048332-A2; WO2018234382-A1.

In some embodiments, proteins of the present disclosure exhibit one or more catalytic activities belonging to Enzyme Commission classification number 3 (EC 3). For example, in some embodiments, proteins of the present disclosure exhibit lipase, triacylglycerol lipase, pectinesterase, phospholipase, lysophospholipase, cutinase, amylase, glucosidase, galactosidase, cellulase, glucanase, xylanase, ceramidase, dextranase, chitinase, chitosanase, galacturonase, fucosidase, lysozymes, xylosidase, lucosidass, pullulanase, mannosidase, amidase, aminidase, maltohydrolases, cellobiosidase, pectinase, mannanase, aminopeptidase, serine peptidase and/or metallopeptidase, asparaginase and/or glutaminase activity useful for a) prevent-ing/treating/suppressing/eliminat-ing/reducing the detrimental effects of infestations/infections of/by various pests, including, but not limited to, phytopathogenic posts, such as acarids, bacteria, fungi, gastropods, insects, nematodes, oomycetes, protozoa, viruses and weeds; b) reducing one or more aspects of disease severity in plants affected by one or more phytopathogenic pests; c) pretreating surfaces/substances that are susceptible to infestation/infection by pests; d) cleaning surfaces/substances that are infested/infected by pests; e) enhancing the environments in which plants are grown by; f) improving nutrient availability in plant growth media; g) reducing the amounts of exogenous fertilizer needed to achieve a desired result; h) improving plant growth, development, and yield characteristics; i) prolonging the shelf-life of harvested plants and plant parts; j) delaying/hastening the ripening of a plant or plant part; k) improving the efficacy of chemical pesticides; and/or l) reducing chemical-pesticide-induced resistance/phytotoxicity.

In some embodiments, proteins of the present disclosure exhibit esterase activity belonging to EC 3 and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 17-90 and 2770-72141.

In some embodiments, proteins of the present disclosure exhibit one or more esterase activities belonging to EC 3.1, such as lipase activities belonging to EC 3.1.1 (e.g., triacylglycerol lipase activity belong to EC 3.1.1.3, phospholipase A₂ activity belonging to EC 3.1.1.4, lysophospholipase activity belonging to 3.1.1.5, pectinesterase activity belonging to 3.1.1.11, phospholipase A₁ activity belonging to 3.1.1.32, lipoprotein lipase activity belonging to EC 3.1.1.34, cutinase activity belonging to 3.1.1.74), phosphatase activities belonging to EC 3.1.3 (e.g., alkaline phosphatase activity belonging to EC 3.1.3.1, acid phosphatase activity belonging to EC 3.1.3.2, 3-phytase activity belonging to EC 3.1.3.8, glucose-6-phosphatase activity belonging to EC 3.1.3.9, glucose-1-phosphatase activity belonging to EC 3.1.3.10, fructose-biphosphatase activity belonging to EC 3.1.3.11, sugar-phosphatase activity belonging to EC 3.1.3.23, 4-phytase activity belonging to EC 3.1.3.26, fructose-2,6-biphosphate 2-phosphatase activity belonging to EC3.1.3.46, fructose-2,6-biphosphate 6-phosphatase activity belonging to EC 3.1.3.54, 5-phytase activity belonging to EC 3.1.3.72, lipid-phosphate phosphatase activity belonging to EC 3.1.3.76), and/or hydrolase activities belonging to EC 3.1.4 (e.g., phospholipase C activity belonging to 3.1.4.3, phospholipase D activity belonging to EC 3.1.4.4, phoshoinositide phospholipase C activity belonging to 3.1.4.11), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%/identical to one or more of the amino acid sequences set forth herein as SEQ ID NO(s): 17-33, 72147 and 72149.

In some embodiments, the protein is selected from the group consisting of
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 17-22;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 17-22;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 108-113 or the cDNA sequence thereof;
d) a polypeptide derived from any one of SEQ ID NO(s): 17-22 by substitution, deletion, or insertion of one or more amino acids;
e) a polypeptide derived from a mature polypeptide of any one of SEQ ID NO(s): 17-22 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has triacylglycerol lipase activity belonging to EC 3.1.1.3. Examples of proteins that exhibit triacylglycerol lipase activity and have an amino acid sequence that is at least 70% identical to one or more of SEQ ID NOs: 17-22 are set forth herein as SEQ ID NOs: 2778-10223.

In some embodiments, the protein is derived from Aspergillus, Bacillus, Cryphonectria, Fusarium, Haloquadratum, Humicola, Penicillium, Scytalidium, Talaromyces, Thermomyces or Thermus. For example, in some embodiments, the protein is a native Aspergillus, Bacillus, Cryphonectria, Fusarium, Haloquadratum, Humicola, Penicillium, Scytalidium, Talaromyces, Thermomyces or Thermus triacylglycerol lipase or is a fragment/mutant/variant of a native Aspergillus, Bacillus, Cryphonectria, Fusarium, Haloquadratum, Humicola, Penicillium, Scytalidium, Talaromyces, Thermomyces or Thermus triacylglycerol lipase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 23;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 23;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 114 or the cDNA sequence thereof,
d) a polypeptide derived from any one of SEQ ID NO(s): 23 by substitution, deletion, or insertion of one or more amino acids;
e) a polypeptide derived from a mature polypeptide of any one of SEQ ID NO(s): 23 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has triacylglycerol lipase activity belonging to EC 3.1.1.3 and/or phospholipase A₁ activity belonging to EC 3.1.1.32. Examples of proteins that exhibit triacylglycerol lipase activity and/or phospholipase A₁ activity and have an amino acid sequence that is at least 70% identical to SEQ ID NO: 23 are set forth herein as SEQ ID NOs: 10243-15978.

In some embodiments, the protein is derived from Aspergillus, Bacillus, Fusarium, Struthio, Talaromyces or Thermomyces. For example, in some embodiments, the protein is a native Aspergillus, Bacillus, Fusarium, Struthio, Talaromyces or Thermomyces triacylglycerol lipase or is a fragment/mutant/variant of a native Aspergillus, Bacillus, Fusarium, Struthio, Talaromyces or Thermomyces triacylglycerol lipase.

In some embodiments, the protein is derived from Aspergillus, Bacillus, Fusarium, Struthio, Talaromyces or Thermomyces. For example, in some embodiments, the protein is a native Aspergillus, Bacillus, Fusarium, Struthio, Talaromyces or Thermomyces phospholipase A₁ or is a fragment/mutant/variant of a native Aspergillus, Bacillus, Fusarium, Struthio, Talaromyces or Thermomyces phospholipase A₁.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 24;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 24;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 115 or the cDNA sequence thereof,
d) a polypeptide derived from any one of SEQ ID NO(s): 24 by substitution, deletion, or insertion of one or more amino acids;
e) a polypeptide derived from a mature polypeptide of any one of SEQ ID NO(s): 24 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has lysophospholipase activity belonging to EC 3.1.1.5. Examples of proteins that exhibit lysophospholipase activity and have an amino acid sequence that is at least 70% identical to SEQ ID NO: 24 are set forth herein as SEQ ID NOs: 15979-15981.

In some embodiments, the protein is derived from Aspergillus. For example, in some embodiments, the protein is a native Aspergillus lysophospholipase or is a fragment/mutant/variant of a native Aspergillus lysophospholipase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 25;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 25;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 116 or the cDNA sequence thereof,
d) a polypeptide derived from any one of SEQ ID NO(s): 25 by substitution, deletion, or insertion of one or more amino acids;
e) a polypeptide derived from a mature polypeptide of any one of SEQ ID NO(s): 25 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has pectinesterase activity belonging to EC 3.1.1.11. Examples of proteins that exhibit pectinesterase activity and have an amino acid sequence that is at least 70% identical to SEQ ID NO: 25 are set forth herein as SEQ ID NOs: 2770-2777.

In some embodiments, the protein is derived from Aspergillus. For example, in some embodiments, the protein is a native Aspergillus pectinesterase or is a fragment/mutant/variant of a native Aspergillus pectinesterase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 26;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 26;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 117 or the cDNA sequence thereof,
d) a polypeptide derived from any one of SEQ ID NO(s): 26 by substitution, deletion, or insertion of one or more amino acids;
e) a polypeptide derived from a mature polypeptide of any one of SEQ ID NO(s): 26 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has phospholipase A₁ activity belonging to EC 3.1.1.32. Examples of proteins that exhibit phospholipase A₁ activity and have an amino acid sequence that is at least 70% identical to SEQ ID NO: 26 are set forth herein as SEQ ID NOs: 10224-10242.

In some embodiments, the protein is derived from Evansstolkia. For example, in some embodiments, the protein is a native Evansstolkia phospholipase A₁ or is a fragment/mutant/variant of a native Evansstolkia phospholipase A₁.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 27-31, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 27-31, 72147 and 72149;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 118-122, 72148 and 72150 or the cDNA sequence thereof;
d) a polypeptide derived from any one of SEQ ID NO(s): 27-31, 72147 and 72149 by substitution, deletion, or insertion of one or more amino acids;
e) a polypeptide derived from a mature polypeptide of any one of SEQ ID NO(s): 27-31, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has cutinase activity belonging to EC 3.1.1.74. Examples of proteins that exhibit cutinase activity and have an amino acid sequence that is at least 70% identical to one or more of SEQ ID NOs: 27-31, 72147 and 72149 are set forth herein as SEQ ID NOs: 15982-16004.

In some embodiments, the protein is derived from Acrophialophora, Ascomycota, Chaetomium, Humicola, Hypocrea, Myceliophthora, Myriococcum, Pyrenophora, Thermochaetoides or Trichoderma. For example, in some embodiments, the protein is a native Acrophialophora, Ascomycota, Chaetomium, Humicola, Hypocrea, Myceliophthora, Myriococcum, Pyrenophora, Thermochaetoides or Trichoderma cutinase or is a fragment/mutant/variant of a native Acrophialophora, Ascomycota, Chaetomium, Humicola, Hypocrea, Myceliophthora, Myriococcum, Pyrenophora, Thermochaetoides or Trichoderma cutinase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 32;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 32;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 123 or the cDNA sequence thereof;
d) a polypeptide derived from any one of SEQ ID NO(s): 32 by substitution, deletion, or insertion of one or more amino acids;
e) a polypeptide derived from a mature polypeptide of any one of SEQ ID NO(s): 32 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has phospholipase C activity belonging to EC 3.1.4.3. Examples of proteins that exhibit phospholipase C activity and have an amino acid sequence that is at least 70% identical to SEQ ID NO: 32 are set forth herein as SEQ ID NOs: 16396-16412.

In some embodiments, the protein is derived from Pseudomonas. For example, in some embodiments, the protein is a native Pseudomonas phospholipase C or is a fragment/mutant/variant of a native Pseudomonas phospholipase C.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 33;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 33;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 124 or the cDNA sequence thereof,
d) a polypeptide derived from any one of SEQ ID NO(s): 33 by substitution, deletion, or insertion of one or more amino acids;
e) a polypeptide derived from a mature polypeptide of any one of SEQ ID NO(s): 33 by substitution, deletion, or insertion of one or more amino acids;
f) a polypeptide derived from the polypeptide of any one of a) through c) wherein the N- and/or C-terminal end has been extended by the addition of one or more amino acids; and
g) a fragment of the polypeptide of any one of a) through f)
   wherein the polypeptide has phosphoinositide phospholipase C activity belonging to EC 3.1.4.11. Examples of proteins that exhibit phosphoinositide phospholipase C activity and have an amino acid sequence that is at least 70% identical to one or more of SEQ ID NOs: 33 are set forth herein as SEQ ID NOs: 16005-16395.

In some embodiments, the protein is derived from Bacillus, Komagataella or Lysinibacillus. For example, in some embodiments, the protein is a native Bacillus, Komagataella or Lysinibacillus phosphoinositide phospholipase C or is a fragment/mutant/variant of a native Bacillus, Komagataella or Lysinibacillus phosphoinositide phospholipase C.

Those skilled in the art will understand how to identify, isolate, characterize, produce, recover and formulate proteins having one or more esterase activities belong to EC 3.1. See, e.g., CA2715086-A1; CN102604913-A; CN103045559-A; CN103243038-A; CN103555600-A; CN103865896-A; CN103981160-A; CN104293744-A; CN104878033-A; CN105087614-A; CN106591258-A; CN106632683-A; CN106635846-A; CN106676084-A; CN106884030-A; CN107151660-A; CN107488644-A; CN107488645-A; CN107488646-A; CN107488647-A; CN107488648-A; CN107815460-A; CN108118039-A; CN108239626-A; CN108239627-A; CN108277212-A; CN108315312-A; CN108359655-A; CN108913675-A; CN109321546-A; CN109468301-A; CN109776686-A; CN109929821-A; CN110129301-A; CN110760531-A; CN111378583-A; CN112175976-A; CN112574974-A; CN112592907-A; CN112608912-A; CN113025595-A; CN113025596-A; CN113122520-A; CN113215130-A; CN113338044-A; CN113736817-A; CN113755509-A; CN113846074-A; CN114317490-A; CN114621941-A; CN115521925-A; CN115717134-A; DE102016204813-A1; EP2145904-A1; EP2623586-; 2; EP305216-A; EP3091080-A1; EP3101108-A1; EP3101109-A1; EP3284811-A1; EP3287513-A1; EP3301145-A1; EP3301147-A1; EP3301156-A1; EP3301161-A1; EP3301162-A1; EP3301163-A1; EP3301165-A1; EP3301166-A1; EP3301169-A1; JP09249891-A; JP09249893-A; JP2019165727-A; US2005287250-A1; US2007173430-A1; US2009162480-A1; US2009217463-A1; US2009217464-A1; US2009221033-A1; US2009221034-A1; US2011312057-A1; US2013316458-A; US2018094228-A; US2019093054-A; US2020306342-A1; US2021015105-A1; US2022177812-A1; U.S. Pat. No. 9,102,933-B1; WO2000060063-A1; WO200011211-A1; WO200034450-A1; WO200127251-A1; WO200192502-A1; WO200255679-A2; WO200262973-A2; WO200266622-A2; WO200295127-A2; WO2003060112-A1; WO2003089620-A2; WO2004099400-A2; WO2004111216-A2; WO2005032496-A2; WO2005086900-A2; WO2006084470-A2; WO2006136159-A2; WO2006136160-A2; WO2007080197-A2; WO2007087243-A2; WO2007087318-A2; WO2007087319-A2; WO2007087508-A2; WO2008073169-A2; WO2008079685-A2; WO2008122640-A2; WO2009071550-A1; WO2009083607-A1; WO2009108941-A2; WO2009133177-A1; WO2011046812-A1; WO2011046815-A1; WO2012027282-A2; WO2012078741-A2; WO2012129548-A2; WO2012173658-A1; WO2013098205-A2; WO2013113622-A1; WO2013149858-A1; WO2013171072-A1; WO2013181760-A1; WO2014055778-A2; WO2014059360-A1; WO2014059541-A1; WO2014081700-A1; WO2014081884-A1; WO2014147219-A1; WO2014162001-A1; WO2014184164-A1; WO2014186464-A1; WO2015010009-A2; WO2015017045-A1; WO2015048332-A2; WO2015067161-A1; WO2015085920-A1; WO2015109405-A1; WO2015110058-A1; WO2015110562-A1; WO2015140275-A1; WO2015144780-A2; WO2015173426-A1; WO2016050661-A1; WO2016087401-A1; WO2016090472-A1; WO2016090473-A1; WO2016090474-A1; WO2016091870-A1; WO2016102356-A1; WO2016107567-A1; WO2016109758-A2; WO2016164596-A2; WO2017001673-A1; WO2017005640-A1; WO2017015233-A1; WO2017093318-A1; WO2017101801-A1; WO2017161091-A1; WO2017182666-A1; WO2018001959-A1; WO2018015295-A1; WO2018127486-A1; WO2018171552-A1; WO2018188667-A1; WO2019014118-A1; WO2019060574-A1; WO2019063499-A1; WO2019110462-A1; WO2019137289-A1; WO2019154951-A1; WO2019154952-A1; WO2019154954-A1; WO2019154955-A1; WO2019215078-A1; WO2019236717-A1; WO2020014407-A1; WO2020046613-A1; WO2020076697-A1; WO2020088393-A1; WO2020103861-A1; WO2020135657-A1; WO2020135658-A1; WO2020173817-A1; WO2020190782-A1; WO2021037878-A1; WO2021119304-A1; WO2021170799-A1; WO2021239267-A1; WO2022063699-A1; WO2022090361-A2; WO2022103725-A1; WO2022173694-A1; WO2023032952-A1; WO2023288294-A1; WO9205249-A; WO9219726-A1; WO9425575-A1; WO9425577-A1; WO9522615-A1; WO9613580-A1; WO9704079-A1; WO9707202-A1; WO9707205-A1; WO9707206-A1.

In some embodiments, proteins of the present disclosure exhibit one or more glycosylase activities belonging to EC 3.2, such as glycosidase activities belonging to EC 3.2.1 (e.g., alpha-amylase activity belong to EC 3.2.1.1, beta-amylase activity belong to EC 3.2.1.2, glucan 1,4-alpha-glucosidase activity belong to 3.2.1.3, cellulase activity belong to 3.2.1.4, endo-1,3(4)-beta-glucanase activity belong to 3.2.1.6, inulinase activity belong to 3.2.1.7, endo-1,4-beta-xylanase activity belong to 3.2.1.8, oligo-1,6-glucosidase activity belong to 3.2.1.10, dextranase activity belong to 3.2.1.11, chitinase activity belong to 3.2.1.14, endo-polygalacturonase (pectinase) activity belong to 3.2.1.15, lysozyme activity belong to 3.2.1.17, alpha-glucosidase activity belong to 3.2.1.20, beta-glucosidase activity belong to 3.2.1.21, alpha-galactosidase activity belong to 3.2.1.22, beta-galactosidase activity belong to 3.2.1.23, alpha-mannosidase activity belong to 3.2.1.24, beta-mannosidase activity belong to 3.2.1.25, beta-fructofuranosidase activity belong to 3.2.1.26, alpha,alpha-trehalase activity belong to 3.2.1.28, endo-1,3-beta-xylanase activity belong to 3.2.1.32, amylo-1,6-glucosidase activity belonging to EC3.2.1.33, xylan 1,4-beta-xylosidase activity belong to 3.2.1.37, glucan endo-1,3-beta-D-glucosidase activity belong to 3.2.1.39, pullulanase activity belong to 3.2.1.41, alpha-L-arabinofuranosidase activity belong to 3.2.1.55, glucan 1,3-beta-glucosidase activity belong to 3.2.1.58, glucan endo-1,3-alpha-glucosidase activity belong to 3.2.1.59, glucan 1,6-alpha-glucosidase activity belonging to EC 3.2.1.70, glucan endo-1,2-beta-glucosidase activity belonging to EC 3.2.1.71, xylan 1,3-beta-xylosidase activity belonging to EC 3.2.1.72, licheninase activity belong to 3.2.1.73, glucan 1,4-beta-glucosidase activity belonging to EC 3.2.1.74, glucan endo-1,6-beta-glucosidase activity belong to 3.2.1.75, mannan 1,2-(1,3)-alpha-mannosidase activity belonging to EC 3.2.1.77, mannan endo-1,4-O-mannosidase activity belonging to 3.2.1.78, glucan 1,3-alpha-glucosidase activity belonging to EC 3.2.1.84, cellulose 1,4-beta-cellobiosidase activity belong to 3.2.1.91, peptidoglycan beta-N-acetylmuramidase activity belonging to EC 3.2.1.92, endo-alpha-N-acetylgalactosaminidase activity belong to 3.2.1.97, mannan 1,4-mannobiosidase activity belonging to EC 3.2.1.100, mannan endo-1,6-alpha-mannosidase activity belong to 3.2.1.101, endogalactosaminidase activity belong to 3.2.1.109, 1,3-alpha-L-fucosidase activity belong to 3.2.1.111, 2-deoxyglucosidase activity belong to 3.2.1.112, glycoprotein endo-alpha-1,2-mannosidase activity belonging to EC 3.2.1.130, chitosanase activity belong to 3.2.1.132, glucan 1,4-alpha-maltohydrolase activity belong to 3.2.1.133, mannan exo-1,2-1,6-alpha-mannosidase activity belonging to EC 3.2.1.137, 1,6-alpha-D-mannosidase activity belonging to 3.2.1.163, 1,4-beta-cellobiosidase activity belonging to 3.2.1.176, galactan endo-beta-1,3-galactanase activity belonging to EC 3.2.1.181, alpha-mannan endo-1,2-alpha-mannanase activity belonging to EC 3.2.1.198, exo-chitinase activity belonging to EC 3.2.1.200, exo-chitinase activity belonging to EC 3.2.1.201), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 34-80, 45906 and 72144.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 34-40;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 34-40;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 125-131 or the cDNA sequence thereof,
d) a polypeptide derived from any one of SEQ ID NO(s): 34-40 by substitution, deletion, or insertion of one or more amino acids;
e) a polypeptide derived from a mature polypeptide of any one of SEQ ID NO(s): 34-40 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has alpha-amylase activity belonging to EC 3.2.1.1. Examples of proteins that exhibit alpha-amylase activity and have an amino acid sequence that is at least 70% identical to one or more of SEQ ID NOs: 34-40 are set forth herein as SEQ ID NOs: 16413-45041.

In some embodiments, the protein is derived from Alicyclobacillus, Alkalihalobacillus, Anoxybacillus, Aspergillus, Bacillus, Cytophaga, Exiguobacterium, Geobacillus, Hamigera, Homo sapiens, Jeotgalibacillus, Neosartorya, Penicillium, Priestia, Pyrococcus, Rasamsonia, Sutcliffiella or Thermoascus.

For example, in some embodiments, the protein is a native Alicyclobacillus, Alkalihalobacillus, Anoxybacillus, Aspergillus, Bacillus, Cytophaga, Exiguobacterium, Geobacillus, Hamigera, Homo sapiens, Jeotgalibacillus, Neosartorya, Penicillium, Priestia, Pyrococcus, Rasamsonia, Sutcliffiella or Thermoascus alpha-amylase or is a fragment/mutant/variant of a native Alicyclobacillus, Alkalihalobacillus, Anoxybacillus, Aspergillus, Bacillus, Cytophaga, Exiguobacterium, Geobacillus, Hamigera, Homo sapiens, Jeotgalibacillus, Neosartorya, Penicillium, Priestia, Pyrococcus, Rasamsonia, Sutcliffiella or Thermoascus alpha-amylase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 41-42;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 41-42;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 132-133 or the cDNA sequence thereof,
d) a polypeptide derived from any one of SEQ ID NO(s): 41-42 by substitution, deletion, or insertion of one or more amino acids;
e) a polypeptide derived from a mature polypeptide of any one of SEQ ID NO(s): 41-42 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucan 1,4-alpha-glucosidase activity belonging to EC 3.2.1.3. Examples of proteins that exhibit glucan 1,4-alpha-glucosidase activity and have an amino acid sequence that is at least 70% identical to one or more of SEQ ID NOs: 41-42 are set forth herein as SEQ ID NOs: 45756-45904.

In some embodiments, the protein is derived from Aspergillus, Bos, Elaphocordyceps, Fusarium, Penicillium, Rasamsonia or Saccharomycopsis. For example, in some embodiments, the protein is a native Aspergillus, Bos, Elaphocordyceps, Fusarium, Penicillium, Rasamsonia or Saccharomycopsis glucan 1,4-alpha-glucosidase or is a fragment/mutant/variant of a native Aspergillus, Bos, Elaphocordyceps, Fusarium, Penicillium, Rasamsonia or Saccharomycopsis glucan 1,4-alpha-glucosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 43-45;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 43-45;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 134-136 or the cDNA sequence thereof;
d) a polypeptide derived from any one of SEQ ID NO(s): 43-45 by substitution, deletion, or insertion of one or more amino acids;
e) a polypeptide derived from a mature polypeptide of any one of SEQ ID NO(s): 43-45 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has cellulase activity belonging to EC 3.2.1.4. Examples of proteins that exhibit cellulase activity and have an amino acid sequence that is at least 70% identical to one or more of SEQ ID NOs: 43-45 are set forth herein as SEQ ID NOs: 45913-48750.

In some embodiments, the protein is derived from Acremonium, Alteromonas, Ascomycota, Aspergillus, Bacillus, Chaetomium, Clonostachys, Corynascus, Cylindrocarpon, Escherichia, Fusarium, Humicola, Madurella, Melanocarpus, Myceliophthora, Neurospora, Podospora, Remersonia, Scytalidium, Sordaria, Staphylotrichum, Thermocarpiscus, Thermochaetoides, Thermothielavioides, Thielavia, Trichocladium, Trichothecium or Triticum. For example, in some embodiments, the protein is a native Acremonium, Alteromonas, Ascomycota. Aspergillus, Bacillus, Chaetomium, Clonostachys, Corynascus, Cylindrocarpon, Escherichia, Fusarium, Humicola, Madurella, Melanocarpus, Myceliophthora, Neurospora, Podospora, Remersonia, Scytalidium, Sordaria, Staphylotrichum, Thermocarpiscus, Thermochaetoides, Thermothielavioides, Thielavia, Trichocladium, Trichothecium or Triticum cellulase or is a fragment/mutant/variant of a native Acremonium, Alteromonas, Ascomycota, Aspergillus, Bacillus, Chaetomium, Clonostachys, Corynascus, Cylindrocarpon, Escherichia, Fusarium, Humicola, Madurella, Melanocarpus, Myceliophthora, Neurospora, Podospora, Remersonia, Scytalidium, Sordaria, Staphylotrichum, Thermocarpiscus, Thermochaetoides, Thermothielavioides, Thielavia, Trichocladium, Trichothecium or Triticum cellulase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 46;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 46;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 137 or the cDNA sequence thereof,
d) a polypeptide derived from any one of SEQ ID NO(s): 46 by substitution, deletion, or insertion of one or more amino acids;
e) a polypeptide derived from a mature polypeptide of any one of SEQ ID NO(s): 46 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has endo-1,3(4)-beta-glucanase activity belonging to EC 3.2.1.6. Examples of proteins that exhibit endo-1,3(4)-beta-glucanase activity and have an amino acid sequence that is at least 70% identical to one or more of SEQ ID NOs: 46 are set forth herein as SEQ ID NOs: 49229-49346.

In some embodiments, the protein is derived from Bacillus, Bispora, Hordeum or Paenibacillus. For example, in some embodiments, the protein is a native Bacillus, Bispora, Hordeum or Paenibacillus endo-1,3(4)-beta-glucanase or is a fragment/mutant/variant of a native Bacillus, Bispora, Hordeum or Paenibacillus endo-1,3(4)-beta-glucanase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 47;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 47;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 138 or the cDNA sequence thereof;
d) a polypeptide derived from any one of SEQ ID NO(s): 47 by substitution, deletion, or insertion of one or more amino acids;
c) a polypeptide derived from a mature polypeptide of any one of SEQ ID NO(s): 47 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has inulinase activity belonging to EC 3.2.1.7. Examples of proteins that exhibit inulinase activity and have an amino acid sequence that is at least 70% identical to one or more of SEQ ID NOs: 47 are set forth herein as SEQ ID NOs: 49347-49419.

In some embodiments, the protein is derived from Aspergillus, Penicillium, Pseudomonas or Talaromyces. For example, in some embodiments, the protein is a native Aspergillus, Penicillium, Pseudomonas or Talaromyces inulinase or is a fragment/mutant/variant of a native Aspergillus, Penicillium, Pseudomonas or Talaromyces inulinase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 48-53;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 48-53;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 139-144 or the cDNA sequence thereof;
d) a polypeptide derived from any one of SEQ ID NO(s): 48-53 by substitution, deletion, or insertion of one or more amino acids;
e) a polypeptide derived from a mature polypeptide of any one of SEQ ID NO(s): 48-53 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has endo-1,4-beta-xylanase activity belonging to EC 3.2.1.8. Examples of proteins that exhibit endo-1,4-beta-xylanase activity and have an amino acid sequence that is at least 70% identical to one or more of SEQ ID NOs: 48-53 are set forth herein as SEQ ID NOs: 49453-49912.

In some embodiments, the protein is derived from Anaerocellum, Aspergillus, Bacillus, Caldicellulosiruptor, Clostridium, Dicytoglomus, Evansstolkia, Hordeum, Lactobacillus, Malbranchea, Neosartorya, Nicotiana, Paecilomyces, Paenibacillus, Penicillium, Pyrococcus, Rasamsonia, Saccharomyces, Talaromyces, Thermoclostridium, Thermomyces, Thermus or Viridiplantae. For example, in some embodiments, the protein is a native Anaerocellum, Aspergillus, Bacillus, Caldicellulosiruptor, Clostridium, Dicytoglomus, Evansstolkia, Hordeum, Lactobacillus, Malbranchea, Neosartorya, Nicotiana, Paecilomyces, Paenibacillus, Penicillium, Pyrococcus, Rasamsonia, Saccharomyces, Talaromyces, Thermoclostridium, Thermomyces, Thermus or Viridiplantae endo-1,4-beta-xylanase or is a fragment/mutant/variant of a native Anaerocellum, Aspergillus, Bacillus, Caldicellulosiruptor, Clostridium, Dicytoglomus, Evansstolkia, Hordeum, Lactobacillus, Malbranchea, Neosartorya, Nicotiana, Paecilomyces, Paenibacillus, Penicillium, Pyrococcus, Rasamsonia, Saccharomyces, Talaromyces, Thermoclostridium, Thermomyces, Thermus or Viridiplantae endo-1,4-beta-xylanase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 54;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 54;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 145 or the cDNA sequence thereof,
d) a polypeptide derived from any one of SEQ ID NO(s): 54 by substitution, deletion, or insertion of one or more amino acids;
e) a polypeptide derived from a mature polypeptide of any one of SEQ ID NO(s): 54 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has dextranase activity belonging to EC 3.2.1.11. Examples of proteins that exhibit dextranase activity and have an amino acid sequence that is at least 70% identical to one or more of SEQ ID NOs: 54 are set forth herein as SEQ ID NOs: 45063-45071.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 56;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 56;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 147 or the cDNA sequence thereof,
d) a polypeptide derived from any one of SEQ ID NO(s): 56 by substitution, deletion, or insertion of one or more amino acids;
e) a polypeptide derived from a mature polypeptide of any one of SEQ ID NO(s): 56 by substitution, deletion, or insertion of one or more amino acids;
f) a polypeptide derived from the polypeptide of any one of a) through c) wherein the N- and/or C-terminal end has been extended by the addition of one or more amino acids; and
g) a fragment of the polypeptide of any one of a) through f)
   wherein the polypeptide has lysozyme activity belonging to EC 3.2.1.17. Examples of proteins that exhibit lysozyme activity and have an amino acid sequence that is at least 70% identical to one or more of SEQ ID NOs: 56 are set forth herein as SEQ ID NOs: 45443-45456.

In some embodiments, the protein is derived from Clonostachys or Sodiomyces. For example, in some embodiments, the protein is a native Clonostachys or Sodiomyces lysozyme or is a fragment/mutant/variant of a native Clonostachys or Sodiomyces lysozyme.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 57;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 57;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 148 or the cDNA sequence thereof,
d) a polypeptide derived from any one of SEQ ID NO(s): 57 by substitution, deletion, or insertion of one or more amino acids;
e) a polypeptide derived from a mature polypeptide of any one of SEQ ID NO(s): 57 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has beta-glucosidase activity belonging to EC 3.2.1.21. Examples of proteins that exhibit beta-glucosidase activity and have an amino acid sequence that is at least 70% identical to one or more of SEQ ID NOs: 57 are set forth herein as SEQ ID NOs: 45457-45754.

In some embodiments, the protein is derived from Aspergillus, Coccidioides, Evansstolkia, Neosartorya, Penicillium, Rasamsonia, Schizosaccharomyces or Thermoascus. For example, in some embodiments, the protein is a native Aspergillus, Coccidioides, Evansstolkia, Neosartorya, Penicillium, Rasamsonia, Schizosaccharomyces or Thermoascus beta-glucosidase or is a fragment/mutant/variant of a native Aspergillus, Coccidioides, Evansstolkia, Neosartorya, Penicillium, Rasamsonia, Schizosaccharomyces or Thermoascus beta-glucosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 58;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 58;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 149 or the cDNA sequence thereof;
d) a polypeptide derived from any one of SEQ ID NO(s): 58 by substitution, deletion, or insertion of one or more amino acids;
e) a polypeptide derived from a mature polypeptide of any one of SEQ ID NO(s): 58 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has alpha-mannosidase activity belonging to EC 3.2.1.24. Examples of proteins that exhibit alpha-mannosidase activity and have an amino acid sequence that is at least 70% identical to one or more of SEQ ID NOs: 58 are set forth herein as SEQ ID NOs: 45755.

In some embodiments, the protein is derived from Neobacillus. For example, in some embodiments, the protein is a native Neobacillus alpha-mannosidase or is a fragment/mutant/variant of a native Neobacillus alpha-mannosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 59-60 and 45906;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ TD NO(s): 59-60 and 45906;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 150-151 and 72146 or the cDNA sequence thereof;
d) a polypeptide derived from any one of SEQ ID NO(s): 59-60 and 45906 by substitution, deletion, or insertion of one or more amino acids;
e) a polypeptide derived from a mature polypeptide of any one of SEQ ID NO(s): 59-60 and 45906 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucan endo-1,3-beta-D-glucosidase activity belonging to EC 3.2.1.39. Examples of proteins that exhibit glucan endo-1,3-beta-D-glucosidase activity and have an amino acid sequence that is at least 70% identical to one or more of SEQ ID NOs: 59-60 and 45906 are set forth herein as SEQ ID NOs: 45905-45912.

In some embodiments, the protein is derived from Trichoderma. For example, in some embodiments, the protein is a native Trichoderma glucan endo-1,3-beta-D-glucosidase or is a fragment/mutant/variant of a native Trichoderma glucan endo-1,3-beta-D-glucosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 61;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 61;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 152 or the cDNA sequence thereof;
d) a polypeptide derived from any one of SEQ ID NO(s): 61 by substitution, deletion, or insertion of one or more amino acids;
e) a polypeptide derived from a mature polypeptide of any one of SEQ ID NO(s): 61 by substitution, deletion, or insertion of one or more amino acids;
f) a polypeptide derived from the polypeptide of any one of a) through f) wherein the N- and/or C-terminal end has been extended by the addition of one or more amino acids; and
g) a fragment of the polypeptide of any one of a) through g)
   wherein the polypeptide has pullulanase activity belonging to EC 3.2.1.41. Examples of proteins that exhibit pullulanase activity and have an amino acid sequence that is at least 70% identical to one or more of SEQ ID NOs: 61 are set forth herein as SEQ ID NOs: 48751-49148.

In some embodiments, the protein is derived from Bacillus, Geobacillus or Pulluanibacillus. For example, in some embodiments, the protein is a native Bacillus, Geobacillus or Pulluanibacillus pullulanase or is a fragment/mutant/variant of a native Bacillus, Geobacillus or Pulluanibacillus pullulanase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 62;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 62;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 153 or the cDNA sequence thereof,
d) a polypeptide derived from any one of SEQ ID NO(s): 62 by substitution, deletion, or insertion of one or more amino acids;
e) a polypeptide derived from a mature polypeptide of any one of SEQ ID NO(s): 62 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has alpha-L-arabinofuranosidase activity belonging to EC 3.2.1.55. Examples of proteins that exhibit alpha-L-arabinofuranosidase activity and have an amino acid sequence that is at least 70% identical to one or more of SEQ ID NOs: 62 are set forth herein as SEQ ID NOs: 49149-49209.

In some embodiments, the protein is derived from Acremonium, ACrophialophora, Actinomadura, Actinoplanes, Aspergillus, Aureobasidium, Chaetomium, Gliomastix, Humicola, Hypocrea, Microdochium, Myceliophthora, Oculimacula, Penicillium, Streptomyces, Streptosporangium, Talaromyces, Thielavia, Trichoderma or Xylanibacterium. For example, in some embodiments, the protein is a native Acremonium, ACrophialophora, Actinomadura, Actinoplanes, Aspergillus, Aureobasidium, Chaetomium, Gliomastix, Humicola, Hypocrea, Microdochium, Myceliophthora, Oculimacula, Penicillium, Streptomyces, Streptosporangium, Talaromyces, Thielavia, Trichoderma or Xylanibacterium alpha-L-arabinofuranosidase or is a fragment/mutant/variant of a native Acremonium, ACrophialophora, Actinomadura, Actinoplanes, Aspergillus, Aureobasidium, Chaetomium, Gliomastix, Humicola, Hypocrea, Microdochium, Mvceliophthora, Oculimacula, Penicilhum, Streptomyces, Streptosporangium, Talaromyces, Thielavia, Trichoderma or Xylanihacterium alpha-L-arabinofuranosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 63-64;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 63-64;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 154-155 or the cDNA sequence thereof,
d) a polypeptide derived from any one of SEQ ID NO(s): 63-64 by substitution, deletion, or insertion of one or more amino acids;
e) a polypeptide derived from a mature polypeptide of any one of SEQ ID NO(s): 63-64 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucan endo-1,3-alpha-glucosidase activity belonging to EC 3.2.1.59. Examples of proteins that exhibit glucan endo-1,3-alpha-glucosidase activity and have an amino acid sequence that is at least 70% identical to one or more of SEQ ID NOs: 63-64 are set forth herein as SEQ ID NOs: 49210-49228.

In some embodiments, the protein is derived from Clonostachys, Hypocrea, Myceliophthora or Trichoderma. For example, in some embodiments, the protein is a native Clonostachys, Hypocrea, Myceliophthora or Trichoderma glucan endo-1,3-alpha-glucosidase or is a fragment/mutant/variant of a native Clonostachys, Hypocrea, Myceliophthora or Trichoderma glucan endo-1,3-alpha-glucosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 65;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 65;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 156 or the cDNA sequence thereof;
d) a polypeptide derived from any one of SEQ ID NO(s): 65 by substitution, deletion, or insertion of one or more amino acids;
e) a polypeptide derived from a mature polypeptide of any one of SEQ ID NO(s): 65 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has licheninase activity belonging to EC 3.2.1.73. Examples of proteins that exhibit licheninase activity and have an amino acid sequence that is at least 70% identical to one or more of SEQ ID NOs: 65 are set forth herein as SEQ ID NOs: 49420-49446.

In some embodiments, the protein is derived from Bacillus or Salipaludibacillus. For example, in some embodiments, the protein is a native Bacillus or Salipaludibacillus licheninase or is a fragment/mutant/variant of a native Bacillus or Salipaludibacillus licheninase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 66-67;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 66-67;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 157-158 or the cDNA sequence thereof,
d) a polypeptide derived from any one of SEQ ID NO(s): 66-67 by substitution, deletion, or insertion of one or more amino acids;
e) a polypeptide derived from a mature polypeptide of any one of SEQ ID NO(s): 66-67 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucan endo-1,6-beta-glucosidase activity belonging to EC 3.2.1.75. Examples of proteins that exhibit glucan endo-1,6-beta-glucosidase activity and have an amino acid sequence that is at least 70% identical to one or more of SEQ ID NOs: 66-67 are set forth herein as SEQ ID NOs: 49447-49451.

In some embodiments, the protein is derived from Trichoderma. For example, in some embodiments, the protein is a native Trichoderma glucan endo-1,6-beta-glucosidase or is a fragment/mutant/variant of a native Trichoderma glucan endo-1,6-beta-glucosidase.

In some embodiments, the protein is selected from the group consisting of
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 68 and 72144;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 68 and 72144;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 159 and 72145 or the cDNA sequence thereof;
d) a polypeptide derived from any one of SEQ ID NO(s): 68 and 72144 by substitution, deletion, or insertion of one or more amino acids;
e) a polypeptide derived from a mature polypeptide of any one of SEQ ID NO(s): 68 and 72144 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has mannan endo-1,4-beta-mannosidase activity belonging to EC 3.2.1.78. Examples of proteins that exhibit mannan endo-1,4-beta-mannosidase activity and have an amino acid sequence that is at least 70% identical to one or more of SEQ ID NOs: 68 and 72144 are set forth herein as SEQ ID NOs: 49452.

In some embodiments, the protein is derived from Alkalihalobacillus. For example, in some embodiments, the protein is a native Alkalihalobacillus mannan endo-1,4-beta-mannosidase or is a fragment/mutant/variant of a native Alkalihalobacillus mannan endo-1,4-beta-mannosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 69;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 69;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 160 or the cDNA sequence thereof,
d) a polypeptide derived from any one of SEQ ID NO(s): 69 by substitution, deletion, or insertion of one or more amino acids;
e) a polypeptide derived from a mature polypeptide of any one of SEQ ID NO(s): 69 by substitution, deletion, or insertion of one or more amino acids;
f) a polypeptide derived from the polypeptide of any one of a) through c) wherein the N- and/or C-terminal end has been extended by the addition of one or more amino acids; and
g) a fragment of the polypeptide of any one of a) through f)
   wherein the polypeptide has mannan endo-1,6-alpha-mannosidase activity belonging to EC 3.2.1.101.

Examples of proteins that exhibit mannan endo-1,6-alpha-mannosidase activity and have an amino acid sequence that is at least 70% identical to one or more of SEQ ID NOs: 69 are set forth herein as SEQ ID NOs: 45042.

In some embodiments, the protein is derived from Talaromyces. For example, in some embodiments, the protein is a native Talaromyces mannan endo-1,6-alpha-mannosidase or is a fragment/mutant/variant of a native Talaromyces mannan endo-1,6-alpha-mannosidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 70-75;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 70-75;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 161-166 or the cDNA sequence thereof,
d) a polypeptide derived from any one of SEQ ID NO(s): 70-75 by substitution, deletion, or insertion of one or more amino acids;
e) a polypeptide derived from a mature polypeptide of any one of SEQ ID NO(s): 70-75 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has endogalactosaminidase activity belonging to EC 3.2.1.109. Examples of proteins that exhibit endogalactosaminidase activity and have an amino acid sequence that is at least 70% identical to one or more of SEQ ID NOs: 70-75 are set forth herein as SEQ ID NOs: 45043-45062.

In some embodiments, the protein is derived from Bjerkandera, Diaporthe, Fusarium, Neonectria, Ostropa, Pseudoplectania, Stenocarpella or Urunula. For example, in some embodiments, the protein is a native Bjerkandera, Diaporthe, Fusarium, Neonectria, Ostropa, Pseudoplectania, Stenocarpella or Urunula endogalactosaminidase or is a fragment/mutant/variant of a native Bjerkandera, Diaporthe, Fusarium, Neonectria, Ostropa, Pseudoplectania, Stenocarpella or Urunula endogalactosaminidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 76-77;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 76-77;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 167-168 or the cDNA sequence thereof;
d) a polypeptide derived from any one of SEQ ID NO(s): 76-77 by substitution, deletion, or insertion of one or more amino acids;
e) a polypeptide derived from a mature polypeptide of any one of SEQ ID NO(s): 76-77 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glycoprotein endo-alpha-1,2-mannosidase activity belonging to EC 3.2.1.130 and/or alpha-mannan endo-1,2-alpha-mannanase activity belonging to EC 3.2.1.198.

In some embodiments, the protein is derived from Chryseobacterium or Lysobacter. For example, in some embodiments, the protein is a native Chryseobacterium or Lysobacter glycoprotein endo-alpha-1,2-mannosidase or is a fragment/mutant/variant of a native Chryseobacterium or Lysobacter glycoprotein endo-alpha-1,2-mannosidase. Similarly, in some embodiments, the protein is a native Chryseobacterium or Lysobacter alpha-mannan endo-1,2-alpha-mannanase or is a fragment/mutant/variant of a native Chryseobacterium or Lysobacter alpha-mannan endo-1,2-alpha-mannanase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 78;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 78;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 169 or the cDNA sequence thereof;
d) a polypeptide derived from any one of SEQ ID NO(s): 78 by substitution, deletion, or insertion of one or more amino acids;
c) a polypeptide derived from a mature polypeptide of any one of SEQ ID NO(s): 78 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glucan 1,4-alpha-maltohydrase activity belonging to EC 3.2.1.133. Examples of proteins that exhibit glucan 1,4-alpha-maltohydrase activity and have an amino acid sequence that is at least 70% identical to one or more of SEQ ID NOs: 78 are set forth herein as SEQ ID NOs: 45072-45408.

In some embodiments, the protein is derived from Alicyclobacillus, Bacillus, Effusibacillus or Geobacillus. For example, in some embodiments, the protein is a native Alicyclobacillus, Bacillus, Effusibacillus or Geobacillus glucan 1,4-alpha-maltohydrase or is a fragment/mutant/variant of a native Alicyclobacillus, Bacillus, Effusibacillus or Geobacillus glucan 1,4-alpha-maltohydrase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 79;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 79;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 170 or the cDNA sequence thereof,
d) a polypeptide derived from any one of SEQ ID NO(s): 79 by substitution, deletion, or insertion of one or more amino acids;
e) a polypeptide derived from a mature polypeptide of any one of SEQ ID NO(s): 79 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has 1,6-alpha-D-mannosidase activity belonging to EC 3.2.1.163. Examples of proteins that exhibit 1,6-alpha-D-mannosidase activity and have an amino acid sequence that is at least 70% identical to one or more of SEQ ID NOs: 79 are set forth herein as SEQ ID NOs: 45437-45442.

In some embodiments, the protein is derived from Aspergillus, Evansstolkia, Penicillium, Rasamsonia, Talaromyces or Thermomyces. For example, in some embodiments, the protein is a native Aspergillus, Evansstolkia, Penicillium, Rasamsonia, Talaromyces or Thermomyces 1,6-alpha-D-mannosidase or is a fragment/mutant/variant of a native Aspergillus, Evansstolkia, Penicillium, Rasamsonia, Talaromyces or Thermomyces 1,6-alpha-D-mannosidase.

Those skilled in the art will understand how to identify, isolate, characterize, produce, recover and formulate proteins having one or more glycosylase activities belong to EC 3.2. See, e.g., AU2016101771-A4; AU2017203481-A1; BY20193-C1; CN101134949-A; CN101139556-A; CN101173226-A; CN101298604-A; CN101457230-A; CN101492661-A; CN101659948-A; CN101824401-A; CN101870956-A; CN101899458-A; CN101962633-A; CN102021191-A; CN102477437-A; CN102477438-A; CN102492707-A; CN102703480-A; CN102796751-A; CN102827821-A; CN102851266-A; CN102994475-A; CN103060290-A; CN103184163-A; CN103194419-A; CN103343111-A; CN103525793-A; CN103555751-A; CN103789285-A; CN103805579-A; CN103834606-A; CN103966188-A; CN104130988-A; CN104293748-A; CN104313000-A; CN104450650-A; CN104450651-A; CN104480087-A; CN104498456-A; CN104561060-A; CN104789543-A; CN104862290-A; CN104928270-A; CN105039288-A; CN105062991-A; CN105155324-A; CN105176947-A; CN105177084-A; CN105441415-A; CN105623937-A; CN105671019-A; CN105671022-A; CN105695435-A; CN105734034-A; CN105802940-A; CN105802943-A; CN105886520-A; CN105907775-A; CN105969783-A; CN106084016-A; CN106148235-A; CN106190934-A; CN106754825-A; CN106754826-A; CN106801046-A; CN106929495-A; CN107022588-A; CN107058264-A; CN107236692-A; CN107312763-A; CN107326020-A; CN107475219-A; CN107513527-A; CN107586767-A; CN107603965-A; CN108102934-A; CN108588056-A; CN108611339-A; CN108623652-A; CN108795781-A; CN108823186-A; CN108841809-A; CN109022396-A; CN109055439-A; CN109207458-A; CN109234339-A; CN109321552-A; CN109370973-A; CN109439607-A; CN109439641-A; CN109486689-A; CN109486791-A; CN109777795-A; CN109810961-A; CN109825466-A; CN109897842-A; CN110066777-A; CN110157688-A; CN110229800-A; CN110343687-A; CN110423737-A; CN110495549-A; CN110592051-A; CN110628748-A; CN110699337-A; CN110713999-A; CN110724645-A; CN110724646-A; CN110951628-A; CN111454929-A; CN111500558-A; CN111518792-A; CN111621490-A; CN111793613-A; CN111826377-A; CN112063678-A; CN112342208-A; CN112522238-A; CN112552382-A; CN112680433-A; CN112795554-A; CN112795555-A; CN113151327-A; CN113234705-A; CN113584075-A; CN113667662-A; CN113717864-A; CN113817709-A; CN113881654-A; CN114350637-A; CN114369588-A; CN114457056-A; CN114606216-A; CN114686386-A; CN114752583-A; CN114790451-A; CN115029334-A; CN115074345-A; CN115125226-A; CN115197924-A; CN115247165-A; CN115478061-A; CN115703996-A; CN115704017-A; CN115704018-A; CN115704019-A; CN1465699-A; CN1594542-A; CN1834249-A; DD272102-A; DE102004047777-A1; DE102005062984-A1; DE102014018149-A1; DE102014212640-A1; DE102014212643-A1; DE102014225472-1; DE102014225473-A1; DE102020205400-A1; DE10309803-A1; EP1764411-A1; EP208491-; EP2100948-A1; EP2100950-A1; EP2116136-A1; EP2166092-A1; EP2292773-A1; EP2295582-A2; EP2298904-A1; EP2314698-A1; EP2357220-A1; EP2404928-A1; EP2404930-A1; EP2486799-A1; EP2540825-A2; EP2551335-A1; EP260160-; EP285123-A; EP2993230-A1; EP3034592-A1; EP3121270-A2; EP3228703-A1; EP3241890-A1; EP3241891-A1; EP3301154-A1; EP3330349-A1; EP3339422-A1; EP3428260-A2; EP3485734-A1; EP3502242-A1; EP3502243-A1; EP3653705-A1; EP3653706-A1; EP3730595-A2; EP4001389-A1; EP4047088-A1; EP409299-A; EP495257-1; EP540784-A1; EP605040-A1; EP633311-A1; EP683228-A2; EP828002-A2; FR2665178-A; FR2676456-A1; GB2479462-A; IN194800-B; IN201811045754-A; IN9700577-11; IN9702287-14; JP04058889-A; JP07059574-A; JP2000135093-A; JP2000245466-A; JP2004313022-A; JP2005171409-A; JP2011167076-A; JP2011223962-A; JP2016015894-A; JP2019146520-A; JP2020065514-A; JP2020184937-A; JP62104580-A; KR2001027418-A; KR2004006812-A; KR2011017208-A; KR2011106174-A; KR2012140565-A; KR2013112172-A; KR834708-B1; NZ524303-A; RU2574206-C1; TR201404579-A; TW200811294-A; U.S. Ser. No. 10/563,185-B1; US2004191864-A1; US2005108791-A1; US2005196853-A1; US2005214410-A1; US2006246566-A1; US2007044171-A1; US2007244020-A1; US2008044858-A1; US2008148432-A1; US2009111155-A1; US2009117642-A1; US2009158452-A1; US2009181874-A1; US2009203109-A1; US2009209026-A1; US2009238923-A1; US2009252828-A1; US2010021587-A1; US2010124769-A1; US2010204080-A1; US2010323448-A1; US2011039751-A1; US2011099671-A1; US2011195481-A1; US2011252501-A1; US2011269210-A1; US2011287516-A1; US2012156734-A1; US2012252095-A1; US2013011882-A1; US2013074202-A1; US2013252850-A1; US2013269061-A1; US2014017737-A1; US2014127753-A1; US2014256018-A1; US2015064766-A1; US2015299720-A1; US2015337329-A1; US2016032267-A1; US2017247730-A1; US2017342433-A1; US2018010111-A1; US2018155744-A1; US2018163191-A1; US2018216039-A1; US2018258442-A1; US2019194634-A1; US2019359961-A1; US2019360012-A1; US2020181593-A1; US2020270593-A1; US2020306342-A1; US2020339917-A1; US2021122998-A1; US2021163995-A1; US2021230644-A1; US2021254115-A1; US2021292688-A1; US2021317431-A1; US2022154236-A1; US2022204956-A1; US2022347262-A1; US2023002748-A1; U.S. Pat. No. 5,824,532-A; U.S. Pat. No. 5,849,549-A; U.S. Pat. No. 6,682,923-B1; U.S. Pat. No. 6,887,986-B1; U.S. Pat. No. 7,504,490-B1; U.S. Pat. No. 8,409,836-B2; WO200001796-A2; WO200029560-A1; WO200034452-A1; WO200043504-A1; WO200060058-A2; WO200060059-A2; WO200109294-A1; WO200116349-A1; WO200136586-A2; WO200142433-A2; WO200147956-A2; WO200151620-A2; WO200159141-A2; WO200164852-A1; WO200183559-A2; WO200188107-A2; WO200210355-A2; WO200212511-A1; WO200240997-A2; WO200268589-A2; WO200268597-A2; WO2003012071-A2; WO2003014358-A2; WO2003016535-A2; WO2003018766-A2; WO2003089614-A2; WO2004080923-A2; WO2004081171-A2; WO2004091544-A2; WO2004113551-A1; WO2005001036-A2; WO2005001064-A2; WO2005003311-A2; WO2005019443-A2; WO2005045018-A1; WO2005052148-A2; WO2005054475-A1; WO2005056787-A1; WO02005073368-A1; WO2005096804-A2; WO2005108537-A1; WO2005111203-A2; WO2005113785-A2; WO2005117756-A2; WO2006002643-A2; WO2006012899-A1; WO2006012902-A2; WO2006031554-A2; WO2006037483-A2; WO2006066594-A2; WO2006066596-A2; WO2006069290-A2; WO2006117432-A1; WO2006136159-A2; WO2006136160-A2; WO2007028088-A2; WO2007071820-A1; WO2007113292-A2; WO2007146944-A2; WO2007149699-A2; WO2008000632-A1; WO2008006881-A1; WO2008015861-A1; WO2008024372-A2; WO2008057637-A2; WO2008080093-A2; WO2008112459-A2; WO2008148845-A2; WO02008153805-A2; WO2008153934-A2; WO02008153935-A2; WO2009026397-A2; WO2009037279-A1; WO2009061379-A2; WO2009062942-A2; WO2009071550-A1; WO2009074650-A2; WO2009075682-A1; WO2009076655-A2; WO2009098229-A2; WO2009100102-A2; WO2009100138-A2; WO2009100990-A1; WO2009108941-A2; WO2009112992-A1; WO2009126773-A1; WO02009133096-A2; WO2009134670-A2; WO2009149130-A2; WO2009149271-A2; WO2009149395-A2; WO2009158694-A1; WO2009158716-A1; WO2010022518-A1; WO2010027857-A2; WO2010036515-A1; WO2010036970-A2; WO2010072224-A1; WO2010072225-A1; WO2010074999-A1; WO2010102982-A1; WO2010104675-A1; WO2010115021-A2; WO2010115156-A2; WO2010121933-A1; WO2010132157-A2; WO2010135836-A1; WO2010138754-A1; WO2010148148-A2; WO2011020852-A1; WO2011041391-A1; WO2011048852-A1; WO2011057101-A1; WO2011057140-A1; WO2011057159-A1; WO2011057163-A2; WO2011066187-A1; WO2011076123-A1; WO2011076897-A1; WO2011080352-A1; WO2011080353-A1; WO2011080354-A1; WO2011082425-A2; WO2011082429-A1; WO2011127820-A1; WO2011128712-A1; WO2011153516-A2; WO2012013197-A2; WO2012016960-A1; WO2012021399-A1; WO2012021400-A1; WO2012021883-A2; WO2012027282-A2; WO2012027374-A2; WO2012027395-A2; WO2012044835-A1; WO2012044836-A1; WO2012044915-A2; WO2012088303-A2; WO2012088467-A2; WO2012089024-A1; WO2012101206-A2; WO2012106824-A1; WO2012122308-A2; WO2012125865-A1; WO2012125925-A2; WO2012127001-A1; WO2012128260-A1; WO2012129697-A1; WO2012129699-A1; WO2012134626-A2; WO2012159007-A1; WO2013001078-A1; WO2013001087-A2; WO2013023938-A1; WO2013029496-A1; WO2013034106-A1; WO2013039776-A1; WO2013055676-A1; WO2013057141-A2; WO2013057143-A2; WO2013063460-A2; WO2013074956-A2; WO2013076253-A1; WO2013082486-A1; WO2013089889-A2; WO2013096294-A1; WO2013115305-A1; WO2013148993-A1; WO2013160316-A1; WO2013181760-A1; WO2013182671-A1; WO2013184577-A1; WO2014007921-A1; WO2014013073-A1; WO2014013074-A1; WO2014019219-A1; WO2014029808-A1; WO2014055778-A2; WO2014055782-A1; WO2014059541-A1; WO2014060380-A1; WO2014068109-A2; WO2014070841-A1; WO2014081700-A1; WO2014081884-A1; WO2014085439-A1; WO2014093123-A1; WO2014099416-A1; WO2014099653-A1; WO2014101753-A1; WO2014106593-A1; WO2014124927-A2; WO2014131842-A1; WO2014138672-A1; WO2014138983-A1; WO2014140165-A1; WO2014151805-A2; WO2014152878-A2; WO2014161987-A1; WO2014162001-A1; WO2014164777-A1; WO2014164800-A1; WO2014164834-A1; WO2014169101-A1; WO2014169129-A1; WO2014176311-A1; WO2014182990-A1; WO2014183920-A1; WO2014183921-A1; WO2014195356-A2; WO2014197296-A1; WO2014202616-A2; WO2014202622-A2; WO2014209800-A1; WO2015007639-A1; WO2015035914-A1; WO2015042064-A1; WO2015044448-A1; WO2015048332-A2; WO2015048340-A2; WO2015048342-A2; WO2015050723-A1; WO2015058700-A1; WO2015065871-A1; WO2015066492-A1; WO2015077126-A1; WO2015079063-A1; WO2015091959-A2; WO2015091990-A1; WO2015094809-A1; WO2015105835-A1; WO2015109405-A1; WO2015110473-A2; WO2015118123-A1; WO2015143144-A1; WO2015143324-A1; WO2015144782-A1; WO2015144824-A1; WO2015150457-A1; WO2015157656-A1; WO2015162038-A1; WO2015187697-A2; WO2015189370-A1; WO2015189371-A1; WO2015189372-A1; WO2015200659-A1; WO2016020478-A1; WO2016029107-A1; WO2016030448-A1; WO2016036648-A1; WO2016036834-A1; WO2016040464-A1; WO2016062875-A2; WO2016066896-A1; WO2016079110-A2; WO2016079305-A1; WO2016087327-A1; WO2016087445-A1; WO2016090472-A1; WO2016090473-A1; WO2016090474-A1; WO2016095856-A1; WO2016100837-A1; WO2016109758-A2; WO2016117549-A1; WO2016124651-A1; WO2016126294-A1; WO2016145350-A1; WO2016153924-A1; WO2016167235-A1; WO2016180748-A1; WO2016180749-A1; WO2016180792-A1; WO2016196202-A1; WO2016203064-A2; WO2016206621-A1; WO2016208492-A1; WO2016210238-A1; WO2017010833-A1; WO2017014974-A1; WO2017019490-A1; WO2017029238-A2; WO2017083226-A1; WO2017084560-A1; WO2017088820-A1; WO2017097861-A1; WO2017097866-A1; WO2017097896-A1; WO2017097897-A1; WO2017106676-A1; WO2017112540-A1; WO2017112631-A1; WO2017112643-A1; WO2017114891-A1; WO2017129754-A1; WO2017144008-A1; WO2017147690-A1; WO2017152169-A1; WO2017161091-A1; WO2017186928-A1; WO2017191109-A1; WO2017191160-A1; WO2017194487-A1; WO2017205337-A1; WO2017217453-A1; WO2017220422-A1; WO2018002261-A1; WO2018015303-A1; WO2018027131-A1; WO2018053220-A1; WO2018075430-A1; WO2018085524-A2; WO2018094181-A1; WO2018098124-A1; WO2018098381-A1; WO2018102348-A1; WO2018106656-A1; WO2018112123-A1; WO2018127486-A1; WO2018161899-A1; WO2018165594-A1; WO2018167669-A1; WO2018167670-A1; WO2018184004-A1; WO2018185048-A1; WO2018204483-A2; WO2018209345-A1; WO2018219854-A1; WO2018224544-A1; WO2019014118-A1; WO2019036721-A2; WO2019042971-A1; WO2019063543-A1; WO2019068850-A1; WO2019083831-A1; WO2019099235-A1; WO2019110538-A1; WO2019113413-A1; WO2019113415-A1; WO2019121930-A1; WO2019121937-A1; WO2019137289-A1; WO2019161227-A1; WO2019163886-A1; WO2019193102-A1; WO2019201725-A1; WO2019201783-A1; WO2019201785-A1; WO2019228448-A1; WO2019229228-A1; WO2020002574-A1; WO2020007875-A1; WO2020023411-A1; WO2020028443-A1; WO2020053276-A1; WO2020058228-A1; WO2020070199-A1; WO2020076697-A1; WO2020077331-A2; WO2020086430-A2; WO2020088393-A1; WO2020096905-A1; WO2020096907-A1; WO2020106600-A1; WO2020114965-A1; WO2020123463-A1; WO2020127775-A1; WO2020131691-A2; WO2020154387-A1; WO2020156903-A1; WO2020169840-A1; WO2020174070-A1; WO2020187883-A1; WO2020188095-A1; WO2020193532-A1; WO2020193534-A2; WO2020193535-A2; WO2020201403-A1; WO2020208056-A1; WO2020260223-A1; WO2020264552-A1; WO2021025872-A1; WO2021026201-A1; WO2021032881-A1; WO2021055395-A1; WO2021058022-A1; WO2021080948-A2; WO2021099457-A1; WO2021119304-A1; WO2021123184-A2; WO2021127319-A1; WO2021130167-A1; WO2021133658-A1; WO2021133701-A1; WO2021144342-A1; WO2021148364-A1; WO2021152120-A1; WO2021152123-A1; WO2021163030-A2; WO2021163036-A1; WO2021202479-A1; WO2021204838-A1; WO2021212095-A1; WO2021214059-A1; WO2021231621-A1; WO2021231623-A1; WO2021236715-A1; WO2021248045-A2; WO2022014428-A1; WO2022020665-A2; WO2022043273-A2; WO2022043745-A1; WO2022044629-A1; WO2022047264-A1; WO2022071451-A1; WO2022074037-A2; WO2022089571-A1; WO2022171780-A2; WO2022173694-A1; WO2022175435-A1; WO2022214457-A1; WO2022214459-A1; WO2022225696-A2; WO2022226376-A2; WO2022261003-A1; WO2022268885-A1; WO2023002065-A2; WO2023023642-A2; WO2023274282-A1; WO2023288294-A1; WO8402921-A; WO9009436-A; WO9100353-A; WO9117244-A; WO9203540-A; WO9218688-A1; WO9219726-A1; WO9402597-A1; WO9407998-A1; WO9418314-A1; WO9502675-A1; WO9518219-A1; WO9524471-A1; WO9526397-A1; WO9534662-A1; WO9535382-A2; WO9605295-A2; WO9617994-A1; WO9623873-A1; WO9623874-A1; WO9629397-A1; WO9630481-A1; WO9639528-A2; WO9714804-A1; WO9728256-A1; WO9743409-A2; WO9743424-A1; WO9803639-A1; WO9804663-A1; WO9811239-A1; WO9812307-A1; WO9816633-A1; WO9818437-A1; WO9826078-A1; WO9844126-A1; WO9909183-A1; WO9919467-A1; WO9923211-A1; WO9929876-A2; WO9945124-A2; WO9946399-A1; WO9957256-A1; WO9957260-A1.

In some embodiments, proteins of the present disclosure exhibit one or more peptidase activities belonging to EC 3.4, such as aminopeptidase activities belonging to EC 3.4.11, serine endopeptidase activities belonging to EC 3.4.21 (e.g., chymotrypsin activity belonging to EC 3.4.21.1, trypsin activity belonging to EC 3.4.21.4, alpha-lytic endopeptidase activity belonging to EC 3.4.21.12, glutamyl endopeptidase activity belonging to EC 3.4.21.19, cucumisin activity belonging to EC 3.4.21.25, chymase activity belonging to EC 3.4.21.39, lysyl endopeptidase activity belonging to EC 3.4.21.50, leucyl endopeptidase activity belonging to EC 3.4.21.57, subtilisin activity belonging to 3.4.21.62), cysteine endopeptidase activities belonging to 3.4.22 (e.g., papain activity belonging to EC 3.4.22.2, actinidain activity belonging to EC 3.4.22.14, caricain activity belonging to EC 3.4.22.30, ananain activity belonging to EC 3.4.22.31, bromelain activity belonging to EC 3.4.22.32, bromelain activity belonging to EC 3.4.22.33, legumain activity belonging to EC 3.4.22.34, zingipain activity belonging to EC 3.4.22.67), aspartic endopeptidase activities belonging to EC 3.4.23 (e.g., phytepsin activity belonging to EC 3.4.23.40), and/or metalloendopeptidase activities belonging to EC 3.4.24 (e.g., bacillolysin activity belonging to EC 3.4.24.28), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 80-88.

In some embodiments, proteins of the present disclosure exhibit serine endoprotease activity belonging to EC 3.4.21 and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 80-86.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 80-81;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 80-81;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 171-172 or the cDNA sequence thereof;
d) a polypeptide derived from any one of SEQ ID NO(s): 80-81 by substitution, deletion, or insertion of one or more amino acids;
e) a polypeptide derived from a mature polypeptide of any one of SEQ ID NO(s): 80-81 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has serine endopeptidase activity belonging to EC 3.4.21. Examples of proteins that exhibit serine endopeptidase activity and have an amino acid sequence that is at least 70% identical to one or more of SEQ ID NOs: 80-81 are set forth herein as SEQ ID NOs: 49913-50777.

In some embodiments, the protein is derived from Cinereomyces, Dichomitus, Ganoderma, Grifola, Lenzites, Meripilus, Neolentinus, Nocardiopsis, Polyporus or Trametes. For example, in some embodiments, the protein is a native Cinereomyces, Dichomitus, Ganoderma, Grifola, Lenzites, Meripilus, Neolentinus, Nocardiopsis, Polyporus or Trametes serine endopeptidase or is a fragment/mutant/variant of a native Cinereomyces, Dichomitus, Ganoderma, Grifola, Lenzites, Meripilus, Neolentinus, Nocardiopsis, Polyporus or Trametes serine endopeptidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 82;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 82;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 173 or the cDNA sequence thereof;
d) a polypeptide derived from any one of SEQ ID NO(s): 82 by substitution, deletion, or insertion of one or more amino acids;
e) a polypeptide derived from a mature polypeptide of any one of SEQ ID NO(s): 82 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has glutamyl endopeptidase activity belonging to EC 3.4.21.19. Examples of proteins that exhibit glutamyl endopeptidase activity and have an amino acid sequence that is at least 70% identical to one or more of SEQ ID NOs: 82 are set forth herein as SEQ ID NOs: 50778-50953.

In some embodiments, the protein is derived from Bacillus. For example, in some embodiments, the protein is a native Bacillus glutamyl endopeptidase or is a fragment/mutant/variant of a native Bacillus glutamyl endopeptidase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 83-87;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 83-87;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SQ ID NO(s): 174-178 or the cDNA sequence thereof;
d) a polypeptide derived from any one of SEQ ID NO(s): 83-87 by substitution, deletion, or insertion of one or more amino acids;
e) a polypeptide derived from a mature polypeptide of any one of SEQ ID NO(s): 83-87 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has subtilisin activity belonging to EC 3.4.21.62. Examples of proteins that exhibit subtilisin activity and have an amino acid sequence that is at least 70% identical to one or more of SEQ ID NOs: 83-87 are set forth herein as SEQ ID NOs: 50954-70838.

In some embodiments, the protein is derived from Alkalihalobacillus, Aspergillus, Bacillus, Brevia, Geomicrobium, Homo, Hordeum, Lederbergia, Saccharomyces, Shouchella or Thermus. For example, in some embodiments, the protein is a native Alkalihalobacillus, Aspergillus, Bacillus, Brevia, Geomicrobium, Homo, Hordeum, Lederbergia, Saccharomyces, Shouchella or Thermus subtilisin or is a fragment/mutant/variant of a native Alkalihalobacillus, Aspergillus, Bacillus, Brevia, Geomicrobium, Homo, Hordeum, Lederbergia, Saccharomyces, Shouchella or Thermus subtilisin.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 88;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 88;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 179 or the cDNA sequence thereof,
d) a polypeptide derived from any one of SEQ ID NO(s): 88 by substitution, deletion, or insertion of one or more amino acids;
e) a polypeptide derived from a mature polypeptide of any one of SEQ ID NO(s): 88 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has bacillolycin activity belonging to EC 3.4.24.28. Examples of proteins that exhibit bacillolycin activity and have an amino acid sequence that is at least 70% identical to one or more of SEQ ID NOs: 88 are set forth herein as SEQ ID NOs: 70839-71903.

In some embodiments, the protein is derived from Bacillus. For example, in some embodiments, the protein is a native Bacillus bacillolycin or is a fragment/mutant/variant of a native Bacillus bacillolycin.

Those skilled in the art will understand how to identify, isolate, characterize, produce, recover and formulate proteins having one or more peptidase activities belong to EC 3.4. See, e.g., CA2829859-A1; CN102676561-A; CN102703482-A; CN103013960-A; CN103243081-A; CN103255156-A; CN103602653-A; CN104232610-A; CN105132397-A; CN105176951-A; CN106350530-; CN106801048-A; CN107384897-A; CN107828765-A; CN107937372-A; CN107937374-A; CN108004220-; CN108359659-A; CN108384771-A; CN108570461-A; CN108570462-A; CN109456957-A; CN109593746-; CN110746494-A; CN110777136-A; CN110819612-A; CN110862979-A; CN110923221-A; CN110923222-; CN111004794-A; CN111334494-A; CN111575265-A; CN111893126-A; CN111909979-A; CN112301023-; CN112458072-A; CN112501149-A; CN112574978-A; CN112662652-A; CN112662653-A; CN113528493-; CN113699138-A; CN113832130-A; CN113832131-A; CN113862244-A; CN114107266-A; CN114317502-; CN114540330-A; CN114561375-A; CN114574469-A; CN114591935-A; CN114774396-A; CN114806990-; CN114836408-A; CN114908074-A; CN114958897-A; CN115029337-A; CN115074347-A; CN115247166-; CN1361279-A; CN1814746-A; CN1814755-A; CN1995343-A; DE102006022224-A1; DE102007044415-1; DE102007049830-A1; DE102007051092-A1; DE102008059446-A1; DE102008059447-A1; DE102009029513-A1; DE102010028951-A; DE102011005354-A; DE102011118032-A1; DE102012215642-A1; DE102012220101-A1; DE102013219467-A1; DE102013221206-A1; DE102014212643-A1; DE102014224825-A1; DE102014226681-A1; DE102015223270-A1; DE102016204814-A1; DE102016204815-A1; DE102016208463-A1; DE102016210628-A1; DE102017215628-A1; DE102017215629-A1; DE102017215631-A1; DE102017223275-A1; DE102018004206-A1; DE102018004207-A1; DE102019111047-A1; DE102019111057-A1; DE102019111075-A1; DE102019210806-A1; DE102020105720-A1; DE102020105721-A1; DE102020205381-A1; DE102020205400-A1; DE10260903-A1; DE19530816-A1; DE3821491-A; DE4224125-A1; DE4411223-A1; EP1160327-A2; EP133756-A; EP2100948-A1; EP2607468-A1; EP3106508-A1; EP3275988-A1; EP3301155-A1; EP3309244-A1; EP3323875-A1; EP3339423-A1; EP3660146-A1; EP3660151-A1; EP398539-A; EP405901-A; EP405902-A; EP415296-A; EP416967-A; EP430637-A; EP443476-A; EP482879-A; EP516200-A1; EP571049-A1; EP687733-A1; JP01137972-A; JP02076586-A; JP11137282-A; JP2004043660-A; JP2004313043-A; JP2008022828-A; JP2011234685-A; JP2016121081-A; JP2017079639-A; JP2022045888-A; US2004091474-A1; US2004197894-A1; US2004223962-A1; US2009087888-A1; US2012067373-A1; US2013123162-A1; US2015125925-A1; US2016032267-A1; US2019185788-A1; US2019185789-A1; US2020306342-A1; US2021122997-A1; US2022098524-A1; U.S. Pat. No. 4,980,288-A; U.S. Pat. No. 5,260,207-A; U.S. Pat. No. 5,316,935-A; U.S. Pat. No. 5,316,941-A; U.S. Pat. No. 5,371,008-A; U.S. Pat. No. 5,472,855-A; U.S. Pat. No. 5,652,136-A; U.S. Pat. No. 5,677,272-A; U.S. Pat. No. 5,679,630-A; U.S. Pat. No. 5,719,021-A; U.S. Pat. No. 6,017,871-A; U.S. Pat. No. 6,271,012-1; U.S. Pat. No. 6,312,936-B1; U.S. Pat. No. 6,440,717-B1; WO200022103-A1; WO200024924-A2; WO200037599-A1; WO200037621-A1; WO200037622-A1; WO200037623-A1; WO200037624-A1; WO200037625-A1; WO200037626-A1; WO200037627-A1; WO200071683-A1; WO200071684-A1; WO200071685-A1; WO200071686-A1; WO200071687-A1; WO200071688-A1; WO200071689-A1; WO200071690-A1; WO200071691-A1; WO200116285-A2; WO200168821-A2; WO200175087-A2; WO200183559-A2; WO200210183-A1; WO200216547-A2; WO200218588-A1; WO200222796-A1; WO200231133-A1; WO200240997-A2; WO200277289-A1; WO200288340-A2; WO2003038082-A2; WO2003054184-A1; WO2003054185-A1; WO2003055974-A2; WO2003057713-A2; WO2003062381-A2; WO2003093453-A2; WO2004003186-A2; WO2004016752-A2; WO2004042049-A1; WO2004064744-A2; WO2004099401-A1; WO2004111219-A1; WO2005024002-A1; WO2005035747-A1; WO2005078074-A2; WO2005079826-A1; WO2005095592-A2; WO2005115445-A1; WO2005118793-A2; WO2005123911-A2; WO2005123914-A1; WO2005124012-A1; WO2006088325-A1; WO2006136159-A2; WO2006136160-A2; WO2007006305-A1; WO2007019858-A2; WO2007044993-A2; WO2007080197-A2; WO2007122175-A1; WO2008010925-A2; WO2008112258-A2; WO2008141281-A1; WO02008153934-A2; WO2008153935-A2; WO2009005647-A2; WO2009021867-A2; WO2009058518-A1; WO2009071550-A1; WO2009074650-A2; WO2009149144-A2; WO2009149145-A2; WO2009149200-A2; WO2010056640-A2; WO2010056653-A2; WO2010056671-A1; WO2010078462-A1; WO2010123754-A1; WO2011014278-A1; WO2011036263-A1; WO2011072099-A2; WO2011072117-A1; WO2011091370-A1; WO2011110625-A1; WO2011130076-A1; WO2011130222-A2; WO2011140316-A1; WO2011140364-A1; WO2012080202-A1; WO2012151480-A2; WO2012151534-A1; WO2013086219-A1; WO2013092635-A1; WO2013110766-A1; WO2013159032-A1; WO2014037438-A1; WO2014055778-A2; WO2014055782-A1; WO2014081884-A1; WO2014194034-A2; WO2014207228-A1; WO2015038792-A1; WO2015048332-A2; WO2015048339-A2; WO2015065871-A1; WO2015089441-A1; WO2015089447-A1; WO2015143360-A2; WO2015144932-A1; WO2015144936-A1; WO2015185689-A1; WO2016040464-A1; WO2016046234-A2; WO2016069552-A1; WO2016069557-A1; WO2016069563-A1; WO2016069569-A2; WO2016097405-A1; WO2016145428-A1; WO2016164096-A1; WO2016174234-A2; WO2016180928-A1; WO2016183509-A1; WO2016203064-A2; WO2016205710-A1; WO2016205755-A1; WO2017006266-A1; WO2017050291-A1; WO2017081274-A1; WO2017089093-A1; WO2017089366-A1; WO2017089456-A1; WO2017106676-A1; WO2017177153-A1; WO2017189720-A1; WO2017192692-A1; WO2017210295-A1; WO2017219011-A1; WO2018015303-A1; WO2018015304-A1; WO2018118917-A1; WO2018118950-A1; WO2018161899-A1; WO2018188667-A1; WO2018222990-A1; WO2019048486-A1; WO2019048495-A1; WO2019108599-A1; WO2019180111-A1; WO2019238761-A1; WO2019245704-A1; WO2019245705-A1; WO2019245838-A1; WO2019245839-A1; WO2020002255-A1; WO2020007863-A1; WO2020023411-A1; WO2020076697-A1; WO2020112599-A1; WO2020114968-A1; WO2020156419-A1; WO2020169564-A1; WO2020176443-A1; WO2020188095-A1; WO2020200198-A1; WO2020201403-A1; WO2020207944-A1; WO2020243738-A1; WO2021013685-A1; WO2021013686-A1; WO2021025872-A1; WO2021030400-A1; WO2021080948-A2; WO2021119304-A1; WO2021122120-A2; WO2021130167-A1; WO2021133701-A1; WO2021148364-A1; WO2021209548-A1; WO2021230359-A1; WO2021231621-A1; WO2021231623-A1; WO2021248045-A2; WO2022043547-A1; WO2022043563-A1; WO2022043745-A1; WO2022106400-A1; WO2022129166-A1; WO2022171120-A1; WO2022171667-A1; WO2022175263-A2; WO2022225696-A2; WO2022226158-A2; WO2022261003-A1; WO2023288294-A1; WO8807581-A; WO9102792-A; WO9106637-A; WO9211348-A1; WO9211357-A1; WO9221760-A1; WO9307276-A1; WO9402618-A1; WO9418329-A2; WO9423053-A1; WO9507991-A2; WO9510591-A1; WO9510615-A1; WO9523221-A1; WO9523614-A1; WO9530011-A2; WO9628557-A2; WO9628566-A2; WO9634935-A2; WO9634946-A1; WO9820115-A1; WO9830682-A1; WO9855634-A1; WO9920770-A2; WO9927082-A1; WO9967370-A1.

In some embodiments, proteins of the present disclosure exhibit one or more hydrolase activities belonging to EC 3.5, such as amidohydrolase and amidase activities belonging to EC 3.5.1 (e.g., asparaginase activity belong to EC 3.5.1.1, glutaminase activity belonging to 3.5.1.2, amidase activity belonging to 3.5.1.4, urease activity belonging to 3.5.1.5, biotinidase activity belonging to 3.5.1.12, nicotinamidase activity belonging to 3.5.1.19, N-acetylglucosamine deacetylase activity belonging to EC 3.5.1.33, D-glutaminase activity belonging to 3.5.1.35, glutamin-(asparagin-)ase activity belonging to 3.5.1.38, chitin deacetylase activity belonging to 3.5.1.41, peptidyl-glutaminase activity belonging to 3.5.1.43, protein-glutamine glutaminase activity belonging to 3.5.1.44, pentanamidase activity belonging to 3.5.1.50, peptidoglycan-N-acetylglucosamine deacetylase activity belonging to EC 3.5.1.104) aminidase and deiminase activities belonging to EC 3.5.3 (e.g., arginase activity belonging to EC 3.5.3.1, arginine deiminase activity belonging to EC 3.5.3.6, D-arginase activity belonging to EC 3.5.3.10, protein-arginine deiminase activity belonging to EC 3.5.3.15), and/or deaminase activities belonging to EC 3.5.4 (e.g., cytosine deaminase activity belonging to EC 3.5.4.1, adenine deaminase activity belonging to EC 3.5.4.2, guanine deaminase activity belonging to EC 3.5.4.3, adenosine deaminase activity belonging to EC 3.5.4.4), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 89-90.

In some embodiments, the protein is selected from the group consisting of
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 89-90;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 89-90;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 180-181 or the cDNA sequence thereof;
d) a polypeptide derived from any one of SEQ ID NO(s): 89-90 by substitution, deletion, or insertion of one or more amino acids;
e) a polypeptide derived from a mature polypeptide of any one of SEQ ID NO(s): 89-90 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has asparaginase activity belonging to EC 3.5.1.1. Examples of proteins that exhibit asparaginase activity and have an amino acid sequence that is at least 70% identical to one or more of SEQ ID NOs: 89-90 are set forth herein as SEQ ID NOs: 71904-72141.

In some embodiments, the protein is derived from Aspergillus. For example, in some embodiments, the protein is a native Aspergillus asparaginase or is a fragment/mutant/variant of a native Aspergillus asparaginase.

Those skilled in the art will understand how to identify, isolate, characterize, produce, recover and formulate proteins having one or more hydrolase activities belong to EC 3.5. See, e.g., CN109486689-A; EP2295582-A2; U.S. Pat. No. 7,504,490-B1; WO2004032648-A1; WO2008110513-A1; WO2008128974-A1; WO2008128975-A1; WO2011134916-A1; WO2012068047-A2; WO2014027062-A1; WO2014027063-A1; WO2014147189-A1.

In some embodiments, proteins of the present disclosure exhibit one or more lyase activities belonging to Enzyme Commission classification number 4 (EC 4). For example, in some embodiments, proteins of the present disclosure exhibit lyase activity useful for a) preventing/treating/suppressing/eliminat-ing/reducing the detrimental effects of infestations/infections of/by various pests, including, but not limited to, phytopathogenic pests, such as acarids, bacteria, fungi, gastropods, insects, nematodes, oomycetes, protozoa, viruses and weeds; b) reducing one or more aspects of disease severity in plants affected by one or more phytopathogenic pests; c) pretreating surfaces/substances that are susceptible to infestation/infection by pests; d) cleaning surfaces/substances that are infested/infected by pests; e) enhancing the environments in which plants are grown by; f) improving nutrient availability in plant growth media; g) reducing the amounts of exogenous fertilizer needed to achieve a desired result; h) improving plant growth, development, and yield characteristics; i) prolonging the shelf-life of harvested plants and plant parts; j) delaying/hastening the ripening of a plant or plant part; k) improving the efficacy of chemical pesticides; and/or l) reducing chemical-pesticide-induced resistance/phytotoxicity.

In some embodiments, proteins of the present disclosure exhibit one or more carbon-carbon lyase activities belonging to EC 4.1, such as carboxy-lyase activities belonging to EC 4.1.1 (e.g., aspartate 1-decarboxylase activity belonging to EC 4.1.1.11, aspartate 4-decarboxylase activity belonging to EC 4.1.1.12, valine decarboxylase activity belonging to EC 4.1.1.14, glutamate decarboxylase activity belonging to EC 4.1.1.15, lysine decarboxylase activity belonging to EC 4.1.1.18, arginine decarboxylase activity belonging to EC 4.1.1.19, histidine decarboxylase activity belonging to EC 4.1.1.22, tyrosine decarboxylase activity belonging to EC 4.1.1.25, phenylalanine decarboxylase activity belonging to EC 4.1.1.53, methionine decarboxylase activity belonging to EC 4.1.1.57, L-tryptophan decarboxylase activity belonging to EC 4.1.1.105) and/or carbon-carbon lyase activities belonging to EC 4.1.99 (e.g., tryptophanase activity belonging to EC 4.1.99.1, tyrosine phenol-lyase activity belonging to EC 4.1.99.2).

Those skilled in the art will understand how to identify, isolate, characterize, produce, recover and formulate proteins having one or more carbon-carbon lyase activities belong to EC 4.1.

In some embodiments, proteins of the present disclosure exhibit one or more carbon-oxygen lyase activities belonging to EC 4.2, such as polysaccharide lyase activities belonging to EC 4.2.2 (e.g., pectate lyase activity belong to EC 4.2.2.2, pectin lyase activity belonging to EC 4.2.2.10, glucan lyase activity belonging to EC 4.2.2.13, gellan lyase activity belonging to EC 4.2.2.25, oligo-alginase lyase activity belonging to EC 4.2.2.26, pectin monosaccharide-lyase activity belonging to EC 4.2.2.27), and, optionally, comprise, consist essentially of, or consist of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to the amino acid sequence set forth herein as SEQ ID NO(s): 91 and 55.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 91;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 91;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 182 or the cDNA sequence thereof,
d) a polypeptide derived from any one of SEQ ID NO(s): 91 by substitution, deletion, or insertion of one or more amino acids;
e) a polypeptide derived from a mature polypeptide of any one of SEQ ID NO(s): 91 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has pectate lyase activity belonging to EC 4.2.2.2. Examples of proteins that exhibit pectate lyase activity and have an amino acid sequence that is at least 70% identical to one or more of SEQ ID NOs: 91 are set forth herein as SEQ ID NOs: 72142-72143.

In some embodiments, the protein is derived from Bacillus. For example, in some embodiments, the protein is a native Bacillus pectate lyase or is a fragment/mutant/variant of a native Bacillus pectate lyase.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 55;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 55;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 146 or the cDNA sequence thereof;
d) a polypeptide derived from any one of SEQ ID NO(s): 55 by substitution, deletion, or insertion of one or more amino acids;
e) a polypeptide derived from a mature polypeptide of any one of SEQ ID NO(s): 55 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide has pectin lyase activity belonging to EC 4.2.2.10. Examples of proteins that exhibit pectin lyase activity and have an amino acid sequence that is at least 70% identical to one or more of SEQ ID NOs: 55 are set forth herein as SEQ ID NOs: 45409-45436.

In some embodiments, the protein is derived from Aspergillus, Botrytis, Neosartorya, Penicillium or Pseudomyllocerus. For example, in some embodiments, the protein is a native Aspergillus, Botrytis, Neosartorya, Penicillium or Pseudomyllocerus pectin lyase or is a fragment/mutant/variant of a native Aspergillus, Botrytis, Neosartorya, Penicillium or Pseudomyllocerus pectin lyase.

Those skilled in the art will understand how to identify, isolate, characterize, produce, recover and formulate proteins having one or more carbon-oxygen lyase activities belong to EC 4.2. See, e.g., US2015045535-A1; WO9927083-A1.

In some embodiments, proteins of the present disclosure exhibit one or more carbon-nitrogen lyase activities belonging to EC 4.3, such as ammonia-lyase activities belonging to EC 4.3.1 (e.g., aspartate ammonia-lyase activity belonging to EC 4.3.1.1, histidine ammonia-lyase activity belong to EC 4.3.1.3, L-serine ammonia-lyase activity belonging to EC 4.3.1.17, D-serine ammonia-lyase activity belonging to EC 4.3.1.18, threonine ammonia-lyase activity belonging to EC 4.3.1.19, tyrosine ammonia-lyase activity belonging to EC 4.3.1.23, phenylalanine ammonia-lyase activity belonging to EC 4.3.1.24, phenylalanine/tyrosine ammonia-lyase activity belonging to EC 4.3.1.25, L-lysine cyclodeaminase activity belonging to EC 4.3.1.28, L-tryptophan ammonia-lyase activity belonging to EC 4.3.1.31).

Those skilled in the art will understand how to identify, isolate, characterize, produce, recover and formulate proteins having one or more carbon-nitrogen lyase activities belong to EC 4.3.

In some embodiments, proteins of the present disclosure exhibit one or more carbon-sulfur lyase activities belonging to 4.4, such as carbon-sulfur lyase activities belonging to EC 4.4.1 (e.g., cysteine lyase activity belonging to EC 4.4.1.10, methionine gamma-lyase activity belonging to EC 4.4.1.11, L-cysteine desulfidase activity belonging to EC 4.4.1.28, L-cysteine beta-lyase activity belonging to EC 4.4.1.35).

Those skilled in the art will understand how to identify, isolate, characterize, produce, recover and formulate proteins having one or more carbon-sulfur lyase activities belong to EC 4.4.

In some embodiments, the protein comprises, consists essentially of, or consists of a wild-type polypeptide. For example, in some embodiments, the protein comprises, consists essentially of, or consists of a wild-type polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149 or a mature polypeptide thereof.

In some embodiments, the protein comprises, consists essentially of, or consists of a variant polypeptide. For example, in some embodiments, the protein comprises, consists essentially of, or consists of a variant polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149 or a mature polypeptide thereof.

In some embodiments, the protein comprises, consists essentially of or consists of a catalytic domain, a binding module and a linker between said catalytic domain and said binder module.

The present disclosure extends to proteins capable of exhibiting two, three, four, five or more distinct catalytic activities, including, but not limited to, proteins that inherently exhibit two or more distinct catalytic activities and fusion proteins comprising two or more polypeptides that exhibit distinct catalytic activities.

In some embodiments, the protein is a hybrid protein.

In some embodiments, the protein comprises two or more catalytic domains.

In some embodiments, the protein comprises two or more binding modules.

In some embodiments, the protein is a fusion protein (e.g., a fusion protein comprising a first polypeptide having a first enzymatic activity and a second polypeptide having a second enzymatic activity).

In some embodiments, the protein is a fusion protein comprising a first polypeptide and a second polypeptide, said second polypeptide being distinct from said first polypeptide, wherein said first polypeptide is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
d) a polypeptide derived from any one of SEQ ID NO(s): 1-91, 45906, 72144, 72147 and 72149 by substitution, deletion, or insertion of one or more amino acids;
e) a polypeptide derived from a mature polypeptide any one of SEQ ID NO(s): 1-91, 45906, 72144, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f), and, optionally, wherein said second polypeptide is selected from the group consisting of:
h) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
i) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
j) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
k) a polypeptide derived from any one of SEQ ID NO(s): 1-91, 45906, 72144, 72147 and 72149 by substitution, deletion, or insertion of one or more amino acids;
l) a polypeptide derived from a mature polypeptide any one of SEQ ID NO(s): 1-91, 45906, 72144, 72147 and 72149 by substitution, deletion, or insertion of one or more amino acids;
m) a polypeptide derived from the polypeptide of any one of h) through 1) wherein the N- and/or C-terminal end has been extended by the addition of one or more amino acids; and
n) a fragment of the polypeptide of any one of h) through m).

In preferred embodiments, proteins of the present disclosure comprise, consist essentially of or consist of the amino acid sequence of any one of SEQ ID NO(s): 1-91, 45906, 72144, 72147 and 72149 or a mature polypeptide thereof.

In preferred embodiments, proteins of the present disclosure are encoded by a polynucleotide that comprises, consists essentially of or consists of one of SEQ ID NO(s): 92-182, 72145-72146, 72148 and 72150 or the cDNA thereof.

In some embodiments, proteins of the present disclosure comprise, consist essentially of or consist of a fragment of one of SEQ ID NO(s): 1-91, 45906, 72144, 72147 and 72149 or a mature peptide thereof. For example, the protein may be a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

As noted above, in some embodiments, proteins of the present disclosure comprise, consist essentially of or consist of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are evaluated for catalytic activity to identify amino acid residues that are critical to the activity of the molecule. See also, Hilton et al., 1996, J Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Table 1 provides a non-exhaustive list of proteins useful in compositions and methods of the present disclosure.

TABLE 1
Exemplary proteins of the present disclosure

| | | Polypeptide | Polynucleotide |
|---|---|---|---|
| Enzyme Class | EC # | SEQ ID NO: | SEQ ID NO: |
| glucose oxidase | 1.1.3.4 | 1 | 92 |
| glucose oxidase | 1.1.3.4 | 2 | 93 |
| glucose oxidase | 1.1.3.4 | 3 | 94 |
| glucose oxidase | 1.1.3.4 | 4 | 95 |
| glucose oxidase | 1.1.3.4 | 5 | 96 |
| cellobiose oxidase | 1.1.99.18 | 6 | 97 |
| cellobiose oxidase | 1.1.99.18 | 7 | 98 |
| cellobiose oxidase | 1.1.99.18 | 8 | 99 |
| laccase | 1.10.3.2 | 9 | 100 |
| catalase | 1.11.1.6 | 10 | 101 |
| catalase | 1.11.1.6 | 11 | 102 |
| catalase | 1.11.1.6 | 12 | 103 |
| peroxidase | 1.11.1.7 | 13 | 104 |
| lytic cellulose monooxygenase | 1.14.99.56 | 14 | 105 |
| lytic cellulose monooxygenase | 1.14.99.56 | 15 | 106 |
| gamma-glutamyl transferase | 2.3.2.2 | 16 | 107 |
| triacylglycerol lipase | 3.1.1.3 | 17 | 108 |
| triacylglycerol lipase | 3.1.1.3 | 18 | 109 |
| triacylglycerol lipase | 3.1.1.3 | 19 | 110 |
| triacylglycerol lipase | 3.1.1.3 | 20 | 111 |
| triacylglycerol lipase | 3.1.1.3 | 21 | 112 |
| triacylglycerol lipase | 3.1.1.3 | 22 | 113 |
| triacylglycerol lipase | 3.1.1.3 | 23 | 114 |
| phospholipase A₁ | 3.1.1.32 | | |
| lysophospholipase | 3.1.1.5 | 24 | 115 |
| pectinesterase | 3.1.1.11 | 25 | 116 |
| phospholipase A₁ | 3.1.1.32 | 26 | 117 |
| cutinase | 3.1.1.74 | 27 | 118 |
| cutinase | 3.1.1.74 | 28 | 119 |
| cutinase | 3.1.1.74 | 29 | 120 |
| cutinase | 3.1.1.74 | 30 | 121 |
| cutinase | 3.1.1.74 | 31 | 122 |
| cutinase | 3.1.1.74 | 72147 | 72148 |
| cutinase | 3.1.1.74 | 72149 | 72150 |
| phospholipase C | 3.1.4.3 | 32 | 123 |
| phosphoinositide phospholipase C | 3.1.4.11 | 33 | 124 |
| alpha-amylase | 3.2.1.1 | 34 | 125 |
| alpha-amylase | 3.2.1.1 | 35 | 126 |
| alpha-amylase | 3.2.1.1 | 36 | 127 |
| alpha-amylase | 3.2.1.1 | 37 | 128 |
| alpha-amylase | 3.2.1.1 | 38 | 129 |
| alpha-amylase | 3.2.1.1 | 39 | 130 |
| alpha-amylase | 3.2.1.1 | 40 | 131 |
| glucan 1,4-alpha-glucosidase | 3.2.1.3 | 41 | 132 |
| glucan 1,4-alpha-glucosidase | 3.2.1.3 | 42 | 133 |
| cellulase | 3.2.1.4 | 43 | 134 |
| cellulase | 3.2.1.4 | 44 | 135 |
| cellulase | 3.2.1.4 | 45 | 136 |
| endo-1,3(4)-beta-glucanase | 3.2.1.6 | 46 | 137 |
| inulinase | 3.2.1.7 | 47 | 138 |
| endo-1,4-beta-xylanase | 3.2.1.8 | 48 | 139 |
| endo-1,4-beta-xylanase | 3.2.1.8 | 49 | 140 |
| endo-1,4-beta-xylanase | 3.2.1.8 | 50 | 141 |
| endo-1,4-beta-xylanase | 3.2.1.8 | 51 | 142 |
| endo-1,4-beta-xylanase | 3.2.1.8 | 52 | 143 |
| endo-1,4-beta-xylanase | 3.2.1.8 | 53 | 144 |
| dextranase | 3.2.1.11 | 54 | 145 |
| lysozyme | 3.2.1.17 | 56 | 147 |
| beta-glucosidase | 3.2.1.21 | 57 | 148 |
| alpha-mannosidase | 3.2.1.24 | 58 | 149 |
| glucan endo-1,3-beta-D-glucosidase | 3.2.1.39 | 59 | 150 |
| glucan endo-1,3-beta-D-glucosidase | 3.2.1.39 | 60 | 151 |
| glucan endo-1,3-beta-D-glucosidase | 3.2.1.39 | 45906 | 72146 |
| pullulanase | 3.2.1.41 | 61 | 152 |
| alpha-L-arabinofuranosidase | 3.2.1.55 | 62 | 153 |
| glucan endo-1,3-alpha-glucosidase | 3.2.1.59 | 63 | 154 |
| glucan endo-1,3-alpha-glucosidase | 3.2.1.59 | 64 | 155 |
| licheninase | 3.2.1.73 | 65 | 156 |
| glucan endo-1,6-beta-glucosidase | 3.2.1.75 | 66 | 157 |
| glucan endo-1,6-beta-glucosidase | 3.2.1.75 | 67 | 158 |
| mannan endo-1,4-beta-mannosidase | 3.2.1.78 | 68 | 159 |
| mannan endo-1,4-beta-mannosidase | 3.2.1.78 | 72144 | 72145 |
| mannan endo-1,6-alpha-mannosidase | 3.2.1.101 | 69 | 160 |
| endogalactosaminidase | 3.2.1.109 | 70 | 161 |
| endogalactosaminidase | 3.2.1.109 | 71 | 162 |
| endogalactosaminidase | 3.2.1.109 | 72 | 163 |
| endogalactosaminidase | 3.2.1.109 | 73 | 164 |
| endogalactosaminidase | 3.2.1.109 | 74 | 165 |
| endogalactosaminidase | 3.2.1.109 | 75 | 166 |
| glycoprotein endo-alpha-1,2-mannosidase | 3.2.1.130 | 76 | 167 |
| alpha-mannan endo-1,2-alpha-mannanase | 3.2.1.198 | | |
| glycoprotein endo-alpha-1,2-mannosidase | 3.2.1.130 | 77 | 168 |
| alpha-mannan endo-1,2-alpha-mannanase | 3.2.1.198 | | |
| glucan 1,4-alpha-maltohydrolasc | 3.2.1.133 | 78 | 169 |
| 1,6-alpha-D-mannosidase | 3.2.1.163 | 79 | 170 |
| serine endopeptidase | 3.4.21 | 80 | 171 |
| serine endopeptidase | 3.4.21 | 81 | 172 |
| glutamyl endopeptidase | 3.4.21.19 | 82 | 173 |
| subtilisin | 3.4.21.62 | 83 | 174 |
| subtilisin | 3.4.21.62 | 84 | 175 |
| subtilisin | 3.4.21.62 | 85 | 176 |
| subtilisin | 3.4.21.62 | 86 | 177 |
| subtilisin | 3.4.21.62 | 87 | 178 |
| bacillolycin | 3.4.24.28 | 88 | 179 |
| asparaginase | 3.5.1.1 | 89 | 180 |
| asparaginase | 3.5.1.1 | 90 | 181 |
| pectate lyase | 4.2.2.2 | 91 | 182 |
| pectin lyase | 4.2.2.10 | 55 | 146 |

Proteins of the present disclosure may be derived from microorganisms of any genus.

In some embodiments, the protein is derived from and/or obtained from a Gram-negative bacterium, such as Campylobacter, Chryseobacterium (e.g., C. viscerum), Dicytoglomus (e.g., D. thermophilum), Escherichia (e.g., E. coli), Flavobacterium, Fusobacterium, Helicobacter, Ilyobacter, Lysobacter (e.g., L. gummosus), Neisseria, Pseudomonas, Salmonella or Ureaplasma.

In some embodiments, the protein is derived from a Gram-positive bacterium, such as Alkalihalobacillus (e.g., A. akibai, A. clausii), Bacillus (e.g., B. agaradhaerens, B. alkalophilus, B. amyloliquefaciens, B. brevis, B. circulans, B. clausii, B. coagulans, B. deramificans, B. firmus, B. lautus, B. lentus, B. licheniformis, B. megaterium, B. pumilus, B. stearothermophilus, B. subtilis, B. thuringiensis), Clostridium, Effusibacillus (e.g., E. pohliae), Enterococcus, Geobacillus (e.g., G. stearothermophilus), Lactobacillus, Lactococcus, Lederbergia (e.g., L. lenta), Neobacillus (e.g., N. novalis), Nocardiopsis, Oceanobacillus (e.g., O. barbara), Staphylococcus, Streptococcus (e.g., S. equisimilis, S. pyogenes, S. uberis, and S. equi subsp. Zooepidemicus) or Streptomyces (e.g., S. achromogenes, S. avermitilis, S. coelicolor, S. griseus, S. lividans), Sutcliffiella (e.g., S. halmapala).

In some embodiments, the protein is derived from a fungus, such as Acremonium, Acrophialophora (e.g., A. fusispora), Aspergillus (e.g., A. aculeatus, A. awamori, A. chevalieri, A. foetidus, A. fumigatus, A. japonicus, A. nidulans, A. niger, A. niveoglaucus, A. oryzae, A. tubingensis), Aureobasidium, Bjerkandera (e.g., B. adusta, B. fumosa), Ceriporiopsis (e.g., C. aneirina, C. caregiea, C. gilvescens, C. pannocinta, C. rivulosa, Ceriporiopsis subrufa, C. subvermispora), Chaetomium (e.g., C. erraticum, C. globosum), Chrysosporium (e.g., C. inops, C. keratinophilum, C. lucknowense, C. merdarium, C. pannicola, C. queenslandicum, C. tropicum, C. zonatum), Colletotrichum (e.g., C. graminicola), Coprinopsis (e.g., C. cinereus), Coprinus (e.g., C. cinereus), Coriolus (e.g., C. hirsutus), Cryphonectria (e.g., C. parasitica), Cryptococcus, Evansstolkia (e.g., E. leycettana), Filibasidium, Fusarium (e.g., F. bactridioides. F. cerealis, F. crookwellense, F. culmorum, F. graminearum, F. graminum, F. heterosporum, F. longipes, F. negundi, F. oxysporum, F. reticulatum, F. roseum, F. sambucinum, F. sarcochroum, F. solani, F. sporotrichioides, F. sulphureum, F. torulosum, F. trichothecioides, F. venenatum), Humicola (e.g., H. insolens, H. lanuginosa), Magnaporthe, Microdochium (e.g., M. nivale), Mucor (e.g., M. miehei), Myceliophthora (e.g., M. thermophila), Neocallimastix, Neurospora (e.g., Neurospora crassa), Ostropa (e.g., O. barbara), Paecilomyces, Penicillium (e.g., P. emersonii, P. purpurogenum, P. thomii, P. viridicatum), Phanerochaete (e.g., P. chrysosporium), Phlebia (e.g., Phlebia radiata), Piromyces, Pleurotus (e.g., Pleurotus eryngii), Pseudoplectania (e.g., P. vogesiaca), Schizophyllum, Sodiomyces (e.g., S. alcalophilus), Stenocarpella (e.g., S. maydis), Talaromyces (e.g., T. bacillisporus, T. emersonii, T. pinophilus), Thermoascus (e.g., T. aurantiacus), Themochaetoides (e.g., T. thermophila), Thermomyces (e.g., T lanuginosus), Thermothielavioides (e.g., T. terrestris), Thermothelomyces (e.g., T. thermophilus), Thielavia (e.g., T. terrestris), Tolypocladium, Trametes (e.g., T. hirsuta, T villosa, T. versicolor), Trichoderma (e.g., T. atroviride, T. harzianum, T. koningii, T. longibrachiatum, T. reesei, T. viride), Trichophaea (e.g., T. saccata), or Urnula (e.g., U. criterium).

In some embodiments, the protein is derived from a yeast, such as Candida, Hansenula, Komagataella (e.g., K. phaffi.), Kluyveromyces (e.g., Kluyveromyces lactis), Pichia (e.g., P. pastoris), Saccharomyces (e.g., S. carlsbergensis, S. cerevisiae, S. diastaticus, S. douglasii, S. kluyveri, S. norbensis, S. oviformis), Schizosaccharomyces, or Yarrowia (e.g., Yarrowia lipolytica).

It will be understood that for the aforementioned species, the disclosure encompasses both the perfect and imperfect states, and other taxonomic equivalents, e.g., anamorphs, regardless of the species name by which they are known. Those skilled in the art will readily recognize the identity of appropriate equivalents.

The proteins may be identified and derived from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the protein may then be obtained by similarly screening a genomic DNA or cDNA library of another microorganism or mixed DNA sample. Once a polynucleotide encoding a protein has been detected with the probe(s), the polynucleotide can be isolated or cloned by utilizing techniques that are known to those of ordinary skill in the art (see, e.g., Davis et al., 2012, Basic Methods in Molecular Biology, Elsevier).

Proteins of the present disclosure may likewise be derived from plants of any genus.

In some embodiments, the protein is derived from a plant selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Caricaceae (e.g., papaya), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape). Proteins of the present disclosure may be produced by and obtained from using any suitable method(s), including, but not limited to, shake flask cultivation and large-scale fermentation (including continuous, batch, fed-batch, solid-state and/or microcarrier-based fermentation) methods.

The present disclosure extends to methods of producing a protein of the present disclosure, comprising (a) cultivating a cell, which in its wild-type form produces the protein, under conditions conducive for production of the protein; and optionally, (b) recovering the protein.

The present disclosure also extends to methods of producing a protein of the present disclosure, comprising (a) cultivating a recombinant host cell of the present disclosure under conditions conducive for production of the protein; and optionally, (b) recovering the protein.

In some embodiments, the protein is produced by and obtained from a Gram-negative bacterium, such as Campylobacter, Chryseobacterium (e.g., C. viscerum), Dicytoglomus (e.g., D. thermophilum), Escherichia (e.g., E. coli), Flavobacterium, Fusobacterium, Helicobacter, Ilyobacter, Lysobacter (e.g., L. gummosus), Neisseria, Pseudomonas, Salmonella or Ureaplasma.

In some embodiments, the protein is produced by and obtained from a Gram-positive bacterium, such as Alkalihalobacillus (e.g., A. akibai, A. clausii), Bacillus (e.g., B. agaradhaerens, B. alkalophilus, B. amyloliquefaciens, B. brevis, B. circulans, B. clausii, B. coagulans, B. deramifcans, B. firmus, B. lautus, B. lentus, B. licheniformis, B. megaterium, B. pumilus, B. stearothermophilus, B. subtilis, B. thuringiensis), Clostridium, Effusibacillus (e.g., E. pohliae), Enterococcus, Geobacillus (e.g., G. stearothermophlus), Lactobacillus, Lactococcus, Lederbergia (e.g., L. lenta), Neobacillus (e.g., N. novalis), Nocardiopsis, Oceanobacillus (e.g., O. barbara), Staphylococcus, Streptococcus (e.g., S. equisimilis, S. pyogenes, S. uberis, and S. equi subsp. Zooepidemicus) or Streptomyces (e.g., S. achromogenes, S. avermitilis, S. coelicolor, S. griseus, S. lividans), Sutcliffiella (e.g., S. halmapala).

In some embodiments, the protein is produced by and obtained from a fungal cell, such as Acremonium, Acrophialophora (e.g., A. fusispora), Aspergillus (e.g., A. aculeatus, A. awamori, A. chevalieri, A. foetidus, A. fumigatus, A. japonicus, A. nidulans, A. niger, A. niveoglaucus, A. oryzae, A. tubingensis), Aureobasidium, Bjerkandera (e.g., B. adusta, B. fumosa), Ceriporiopsis (e.g., C. aneirina, C. caregiea, C. gilvescens, C. pannocinta, C. rivulosa, Ceriporiopsis subrufa, C. subvermispora), Chaetomium (e.g., C. erraticum, C. globosum), Chrysosporium (e.g., C. inops, C. keratinophilum, C. lucknowense, C. merdarium, C. pannicola, C. queenslandicum, C. tropicum, C. zonatum), Colletotrichum (e.g., C. graminicola), Coprinopsis (e.g., C. cinereus), Coprinus (e.g., C. cinereus), Coriolus (e.g., C. hirsutus), Cryphonectria (e.g., C. parasitica), Cyptococcus, Evansstolkia (e.g., E. leycettana), Filibasidium, Fusarium (e.g., F. bactridioides, F. cerealis, F. crookwellense, F. culmorum, F. graminearum, F. graminum, F. heterosporum, F. longipes, F. negundi, F. oxysporum, F. reticulatum, F. roseum, F. sambucinum, F. sarcochroum, F. solani, F. sporotrichioides, F. sulphureum, F. torulosum, F. trichothecioides, F. venenatum), Humicola (e.g., H. insolens, H. lanuginosa), Magnaporthe, Microdochium (e.g., M. nivale), Mucor (e.g., M. miehei), Myceliophthora (e.g., M. thermophila), Neocallimastix, Neurospora (e.g., Neurospora crassa), Ostropa (e.g., O. barbara), Paecilomyces, Penicillium (e.g., P. emersonii, P. purpurogenum, P. thomii, P. viridicatum), Phanerochaete (e.g., P. chrysosporium), Phlebia (e.g., Phlebia radiata), Piromyces, Pleurotus (e.g., Pleurotus eryngii), Pseudoplectania (e.g., P. vogesiaca), Schizophyllum, Sodiomyces (e.g., S. alcalophilus), Stenocarpella (e.g., S. maydis), Talaromyces (e.g., T. bacillisporus, T. emersonii, T. pinophilus), Thermoascus (e.g., T. aurantiacus), Themochaetoides (e.g., T. thermophila), Thermomyces (e.g., T. lanuginosus), Thermothielavioides (e.g., T. terrestris), Thermothelomyces (e.g., T. thermophilus), Thielavia (e.g., T. terrestris), Tolypocladium, Trametes (e.g., T. hirsuta, T villosa, T. versicolor), Trichoderma (e.g., T. atroviride, T. harzianum, T. koningii, T. longibrachiatum, T reesei, T viride), Trichophaea (e.g., T. saccata), or Urnula (e.g., U. criterium).

In some embodiments, the protein is produced by and obtained from a yeast cell, such as Candida, Hansenula, Komagataella (e.g., K. phaffii), Kluyveromyces (e.g., K. lactis), Pichia (e.g., P. pastoris), Saccharomyces (e.g., S. carlsbergensis, S. cerevisiae, S. diastaticus, S. douglasii, S. kluyveri, S. norbensis, S. oviformis), Schizosaccharomyces, or Yarrowia (e.g., Y. lipolytica).

In some embodiments, the protein is produced by and obtained from a plant cell selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thahiana), Caricaceae (e.g., papaya), Cucurbitaceae (e.g., cantaloupe, cucumber, honeydew, melon, pumpkin, squash (e.g., a corn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

Cells may be cultivated in a nutrient medium suitable for production of the protein using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the protein to be expressed and/or isolated. Suitable media are available from commercial suppliers or may be prepared according to published compositions (e.g., in catalogues of the American Type Culture Collection). If the protein is secreted into the nutrient medium, the protein can be recovered directly from the medium. If the protein is not secreted, it can be recovered from cell lysates.

The protein may be detected using methods known in the art that are specific for the protein, including, but not limited to, the use of specific antibodies, formation of an enzyme product, disappearance of an enzyme substrate, or an assay determining the relative or specific activity of the protein.

The protein may be recovered from the medium using methods known in the art, including, but not limited to, collection, centrifugation, filtration, extraction, spray-drying, evaporation, or precipitation. In one aspect, a whole fermentation broth comprising the protein is recovered. In another aspect, a cell-free fermentation broth comprising the protein is recovered.

In some embodiments, the protein is secreted extracellularly.

In some embodiments, the protein is isolated.

In some embodiments, the protein is purified.

The protein may be purified by a variety of procedures known in the art to obtain substantially pure proteins and/or protein fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the protein is not recovered.

The present disclosure also provides cells that naturally express native proteins of the present disclosure and cells that have been engineered to express heterologous proteins of the present disclosure (e.g., recombinant host cells comprising a polynucleotide of the present disclosure operably linked to one or more control sequences that direct the production of a protein of the present disclosure), as well as tools and methods for producing such recombinant host cells, including polynucleotides encoding proteins of the present disclosure and nucleic acid constructs comprising such polynucleotides.

In some embodiments, the cell is a Gram-negative bacterium, such as Campylobacter, Chryseobacterium (e.g., C. viscerum), Dicytoglomus (e.g., D. thermophilum), Escherichia (e.g., E. coli), Flavobacterium, Fusobacterium, Helicobacter, Ilyobacter, Lysobacter (e.g., L. gummosus), Neisseria, Pseudomonas, Salmonella or Ureaplasma.

In some embodiments, the cell is a Gram-positive bacterium, such as Alkalihalobacillus (e.g., A. akibai, A. clausii), Bacillus (e.g., B. agaradhaerens, B. alkalophilus, B. amyloliquefaciens, B. brevis, B. circulans, B. clausii, B. coagulans, B. deramificans, B. firmus, B. lautus. B. lentus, B. licheniformis, B. megaterium, B. pumilus, B. stearothermophilus, B. subtilis, B. thuringiensis), Clostridium, Effusibacillus (e.g., E. pohliae), Enterococcus, Geobacillus (e.g., G. stearothermophilus), Lactobacillus, Lactococcus, Lederbergia (e.g., L. lenta), Neobacillus (e.g., N. novalis), Nocardiopsis, Oceanobacillus (e.g., O. barbara), Staphylococcus, Streptococcus (e.g., S. equisimilis, S. pyogenes, S. uberis, and S. equi subsp. Zooepidemicus) or Streptomyces (e.g., S. achromogenes, S. avermitilis, S. coelicolor, S. griseus, S. lividans), Sutcliffiella (e.g., S. halmapala).

In some embodiments, the cell is a fungal cell, such as Acremonium, Acrophialophora (e.g., A. fusispora), Aspergillus (e.g., A. aculeatus, A. awamori, A. chevalieri, A. foetidus, A. fumigatus, A. japonicus, A. nidulans, A. niger, A. niveoglaucus, A. oryzae, A. tubingensis), Aureobasidium, Bjerkandera (e.g., B. adusta, B. fumosa), Ceriporiopsis (e.g., C. aneirina, C. caregiea, C. gilvescens, C. pannocinta, C. rivulosa, Ceriporiopsis subrufa, C. subvermispora), Chaetomium (e.g., C. erraticum, C. globosum), Chrysosporium (e.g., C. inops, C. keratinophilum, C. lucknowense, C. merdarium, C. pannicola, C. queenslandicum, C. tropicum, C. zonatum), Colletotrichum (e.g., C. graminicola), Coprinopsis (e.g., C. cinereus), Coprinus (e.g., C. cinereus), Coriolus (e.g., C. hirsutus), Cryphonectria (e.g., C. parasitica), Cryptococcus, Evansstolkia (e.g., E. leycettana), Filibasidium, Fusarium (e.g., F. bactridioides, F. cerealis, F. crookwellense, F. culmorum, F. graminearum, F. graminum, F. heterosporum, F. longipes, F. negundi, F. oxysporum, F. reticulatum, F. roseum, F. sambucinum, F. sarcochroum, F. solani, F. sporotrichioides, F. sulphureum, F. torulosum, F. trichothecioides, F. venenatum), Humicola (e.g., H. insolens, H. lanuginosa), Magnaporthe, Microdochium (e.g., M. nivale), Mucor (e.g., M. miehei), Myceliophthora (e.g., M. thermophila), Neocallimastix, Neurospora (e.g., Neurospora crassa), Ostropa (e.g., O. barbara), Paecilomyces, Penicillium (e.g., P. emersonii, P. purpurogenum, P. thomii, P. viridicatum), Phanerochaete (e.g., P. chrysosporium), Phlebia (e.g., Phlebia radiata), Piromyces, Pleurotus (e.g., Pleurotus eryngii), Pseudoplectania (e.g., P. vogesiaca), Schizophyllum, Sodiomyces (e.g., S. alcalophilus), Stenocarpella (e.g., S. maydis), Talaromyces (e.g., T. bacillisporus, T. emersonii, T. pinophilus), Thermoascus (e.g., T. aurantiacus), Themochaetoides (e.g., T. thermophila), Thermomyces (e.g., T. lanuginosus), Thermothielavioides (e.g., T. terrestris), Thermothelomyces (e.g., T. thermophilus), Thielavia (e.g., T. terrestris), Tolypocladium, Trametes (e.g., T. hirsuta, T. villosa, T. versicolor), Trichoderma (e.g., T. atroviride, T. harzianum, T. koningii, T. longibrachiatum, T. reesei, T. viride), Trichophaea (e.g., T. saccata), or Urnula (e.g., U. criterium).

In some embodiments, the cell is a yeast cell, such as Candida, Hansenula, Komagataella (e.g., K. phaffi), Kluyveromyces (e.g., Kluyveromyces lactis), Pichia (e.g., P. pastoris), Saccharomyces (e.g., S. carlsbergensis, S. cerevisiae, S. diastaticus, S. douglasii, S. kluyveri, S. norbensis, S. oviformis), Schizosaccharomyces, or Yarrowia (e.g., Yarrowia lipolytica).

In some embodiments, the cell is a plant cell, optionally a plant cell selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Caricaceae (e.g., papaya), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

In some embodiments, the cell expresses:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149; c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
d) a polypeptide derived from any one of SEQ ID NO(s): 1-91, 45906, 72144, 72147 and 72149 by substitution, deletion, or insertion of one or more amino acids;
e) a polypeptide derived from a mature polypeptide any one of SEQ ID NO(s): 1-91, 45906, 72144, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and/or
g) a fragment of the polypeptide of any one of a) through f).

In preferred embodiments, the cell expresses a polypeptide that comprises, consists essentially of or consists of the amino acid sequence of any one of SEQ ID NO(s): 1-91, 45906, 72144, 72147 and 72149 or a mature polypeptide thereof.

In some embodiments, the cell expresses a polypeptide that comprises, consists essentially of or consists of a fragment of one of SEQ ID NO(s): 1-91, 45906, 72144, 72147 and 72149 or a mature peptide thereof. For example, the microorganism may express a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

In some embodiments, the cell expresses a polypeptide that comprises, consists essentially of or consists of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In some embodiments, the cell comprises a homologous or heterologous nucleic acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the nucleic acid sequences set forth herein as SEQ ID NO(s): 92-182, 72145-72146, 72148 and 72150 or the cDNA sequence thereof.

In preferred embodiments, the cell comprises a polynucleotide encodes a polypeptide that comprises, consists essentially of or consists of the amino acid sequence of any one of SEQ ID NO(s): 1-91, 45906, 72144, 72147 and 72149 or a mature polypeptide thereof.

In some embodiments, the cell comprises a polynucleotide that comprises, consists essentially of or consists of a nucleic acid sequence encoding a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149.

In some embodiments, the cell comprises a polynucleotide that comprises, consists essentially of or consists of a nucleic acid sequence encoding a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149.

In preferred embodiments, the cell comprises a polynucleotide that comprises, consists essentially of or consists of the nucleic acid sequence of any one of SEQ ID NO(s): 92-182, 72145-72146, 72148 and 72150 or a cDNA sequence thereof.

In some embodiments, the cell comprises a polynucleotide that encodes a polypeptide that comprises, consists essentially of or consists of a fragment of one of SEQ ID NO(s): 1-91, 45906, 72144, 72147 and 72149 or a mature peptide thereof. For example, the microorganism comprises a polynucleotide that encodes a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

In some embodiments, the cell comprises a polynucleotide that encodes a polypeptide that comprises, consists essentially of or consists of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

The present disclosure thus encompasses plants and plant parts expressing one or more proteins of the present disclosure, including plants and plant parts that have been engineered to (over)express one or more proteins of the present disclosure, as well as methods producing proteins of the present disclosure comprising cultivating a (transgenic) plant or a plant part comprising a polynucleotide that encodes the protein under conditions conducive for production of the protein, and, optionally, recovering the protein. In alternative embodiments, such plants may be used as is to enhance one or more food/feed characteristics (e.g., improve nutritional value, palatability and/or rheological properties, destroy an antinutritive factor, etc.).

In some embodiments, the plant expresses:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
d) a polypeptide derived from any one of SEQ ID NO(s): 1-91, 45906, 72144, 72147 and 72149 by substitution, deletion, or insertion of one or more amino acids;
e) a polypeptide derived from a mature polypeptide any one of SEQ ID NO(s): 1-91, 45906, 72144, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and/or
g) a fragment of the polypeptide of any one of a) through f).

In preferred embodiments, the plant expresses a polypeptide that comprises, consists essentially of or consists of the amino acid sequence of any one of SEQ ID NO(s): 1-91, 45906, 72144, 72147 and 72149 or a mature polypeptide thereof.

In some embodiments, the plant expresses a polypeptide that comprises, consists essentially of or consists of a fragment of one of SEQ ID NO(s): 1-91, 45906, 72144, 72147 and 72149 or a mature peptide thereof. For example, the microorganism may express a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

In some embodiments, the plant expresses a polypeptide that comprises, consists essentially of or consists of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In some embodiments, the plant comprises a homologous or heterologous nucleic acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the nucleic acid sequences set forth herein as SEQ ID NO(s): 92-182, 72145-72146, 72148 and 72150 or the cDNA sequence thereof.

In preferred embodiments, the plant comprises a polynucleotide encodes a polypeptide that comprises, consists essentially of or consists of the amino acid sequence of any one of SEQ ID NO(s): 1-91, 45906, 72144, 72147 and 72149 or a mature polypeptide thereof.

In some embodiments, the plant comprises a polynucleotide that comprises, consists essentially of or consists of a nucleic acid sequence encoding a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149.

In some embodiments, the plant comprises a polynucleotide that comprises, consists essentially of or consists of a nucleic acid sequence encoding a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149.

In preferred embodiments, the plant comprises a polynucleotide that comprises, consists essentially of or consists of the nucleic acid sequence of any one of SEQ ID NO(s): 92-182, 72145-72146, 72148 and 72150 or a cDNA sequence thereof.

In some embodiments, the plant comprises a polynucleotide that encodes a polypeptide that comprises, consists essentially of or consists of a fragment of one of SEQ ID NO(s): 1-91, 45906, 72144, 72147 and 72149 or a mature peptide thereof. For example, the microorganism comprises a polynucleotide that encodes a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

In some embodiments, the plant comprises a polynucleotide that encodes a polypeptide that comprises, consists essentially of or consists of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Also included within the scope of the present disclosure are the progeny of such plants, plant parts, and plant cells.

As noted above, the present disclosure extends to tools and methods for producing such recombinant host cells that express one or more proteins of the present disclosure, including polynucleotides encoding proteins of the present disclosure and nucleic acid constructs comprising such polynucleotides.

The present disclosure provides polynucleotides encoding proteins of the present disclosure, including, but not limited to, nucleic acid constructs and recombinant expression vectors that encode one or more enzymes of the present disclosure, as well as methods of producing such polynucleotides.

The polynucleotide may be a genomic DNA, a cDNA, a synthetic DNA, a synthetic RNA, a mRNA, or a combination thereof.

The polynucleotide may be cloned from any suitable genus, species or strain.

In some embodiments, the protein is cloned from a Gram-negative bacterium, such as Campylobacter, Chryseobacterium (e.g., C. viscerum), Dicytoglomus (e.g., D. thermophilum), Escherichia (e.g., E. coli), Flavobacterium, Fusobacterium, Helicobacter, Ilyobacter, Lysobacter (e.g., L. gummosus), Neisseria, Pseudomonas, Salmonella or Ureaplasma.

In some embodiments, the polynucleotide is cloned from a Gram-positive bacterium, such as Alkalihalobacillus (e.g., A. akibai, A. clausii), Bacillus (e.g., B. agaradhaerens, B. alkalophilus, B. amyloliquefaciens, B. brevis, B. circulans, B. clausii, B. coagulans, B. deramificans, B. firmus, B. lautus, B. lentus, B. licheniformis, B. megaterium, B. pumilus, B. stearothermophilus, B. subtilis, B. thuringiensis), Clostridium, Effusibacillus (e.g., E. pohliae), Enterococcus, Geobacillus (e.g., G. stearothermophilus), Lactobacillus, Lactococcus, Lederbergia (e.g., L. lenta), Neobacillus (e.g., N. novalis), Nocardiopsis, Oceanobacillus (e.g., O. barbara), Staphylococcus, Streptococcus (e.g., S. equisimilis, S. pyogenes, S. uberis, and S. equi subsp. Zooepidemicus) or Streptomyces (e.g., S. achromogenes, S. avermitilis, S. coelicolor, S. griseus, S. lividans), Sutcliffiella (e.g., S. halmapala).

In some embodiments, the polynucleotide is cloned from a fungus, such as Acremonium, Acrophialophora (e.g., A. fusispora), Aspergillus (e.g., A. aculeatus, A. awamori, A. chevalieri, A. foetidus, A. funigatus, A. japonicus, A. nidulans, A. niger, A. niveoglaucus, A. oryzae, A. tubingensis), Aureobasidium, Bjerkandera (e.g., B. adusta, B. fumosa), Ceriporiopsis (e.g., C. aneirina, C. caregiea, C. gilvescens, C. pannocinta, C. rivulosa, Ceriporiopsis subrufa, C. subvermispora), Chaetomium (e.g., C. erraticum, C. globosum), Chrysosporium (e.g., C. inops, C. keratinophilum, C. lucknowense, C. merdarium, C. pannicola, C. queenslandicum, C. tropicum, C. zonatum), Colletotrichum (e.g., C. graminicola), Coprinopsis (e.g., C. cinereus), Coprinus (e.g., C. cinereus), Coriolus (e.g., C. hirsutus), Cryphonectria (e.g., C. parasitica), Cryptococcus, Evansstolkia (e.g., F. leycettana), Filibasidium, Fusarium (e.g., F. bactridioides, F cerealis, F crookwellense, F. culmorum, F. graminearum, F. graminum, F. heterosporum, F. longipes, F. negundi, F. oxysporum, F. reticulatum, F. roseum, F. sambucinum, F. sarcochroum, F. solani, F. sporotrichioides, F. sulphureum, F. torulosum, F. trichothecioides, F. venenatum), Humicola (e.g., H. insolens, H. lanuginosa), Magnaporthe, Microdochium (e.g., M. nivale), Mucor (e.g., M. miehei), Mycehophthora (e.g., M. thermophila), Neocallimastix, Neurospora (e.g., Neurospora crassa), Ostropa (e.g., O. barbara), Paecilomyces, Penicillium (e.g., P. emersonii, P. purpurogenum, P. thomii, P. viridicatum), Phanerochaete (e.g., P. chrysosporium), Phlebia (e.g., Phlebia radiata), Piromyces, Pleurotus (e.g., Pleurotus eryngii), Pseudoplectania (e.g., P. vogesiaca), Schizophyllum, Sodiomyces (e.g., S. alcalophilus), Stenocarpella (e.g., S. maydis), Talaromyces (e.g., T. bacillisporus, T. emersonii, T. pinophilus), Thermoascus (e.g., T. aurantiacus), Themochaetoides (e.g., T. thermophila), Thermomyces (e.g., T. lanuginosus), Thermothielavioides (e.g., T. terrestris), Thermothelomyces (e.g., T. thermophilus), Thielavia (e.g., T. terrestris). Tolypocladium, Trametes (e.g., T. hirsuta, T villosa, T. versicolor), Trichoderma (e.g., T. atroviride, T. harzianum, T. koningii, T. longibrachiatum, T. reesei, T. viride), Trichophaea (e.g., T. saccata), or Urnula (e.g., U. criterium).

In some embodiments, the polynucleotide is cloned from a yeast, such as Candida, Hansenula, Komagataella (e.g., K. phaffii), Kluyveromyces (e.g., K. lactis), Pichia (e.g., P. pastoris), Saccharomyces (e.g., S. carlsbergensis, S. cerevisiae, S. diastaticus, S. douglasii, S. kluyveri, S norbensis, S. oviformis), Schizosaccharomyces, or Yarrowia (e.g., Y. lipolytica).

In some embodiments, the polynucleotide is cloned from a plant cell selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Caricaceae (e.g., papaya), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceac (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

In some embodiments, the polynucleotide encodes:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
c) a polypeptide derived from any one of SEQ ID NO(s): 1-91, 45906, 72144, 72147 and 72149 by substitution, deletion, or insertion of one or more amino acids;
e) a polypeptide derived from a mature polypeptide any one of SEQ ID NO(s): 1-91, 45906, 72144, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and/or
g) a fragment of the polypeptide of any one of a) through f).

In preferred embodiments, the polynucleotide encodes a polypeptide that comprises, consists essentially of or consists of the amino acid sequence of any one of SEQ ID NO(s): 1-91, 45906, 72144, 72147 and 72149 or a mature polypeptide thereof.

In some embodiments, the polynucleotide comprises, consists essentially of or consists of a nucleic acid sequence having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;

In preferred embodiments, the polynucleotide comprises, consists essentially of or consists of the nucleic acid sequence of any one of SEQ ID NO(s): 92-182, 72145-72146, 72148 and 72150 or a cDNA sequence thereof.

In some embodiments, the polynucleotide encodes a polypeptide that comprises, consists essentially of or consists of a fragment of one of SEQ ID NO(s): 1-91, 45906, 72144, 72147 and 72149 or a mature peptide thereof. For example, the polynucleotide may encode a fragment of any one of SEQ ID NO(s): 1-91, 45906, 72144, 72147 and 72149 or a mature peptide thereof, said fragment comprising at least 85, 86, 87, 88, 89, 90, 91, 92, 93, 94, 95, 96, 97, 98 or 99% of the amino acids found in the original protein.

In some embodiments, polynucleotides of the present disclosure encode a polypeptide that comprises, consists essentially of or consists of any one of SEQ ID NO(s): 1-91, 45906, 72144, 72147 and 72149 or a mature polypeptide thereof with an N-terminal extension of one or more amino acids (e.g., 1, 2, 3, 4, 5, 6, 7, 8, 9, 10, 11, 12, 13, 14, 15, 16, 17, 18, 19, 20 or more amino acids), a C-terminal extension of one or more amino acids (e.g., 1, 2, 3, 4, 5, 6, 7, 8, 9, 10, 11, 12, 13, 14, 15, 16, 17, 18, 19, 20 or more amino acids), one or more substitutions (e.g., 1, 2, 3, 4, 5, 6, 7, 8, 9, 10, 11, 12, 13, 14, 15, 16, 17, 18, 19, 20 or more substitutions), one or more insertions (e.g., 1, 2, 3, 4, 5, 6, 7, 8, 9, 10, 11, 12, 13, 14, 15, 16, 17, 18, 19, 20 or more insertions), and/or one or more deletions (e.g., 1, 2, 3, 4, 5, 6, 7, 8, 9, 10, 11, 12, 13, 14, 15, 16, 17, 18, 19, 20 or more deletions). The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Polynucleotides of the present disclosure may be mutated by introduction of nucleotide substitutions that do not result in a change in the amino acid sequence of the protein, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In an aspect, the polynucleotide is isolated.

In another aspect, the polynucleotide is purified.

The present disclosure also provides nucleic acid constructs comprising a polynucleotide of the present disclosure, wherein the polynucleotide is operably linked to one or more control sequences that direct the expression of the coding sequence in a suitable host cell under conditions compatible with the control sequences.

The polynucleotide may be manipulated in a variety of ways to provide for expression of the protein. Manipulation of the polynucleotide prior to its insertion into a vector may be desirable or necessary depending on the expression vector. Techniques for modifying polynucleotides utilizing recombinant DNA methods are well known in the art.

The control sequence may be a promoter, a polynucleotide that is recognized by a host cell for expression of a polynucleotide encoding a protein of the present disclosure. The promoter contains transcriptional control sequences that mediate the expression of the protein. The promoter may be any polynucleotide that shows transcriptional activity in the host cell including mutant, truncated, and hybrid promoters, and may be obtained from genes encoding extracellular or intracellular proteins either homologous or heterologous to the host cell.

Examples of suitable promoters for directing transcription of the polynucleotide of the present disclosure in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present disclosure in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

The control sequence may also be a transcription terminator, which is recognized by a host cell to terminate transcription. The terminator is operably linked to the 3′-terminus of the polynucleotide encoding the protein. Any terminator that is functional in the host cell may be used in the present disclosure.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8: 423-488.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cry/IIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

The control sequence may also be a leader, a non-translated region of an mRNA that is important for translation by the host cell. The leader is operably linked to the 5′-terminus of the polynucleotide encoding the protein. Any leader that is functional in the host cell may be used.

Suitable leaders for bacterial host cells are described by Hambraeus et al., 2000, Microbiology 146(12): 3051-3059, and by Kaberdin and Blasi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a protein and directs the protein into the cell's secretory pathway. The 5′-end of the coding sequence of the polynucleotide may inherently contain a signal peptide coding sequence naturally linked in translation reading frame with the segment of the coding sequence that encodes the protein. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the protein. Any signal peptide coding sequence that directs the expressed protein into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40: 949-955 Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a protein. The resultant protein is known as a proenzyme or proprotein (or a zymogen in some cases). A proprotein is generally inactive and can be converted to an active protein by catalytic or autocatalytic cleavage of the propeptide from the proprotein. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a protein and the signal peptide sequence is positioned next to the N-terminus of the propeptide sequence. Additionally or alternatively, when both signal peptide and propeptide sequences are present, the protein may comprise only a part of the signal peptide sequence and/or only a part of the propeptide sequence. Alternatively, the final or isolated protein may comprise a mixture of mature proteins and proteins which comprise, either partly or in full length, a propeptide sequence and/or a signal peptide sequence.

It may also be desirable to add regulatory sequences that regulate expression of the protein relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GALI system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

The control sequence may also be a transcription factor, a polynucleotide encoding a polynucleotide-specific DNA-binding protein that controls the rate of the transcription of genetic information from DNA to mRNA by binding to a specific polynucleotide sequence. The transcription factor may function alone and/or together with one or more other proteins or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

The present disclosure also provides recombinant expression vectors comprising a polynucleotide of the present disclosure, a promoter, and transcriptional and translational stop signals. The various nucleotide and control sequences may be joined together to produce a recombinant expression vector that may include one or more convenient restriction sites to allow for insertion or substitution of the polynucleotide encoding the protein at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the protein or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present disclosure may be inserted into a host cell to increase production of a protein. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The present disclosure also provides methods for producing recombinant host cells comprising a polynucleotide of the present disclosure operably linked to one or more control sequences that direct the production of a protein of the present disclosure.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the protein and its source. The protein can be native or heterologous to the recombinant host cell. Also, at least one of the one or more control sequences can be heterologous to the polynucleotide encoding the protein. The recombinant host cell may comprise a single copy, or at least two copies, e.g., three, four, five, or more copies of the polynucleotide of the present disclosure.

The host cell may be any cell useful in the recombinant production of a protein of the present disclosure, including, not limited to, prokaryotic cells, fungal cells and plant cells, as described above.

In an aspect, the host cell is isolated.

In another aspect, the host cell is purified.

In some embodiments, the host cell is a Gram-negative bacterium, such as Campylobacter, Chryseobacterium (e.g., C. viscerum), Dicytoglomus (e.g., D. thermophilum), Escherichia (e.g., E. coli), Flavobacterium, Fusobacterium, Helicobacter, Ilyobacter, Lysobacter (e.g., L. gummosus), Neisseria, Pseudomonas, Salmonella or Ureaplasma.

In some embodiments, the host cell is a Gram-positive bacterium, such as Alkalihalobacillus (e.g., A. akibai, A. clausii), Bacillus (e.g., B. agaradhaerens, B. alkalophilus, B. amyloliquefaciens, B. brevis, B. circulans, B. clausii, B. coagulans, B. deramificans, B. firmus, B. lautus, B. lentus, B. licheniformis, B. megaterium, B. pumilus, B. stearothermophilus, B. subtilis, B. thuringiensis), Clostridium. Effusibacillus (e.g., F. pohliae), Enterococcus, Geobacillus (e.g., G. stearothermophilus), Lactobacillus, Lactococcus, Lederbergia (e.g., L. lenta), Neobacillus (e.g., N. novalis), Nocardiopsis, Oceanobacillus (e.g., O. barbara), Staphylococcus, Streptococcus (e.g., S. equisimilis, S. pyogenes, S. uberis, and S. equi subsp. Zooepidemicus) or Streptomyces (e.g., S. achromogenes, S. avermitilis, S. coelicolor, S. griseus, S. lividans), Sutcliffiella (e.g., S. halmapala).

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

In some embodiments, the host cell is a fungal cell, such as Acremonium, Acrophialophora (e.g., A. fusispora), Aspergillus (e.g., A. aculeatus, A. awamori, A. chevalieri, A. foetidus, A. fumigatus, A. japonicus, A. nidulans, A. niger, A. niveoglaucus, A. oryzae, A. tubingensis), Aureobasidium, Bjerkandera (e.g., B. adusta, B. fumosa), Ceriporiopsis (e.g., C. aneirina, C. caregiea, C. gilvescens, C. pannocinta, C. rivulosa, Ceriporiopsis subrufa, C. subvermispora), Chaetomium (e.g., C. erraticum, C. globosum), Chrysosporium (e.g., C. inops, C. keratinophilum, C. lucknowense, C. merdarium, C. pannicola, C. queenslandicum, C. tropicum, C. zonatum), Colletotrichum (e.g., C. graminicola), Coprinopsis (e.g., C. cinereus), Coprinus (e.g., C. cinereus), Coriolus (e.g., C. hirsutus), Cryphonectria (e.g., C. parasitica), Cryptococcus, Evansstolkia (e.g., E. leycettana), Filibasidium, Fusarium (e.g., F. bactridioides, F. cerealis, F. crookwellense, F. culmorum, F. graminearum, F. graminum, F. heterosporum, F. longipes, F. negundi, F. oxysporum, F. reticulatum, F. roseum, F. sambucinum, F. sarcochroum, F. solani, F. sporotrichioides, F. sulphureum, F. torulosum, F. trichothecioides, F. venenatum), Humicola (e.g., H. insolens, H. lanuginosa), Magnaporthe, Microdochium (e.g., M. nivale), Mucor (e.g., M. miehei), Myceliophthora (e.g., M. thermophila), Neocallimastix, Neurospora (e.g., Neurospora crassa), Ostropa (e.g., O. barbara), Paecilomyces, Penicillium (e.g., P. emersonii, P. purpurogenum, P. thomii, P. viridicatum), Phanerochaete (e.g., P. chrysosporium), Phlebia (e.g., Phlebia radiata), Piromyces, Pleurotus (e.g., Pleurotus eryngii), Pseudoplectania (e.g., P. vogesiaca), Schizophyllum, Sodiomyces (e.g., S. alcalophilus), Stenocarpella (e.g., S. maydis), Talaromyces (e.g., T. bacillisporus, T. emersonii, T. pinophilus), Thermoascus (e.g., T. aurantiacus), Themochaetoides (e.g., T. thermophila), Thermomyces (e.g., T. lanuginosus), Thermothielavioides (e.g., T. terrestris), Thermothelomyces (e.g., T. thermophilus), Thielavia (e.g., T. terrestris), Tolypocladium, Trametes (e.g., T. hirsuta, T. villosa, T. versicolor), Trichoderma (e.g., T. atroviride, T. harzianum, T. koningii, T. longibrachiatum, T. reesei, T. viride), Trichophaea (e.g., T. saccata), or Urnula (e.g., U. criterium).

In some embodiments, the host cell is a yeast cell, such as Candida, Hansenula, Komagataella (e.g., K. phaffii), Kluyveromyces (e.g., Kluyveromyces lactis), Pichia (e.g., P. pastoris), Saccharomyces (e.g., S. carlsbergensis, S. cerevisiae, S. diastaticus, S. douglasii, S. kluyveri, S. norbensis, S. oviformis), Schizosaccharomyces, or Yarrowia (e.g., Yarrowia lipolytica).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16: 168 and procedures described in EP 238023, Yelton et al., 1984, Proc. Natl. Acad. Sci. USA 81: 1470-1474, Christensen et al., 1988, Bio Technology 6: 1419-1422, and Lubertozzi and Keasling, 2009, Biotechn. Advances 27: 53-75. However, any method known in the art for introducing DNA into a fungal host cell can be used, and the DNA can be introduced as linearized or as circular polynucleotide.

In some embodiments, the host cell is a plant cell, optionally a plant cell selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Caricaceae (e.g., papaya), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

Transgenic plants and plant cells expressing the protein may be constructed in accordance with methods known in the art. In short, the plant or plant cell is constructed by incorporating one or more expression constructs encoding the protein into the plant host genome or chloroplast genome and propagating the resulting modified plant or plant cell into a transgenic plant or plant cell. In an embodiment, a plant cell does not belong to plant varieties.

The expression construct is conveniently a nucleic acid construct that comprises a polynucleotide encoding a protein, wherein the polynucleotide is operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the protein is desired to be expressed (Sticklen, 2008, Nature Reviews 9: 433-443). For instance, the expression of the gene encoding a protein may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a protein in the plant. For instance, the promoter enhancer element may be an intron that is placed between the promoter and the polynucleotide encoding a protein. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site-specific excision of the selection gene by a specific recombinase.

In addition to direct transformation of a particular plant genotype with a construct of the present disclosure, transgenic plants may be made by crossing a plant having the construct to a second plant lacking the construct. For example, a construct encoding a protein can be introduced into a particular plant variety by crossing, without the need for ever directly transforming a plant of that given variety. Therefore, the present disclosure encompasses not only a plant directly regenerated from cells which have been transformed in accordance with the present disclosure, but also the progeny of such plants. As used herein, progeny may refer to the offspring of any generation of a parent plant prepared in accordance with the present disclosure. Such progeny may include a DNA construct prepared in accordance with the present disclosure. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the disclosure from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present disclosure encompasses methods of producing a mutant of a parent cell, which comprises disrupting or deleting a polynucleotide, or a portion thereof, encoding a protein of the present disclosure, which results in the mutant cell producing less of the protein than the parent cell when cultivated under the same conditions.

The mutant cell may be constructed by reducing or eliminating expression of the polynucleotide using methods well known in the art, for example, one or more nucleotide insertions, one or more gene disruptions, one or more nucleotide replacements, or one or more nucleotide deletions.

The polynucleotide to be modified or inactivated may be, for example, the coding region or a part thereof essential for activity, or a regulatory or control element required for expression of the coding region, e.g., a functional part of a promoter sequence, and/or a regulatory or control element required for the transcription or translation of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues (see J. L. Bose, Springer Protocols 2016, Methods in Molecular Biology, The Genetic Manipulation of Staphylococci).

Additionally or alternatively, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art, or by targeted gene editing using one or more nucleases, e.g., zinc-finger nucleases or CRISPR-associated nucleases. Additionally or alternatively, the modification or inactivation may be achieved by gene silencing, genetic repression, genetic activation, and/or post-translational mutagenesis, e.g., by methods employing non-coding RNA, RNAi, siRNA, miRNA, ribozymes, catalytically inactive nucleases, CRISPRi, nucleotide methylation, and/or histone acetylation. The modification may be transient and/or reversible, irreversible and/or stable, or the modification may be dependent on chemical inducers or dependent on cultivation conditions, such as the cultivation temperature.

The modification may be performed in vivo, i.e., directly on the cell expressing the polynucleotide to be modified, or the modification be performed in vitro.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present disclosure further relates to a mutant cell of a parent cell that comprises a disruption or deletion of a polynucleotide encoding a protein or a control sequence thereof or a silenced gene encoding the protein, which results in the mutant cell producing less of the protein or no protein compared to the parent cell.

Protein-deficient mutant cells are useful as host cells for expression of native and heterologous proteins. Therefore, the present disclosure further relates to methods of producing a native or heterologous protein, comprising (a) cultivating a protein-deficient mutant cell under conditions conducive for production of a desired protein (e.g., a protein of the present disclosure); and (b) recovering the desired protein. The term “heterologous proteins” means proteins that are not native to the host cell, e.g., a variant of a native protein. The host cell may comprise more than one copy of a polynucleotide encoding a desired native or heterologous protein.

In some embodiments, the present disclosure relates to a protein product essentially free from [enzyme] activity that is produced by a method of the present disclosure.

Proteins, polynucleotides and organisms of the present disclosure may be incorporated into any suitable formulation(s), including, but not limited to, formulations comprising one or more seed-compatible carriers, soil-compatible carriers, foliar-compatible carriers, preharvest carriers, and/or postharvest carriers. Selection of appropriate carrier materials will depend on the intended application(s) and the protein(s) to be included in the formulation, as well as any other components that may be present in and/or added to the formulation. In some embodiments, the carrier is a liquid, a gel, a slurry, or a solid. In some embodiments, the carrier consists essentially of or consists of one or more protein-stabilizing compounds.

The present disclosure encompasses granules/particles comprising one or more proteins of the disclosure. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In an embodiment, the core comprises one or more proteins of the present disclosure.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the protein absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 m thick, particularly at least 0.5 μm, at least 1 μm or at least 5 μm. In some embodiments, the thickness of the coating is below 100 m, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 m.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HiPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,

(a) Spray dried products, wherein a liquid protein-containing solution is atomized in a spray drying tower to form small droplets which during their way down the drying tower dry to form a protein-containing particulate material. Exceedingly small particles can be produced this way (Michael S. Showell (editor); Powdered detergents; Surfactant Science Series, 1998; Vol. 71; pages 140-142; Marcel Dekker).

(b) Layered products, wherein the protein is coated as a layer around a pre-formed inert core particle, wherein a protein-containing solution is atomized, typically in a fluid bed apparatus wherein the pre-formed core particles are fluidized, and the protein-containing solution adheres to the core particles and dries up to leave a layer of dry protein on the surface of the core particle. Particles of a desired size can be obtained this way if a useful core particle of the desired size can be found. This type of product is described in, e.g., WO 97/23606.

(c) Absorbed core particles, wherein rather than coating the protein as a layer around the core, the protein is absorbed onto and/or into the surface of the core. Such a process is described in WO 97/39116.

(d) Extrusion or pelletized products, wherein a protein-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, extremely high extrusion pressures when using a small opening increase heat generation in the protein paste, which is harmful to the protein (Michael S. Showell (editor); Powdered detergents; Surfactant Science Series; 1998; Vol. 71; pages 140-142; Marcel Dekker).

(e) Prilled products, wherein a protein-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the protein is uniformly distributed throughout an inert material instead of being concentrated on its surface. U.S. Pat. Nos. 4,016,040 and 4,713,245 describe this technique.

(f) Mixer granulation products, wherein a protein-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the protein. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of protein, fillers and binders etc. are mixed with cellulose fibers to reinforce the particles to produce a so-called T-granulate. Reinforced particles, are more robust, and release less enzymatic dust.

(g) Size reduction, wherein the cores are produced by milling or crushing of larger particles, pellets, tablets, briquettes etc. containing the protein. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.

(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.

(i) The cores may be subjected to drying, such as in a fluid bed drier. Other known methods for drying granules in the feed or enzyme industry can be used by the skilled person. The drying preferably takes place at a product temperature of from 25 to 90° C. For some proteins, it is important the cores comprising the protein contain a low amount of water before coating with the salt. If water sensitive proteins are coated with a salt before excessive water is removed, the excessive water will be trapped within the core and may affect the activity of the protein negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of proteins by the use of co-granulates is disclosed in WO 2013/188331.

The present disclosure also relates to protected proteins prepared according to the method(s) disclosed in EP 238216.

The present disclosure likewise encompasses liquid formulations comprising one or more proteins of the disclosure. The formulation may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4—formylphenyl boronic acid).

In some embodiments, filler(s) or carrier material(s) are included to increase the volume of such formulation. Suitable filler or carrier materials include, but are not limited to, various salts of sulfate, carbonate and silicate as well as talc, clay and the like. Suitable filler or carrier materials for liquid formulation include, but are not limited to, water or low molecular weight primary and secondary alcohols including polyols and diols. Examples of such alcohols include, but are not limited to, methanol, ethanol, propanol and isopropanol. In some embodiments, the formulation contain from about 5% to about 90% of such materials.

In an aspect, the liquid formulation comprises 20-80% w/w of polyol. In one embodiment, the liquid formulation comprises 0.001-2% w/w preservative.

In another embodiment, the disclosure relates to liquid formulations comprising:
(A) 0.001-25% w/w of one or more proteins of the present disclosure;
(B) 20-80% w/w of polyol;
(C) optionally 0.001-2% w/w preservative; and
(D) water.

In another embodiment, the disclosure relates to liquid formulations comprising:
(A) 0.001-25% w/w one or more proteins of the present disclosure;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

It is to be understood that formulations of the present disclosure may comprise combinations of the enzymes, including, but not limited to combinations of enzymes expressly disclosed herein and combinations with enzymes that are not expressly disclosed herein.

In some embodiments, formulations of the present disclosure comprise 2, 3, 4, 5, 6, 7, 8, 9, 10 or more proteins of the present disclosure. For example, in some embodiments, formulations of the present disclosure comprise 2, 3, 4, 5, 6, 7, 8, 9, 10 or more of the polypeptides set forth herein as SEQ ID NO(s): 1-91, 45906, 72144, 72147 and 72149, a mature polypeptide thereof, or a functional fragment/mutant/variant thereof.

In some embodiments, formulations of the present disclosure comprise two or more distinct amylases, cellulases, glucanases, hemicellulases, lipases, mannanases, oxidases, pectinases, peptidases, proteases and/or xylanases.

In some embodiments, formulations of the present disclosure comprise at least one glucanase (e.g., an endo-1,3(4)-beta-glucanase, such as SEQ ID NO: 46 or a functional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one arabanase, at least one cellulase, at least one glucanase (e.g., an endo-1,3(4)-beta-glucanase, such as SEQ ID NO: 46 or afunctional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one glucanase (e.g., glucan 1,4-alpha-glucosidase), at least one lipase (e.g., a lysophospholipase, such as SEQ ID NO: 24 or a functional fragment/mutant/variant thereof) and at least one pullulanase (e.g., SEQ ID NO: 61 or a functional fragment/mutant/variant thereof).

In some embodiments, formulations of the present disclosure comprise at least one glucanase (e.g., an endo-1,3(4)-beta-glucanase, such as SEQ ID NO: 46 or a functional fragment/mutant/variant thereof) and at least one pectinase.

In some embodiments, formulations of the present disclosure comprise at least one catalase (e.g., any one of SEQ ID NOs: 11-12 or a functional fragment/mutant/variant thereof) and at least one glucose oxidase (e.g., any one of SEQ ID NOs: 1-5 or a functional fragment/mutant/variant thereof).

In some embodiments, formulations of the present disclosure comprise at least one amylase (e.g., any one of SEQ ID NOs: 34-40 or a functional fragment/mutant/variant thereof), at least one chitinase, and at least one glucanase (e.g., an endo-1,3(4)-beta-glucanase, such as SEQ ID NO: 46 or a functional fragment/mutant/variant thereof).

In some embodiments, formulations of the present disclosure comprise at least one amylase (e.g., any one of SEQ ID NOs: 34-40 or a functional fragment/mutant/variant thereof), at least one glucanase (e.g., an endo-1,3(4)-beta-glucanase, such as SEQ ID NO: 46 or a functional fragment/mutant/variant thereof), and at least one bacillolycin (e.g., SEQ ID NO: 88 or a functional fragment/mutant/variant thereof).

In some embodiments, formulations of the present disclosure comprise at least one cellulase (e.g., any one of SEQ ID NOs: 43-45 or a functional fragment/mutant/variant thereof), at least one glucanase (e.g., an endo-1,3(4)-beta-glucanase, such as SEQ ID NO: 46 or a functional fragment/mutant/variant thereof), at least one pectinase and at least one pectin lyase (e.g., SEQ ID NO: 88 or a functional fragment/mutant/variant thereof).

In some embodiments, formulations of the present disclosure comprise at least one glucanase (e.g., an endo-1,3(4)-beta-glucanase, such as SEQ ID NO: 46 or a functional fragment/mutant/variant thereof), at least one oxygenase (e.g., a lytic cellulose monooxygenase, such as any one of SEQ ID NOs: 14-15 or a functional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one cellulase(e.g., any one of SEQ ID NOs: 43-45 or a functional fragment/mutant/variant thereof), at least one furanosidase (e.g., an arabinofuranosidase, such as SEQ ID NO: 62 or a functional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one subtilisin (e.g., any one of SEQ ID NOs: 83-87 or a functional fragment/mutant/variant thereof), and at least one bacillolysin (e.g., SEQ ID NO: 88 or a functional fragment/mutant/variant thereof).

In some embodiments, formulations of the present disclosure comprise at least one catalase (e.g., any one of SEQ ID NOs: 11-12 or a functional fragment/mutant/variant thereof) and at least one peptidase (e.g., a bacillolysin, SEQ ID NO: 88 or a functional fragment/mutant/variant thereof; a serine endopeptidase, such as any one of SEQ ID NOs: 80-81 or a functional fragment/mutant/variant thereof).

In some embodiments, formulations of the present disclosure comprise at least one catalase (e.g., any one of SEQ ID NOs: 11-12 or a functional fragment/mutant/variant thereof), at least one glucanase (e.g., an endo-1,3(4)-beta-glucanase, such as SEQ ID NO: 46 or a functional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one peptidase (e.g., a bacillolysin, SEQ ID NO: 88 or a functional fragment/mutant/variant thereof; a serine endopeptidase, such as any one of SEQ ID NOs: 80-81 or a functional fragment/mutant/variant thereof), at least one glucanase (e.g., an endo-1,3(4)-beta-glucanase, such as SEQ ID NO: 46 or a functional fragment/mutant/variant thereof) and at least one xylanase (e.g., an endo-1,4-beta-xylanase, such as any one of SEQ ID NOs: 48-53 or a functional fragment/mutant/variant thereof).

In some embodiments, formulations of the present disclosure comprise at least one amylase (e.g., any one of SEQ ID NOs: 34-40 or a functional fragment/mutant/variant thereof), at least one glucanase (e.g., an endo-1,3(4)-beta-glucanase, such as SEQ ID NO: 46 or a functional fragment/mutant/variant thereof) and at least one peptidase (e.g., a bacillolysin, SEQ ID NO: 88 or a functional fragment/mutant/variant thereof; a serine endopeptidase, such as any one of SEQ ID NOs: 80-81 or a functional fragment/mutant/variant thereof).

In some embodiments, formulations of the present disclosure comprise a fermentation broth that comprises 2, 3, 4, 5, 6, 7, 8, 9, 10 or more enzymes.

In some embodiments, formulations of the present disclosure comprise a fermentation broth that comprises 2, 3, 4, 5, 6, 7, 8, 9, 10 or more proteins of the present disclosure (2, 3, 4, 5, 6, 7, 8, 9, 10 or more of SEQ ID NOs: 1-91, 45906, 72144, 72147 and 72149 or functional fragments/mutants/variants thereof).

Formulations of the present disclosure may comprise myriad components, including, but not limited to, adhesives (stickers), biological actives, chemical actives, dispersants (spreaders), drying agents, emulsifiers, nutrients, pest attractants and feeding stimulants, pH control components, postharvest treatments, preservatives, rain fasteners, rhealogical agents, safeners, stabilizers, UV protectants and/or wetting agents.

Examples of actives that may be included in formulations of the present disclosure include, but not are not limited to, acaracides and miticides (e.g., carvacrol, sanguinarine, azobenzene, benzoximate, benzyl benzoate, bromopropylate, chlorbenside, chlorfenethol, chlorfenson, chlorfensulphide, chlorobenzilate, chloropropylate, cyflumetofen, DDT, dicofol, diphenyl sulfone, dofenapyn, fenson, fentrifanil, fluorbenside, genit, hexachlorophene, phenproxide, proclonol, tetradifon, tetrasul, benomyl, carbanolate, carbaryl, carbofuran, methiocarb, metolcarb, promacyl, propoxur, aldicarb, butocarboxim, oxamyl, thiocarboxime, thiofanox, bifenazate, binapacryl, dinex, dinobuton, dinocap-4, dinocap-6, dinocton, dinopenton, dinosulfon, dinoterbon, DNOC, amitraz, chlordimeform, chloromebuform, formetanate, formparanate, medimeform, semiamitraz, afoxolaner, fluralaner, sarolaner, tetranactin 20 avermectin acaricides, abamectin, doramectin, eprinomectin, ivermectin, selamectin, milbemectin, milbemycin oxime, moxidectin, clofentezine, cyromazine, diflovidazin, dofenapyn, fluazuron, flubenzimine, flucycloxuron, flufenoxuron, hexythiazox, bromocyclen, camphechlor, DDT, dienochlor, endosulfan, lindane, chlorfenvinphos, crotoxyphos, dichlorvos, heptenophos, mevinphos, monocrotophos, naled, TEPP, tetrachlorvinphos, amidithion, amiton, azinphos-ethyl, azinphos-methyl, azothoate, benoxafos, bromophos, bromophos-ethyl, carbophenothion, chlorpyrifos, chlorthiophos, coumaphos, cyanthoate, demeton-O, demeton-S, demeton-O-methyl, demeton-S-methyl, demeton-S-methylsulphon, dialifos, diazinon, dimethoate, dioxathion, disulfoton, endothion, ethion, ethoate-methyl, formothion, malathion, mecarbam, methacrifos, omethoate, oxydeprofos, oxydisulfoton, parathion, phenkapton, phorate, phosalone, phosmet, phostin, phoxim, pirimiphos-methyl, prothidathion, prothoate, pyrimitate, quinalphos, quintiofos, sophamide, sulfotep, thiometon, triazophos, trifenofos, vamidothion, trichlorfon, isocarbophos, methamidophos, propetamphos, dimefox, mipafox, schradan, azocyclotin, cyhexatin, fenbutatin oxide, phostin, dichlofluanid, dialifos, phosmet, cyenopyrafen, fenpyroximate, pyflubumide, tebufenpyrad, acetoprole, fipronil, vaniliprole, acrinathrin, bifenthrin, brofluthrinate, cyhalothrin, alpha-cypermethrin, fenpropathrin, fenvalerate, flucythrinate, flumethrin, tau-fluvalinate, pemiethrin, halfenprox, pyrimidifen, chlorfenapyr, sanguinarine, chinomethionat, thioquinox, bifujunzhi, fluacrypyrim, flufenoxystrobin, pyriminostrobin, aramite, propargite, spirodiclofen, clofentezine, diflovidazin, flubenzimine, hexythiazox, fenothiocarb, chloromethiuron, diafenthiuron, acequinocyl. amidoflumet, arsenous oxide, clenpirin, closantel, crotamiton, cycloprate, cymiazole, disulfiram, etoxazole, fenazaflor, fenazaquin, fluenetil, mesulfen, MNAF, nifluridide, nikkomycins, pyridaben, sulfiram, sulfluramid, sulfur, thuringiensin, triarathene, and combinations thereof); fungicides (e.g., strobilurins, such as azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester and 2-(2-(3-(2,6-dichlorophenyl)-1-methyl-allylideneaminooxymethyl)-phenyl)-2-methoxyimino-N-methyl-acetamide; carboxamides, such as carboxanilides (e.g., benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyra-zole-4-carboxamide, N-(2-(1,3,3-trimethylbutyl)-phenyl)-1,3-dimethyl-5-fluoro-1H-pyrazole-4-carboxamide), carboxylic morpholides (e.g., dimethomorph, flumorph, pyrimorph), benzoic acid amides (e.g., flumetover, fluopicolide, fluopyram, zoxamide), carpropamid, dicyclomet, mandiproamid, oxytetracyclin, silthiofam and N-(6-methoxy-pyridin-3-yl) cyclopropanecarboxylic acid amide; azoles, such as triazoles (e.g., azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole) and imidazoles (e.g., cyazofamid, imazalil, pefurazoate, prochloraz, triflumizol); heterocyclic compounds, such as pyridines (e.g., fluazinam, pyrifenox (cf.D1b), 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine), pyrimidines (e.g., bupirimate, cyprodinil, diflumetorim, fenarimol, ferimzone, mepanipyrim, nitrapyrin, nuarimol, pyrimethanil), piperazines (e.g., triforine), pirroles (e.g., fenpiclonil, fludioxonil), morpholines (e.g., aldimorph, dodemorph, dodemorph-acetate, fenpropimorph, tridemorph), piperidines (e.g., fenpropidin), dicarboximides (e.g., fluoroimid, iprodione, procymidone, vinclozolin), non-aromatic 5-membered heterocycles (e.g., famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester), acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole and 5-chloro-7-(4-methylpiperidin-1-yl)-6-(2,4,6-trifluorophenyl)-[1,2,4]triazolo-[1,5-a]pyrimidine; benzimidazoles, such as carbendazim; and other active substances, such as guanidines (e.g., guanidine, dodine, dodine free base, guazatine, guazatine-acetate, iminoctadine), iminoctadine-triacetate and iminoctadine-tris(albesilate); antibiotics (e.g., kasugamycin, kasugamycin hydrochloride-hydrate, streptomycin, polyoxine and validamycin A); nitrophenyl derivates (e.g., binapacryl, dicloran, dinobuton, dinocap, nitrothal-isopropyl, tecnazen); organometal compounds (e.g., fentin salts, such as fentin-acetate, fentin chloride, fentin hydroxide); sulfur-containing heterocyclyl compounds (e.g., dithianon, isoprothiolane); organophosphorus compounds (e.g., edifenphos, fosctyl, fosctyl-aluminum, iprobenfos, phosphorus acid and its salts, pyrazophos, tolclofos-methyl); organochlorine compounds (e.g., chlorothalonil, dichlofluanid, dichlorophen, flusulfamide, hexachlorobenzene, pencycuron, pentachlorphenole and its salts, phthalide, quintozene, thiophanate-methyl, thiophanate, tolylfluanid, N-(4-chloro-2-nitro-phenyl)-N-ethyl-4-methyl-benzenesulfonamide), inorganic active substances (e.g., Bordeaux mixture, copper acetate, copper hydroxide, copper oxychloride, basic copper sulfate, phosphite salt, sulfur, zinc sulfate), natamycin, and combinations thereof); gastropodicides (e.g., methiocarb, metaldehyde, carbaryl, spinosad, copper sulfate in combination with lime, boric acid, iron phosphate, and combinations thereof); herbicides (e.g., 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluro, clethodim, clodinafop, clomazone, cyanazine, cycloxydim, cyhalofop, desmedipham, desmetryn, dicamba, diclofop, dimefuron, diuron, dithiopyr, fenoxaprop, fluazifop, fluazifop-P, fluometuron, flufenpyr-ethyl, flumiclorac-pentyl, flumioxazin, fluoroglycofen, fluthiacet-methyl, fomesafe, fomesafen, glyphosate, glufosinate, haloxyfop, hexazinone, imazamox, imazaquin, imazethapyr, ioxynil, isoproturon, isoxaflutole, lactofen, linuron, mecoprop, mecoprop-P, mesotrion, metamitron, metazochlor, methibenzuron, metolachlor (and S-metolachlor), metoxuron, metribuzin, monolinuron, oxadiargyl, oxadiazon, oxyfluorfen, phenmedipham, pretilachlor, profoxydim, prometon, prometry, propachlor, propanil, propaquizafop, propisochlor, pyraflufen-ethyl, pyrazon, pyrazolynate, pyrazoxyfen, pyridate, quizalofop, quizalofop-P (e.g., quizalofop-ethyl, quizalofop-P-ethyl, clodinafop-propargyl, cyhalofop-butyl, diclofop-methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins described in U.S. Pat. No. 7,989,393), thenylchlor, tralkoxydim, triclopyr, trietazine, tropramezone, salts and esters thereof; racemic mixtures and resolved isomers thereof and combinations thereof); and insecticides and nematicides (e.g., antibiotic insecticides such as allosamidin and thuringiensin; macrocyclic lactone insecticides such as spinosad, spinetoram, and other spinosyns including the 21-butenyl spinosyns and their derivatives; avermectin insecticides such as abamectin, doramectin, emamectin, eprinomectin, ivermectin and selamectin; milbemycin insecticides such as lepimectin, milbemectin, milbemycin oxime and moxidectin; arsenical insecticides such as calcium arsenate, copper acetoarsenite, copper arsenate, lead arsenate, potassium arsenite and sodium arsenite; other biological insecticides, plant incorporated protectant insecticides such as Cry1Ab, Cry1Ac, Cry1F, Cry1A.105, Cry2Ab2, Cry3A, mir Cry3A, Cry3Bb1, Cry34, Cry35, and VIP3A; botanical insecticides such as anabasine, azadirachtin, d-limonene, nicotine, pyrethrins, cinerins, cinerin I, cinerin II, jasmolin I, jasmolin II, pyrethrin I, pyrethrin II, quassia, rotenone, ryania and sabadilla; carbamate insecticides such as bendiocarb and carbaryl; benzofuranyl methylcarbamate insecticides such as benfuracarb, carbofuran, carbosulfan, decarbofuran and furathiocarb; dimethylcarbamate insecticides dimitan, dimetilan, hyquincarb and pirimicarb; oxime carbamate insecticides such as alanycarb, aldicarb, aldoxycarb, butocarboxim, butoxycarboxim, methomyl, nitrilacarb, oxamyl, tazimcarb, thiocarboxime, thiodicarb and thiofanox; phenyl methylcarbamate insecticides such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC and xylylcarb; dinitrophenol insecticides such as dinex, dinoprop, dinosam and DNOC; fluorine insecticides such as barium hexafluorosilicate, cryolite, sodium fluoride, sodium hexafluorosilicate and sulfluramid; formamidine insecticides such as amitraz, chlordimeform, formetanate and formparanate; fumigant insecticides such as acrylonitrile, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride and tetrachloroethane; inorganic insecticides such as borax, calcium polysulfide, copper oleate, mercurous chloride, potassium thiocyanate and sodium thiocyanate; chitin synthesis inhibitors such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron and triflumuron; juvenile hormone mimics such as epofenonane, fenoxycarb, hydroprene, kinoprene, methoprene, pyriproxyfen and triprene; juvenile hormones such as juvenile hormone I, juvenile hormone II and juvenile hormone III; moulting hormone agonists such as chromafenozide, halofenozide, methoxyfenozide and tebufenozide; moulting hormones such as alpha.-ecdysone and ecdysterone; moulting inhibitors such as diofenolan; precocenes such as precocene I, precocene II and precocene III; unclassified insect growth regulators such as dicyclanil; nereistoxin analogue insecticides such as bensultap, cartap, thiocyclam and thiosultap; nicotinoid insecticides such as flonicamid; nitroguanidine insecticides such as clothianidin, dinotefuran, imidacloprid and thiamethoxam; nitromethylene insecticides such as nitenpyram and nithiazine; pyridylmethylamine insecticides such as acetamiprid, imidacloprid, nitenpyram and thiacloprid; organochlorine insecticides such as bromo-DDT, camphechlor, DDT, pp′-DDT, ethyl-DDD, HCH, gamma-HCH, lindane, methoxychlor, pentachlorophenol and TDE; cyclodiene insecticides such as aldrin, bromocyclen, chlorbicyclen, chlordane, chlordecone, dieldrin, dilor, endosulfan, endrin, HEOD, heptachlor, HHDN, isobenzan, isodrin, kelevan and mirex; organophosphate insecticides such as bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP and tetrachlorvinphos; organothiophosphate insecticides such as dioxabenzofos, fosmethilan and phenthoate; aliphatic organothiophosphate insecticides such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, IPSP, isothioate, malathion, methacrifos, oxydemeton-methyl. oxydeprofos, oxydisulfoton, phorate, sulfotep, terbufos and thiometon; aliphatic amide organothiophosphate insecticides such as amidithion, cyanthoate, dimethoate, ethoate-methyl, formothion, mecarbam, omethoate, prothoate, sophamide and vamidothion; oxime organothiophosphate insecticides such as chlorphoxim, phoxim and phoxim-methyl; heterocyclic organothiophosphate insecticides such as azamethiphos, coumaphos, coumithoate, dioxathion, endothion, menazon, morphothion, phosalone, pyraclofos, pyridaphenthion and quinothion; benzothiopyran organothiophosphate insecticides such as dithicrofos and thicrofos; benzotriazine organothiophosphate insecticides such as azinphos-ethyl and azinphos-methyl; isoindole organothiophosphate insecticides such as dialifos and phosmet; isoxazole organothiophosphate insecticides such as isoxathion and zolaprofos; pyrazolopyrimidine organothiophosphate insecticides such as chlorprazophos and pyrazophos; pyridine organothiophosphate insecticides such as chlorpyrifos and chlorpyrifos-methyl; pyrimidinc organothiophosphate insecticides such as butathiofos, diazinon, etrimfos, lirimfos, pirimiphos-ethyl, pirimiphos-methyl, primidophos, pyrimitate and tebupirimfos; quinoxaline organothiophosphate insecticides such as quinalphos and quinalphos-methyl; thiadiazole organothiophosphate insecticides such as athidathion, lythidathion, methidathion and prothidathion; triazole organothiophosphate insecticides such as isazofos and triazophos; phenyl organothiophosphate insecticides such as azothoate, bromophos, bromophos-ethyl, carbophenothion, chlorthiophos, cyanophos, cythioate, dicapthon, dichlofenthion, etaphos, famphur, fenchlorphos, fenitrothion fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3 and trifenofos; phosphonate insecticides such as butonate and trichlorfon; phosphonothioate insecticides such as mecarphon; phenyl ethylphosphonothioate insecticides such as fonofos and trichloronat; phenyl phenylphosphonothioate insecticides such as cyanofenphos, EPN and leptophos; phosphoramidate insecticides such as crufomate, fenamiphos, fosthietan, imicyafos, mephosfolan, phosfolan and pirimetaphos; phosphoramidothioate insecticides such as acephate, isocarbophos, isofenphos, methamidophos and propetamphos; phosphorodiamide insecticides such as dimefox, mazidox, mipafox and schradan; oxadiazine insecticides such as indoxacarb; phthalimide insecticides such as dialifos, phosmet and tetramethrin; pyrazole insecticides such as acetoprole, ethiprole, fipronil, pyrafluprole, pyriprole, tebufenpyrad, tolfenpyrad and vaniliprole; pyrethroid ester insecticides such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, biopermethrin, cismethrin, tefluthrin, terallethrin, tetramethrin, tralomethrin and transfluthrin; pyrethroid ether insecticides such as etofenprox, flufenprox, halfenprox, protrifenbute and silafluofen; pyrimidinamine insecticides such as flufenerim and pyrimidifen; pyrrole insecticides such as chlorfenapyr; tetronic acid insecticides such as spirodiclofen, spiromesifen and spirotetramat; thiourea insecticides such as diafenthiuron; urea insecticides such as flucofuron and sulcofuron; and unclassified insecticides such as AKD-3088, chlorantraniliprole, closantel, crotamiton, cyflumetofen, E2Y45, EXD, fenazaflor, fenazaquin, fenoxacrim, fenpyroximate, FKI-1033, flubendiamide, HGW86, hydramethylnon, IKI-2002, isoprothiolane, malonoben, metaflumizone, metoxadiazone, nifluridide, NNI-9850, NNI-0101, pymetrozine, pyridaben, pyridalyl, pyrifluquinazon, Qcide, rafoxanide, Rynaxypyr™, SYJ-159, triarathene and triazamate, and combinations thereof).

Non-limiting examples of actives that may be incorporated into formulations of the present disclosure—or into which proteins and other compositions of the present disclosure may be incorporated—include, but are not limited to, commercial products sold under the tradenames ABACUS®, ACROBAT®, ACRONIS®, ADHERE®, ADMIRAL®, AGCELENCE®, AGMUSA®, ALLEGRO®, ALITE 27®, ALTREVIN®, AMP®, AMPLEXUS®, AMPLO®, ARMEZON®, ARESENAL®, ASSIST®, ATECTRA®, ATIVUM®, AUMENAX®, AURA®, BASAGRAN®, BELLIS®, BEYOND®, BLAVITY®, BLITZ®, BOMVORO®, BRIO®, CABRIO®, CARAMBA®, CADRE®, CANTUS®, CAPACITY®, CARAMBA®, CAURIFIX®, CEPTIVA®, CEYVA®, CHOPPER®, CLARITY®, CLEARFIELD®, CLEARPATH®, CLEARSOL®, COLLIS®, COMET®, CONTAIN®, CONVEY®, COPEO®, CREDENZ®, CUPRODUL®, CYCOCEL®, DASH®, DELAN®, DISTINCT®, DORMEX®, DUETT®, DURAVEL®, ENDURA®, ENGENIA®, ENTIGRIS®, EXTREME®, F 500®, FACET®, FASTAC®, FENDONA®, FIBERMAX®, FINALE®, FORUM®, GELFIX®, GESTUS®, GLYTOL®, GRANOURO®, GREEN LAWNGER®, HEADLINER, HEAT®, HERBADOX®, HI-LIGHT®, HICOAT®, HIDROCUP®, HISTICK®, ILEVO®, IMUNIT®, INITIUM®, INTERFIELD®, KIFIX®, KIXOR®, KUMULUS®, LACTOSILO®, LAWNGER®, LIBERTY®, LIBERTYLINK®, LIDERO®, LUPRO-GRAIN®, MEES®, MERIVONR, MUNEO®, NEALTA®, NEPAXIR®, NEWPATH®, NEXICOR®, NODULATOR®, NOMOLT®, OBVIUS®, ONDUTY®, ONLY®, OPERA®, OPTILL®, ORKESTRA®, ORQUESTAR, OUTLOOK®, PENDULUM®, PIRATE®, PIVOT®, PIX®, PLATEAU®, POAST®, POLYACER®, POLYRAM®, PONCHO®, PREMIS®, PRIAXOR®, PRISTINE®, PROVISIA®, PROVYSOL®, PROWL®, PURSUTI®, RAK®, RAPTOR®, REGENT®, RELENYA®, RELY®, RENESTRA®, REVYSOL®, REVYTEK®, RHIZO-FLO®, SEFINA®, SELTIMA®, SEPIRET®, SERIFEL®, SHARPEN®, SISTEMA®, SISTIVA®, SOYTECH®, SPHAEREX®, SPOT®, STAMINA®, STANDAK®, STATUS®, STORM®, STROBY®, SUNFIRE®, SYSTIVA®, TACAZOR, TAJ®, TERAXXA®, TREEVIX®, TUIT®, TUTOR®, TWINLINK®, VABORO®, VALEOS®, VARISTO®, VAULTC, VELTYMA®, VERDICT®, VERISMO®, VERSATILIS®, VERSYS®, VIVANDO®, VOTIVO®, XANTHION®, XEMIUM®, ZAMPOR®, ZIDUA® and ZYNION® from BASF (Ludwigshafen, Germany); CORVUS®, POWERMAX®, DELARO®, PROSARO®, BAYTHROID®, SIVANTO®, FINISH®, GINSTAR®, ACCELERON®, RAXIL®, AERIS®, EVERGOL®, TRILEX®, ALLEGIANCE®, BUTEO, EMESTO®, GAUCHOR and THIRAM® from Bayer Crop Science (Creve Coeur, MO. USA); AGREER, AGRIPHAGE™, AGSIL®, ANCORA, AZATIN®, BOTANIGARD®, BOTEGHA®, BUG-N-SLUGGO®, CARB-O-NATOR®, CRYMAX®, CUEVA®, CYD-X®, DEFGUARD®, DELIVER®, DES-X®, DOUBLE NICKEL®, FIREFIGHTER™, GEMSTAR®, GROTTO®, HOMEPLATE®, JAVELIN®, KALMOR®, KOCIDE®, LIFEGARD®, MADEXR, MELOCON®, MYCOTROL®, NEEMIX®, OSO™, PFR-97™, SEDUCE™, SIL-MATRIX®, SLUGGO®, SOILGARD®, THURICIDE®, TRIACT®, TRIATHLON® and TRILOGY® from Certis (Columbia, MD); ABUNDIT®, ACCENT®, AFFORIA®, APROACH®, BASIS®, BEXFOND®, BLACKHAWK®, CANOPY®, CINCH®, CLINHER®, CURTAIL®, CURZATE®, DELEGATE®, RAINSHIELD®, DITHANE®, FEXAPAN®, VAPORGRIP®, LANNATE®, TANOS®, DURANGO®, DMA®, ELEVORE®, EMBEDK, ENABLE®, ENLIST DUO®, ENLIST ONE®, ENLITE®, ENTRUST®, ENVIVE®, EVERPLEX®, FONTELIS®, FULTIME®, GOLDSKY®, GRANDSTAND®, GRANITE®, GRASP®, HEARKEN®, INDAR®, NXTGENC, INSTINCT®, INTREPID 2F®, INTREPID EDGE®, KERB®, KEYSTONE®, KYBER®, LEADOFF®, LOYANT®, MATRIX®, N-SERVE®, NOVIXID®, OPENSKYC, PERFECTMATCH®, PINDAR®, PIXXARO®, POWERFLEX®, QUELEX®, RADIANT®, RALLY®, REALM®, REBELEX®, RESICORE®, RESOLVE®, REVULIN®, REZUVANT®, RIDGEBACK®, SEQUOIA®, SIMPLICITY®, SONIC®, STARANE®, STEADFAST®, STINGER®, STRONGARM®, SUCCESS®, SURESTART®, SURPASS®, SURVEIL®, SYNCHRONY®, TARZEC®, TRANSFORM®, TRELLIS®, TRIVENCE®, UTRISHA®, VERTISAN®, VYDATE®, WIDEARMATCH®, WIDEMATCHR and ZEST® from Corteva Agroscience (Indianapolis, IN, USA); BIO-SAVE® from Decco U.S. Post-Harvest, Inc. (Monrovia, CA, USA); ACCUDO®, AFFINITYK, AGILITY®, AIM®, ALLY®, ALTACOR®, ANTHEM®, ATHENA®, AUTHORITY®, AVAUNT®, BELEAF®, BRIGADE®, CADET®, CAPTURE®, CARBINE®, COMMAND®, CORAGEN®, DISPLAY®, ELEVEST®, ETHOS®, EXIREL®, EXPRESS®, FINESSE®, FIRSTSHOT®, FURAGRO®, GLADIATOR®, HARMONY®, HERO®, LUCENTO®, MARVEL®, MUSTANG®, OBEY®, PANOFLEX®, PRESENCE®, PREVATHON®, QUARTZO®, RHYME®, ROVRAL®, SEAMAC®, SHARK®, SOLIDA®, SPARTAN®, STEWARD®, TEMITRY®, TERRAC®, TOPGUARD®, UPBEET®, VANTACOR®, VERIMARK®, XYWAY®, ZEUS® and ZIRONAR® from FMC Corporation (Philadelphia, PA, USA); PENTIA®, ABAMEX®, AGRI TIN®, CHAMP®, CHIPTOX®, GIN OUT®, KAISO®, MEPEX®, NUPRID®, RAPPORT®, TERMINATE®, THISTROL®, ULTRA FLOURISH®, GOAL®, GOALTENDER®, GRAPPLE®, TUSCANY®, CHAMPION++, AGRI-MYCIN®, PHOSTROL®, BLIGHTBAN®, CHEETAH®, MYCOSHIELD®, RITEWAY®, TAZER®, MYSTIC®, CUPROXAT® and TYPY® from Nufarm Limited (Victoria, Australia); BIOSPECTRA®, PACRITE®, EFOG®, SHIELD-BRITE®, FUNGAFLOR®, PENBOTEC®, and SOPP from Pace International (Wapato, WA, USA); ACTARA®, ACTELLIC®, ACTIGARD®, ACURON™, ADVION®, AFLAGUARD®, AGRIPRO®, ALTO®, ALUMNI®, AMISTAR®, APIRO®, APRON®, AVICTA®, AWARD®, AXIAL™, AXORIS®, BANNER®, BANVEL®, BARRICADE®, BEACON®, BICEP II MAGNUM®, BION®, BONZI®, BOUNDARY®, BOXER®, BRAVO®, C. C. BENOIST®, CADENCE®, CALARIS®, CALLISTO®, CAMIX®, CAPTORA®, CASPER®, CELEST®, CHAIRMAN®, CHESS®, CITATION®, CLARIVA®, COLZOR®, CRUISER®, CULTAR®, CURACON®, DACONIL®, DISCOVER®, DIVIDEND®, DUAL®, DUAXO®, DURIVO®, DYNASTY®, EDDUS®, ELATIS®, ELUMIS®, ENDEAVOR®, ENVOKE®, EPERON®, EPIVO®, ERIJAN®, FARMORE®, FLAGSHIP®, FLEX®, FLEXSTAR®, FLORIPRO SERVICES®, FOLIO®, FORCE®, FORTENZA®, FUSIFLEX®, FUSILADE®, GESAGARD®, GESAPAX®, GESAPRIM®, GOLD®, GOLDEN HARVESTC, GRADUATE®, GRADUATEA+®, GRAMOXONE®, HALEX®, HERITAGE®, HILLESHOG®, HORIZON®, HYVIDO®, INSEGARR, ISABION®, KARATE ZEON®, LENTAGRAN®, LISTEGO®, LOGRAN®, LUMAX®, MAAG®, MATCH®, MAXIM®, MAXX®, MENTOR®, MERTECT®, MILAGRO®, MINECTO®, MIRAVISR, MODDUS®, NEMATHORIN®, NK®, ORDRAM®, ORONDIS®, PALISADE®, PEAK®, PEGASUS®, PIRIMOR®, POLO®, PREFIX®, PRIMO®, PROCLAIM®, QUANTIS®, REFLECT®, REFLEX®, REGLONE®, RESOLVA®, REVUS®, RIFIT®, ROGERS®, S&G®, SAKALIA®, SALTRO®, SCHOLAR®, SCIMITAR®, SCORE®, SEGURIS®, SEQUESTRENE®, SETOFF®, SOFIT®, SOLVIGO®, STADIUM®, SUPREN®, SWITCH®, SYMETRA®, SYNGENTA®, TAEGRO®, TAVIUM®, TERVIGO®, TILT®, TIMOREX®, TOPIK®, TOPREX®, TRIGARD®, TRIMMIT®, TOUCHDOWN®, UNIX®, VAYANTIS®, VERTIMAC®, VIBRANCE®, WEATHER STIK®, from Syngenta Crop Protection (Basel, Switzerland); and ASULOX®, BALISTIK®, BEETUP®, BELLMAC®, BETASANA®, BETTIX®, BUGUIS®, CENTURION®, CLIOPHAR®, COLZAMID®, CORZAL®, DEFIANT®, DEVRINOL®, MINSTREL®, AFFIX®, AXIDOR®, BUZZ®, MIMIX®, DIOZINOS®, DIPROSPERO®, EVITO®, MANZATE®, MICROTHIOL®, NAUTILE®, PENNCOZEB®, PROMESS®, PROPLANT®, PROXANIL®, PYRUS®, SACRON®, SYLLIT®, TEBUZOL®, THIOPRON®, TOKYO®, UNIZEB®, VACCIPLANT®, VIDEO®, ZOXIS®, CYTHRIN®, DIMILIN®, FORESTER®, FUMICYP®, TALISMA®, B-NINE®, FAZOR®, GYRO®, HIMALAYA®, ICENI®, TRINEXIS®, IODUS®, AUDIT®, BASAGRAN®, BATLIUM®, BOYCOTT®, BROADLOOM®, COYOTE®, COLLIDE®, DUET®, ETHOTRON®, EVEREST®, IMIFLEX®, LIFELINE®, METRICOR®, MOCCASIN®, MOTIF®, PRE-PARE®, SATELLITE®, SHADOW®, SHUTDOWN®, STAM®, SUPERWHAM!®, SUPREMACY®, TRICOR®, TRIZENTA®, CUPROFIX®, DEXTER®, ELEVATE®, ELIXIR®, FORTIX®, FROGHORN®, METEOR®, MICROTHI®, ORANIL®, PH-D®, PROCURE®, RANCOVA®, TEPERA®, TERRAGUARD®, TERRAMASTER®, TERRAZOLE®, TOPSIN®, TRIONIC®, ZIRAM®, ZOLERA®, ADIOS®, GOLDWING®, OFF-SHOOT-T®, PACZOL®, ROYAL®, ROYALTAC®, ACENTHRIN®, ACEPHATE®, ACRAMITE®, ADEPT®, ARGYLE®, ASSAIL®, BANTER®, BIFENTURE®, BIOMITE®, COMITE®, DIMILIN®, ENKOUNTER®, INTRUDER®, KANEMITE®, LAMBDA-CY®, MICROMITE®, OMITE®, PEDESTAL®, PERM-UP®, RIMON®, STRAFER®, TURNSTYLE®, UP-CYDE®, VENDEX®, VIGILANT®, ZYLO®, ATTENDANT®, BEAN GUARD®, ALLEGIANCE®, BELMONT®, ENHANCE®, GRAINGUARD®, MESH®, PRO-GRO®, RANCONA®, STARTUP®, THIRAM®, VITAFLO®, VITAVAX®, MAGNAPHOS®, WEEVIL-CIDE®, AQUASTRIKE®, AQUATHOL®, PEGASUS, GOLIATH, POACONSTRICTOR, RAVEN, T-BIRD, UP-END®, UP-START®, ETHEPHON PEGASUS, GOLIATH, POACONSTRICTOR, RAVEN, T-BIRD, UP-END®, UP-START®, ZEBA® and FLORAMITE® from UPL Limited (Mumbai, Maharashtra, India).

Formulations of the present disclosure may comprise any suitable combination of actives and may therefore comprise two, three, four, five, six, seven, eight, nine, ten or more of the aforementioned actives. Conversely, in some embodiments, one, two, three, four, five, six, seven, eight, nine, ten or more of the aforementioned actives are expressly excluded from formulations of the present disclosure.

Examples of adhesives (stickers) that may be included in formulations of the present disclosure include, but not are not limited to, disaccharides (e.g. maltose, sucrose, trehalose), gums (e.g., cellulose gum, guar gum, gum arabic, gum combretum, xantham gum), maltodextrins (e.g., maltodextrins having a DEV of about 10 to about 20), monosaccharides, oils (e.g., mineral oil, olive oil, peanut oil, soybean oil and/or sunflower oil), and oligosaccharides. See generally, e.g., POWERBLOX™ (Dow, Midland, MI, USA), such as POWERBLOX™ ADJ-65 and POWERBLOX™ ADJ-65; EPO 0245970; U.S. Pat. No. 5,496,568; WO 2008/144024; WO 2009/135049; WO 2011/126832; WO 2017/083049; WO 2020/225276; WO 2021/055316; WO 2022/096688; WO 2022/096691; WO 2022/096692; WO 2022/096693; WO 2022/096694; WO/2022/096695; WO 2022/096696.

Formulations of the present disclosure may comprise any suitable combination of adhesives (stickers) and may therefore comprise two, three, four, five, six, seven, eight, nine, ten or more of the aforementioned adhesives (stickers). Conversely, in some embodiments, one, two, three, four, five, six, seven, eight, nine, ten or more of the aforementioned adhesives (stickers) are expressly excluded from formulations of the present disclosure.

Examples of dispersants (spreaders) that may be included in formulations of the present disclosure include, but not are not limited to, anionic surfactants, cationic surfactants and non-ionic surfactants. See generally, e.g., EP 0245970; U.S. Pat. No. 5,496,568; WO 2008/144024; WO 2009/135049; WO 2011/126832; WO 2017/083049; WO 2020/225276; WO 2021/055316; WO 2022/096688; WO 2022/096691; WO 2022/096692; WO 2022/096693; WO 2022/096694; WO/2022/096695; WO 2022/096696.

In some embodiments, formulations of the present disclosure comprise one or more anionic surfactants. For example, in some embodiments, formulations of the present disclosure comprise one or more anionic surfactants selected from the group consisting of alkyl carboxylates (e.g., sodium stearate), alkyl sulfates (e.g., alkyl lauryl sulfate, sodium lauryl sulfate), alkyl ether sulfates, alkyl amido ether sulfates, alkyl aryl polyether sulfates, alkyl aryl sulfates, alkyl aryl sulfonates, alkyl sulfonates, alkyl amide sulfonates, alkyl aryl sulfonates, alkyl benzene sulfonates, alkyl diphenyloxide sulfonate, alpha-olefin sulfonates, alkyl naphthalene sulfonates, paraffin sulfonates, alkyl sulfosuccinates, alkyl ether sulfosuccinates, alkylamide sulfosuccinates, alkyl sulfosuccinamates, alkyl sulfoacetates, alkyl phosphates, alkyl ether phosphates, acyl sarconsinates, acyl isethionates, N-acyl taurates, N-acyl-N-alkyltaurates, benzene sulfonates, cumene sulfonates, dioctyl sodium sulfosuccinate, ethoxylated sulfosuccinates, lignin sulfonates, linear alkylbenzene sulfonates, monoglyceride sulfates, perfluorobutanesulfonate, perfluorooctanesulfonate, phosphate ester, styrene acrylic polymers, toluene sulfonates and xylene sulfonates.

In some embodiments, formulations of the present disclosure comprise one or more cationic surfactants. For example, in some embodiments, formulations of the present disclosure comprise one or more cationic surfactants selected from the group consisting of alkyltrimethylammonium salts (e.g., cetyl trimethylammonium bromide, cetyl trimethylammonium chloride), cetylpyridinium chloride, benzalkonium chloride, benzethonium chloride, 5-Bromo-5-nitro-1,3-dioxane, dimethyldioctadecylammonium chloride, cetrimonium bromide, dioctadecyldimethylammonium bromide and/or octenidine dihydrochloride.

In some embodiments, formulations of the present disclosure comprise one or more nonionic surfactants. For example, in some embodiments, formulations of the present disclosure comprise one or more nonionic surfactants selected from the group consisting of alcohol ethoxylates (e.g., TERGITOL™ 15-S surfactants (The Dow Chemical Company, Midland, MI), such as TERGITOL™15-S-9, alkanolamides, alkanolamine condensates, carboxylic acid esters, cetostcaryl alcohol, ectyl alcohol, cocamide DEA, dodecyldimethylamine oxides, ethanolamides, ethoxylates of glycerol ester and glycol esters, ethylene oxide polymers, ethylene oxide-propylene oxide copolymers, glucoside alkyl ethers, glycerol alkyl ethers, glycerol esters, glycol alkyl ethers (e.g., polyoxyethylene glycol alkyl ethers, polyoxypropylene glycol alkyl ethers), glycol alkylphenol ethers (e.g., polyoxyethylene glycol alkylphenol ethers,), glycol esters, monolaurin, pentaethylene glycol monododecyl ethers, poloxamer, polyamines, polyglycerol polyricinoleate, polysorbate, polyoxyethylenated fatty acids, polyoxyethylenated mercaptans, polyoxyethylenated polyoxyproylene glycols, polyoxyethylene glycol sorbitan alkyl esters, polyethylene glycol-polypropylene glycol copolymers, polyoxyethylene glycol octylphenol ethers, polyvinyl pynolidones, sugar-based alkyl polyglycosides, sulfoanylamides, sorbitan fatty acid alcohol ethoxylates, sorbitan fatty acid ester ethoxylates, sorbitan fatty acid ester and/or tertiary acetylenic glycols.

In some embodiments, formulations of the present disclosure comprise one or more zwitterionic surfactants. For example, in some embodiments, formulations of the present disclosure comprise one or more zwitterionic surfactants selected from the group consisting of 3-[(3-cholamidopropyl)dimethylammonio]-1-propanesulfonate, cocamidopropyl betaine, cocamidopropyl hydroxysultaine, phosphatidylserine, phosphatidylethanolamine, phosphatidylcholine and/or one or more sphingomyelins.

In some embodiments, formulations of the present disclosure comprise one or more soaps and/or organosilicone surfactants.

Non-limiting examples of dispersants that may be incorporated into formulations of the present disclosure—or into which proteins and other compositions of the present disclosure may be incorporated—include ATLOX™ (e.g., 4916, 4991; Croda International PLC, Edison, NJ), ATLOX METASPERSE™ (Croda International PLC, Edison, NJ), BIO-SOFT® (e.g., N series, such as N1-3, N1-7, N1-5, N1-9, N23-3, N2.3-6.5, N25-3, N25-7, N25-9, N91-2.5, N91-6, N91-8; Stepan Company, Northfield, IL), MAKON® nonionic surfactants (e.g., DA-4, DA-6 and DA-9; Stepan Company, Northfield, IL), MORWET® powders (Akzo Nobel Surface Chemistry LLC, Chicago, IL), MULTIWET™ surfactants (e.g., MO-85P-PW-(AP); Croda International PLC, Edison, NJ), SAFER® soaps (Woodstream Corporation, Inc., Lancaster, PA), SILWET® surfactants (Momentive Performance Materials, Inc., Niskayuna, NY), SPAN™ surfactants (e.g., 20, 40, 60, 65, 80 and 85; Croda Inc., Edison NJ), TAMOL™ dispersants (The Dow Chemical Company, Midland, MI), TERGITOL™ surfactants (e.g., TMN-6 and TMN-100X; The Dow Chemical Company, Midland, MI), TERSPERSE surfactants (e.g., 2001, 2020, 2100, 2105, 2158, 2700, 4894 and 4896; Hunstman Corp., The Woodlands, TX), TRITON™ surfactants (e.g., X-100; The Dow Chemical Company, Midland, MI), TWEEN® surfactants (e.g., TWEEN® 20 (polyoxyethylenesorbitan monolaurate), 21, 22, 23, 28, 40, 60, 61, 65, 80, 81 and 85; Croda International PLC, Edison, NJ) and combinations thereof. Additional examples of dispersants may be found in BAIRD & ZUBLENA. 1993. SOIL FACTS: USING WETTING AGENTS (NONIONIC SURFACTANTS) ON SOIL (North Carolina Cooperative Extension Service Publication AG-439-25) (1993); BURGES, FORMULATION OF MICROBIAL BIOPESTICIDES: BENEFICIAL MICROORGANISMS, NEMATODES AND SEED TREATMENTS (Springer Science & Business Media) (2012); MCCARTY, WETTING AGENTS (Clemson University Cooperative Extension Service Publication) (2001).

Formulations of the present disclosure may comprise any suitable combination of dispersants (spreaders) and may therefore comprise two, three, four, five, six, seven, eight, nine, ten or more of the aforementioned dispersants (spreaders). Conversely, in some embodiments, one, two, three, four, five, six, seven, eight, nine, ten or more of the aforementioned dispersants (spreaders) are expressly excluded from formulations of the present disclosure.

Examples of drying agents that may be included in formulations of the present disclosure include, but not are not limited to, calcium stearate, clay (e.g., attapulgite clay, montmorillonite clay), graphite, magnesium stearate, magnesium sulfate, powdered milk, silica (e.g., fumed silica, hydrophobically-coated silica, precipitated silica), soy lecithin and talc. Additional examples of drying agents may be found in Burges, Formulation of Microbial Biopesticides: Beneficial Microorganisms, Nematodes and Seed Treatments (Springer Science & Business Media) (2012).

Non-limiting examples of drying agents that may be incorporated into formulations of the present disclosure—or into which proteins and other compositions of the present disclosure may be incorporated—include, but are not limited to, commercial products sold under the tradenames AEROSIL® and SIPERNAT® from Evonik Corporation (Parsippany, NJ), BENTOLITE® from BYK-Chemie GmbH (Wesel, Germany), and INCOTEC® from INCOTEC Inc. (Salinas, CA).

Formulations of the present disclosure may comprise any suitable combination of drying agents and may therefore comprise two, three, four, five, six, seven, eight, nine, ten or more of the aforementioned drying agents. Conversely, in some embodiments, one, two, three, four, five, six, seven, eight, nine, ten or more of the aforementioned drying agents are expressly excluded from formulations of the present disclosure.

Examples of microbes that may be included in formulations of the present disclosure include, but not are not limited to, diazotrophs, phosphate-solubilizing microorganisms and biopesticides. See generally, e.g., WO 92/08355; US 2003/082164; US 2008/320615; US 2016/345588; US 2018/168168; US 2005/187107; US 2006/258534; US 2018/279624; U.S. Ser. No. 10/820,594; US 2019/014786; U.S. Ser. No. 10/874,109; U.S. Ser. No. 10/856,552; US 2019/014787; U.S. Ser. No. 11/076,603; US 2020/093125; US 2020/085065; US 2020/000098; US 2019/345572; US 2020/263734; WO 2021/101949; WO 2021/101937; WO 2016/201284; WO 2018/186307; WO 2007/142543; WO 2017/205800; WO 2015/003908; WO 2021/018321; WO 2003/016510; WO 2016/044542; WO 92/11856.

In some embodiments, formulations of the present disclosure comprise one or more of the following: Azospirillum brasilense Ab-V5, Azospirillum brasilense Ab-V6, Azospirillum brasilense INTA Az-39, Bacillus amyloliquefaciens D747, Bacillus amyloliquefaciens NRRL B-50349, Bacillus amyloliquefaciens TJ1000, Bacillus amyloliquefaciens FZB24, Bacillus amyloliquefaciens FZB42, Bacillus amyloliquefaciens IN937a, Bacillus amyloliquefaciens IT-45, Bacillus amyloliquefaciens TJ1000, Bacillus amyloliquefaciens MBI600, Bacillus amyloliquefaciens BS27 (deposited as NRRL B-5015), Bacillus amyloliquefaciens BS2084 (deposited as NRRL B-50013), Bacillus amyloliquefaciens 15AP4 (deposited as ATCC PTA-6507), Bacillus amyloliquefaciens 3AP4 (deposited as ATCC PTA-6506), Bacillus amyloliquefaciens LSSA01 (deposited as NRRL B-50104), Bacillus amyloliquefaciens ABP278 (deposited as NRRL B-50634), Bacillus amyloliquefaciens 1013 (deposited as NRRL B-50509), Bacillus amyloliquefaciens 918 (deposited as NRRL B-50508), Bacillus amyloliquefaciens 22CP1 (deposited as ATCC PTA-6508) and Bacillus amyloliquefaciens BS18 (deposited as NRRL B-50633), Bacillus amyloliquefaciens SB3778, Bacillus cereus 1-1562, Bacillus firmus I-1582, Bacillus lichenformis BA842 (deposited as NRRL B-50516), Bacillus lichenformis BL21 (deposited as NRRL B-50134), Bacillus megaterium NRRL B-67352, Bacillus megaterium NRRL B-67357, Bacillus megaterium NRRL B-67521, Bacillus megaterium NRRL B-67522, Bacillus megaterium NRRL B-67533, Bacillus megaterium NRRL B-67534, Bacillus megaterium NRRL B-67525, Bacillus megaterium NRRL B-67526, Bacillus megaterium NRRL B-67527, Bacillus megaterium NRRL B-67528, Bacillus megaterium NRRL B-67529, Bacillus megaterium NRRL B-67530, Bacillus mycoides NRRL B-21664, Bacillus pumilus NRRL B-30087, Bacillus pumilus NRRL B 21662, Bacillus pumilus NRRL B-30087, Bacillus pumilus ATCC 55608, Bacillus pumilus ATCC 55609, Bacillus pumilus GB34, Bacillus pumilus KFP9F, Bacillus pumilus QST 2808, Bacillus sp. AQ175 (deposited as ATCC 55608), Bacillus sp. AQ177 (deposited as ATCC 55609), Bacillus subtilis AQ713 (deposited as NRRL B-21661), Bacillus subtilis AQ743 (deposited as NRRL B-21665), Bacillus subtilis ATCC 55078, Bacillus subtilis ATCC 55079, Bacillus subtilis MBI 600, Bacillus subtilis NRRL B-21661, Bacillus subtilis NRRL B-21665, Bacillus subtilis CX-9060, Bacillus subtilis GB03, Bacillus subtilis GB07, Bacillus subtilis QST-713, Bacillus subtilis FZB24, Bacillus subtilis D747, Bacillus subtilis 3BP5 (deposited as NRRL B-50510), Bacillus thuringiensis AQ52 (deposited as NRRL B-21619), Bacillus thuringiensis ATCC 13367, Bacillus thuringiensis GC-91, Bacillus thuringiensis NRRL B-21619, Bacillus thuringiensis ABTS-1857, Bacillus thuringiensis SAN 401 I, Bacillus thuringiensis ABG-6305, Bacillus thuringiensis ABG-6346, Bacillus thuringiensis AM65-52, Bacillus thuringiensis SA-12, Bacillus thuringiensis SB4, Bacillus thuringiensis ABTS-351, Bacillus thuringiensis HD-1, Bacillus thuringiensis EG 2348, Bacillus thuringiensis EG 7826, Bacillus thuringiensis EG 7841, Bacillus thuringiensis DSM 2803, Bacillus thuringiensis NB-125, Bacillus thuringiensis NB-176, Bradyrhizobium spp. 8A57, Bradyrhizobium elkanii SEMIA 501, Bradyrhizobium elkanii SEMIA 587, Bradyrhizobium elkanii SEMIA 5019, Bradyrhizobium japonicum 61A227. Bradyrhizobium japonicum 61A228, Bradyrhizobium japonicum 61A273, Bradyrhizobium japonicum E-109, Bradyrhizobium japonicum NRRL B-50586 (also deposited as NRRL B-59565), Bradyrhizobium japonicum NRRL B-50587 (also deposited as NRRL B-59566), Bradyrhizobium japonicum NRRL B-50588 (also deposited as NRRL B-59567), Bradyrhizobium japonicum NRRL B-50589 (also deposited as NRRL B-59568), Bradyrhizobium japonicum NRRL B-50590 (also deposited as NRRL B-59569), Bradyrhizobium japonicum NRRL B-50591 (also deposited as NRRL B-59570), Bradyrhizobium japonicum NRRL B-50592 (also deposited as NRRL B-59571), Bradyrhizobium japonicum NRRL B-50593 (also deposited as NRRL B-59572), Bradyrhizobium japonicum NRRL B-50594 (also deposited as NRRL B-50493), Bradyrhizobium japonicum NRRL B-50608, Bradyrhizobium japonicum NRRL B-50609, Bradyrhizobium japonicum NRRL B-50610, Bradyrhizobium japonicum NRRL B-50611, Bradyrhizobium japonicum NRRL B-50612, Bradyrhizobium japonicum NRRL B-50726, Bradyrhizobium japonicum NRRL B-50727, Bradyrhizobium japonicum NRRL B-50728, Bradyrhizobium japonicum NRRL B-50729, Bradyrhizobium japonicum NRRL B-50730, Bradyrhizobium japonicum SEMIA 566, Bradyrhizobium japonicum SEMIA 5079, Bradyrhizobium japonicum SEMIA 5080, Bradyrhizobium japonicum USDA 6, Bradyrhizobium japonicum USDA 110, Bradyrhizobium japonicum USDA 122, Bradyrhizobium japonicum USDA 123, Bradyrhizobium japonicum USDA 127, Bradyrhizobium japonicum USDA 129, Bradyrhizobium japonicum USDA 532C, Erwinia billingiae NRRL B-67766, Gliolcadium virens ATCC 52045, Gliocladium virens GL-21, Glomus intraradices RTI-801, Lysinibacillus sphaericus NRRL B-67350, Lysinibacillus sphaericus NRRL B-67351, Lysinibacillus sphaericus NRRL B-67486, Metarhizium anisopliae F52, Paenibacillus graminis NRRL B-68249, Paenibacillus kribbensis NRRL B-68250, Paenibacillus peoriae NRRL B-67884, Paenibacillus peoriae NRRL B-67885, Paenibacillus sonchi NRRL B-68251, Penicillium bilaiae ATCC 18309, Penicillium bilaiae ATCC 20851, Penicillium bilaiae ATCC 22348, Penicillium bilaiae NRRL 50162, Penicillium bilaiae NRRL 50169, Penicillium bilaiae NRRL 50776, Penicillium bilaiae NRRL 50777, Penicillium bilaiae NRRL 50778, Penicillium bilaiae NRRL 50777, Penicillium bilaiae NRRL 50778, Penicillium bilaiae NRRL 50779, Penicillium bilaiae NRRL 50780, Penicillium bilaiae NRRL 50781, Penicillium bilaiae NRRL 50782, Penicillium bilaiae NRRL 50783, Penicillium bilaiae NRRL 50784, Penicillium bilaiae NRRL 50785, Penicillium bilaiae NRRL 50786, Penicillium bilaiae NRRL 50787, Penicillium bilaiae NRRL 50788, Penicillium bilaiae NRRL 67154, Penicillium bilaiae NRRL 67155, Penicillium bilaiae NRRL 67156, Penicillium bilaiae NRRL 67157, Penicillium bilaiae NRRL 67158, Penicillium bilaiae NRRL 67159, Penicillium bilaiae RS7B-SD1, Penicillium brevicompactum AgRF18, Penicillium canescens ATCC 10419, Penicillium expansum ATCC 24692, Penicillium expansum YT02, Penicillium fellatanum ATCC 48694, Penicillium gaestrivorus NRRL 50170, Penicillium glabrum DAOM 239074, Penicillium glabrum CBS 229.28, Penicillium janthinellum ATCC 10455, Penicillium lanosocoeruleum ATCC 48919, Penicillium radicum ATCC 201836, Penicillium radicum FRR 4717, Penicillium radicum FRR 4719, Penicillium radicum N93/47267, Penicillium raistrickii ATCC 10490, Pseudomonas jessenii PS06, Pseudomonas koreensis NRRL B-67883, Rhizobium leguminosarum SO12A-2 (IDAC 080305-01), Sinorhizobium fredii CCBAU114, Sinorhizobium fredii USDA 205. Streptomyces sp. NRRL B-30145, Streptomyces sp. M1064, Streptomyces WYE 53, Streptomyces glabus NRRL 30232, Streptomyces lydicus WYEC 108 (deposited as ATCC 55445), Streptomyces violaceusniger YCED 9, Trichoderma asperellum SKT-1, Trichoderma asperellum ICC 012, Trichoderma atroviride LC52, Trichoderma atroviride CNCM 1-1237, Trichoderma fertile JM41R, Trichoderma gamsii ICC 080, Trichoderma hamatum ATCC 52198, Trichoderma harzianum ATCC 52445, Trichoderma harzianum KRL-AG2, Trichoderma harzianum T-22, Trichoderma harzianum TH-35, Trichoderma harzianum T-39, Trichoderma harzianum ICC012, Trichoderma reesi ATCC 28217, Trichoderma virens ATCC 58678, Trichoderma virens GI-3, Trichoderma virens GI-21, Trichoderma virens GL-21, Trichoderma virens G-41, Trichoderma viridae ATCC 52440, Trichoderma viridae ICC080, Trichoderma viride TV1, Yersinia entomophaga MH96, Yersinia entomophaga NRRL B-67598, Yersinia entomophaga NRRL B-67599, Yersinia entomophaga NRRL B-67600 and Yersinia entomophaga NRRL B-67601.

Non-limiting examples of microbial compositions that may be incorporated into formulations of the present disclosure—or into which proteins and other compositions of the present disclosure may be incorporated—include, but are not limited to, commercial products sold under the tradenames SERIFEL® from BASF (Ludwigshafen, Germany); VOTIVO® from Bayer Crop Science (Creve Coeur, MO, USA); AGREE®, AGRIPHAGE™, AGSIL®, ANCORA, AZATIN®, BOTANIGARD®, BOTEGHA®, BUG-N-SLUGGO®, CARB-O-NATOR®, CRYMAX®, CUEVA®, CYD-X®, DEFGUARD®, DELIVER®, DES-X®, DOUBLE NICKEL®, FIREFIGHTER™, GEMSTAR®, GROTTO®, HOMEPLATE®, JAVELIN®, KALMORi, KOCIDE®, LIFEGARD®, MADEX®, MELOCON®, MYCOTROL®, NEEMIX®, OSO™, PFR-97™ SEDUCE™, SIL-MATRIX®, SLUGGO®, SOILGARD®, THURICIDE®, TRIACT®, TRIATHLON® and TRILOGY® from Certis (Columbia, MD); BEXFOND™ and HEARKEN® from Corteva Agroscience (Indianapolis, IN, USA); ACCUDO®, FURAGRO®, PRESENCE®, QUARTZO®, SEAMAC® and ZIRONAR® from FMC Corporation (Philadelphia, PA, USA); B.SUB™, BAM™, BIO-N™, BIO-P™, BIO-PLEX™, MICRO-FORCE™, MYCO-FORCE™, PLATFORM™, ROOT-GUARD™, and TRICHO-SHIELD™ from Nutri-Tech Solutions Pty Ltd. (Yandina, Queensland, Australia); PROVEN® and RETURN® from Pivot Bio (Berkeley, CA); ACTINOVATE®, AZOMAX®, B300®, BIONIQ®, CELL-TECH®, CTS-500®, GLYCIMAX®, JUMPSTART®, MYCOPLEX®, NITRAGIN®, OPTIMIZE®, QUICKROOTS®, RHIZOMAX®, RHIZOMYCO®, TAEGRO® and TAGTEAM® from Novozymes.

Formulations of the present disclosure may comprise any suitable combination of microbial compositions and may therefore comprise two, three, four, five, six, seven, eight, nine, ten or more of the aforementioned microbial compositions. Conversely, in some embodiments, one, two, three, four, five, six, seven, eight, nine, ten or more of the aforementioned microbial compositions are expressly excluded from formulations of the present disclosure.

Examples of nutrients that may be included in formulations of the present disclosure include, but not are not limited to, organic acids (e.g., acetic acid, citric acid, lactic acid, malic acid, taurine, etc.), macrominerals (e.g., phosphorous, calcium, magnesium, potassium, sodium, iron, etc.), trace minerals (e.g., boron, cobalt, chloride, chromium, copper, fluoride, iodine, manganese, molybdenum, selenium, zinc, etc.), vitamins, (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.), and combinations thereof. See also, generally, e.g., US 2014/235447; WO 2022/029224; WO 2022/029221; WO 2021/255118; WO 2021/247915; US 2021/300837; US 2020/148605; US 2012/247164; US 2016/355443; US 2020/055794; US 2011/154873; US 2006/243009; US 2017/088474.

Formulations of the present disclosure may comprise any suitable combination of nutrients and may therefore comprise two, three, four, five, six, seven, eight, nine, ten or more of the aforementioned nutrients. Conversely, in some embodiments, one, two, three, four, five, six, seven, eight, nine, ten or more of the aforementioned nutrients are expressly excluded from formulations of the present disclosure.

Examples of pest attractants and feeding stimulants that may be included in formulations of the present disclosure include, but not are not limited to, brevicomin, ceralure, codlelure, cue-lure, disparlure, dominicalure, eugenol, frontalin, gossyplure, grandlure, hexalure, ipsdienol, ipsenol, japonilure, latitlure, lineatin, litlure, looplure, medlure, megatomic acid, methyl eugenol, moguchun, α-multistriatin, muscalure, orfalure, oryctalure, ostramone, rescalure, siglure, sulcatol, trimedlure, trunc-call, and combinations thereof. See generally, e.g., WO 00/28824; U.S. Pat. Nos. 5,607,684; 4,510,133; 5,290,556; U.S. 60/774,634; U.S. Pat. No. 6,773,727; WO 2022/051661; EP 0563963; WO 2013/164384; WO 2003/020030; U.S. Pat. Nos. 8,420,070; 5,401,506; WO 92/11856.

Formulations of the present disclosure may comprise any suitable combination of pest attractants and/or feeding stimulants and may therefore comprise two, three, four, five, six, seven, eight, nine, ten or more of the aforementioned pest attractants and/or feeding stimulants. Conversely, in some embodiments, one, two, three, four, five, six, seven, eight, nine, ten or more of the aforementioned pest attractants and/or feeding stimulants are expressly excluded from formulations of the present disclosure.

Examples of pH control components that may be included in formulations of the present disclosure include phosphate and other salts capable of buffering at the desired pH, and having an aqueous solubility of more than 1% w/w. A preferred pH control component is a phosphate buffer containing the ionic species HPO₄²⁻ and H₂PO₄⁻.

A pH control component may be a single ionic species that can maintain a constant pH but only provide a buffering effect towards either acidification or basification. An example of such, is HPO₄²⁻ which can ensure an alkaline pH (of approximately 9) and provide a buffering effect against acidification. This may be beneficial in an agricultural setting to keep the pH constant at an alkaline pH, as most environmental factors will cause acidification of the droplet and deposit.

In preferred embodiments, the pH control component does not significantly change pH (+/−0.5 pH units) or change in a desired direction upon drying when the solvent evaporates from the droplet on the leaf surface. Some buffers will, upon drying, change pH as a result of differences in solubility of the buffer components. As an example, the pH of a sodium phosphate buffer constituting of Na₂HPO₄ and NaH₂PO₄ can reduce to pH 4 or lower upon drying since the dibasic form (Na₂HPO₄) will crystallize to a larger degree. On the contrary, the pH of a potassium phosphate buffer constituting of K₂HPO₄ and KH₂PO₄ will approach pH 9 upon drying since the monobasic form (KH₂PO₄) has the lowest solubility (Sarciaux 1999).

A pH control component is most effective (highest buffer capacity) when the pKa is close to the desired pH of the composition. This will reduce the amount of buffer needed to maintain a desired pH. In an embodiment, the buffer includes salts having a neutral/alkaline pKa, such as a pKa in the range of 6.5 to 10.

As a rule of thumb, a pH control component can be used to control the pH of a solution at a pH+/−1 pH-unit from its pKa value. pH control components with a pKa value above 6.5 are useful for controlling the pH at 7.5 or above. Examples of suitable pH control component includes, but are not limited to: Sodium/potassium phosphate (pKa₁ 2.12, pKa₂ 7.21, pKa₃ 12.67), sodium/potassium carbonate (pKa₁ 6.37, pKa₂ 10.32), 2-amino-2-(hydroxymethyl)-1,3-propanediol (TRIS) (pKa 8.1), [Bis(2-hydroxyethyl)amino]acetic acid (Bicine) (pKa 8.35), N-[tris(hydroxymethyl)methyl]glycine (Tricine) (pKa 8.15), 4-(2-hydroxyethyl)-1-piperazineethanesulfonic acid (HEPES) (pKa₁ 3.0, pKa₂ 7.5), N-[tris(hydroxymethyl)methyl]-2-aminoethanesulfonic acid (TES) (pKa 7.55), 3-(N-morpholino)propanesulfonic acid (MOPS) (pKa 7.2), tris(hydroxymethyl)methylamino]propanesulfonic acid (TAPS) (pKa 8.44), N-[tris(hydroxymethyl)methyl]-3-amino-2-hydroxypropanesulfonic acid (TAPSO) (pKa 7.6), glycylglycine (pKa₁ 3.14 pKa₂ 8.17), 2-(N-cyclohexylamino)ethanesulfonic acid (CHES) (pKa 9.3), sodium/potassium borate (pKa₁ 9.24, pKa₂ 12.4, pKa₃ 13.3), 2-amino-2-methyl-1,3-propanediol (ammediol) (pKa 8.8), triethanol amine (pKa 7.74), 2-amino-2-methyl-1-propanol (pKa 9.7), glycine (pKa₁ 2.34, pKa₂ 9.6), histidine (pKa₁ 1.82, pKa2 6.00, pKa₃ 9.17), and other amino acid buffers.

Non-preferred pH control components include, but are not limited to, pH control components with an unfavorable pKa (pKa <6.5 for an enzyme that requires an alkaline pH), volatile pH control component, pH control component that display significant phytotoxicity (this may sometimes include the above-mentioned “suitable” pH control components, as phytotoxicity is depended on buffer concentration, pH and target crop), and pH control components that are unwanted in the environment and therefore regulated by authorities (this may sometimes include the above-mentioned “suitable” pH control component, as regulations varies throughout the world).

In some embodiments, formulations of the present disclosure comprise one or more pH control components in an amount of about/at least 0.01-10% w/w, preferably about/at least 0.05-5% w/w.

In some embodiments, formulations of the present disclosure can maintain an alkaline pH. pH control components may be used to obtain such formulations. For example, in some preferred embodiments, formulations of the present disclosure comprise one or more pH control components selected to provide a composition having an alkaline pH within the operable pH range(s) of each enzyme in the formulation, most preferably within +/−1 pH-unit from the optimal pH value of each enzyme in the formulation. Thus, in some embodiments, formulations of the present disclosure comprise a pH control component, such as a buffer, where an 1% w/w aqueous solution of the pH control component (buffer) has an alkaline pH in which each enzyme in the formulation is operable.

In some embodiments, formulations of the present disclosure can maintain an acidic pH. pH control components may be used to obtain such formulations. For example, in some preferred embodiments, formulations of the present disclosure comprise one or more pH control components selected to provide a composition having an acidic pH within the operable pH range(s) of each enzyme in the formulation, most preferably within +/−1 pH-unit from the optimal pH value of each enzyme in the formulation. Thus, in some embodiments, formulations of the present disclosure comprise a pH control component, such as a buffer, where an 1% w/w aqueous solution of the pH control component (buffer) has an acidic pH in which each enzyme in the formulation is operable.

Formulations of the present disclosure may comprise any suitable combination of pH control components and may therefore comprise two, three, four, five, six, seven, eight, nine, ten or more of the aforementioned pH control components. Conversely, in some embodiments, one, two, three, four, five, six, seven, eight, nine, ten or more of the aforementioned pH control components are expressly excluded from formulations of the present disclosure.

Examples of postharvest treatments that may be included in formulations of the present disclosure include, but not are not limited to, essential oils, ethylene biosynthesis inhibitors (e.g., cyclopropenes), pesticides and waxes. See generally, e.g., WO 00/10386; WO 01/43548; US 2002/058592; US 2002/061822; US 2002/043730; US 2002/198107; US 2004/072694; US 2003/100450; US 2004/077502; US 2005/043179; US 2005/250649; US 2005/261131; US 2005/261132; US 2005/288189; CA 2512254; CA 2512256; US 2007/117720; US 2007/265166; US 2008/113867; US 2010/144533; US 2008/206823; US 2009/035380; US2009/077684; US 2009/118492; US 2009/230350; US 2010/047408; US 2011/321191; US 2011/034335; US 2011/014334; US 2012/272572; US 2012/282380; US2011/293801; US2012/004108; US2013/065764; US2012/258220; US2012/142534; US 2014/127309; US 2013/004634; US 2014/011679; US 2015/208679; WO 2014/120715; WO 2015/175157; WO 2017/180695; US 2014/080710; US 2015/237877; US 2015/272115; US 2016/000072; US 2015/366189; US 2014/242235; US 2014/271758; US 2016/066568; US 2016/095311; US 2015/018430; US 2015/087520; US 2015/231588; US 2015/366230; US 2016/235070; US 2016/324147; US 2017/251673; US 2017/251662; US 2017/251669; US 2017/265462; US 2017/318804; US 2018/139975; US 2018/356384; US 2021/102245; US 2021/238201; WO 2022/094214; EP 2468107; ES 2439616; WO 2014/128321; WO 2018/116027; WO 2018/128807; WO 2019/058211; WO 2020/157714; US 2009/253578; US 2009/253579; US 2004/146617; US 2022/087261; WO 2020/016728; US 2010/173773; US 2010/292080; U.S. Pat. No. 5,858,436; EP 0972450; U.S. Pat. Nos. 6,221,414; 6,723,364; WO 00/49880; U.S. Pat. No. 6,403,139; US 2005/129662; US 2006/228458; US 2005/137090; US 2006/276336; US 2008/175926; US 2008/016766; US 2008/145499; US 2010/092631; US 2010/081636; US 2011/003694; US 2011/008475; US 2010/298147; US 2013/072383; US 2013/156835; US 2013/266670; US 2013/236562; US 2013/178489; US 2014/187570; US 2013/306158; US 2013/341809; US 2016/330987; WO 2017/001502; US 2019/159469; WO 2017/220581; US 2020/060300; US 2020/221719; WO 2020/016154; WO 2020/225066; WO 2021/233900; WO 2005/058014.

Non-limiting examples of postharvest treatment compositions that may be incorporated into formulations of the present disclosure—or into which proteins and other compositions of the present disclosure may be incorporated—include commercial products sold under the tradenames ACTISEAL™, ACTISEAL™ CONTORL-TEC™, ETHYLBLOC, FRESHCLOUD™, FRESHSTART, HARVISTA™, SMARTCITRUS, SMARTFRESH™, TEYCER, VITAFRESH from AgroFresh, Inc. (Philadelphia, PA, USA); CERAXEL®, CERASULFUR®, CERAQUINT®, ELIMR®, MUSACARE® and CERAFRUTA from CERADIS Crop Protection (Ceradis B.V., Netherlands); APL-LUSTR®, APL-BRITE, BIO-SAVER, CITRUBLUSH, CITRUS BRITE, CITRUS FIX™, CITRUS LUSTR®, DECCO and DECCONATUR™ from Decco U.S. Post-Harvest, Inc. (Monrovia, CA, USA); SEMPERFRESH, LUSTRE DRY, NATURAL SHINE®, PACRITE®, PRIMAFRESH®, SHIELD-BRITE®, EFOG® and XEDAQUIN from Pace International (Wapato, WA, USA); ALUMNI®, CHAIRMAN®, GRADUATER, GRADUATEA+®, MENTOR®, MERTECT®, SCHOLAR® and STADIUM® from Syngenta Crop Protection (Basel, Switzerland); and MAGNAPHOS, QUICKPHLO-R, QUICKPHOS from UPL Limited (Mumbai, Maharashtra, India).

It is to be understood that many of the compounds described above as “chemical actives” and “chemical active compositions” may be applied to plants and plant parts both preharvest and postharvest and may therefore also be considered “postharvest treatments” and “postharvest treatment compositions.”

Formulations of the present disclosure may comprise any suitable combination of postharvest treatment compositions and may therefore comprise two, three, four, five, six, seven, eight, nine, ten or more of the aforementioned postharvest treatment compositions. Conversely, in some embodiments, one, two, three, four, five, six, seven, eight, nine, ten or more of the aforementioned v are expressly excluded from formulations of the present disclosure.

Examples of preservatives that may be included in formulations of the present disclosure include, but are not limited to, benzoates (e.g., sodium benzoate), benzoic acid, methyl paraben, phenoxy ethanol, proprionates (e.g., ammonium proprionate, calcium proprionate, sodium proprionate), proprionic acid, sorbates (e.g., potassium sorbate, sodium sorbate), and 1,2-benzisothiazolin-3-one (PROXEL®; Basel, Switzerland).

Formulations of the present disclosure may comprise any suitable combination of preservatives and may therefore comprise two, three, four, five, six, seven, eight, nine, ten or more of the aforementioned preservatives. Conversely, in some embodiments, one, two, three, four, five, six, seven, eight, nine, ten or more of the aforementioned preservatives are expressly excluded from formulations of the present disclosure.

Examples of rain fasteners that may be included in formulations of the present disclosure include, but not are not limited to, organo-modified siloxanes (organosiloxanes), such as trisiloxanes and polysiloxanes. See generally EP 0245970; U.S. Pat. No. 5,496,568; WO 2008/144024; WO 2009/135049; WO 2011/126832; WO 2017/083049; WO 2020/225276; WO 2021/055316; WO 2022/096688; WO 2022/096691; WO 2022/096692; WO 2022/096693; WO 2022/096694; WO/2022/096695; WO 2022/096696.

In some embodiments, formulations of the present disclosure comprise one or more organo-modified siloxanes having the general molecular structure of Formula 1:

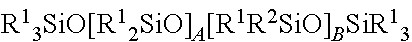

in which
R¹ represents identical or different from each other hydrocarbon substituents of 1-10 carbons or hydrogen, preferred are methyl, ethyl, propyl and phenyl substituents, particularly preferred are methyl substituents;
R² represents identical or different from each other polyether substituents of the general Formula II:

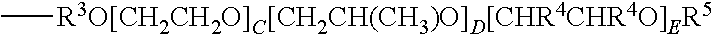

wherein
R³ represents identical or different from each other hydrocarbon moieties of 1-8 carbons, which optionally is interrupted by oxygen atoms. Preferred is linear hydrocarbons of 2-4 carbons, particularly preferred is —CH₂—CH₂—CH₂—
R⁴ represents identical or different from each other hydrocarbon substituents of 1-12 carbons or hydrogen, preferred is methyl, ethyl, phenyl or hydrogen substituents
R⁵ represents identical or different from each other hydrocarbon substituents of 1-16 carbons, which optionally contains urethane, carbonyl or carboxylic acid functionality, or hydrogen.

Methyl or hydrogen substituents are preferred, with hydrogen being most preferred. A is 0-200, preferably 0-1, more preferably 0. B is 0-200, preferably 0.5-2, more preferably 1. In preferred embodiments, A+B>0. C is 0-60, preferably 1-15. D is 0-60, preferably 0-10. E is 0-20, preferably 0-10, more preferably 0. In preferred embodiments, C+D+E>0. A trisiloxane may be defined as a molecule of the general Formula I with A=0 and B=1, whereas a polysiloxane is a molecule of the general formula I with A+B>1 and A>1. Examples of commercially available organo-modified siloxanes include, but are not limited to SILWET™ surfactants (Momentive Performance Materials, Inc., Niskayuna, NY), such as SILWET™ L-77 (polyalkyleneoxide modified heptamethyltrisiloxane), SILWET™ HS-312, SILWET™ 408, SILWET™ 618, SILWET™ 625, SILWET™ 636, SILWET™ 641, SILWET™ 806, SILWET™ DA-40, SILWET™ DRS-60, SILWET™ ECO, SILWET™ HS-604, SILWET™ STIK 2 and SILWET™) and BREAK-THRU® surfactants (Evonik Operations Gmbh, Essen, Germany), such as BREAK-THRU® S 200, BREAK-THRU® S 233, BREAK-THRU® S 240, BREAK-THRU® S 255, BREAK-THRU® S 279, BREAK-THRU® S 301 (polyether trisolxane), BREAK-THRU® SD 260, BREAK-THRU® SF 420, BREAK-THRU® SP 133 (polyglycerol esters and fatty acid esters), BREAK-THRU® UNION, BREAK-THRU® VIBRANT). In some embodiments, formulations of the present disclosure comprise one or more organo-modified siloxanes in an amount of about/at least 0.001-50% w/w, preferably about/at least 0.005-25% w/w.

Formulations of the present disclosure may comprise any suitable combination of rain fasteners and may therefore comprise two, three, four, five, six, seven, eight, nine, ten or more of the aforementioned rain fasteners. Conversely, in some embodiments, one, two, three, four, five, six, seven, eight, nine, ten or more of the aforementioned rain fasteners are expressly excluded from formulations of the present disclosure.

Examples of suitable stabilizers include, but not are not limited to, boric acid, boric acid derivatives, inorganic salts, lactic acid, polyols, sugars, sugar alcohols and reversible protease inhibitors. See generally, e.g., WO 2007/113241; WO 01/04279; WO 2013/004636; WO 95/02046; WO 2009/118375; WO 2020/115179; WO 96/41859; WO 2007/025549; WO 96/23062; WO 2018/130654; WO 96/22366; WO 92/17571; WO 2017/044473; WO 2017/044545, WO 2017/116837, WO 2017/116846, WO 2017/210163, WO 2017/210166, WO 2018/118740, WO 2018/175681, WO 2018/183491, WO 2018/218008, WO 2018/218016; WO 2018/218035.

Formulations of the present disclosure may comprise any suitable combination of stabilizers and may therefore comprise two, three, four, five, six, seven, eight, nine, ten or more of the aforementioned stabilizers.

Conversely, in some embodiments, one, two, three, four, five, six, seven, eight, nine, ten or more of the aforementioned stabilizers are expressly excluded from formulations of the present disclosure.

Examples of suitable UV protectants include, but not are not limited to, aromatic amino acids (e.g., tryptophan, tyrosine), carotenoids, cinnamates, lignosulfonates (e.g., calcium lignosulfonate, sodium lignosulfonate), melanins, mycosporines, polyphenols and/or salicylates). Non-limiting examples of UV protectants include Borregaard LignoTech™ lignosulfonates (e.g., Borresperse 3A, Borresperse CA, Borresperse NA, Marasperse AG, Norlig A, Norlig 11D, Ufoxane 3A, Ultrazine NA, Vanisperse CB; Borregaard Lignotech, Sarpsborg, Norway) and combinations thereof. Additional examples of UV protectants may be found in BURGES, FORMULATION OF MICROBIAL BIOPESTICIDES: BENEFICIAL MICROORGANISMS, NEMATODES AND SEED TREATMENTS (Springer Science & Business Media) (2012).

Formulations of the present disclosure may comprise any suitable combination of UV protectants and may therefore comprise two, three, four, five, six, seven, eight, nine, ten or more of the aforementioned UV protectants. Conversely, in some embodiments, one, two, three, four, five, six, seven, eight, nine, ten or more of the aforementioned UV protectants are expressly excluded from formulations of the present disclosure.

Examples of suitable wetting agents include, but are not limited to, naphthalene sulfonates, such as alkyl naphthalene sulfonates (e.g., sodium alkyl naphthalene sulfonate), isopropyl naphthalene sulfonates (e.g., sodium isopropyl naphthalene sulfonate) and butyl naphthalene sulfonates (e.g., sodium n-butyl naphthalene sulfonate).

Formulations of the present disclosure may comprise any suitable combination of wetting agents and may therefore comprise two, three, four, five, six, seven, eight, nine, ten or more of the aforementioned wetting agents. Conversely, in some embodiments, one, two, three, four, five, six, seven, eight, nine, ten or more of the aforementioned wetting agents are expressly excluded from formulations of the present disclosure.

As will be understood by those skilled in the art, compositions of the present disclosure have many uses, including, but not limited to a) preventing, treating, suppressing and/or eliminating infestations/infections of/by myriad pests, including, but not limited to, phytopathogenic pests, such as arachnids, bacteria, fungi, gastropods, insects, nematodes, oomycetes, protozoa, viruses and weeds; b) treating surfaces/substances that are susceptible to infestation/infection; c) cleansing infested/infected surfaces/substances; d) reducing disease severity in plants and plant parts affected directly or indirectly by phytopathogenic pests; e) enhancing plant growth environments; f) improving nutrient availability/uptake/accumulation; g) reducing the amount(s) of exogenous fertilizer needed to achieve a desired result; h) improving plant growth, development and yield characteristics; i) prolonging the shelf-life of harvested plants and plant parts; j) delaying the ripening of plants and plant parts; k) hastening the ripening of plants and plant parts; l) improving the efficacy of biological/chemical pesticides; m) preventing, treating, suppressing and/or eliminating pesticide-induced resistance/phytotoxicity.

Compositions of the present disclosure may be particularly useful for protecting plants from environmental pathogens.

In some embodiments, compositions of the present disclosure are useful for preventing, treating, suppressing, eliminating and/or reducing the severity of acarid infestations by, for example, reducing attraction of an acarid to a surface, inhibiting entry of an acarid into a material, inhibiting inhabitation by an acarid, inhibiting feeding of an acarid, inhibiting amino acid production, degrading one or more amino acids, preventing an acarid from utilizing one or more essential amino acids, producing ammonia in the gut of an acarid, inhibiting the growth of an acarid, inhibiting the reproduction and/or proliferation of an acarid, degrading on one or more structural components of an acarid (e.g. procuticle components, such as chitin, and epicuticle components, such as lipoproteins, hydrocarbons, polyphenols and esters of fatty acids and alcohols), killing an acarid, and/or reducing one or more symptoms of an acarid infestation. In some embodiments, such inhibition is complete or substantially complete, such that the acarid fails to inhabit/feed/grow/reproduce/proliferate at a rate effective to initiate and/or sustain an appreciable infestation. For example, spraying a plant with an enzyme of the present disclosure may reduce the attractiveness of the plant to an acarid, reduce an acarid's desire/ability to inhabit the plant, inhibit an acarid's desire/ability to feed on the plant, inhibit production of one or more amino acids, degrade one or more amino acids, prevent an acarid from utilizing one or more essential amino acids, produce ammonia in the gut of an acarid, inhibiting the growth of an acarid after it inhabits/feeds on the plant, inhibit the reproduction and/or proliferation of an acarid on/in the plant, degrade one or more structural components of an acarid (e.g. one or more chitins, one or more lipoproteins and/or one or more long chain hydrocarbons) on/in the plant, and/or kill an acarid on/in the plant, thereby reducing one or more symptoms of infestation and/or enhancing one or more characteristics of growth and/or yield in the plant, as compared to an untreated control plant.

Compositions of the present disclosure may be used to prevent, treat, suppress, eliminate and/or reduce the severity of infestations of/by myriad acarids, including, but not limited to, herbivorous acarids, such as broad mites, bulb mites, cyclamen mites, earth mites, eriophyid mites, Lewis mites, russet mites, rust mites, and spider mites. In some embodiments, enzymes of the present disclosure are used to prevent and/or treat an infestation of a plant or plant part by one or more Auculops, (e.g., A. lycopersici), Calacarus (e.g., C. flagelliseta), Eutetranychus (e.g., E. lewesi), Halotydeus (e.g., H. destructor), Polyphagotarsonemus (e.g., P. latus), Rhizoglyphus, Tarsonemus (e.g., T. pallidus), and/or Tetranychus (e.g., T. cinnabarinus, T. evansi, T. ludeni, T urticae).

In some embodiments, compositions of the present disclosure are useful for preventing, treating, suppressing, eliminating and/or reducing the severity of bacterial infestations/infections by, for example, inhibiting adhesion of a bacterium to a surface, inhibiting entry of a bacterium into a material, inhibiting habitation by a bacterium, inhibiting the growth of a bacterium, inhibiting the reproduction and/or proliferation of a bacterium, degrading on one or more structural components of a bacterium (e.g. cell wall components, such as peptidoglycans and lipopolysaccharides), killing a bacterium, and/or reducing one or more symptoms of a bacterial infestation/infection. In some embodiments, such inhibition is complete or substantially complete, such that the bacterium fails to inhabit/feed/grow/reproduce/proliferate at a rate effective to initiate and/or sustain an appreciable infestation/infection. For example, spraying a plant with an enzyme of the present disclosure may inhibit a bacterium's ability to adhere to the surface of a plant, inhibit a bacterium's ability to enter into the plant, inhibit a bacterium's ability to inhabit the plant, inhibit growth of a bacterium on/in the plant, inhibit the reproduction and/or proliferation of a bacterium on/in the plant, degrade one or more structural components of a bacterium (e.g. one or more peptidoglycans and/or one or more lipopolysaccharides) on/in the plant, and/or kill a bacterium, thereby reducing one or more symptoms of infestation and/or enhancing one or more characteristics of growth and/or yield in the plant, as compared to an untreated control plant.

Compositions of the present disclosure may be used to prevent, treat, suppress, eliminate and/or reduce the severity of infestations/infections of/by myriad phytopathogenic bacteria, including, but not limited to, phytopathogenic Erwiniaceae and Xanthomonadales. In some embodiments, enzymes of the present disclosure are used to prevent, treat, suppress, eliminate and/or reduce the severity of an infestation/infection of a plant or plant part by one or more Agrobacterium (e.g., A. rhizogenes, A. tumefaciens, A. vitis), Burkholderia (e.g., B. gladioli), Clostridium, Dickeya (e.g., D. dadantii, D. solani), Erwinia (e.g., E. amylovora, E. aphidicola, E. carotovora, E. chrysanthemi, E. papayae, E. persicina, E. psidii, E. pyrifoliae, E. rhapontici, E. tracheiphila), Pectobacterium (e.g., P. atrosepticum, P. carotovorum), Pseudomonas (e.g., P. agarici, P. amygdali, P. avellanae, P. cannabina, P. caricapapayae, P. cichorii, P. coronafaciens, P. costantinii, P. ficuserectae, P. fuseovaginae, P. helianthin, P. meliae, P. savastanoi, P. syringae, P. tolaasii, P. tomato, P. turbinellae, P. viridiflava), Ralstonia (e.g., R. solanacearum), Xanthomonas (e.g., X. alfalfae, X. ampelina, X. arboricola, X. axonopodia, X. boreopolis, X. badrii, X. bromi, X. campestris, X. cassavae, X. citri, X. cucrurbitae, X cyanopsidis, X. cynarae, X. euvesicatoria, X. frageriae, Xigardneri, X. holcicola, X. hortorum, X. hyacinthi, X maliensis, X. malvacearum, X. manihotis, X. melonis, X. oryzae, X. papavericola, X. perforans, X. phaseoli, X pisi, X. populi, X. sacchari, X. theicola, X translucens, X. vasicola, X. vesicatoria), and/or Xylella (e.g., X. fastidiosa).

In some embodiments, compositions of the present disclosure are useful for preventing, treating, suppressing, eliminating and/or reducing the severity of fungal infestations/infections by, for example, inhibiting adhesion of a fungus to a surface, inhibiting entry of a fungus into a material, inhibiting habitation by a fungus, inhibiting production of one or more amino acids, degrading one or more amino acids, inhibiting the growth of a fungus, inhibiting the reproduction and/or proliferation of a fungus, degrading on one or more structural components of a fungus (e.g. cell wall components, such as chitins, glucans and mannans, and cell membrane components, such as ergosterols), killing a fungus, and/or reducing one or more symptoms of a fungal infestation/infection. In some embodiments, such inhibition is complete or substantially complete, such that the fungus fails to inhabit/feed/grow/reproduce/proliferate at a rate effective to initiate and/or sustain an appreciable infestation/infection. For example, spraying a plant with an enzyme of the present disclosure may inhibit a fungus' ability to adhere to the surface of a plant, inhibit a fungus' ability to enter into the plant, inhibit a fungus' ability to inhabit the plant, inhibit the ability of a fungus to produce one or more amino acids, degrade one or more amino acids, inhibit growth of a fungus on/in the plant, inhibit the reproduction and/or proliferation of a fungus on/in the plant, degrade one or more structural components of a fungus (e.g. one or more chitins, one or more glucans and/or one or more mannans) on/in the plant, and/or kill a fungus, thereby reducing one or more symptoms of infestation and/or enhancing one or more characteristics of growth and/or yield in the plant, as compared to an untreated control plant.

Compositions of the present disclosure may be used to prevent, treat, suppress, eliminate and/or reduce the severity of infestations/infections of/by myriad phytopathogenic fungi, including, but not limited to, phytopathogenic Ascomycetes, Basidiomycetes, Chytridiomycota, Deueromycota, Peronosporomycota, Plasmodiophoromycota and Zygomycota, such as blasts, blights, bunts, galls, mildews, molds, rots, rusts, scabs, smuts and wilts. In some embodiments, enzymes of the present disclosure are used to prevent, treat, suppress, eliminate and/or reduce the severity of an infestation/infection of a plant or plant part by one or more Aecidium (e.g., A. aechmantherae, A. amaryllidis, A. breyniae, A. campanulastri, A. cannabis, A. cantensis, A. capsicum, A. foeniculi, A. narcissi), Alternaria (e.g., A. alternata, A. alternantherae, A. arachidis, A. arborescens, A. arbusti, A. blumeae, A. brassicae, A. brassicicola, A. burnsii. A. carotiincultae, A. carthami, A. celosiae, A. cinerariae, A. citri, A. conjuncta, A. cucumerina, A. dauci, A. dianthi, A. dianthicola, A. eichhorniae, A. euphorbiicola, A. gaisen, A. helianthi, A. helianthicola, A. hungarica, A. infectoria, A. japonica, A. leucanthemi, A. linicola, A. longipes, A. mali, A. molesta, A. padwickii, A. panax, A. perpunctulata, A. patroselini, A. porri, A. quericola, A. radicina, A. raphani, A. saponariae, A. selini, A. senecionis, A. smyrnii, A. solani, A. sonchi, A. tenuissima, A. triticina, A. ventricosa, A. zinniae), Ascochyta (e.g., A. asparagina, A. bohemica, A., caricae, A. doronici, A. fabae, A. gossypii, A. graminea, A. hordei, A. humuli, A. medicaginicola, A. pinodes, A. pisi, A. prasadii, A. rabiei, A. rhei, A. sorghi, A. sorghinia, A. spinaciae, A. tarda, A. tritici, A. viciae, A. vindobonensis), Ascospora (e.g., A. ruborum), Aspergillus (e.g., A. aculeatus, A. candidus, A. clavatus, A. fisherianus, A. flavus, A. fumigatus, A. niger. A. parasiticus, A. restrictus, A. sojae, A. solani), Asteroma (e.g., A. caryae), Austropuccinia (e.g., A. psidii), Bipolaris (e.g., B. cactivora, B. cookei, B. incurvata, B. sacchari, B. sorghicola, B. sorokiniana, B. zeae), Blumeria (e.g., B. graminis), Boeremia (e.g., B. lycopersici), Botrytis (e.g., B. allii, B. anthophila, B. cinerea, B. citricola, B. citrina, B. elliptica, B. fabae, B. fabiopsis, B. galanthina, B. gladioli, B. gossypina, B. hormini, B. hyacinthi, B. isabellina, B. latebricola, B. liliorum, B. limacidae, B. luteobrunnea, B. lutescens, B. mali, B. monihoides, B. narcissicola, B. necans, B. paeoniae, B. peronosporoides, B. pistiae, B. platensis, B. pruinosa, B. pseudocinerea, B. pyramidalis, B. rivoltae, B. rosea, B. rubescens, B. rudiculoides, B. sekimotoi, B. septospora, B. setuligera, B. sinoallii, B. sonchina, B. splendida, B. squamosa, B. taxi, B. terrestris, B. tracheiphila, B. trifolii, B. tulipae, B. viciae-hirsutae, B. yuae), Calonectria (e.g., C. ilicicola, C. indusiate, C. kyotensis, C. pteridis, C. pyrochroa, C. quinqueseptata), Camarotella (e.g., C. acrocomiae, C. costaricensis), Candida (e.g., C. albicans), Capnodium (e.g., C. theae), Cephalosporium (e.g., C. gramineum), Ceratocystis (e.g., C. fimbriata), Ceratobasidium (e.g., C. cereale), Cercoseptoria (e.g., C. ocellata), Cercospora (e.g., C. angreci, C. apii, C. apiicola, C. arachidicola, C. asparagi. C. atrofiliformis, C. beticola, C. bolleana, C. brachypus, C. brassicola, C. brunkii, C. canescens, C. cannabis, C. cantuariensis, C. capsici, C. caribaea, C. carotae, C. circumscissa, C. citrulline, C. clemensiae, C. coffeicola, C. coryli, C. corylina, C. eleusine, C. fragariae, C. fuchsiae, C. fusca, C. fusimaculans, C. gerberae, C. halstedii, C. handelii, C. hayi, C. hydrangeae, C. kaki, C. kikuchii, C. lentis, C. liquidambraris, C. longipes, C. longissima, C. malloti, C. mamaonis, C. mangiferae, C. medicaginis, C. melongenae, C. minima, C. minuta, C. musae, C. nicotnanae, C. odontoglossi, C. oryzae, C. papayae, C. penniseti, C. personata, C. piaropi, C. pisa-sativae, C. platanicola, C. puderii, C. pulcherrima, C. rhapidicola, C. rosicola, C. rubrotincta, C. sojina, C. solani, C. solani-tuberosi, C. sorghi, C. theae, C. tuberculans, C. vexans, C. vicosae, C. zeae-maydis, C. zebrina, C. zonata), Cercosporella (e.g., C. rubi), Choanephora (e.g., C. cucurbitarum), Cladosporium (e.g., C. arthropodii, C. brassicae, C. brassicola, C. chrysanthemi, C. citri, C. cladosporioides, C. cucumerinum, C. fulvum, C. gossypiicola, C. herbarum, C. hydrangeae, C. leguminicola, C. musae, C. oncobae, C. orchidis, C. pisi, C. rhododendri, C. salinae, C. spharospermum, C. sorghi, C. syringae, C. syringicola, C. yuccae, C. zeae), Claviceps (e.g., C. africana, C. fusiformis, C. paspali, C. purpurea, C. sorghi, C. zizaniae), Clitocybe (e.g., C. parasitica), Coccidioides (e.g., C. immitus), Cochliobolus (e.g., C. carbonum, C. cymbopogonis, C. hawaiiensis, C. heterostrophus, C. lunatus, C. miyabeanus, C. ravenelii, C. sativus, C. setariae, C. spicifer, C. stenospilus, C. tuberculatus, C. victoriae), Coleosporium (e.g., C. helianthi, C. ipomoeae, C. madiae, C. pacificum, C. tussilaginis), Colletotrichum (e.g., C. acutatum, C. agaves, C. arachidis, C. boninense, C. brasiliense, C. brassicola, C. brevisporum, C. cacao, C. capsici, C. caudatum, C. cereale, C. citri, C. citricola, C. coccodes, C. coffeanum, C. crassipes, C. curcumae, C. dematium, C. derridis, C. destructivum, C. fioriniae, C. fragariae, C. fructi, C. fruticola, C. fructivorum, C. gloeosporioides, C. glycines, C. graminicola, C. gossypii, C. hanaui, C. higginsianum, C. jacksonii, C. kahawae, C. lentis, C. limonicola, C. lindemuthianum, C. lini, C. lupini, C. mangenotii, C. melonis, C. miscanthi, C. musae, C. nicholsonii, C. nigrum, C. orbiculare, C. orchidis, C. paspali, C. pisi, C. pisicola, C. radicis, C. roseum, C. serranegrense, C. sojae, C. spinaceae, C. sublineolum, C. sublineola, C. tabacum, C. trichellum. C. trifolii, C. truncatum, C. zoysiae), Coniella, Coniothecium (e.g., C. chomatosporum), Coniothyrium (e.g., C. henriquesii, C. rosarum, C. wernsdorffiae), Coprinopsis (e.g., C. psychromorbida), Cordana (e.g., C. johnstonii, C. musae), Corticum (e.g., C. theae), Cryphonectria (e.g., C. parasitica), Cylindrocarpon (e.g., C. ianthothele, C. magnusianum, C. musae), Cylindrocladiella (e.g., C. camelliae, C. parva), Cylindrocladium (e.g., C. lanceolatum, C. peruvianum), Cylindrosporium (e.g., C. cannabinum, C. juglandis, C. rubi), Cymadothea (e.g., C. trifolii), Cytospora (e.g, C. palmarum, C. personata, C. sacchari, C. sacculus, C. terebinthi), Cytosporina (e.g., C. ludibunda), Diaporthe (e.g., D. arctii, D. asparagi, D. capsici, D. citri, D. coffeae, D. dulcamarae, D. eres, D. helianthi, D. lagunensis, D. lokoyae, D. melonis, D. musae, D. orthoceras, D. perniciosa, D. phaseolorum, D. rudis, D. tanakae, D. viticola), Diplodia (e.g., D. gossypina), Dreschlera (e.g., D. avenacea, D. campanulata, D. dematioidea, D. gigantea, D. glycines, D. graminea, D. hawaiiensis, D. musae, D. poae, D. teres, D. wirreganensis), Eremothcium (formerly Nematospora) (e.g., E. gossypii), Erysiphe (e.g., E. betae, E. cichoracearum, E. communis, E. cruciferarum, E. flexuosa, E. heraclei, E. necator, E. pisi, E. polygoni, E. robiniae, E. syringae), Exserohilum (e.g., E. oryzicola, E. oryzinum), Fusarium (e.g., F. affine, F arthrosporioides, F avenaceum, F circinatum, F crookwellense, F. culmorum, F. fujikuroi, F. graminearum, F. incarnatum, F. langsethiae, F. mangiferae, F. merismoides, F. moniliforme, F. oxysporum, F. pallidoroseum, F. poae, F proliferatum, F redolens, F. roseum, F. sacchari, F. solani, F. sporotrichioides, F. sterilihyphosum, F. subglutinans, F. sulphureum, F. tricinctum, F. verticillioides, F. virguliforme), Gaeumannomyces (e.g., G. graminis), Geotrichum (e.g., G. candidum), Gibberella (e.g., G. fujikuroi, G. pulicaris, G. stilboides, G. tricincta. G. xvlarioides, G. zeae), Gilbertella (e.g., G. persicaria), Glomerella (e.g., G. cingulata), Gymnosporangium (e.g., G. junmperi-virginianae), Helminthosporium (e.g., H. oryzae, H. solani), Hemileia (e.g., H. coffeicola, H. vastatrix), Laetisaria (e.g., L. fuciformis), Leptosphaeria (e.g., L. acuta, L. asparagi, L. cannabina, L. coffaeicida, L. coniothyrium, L. glyceriae, L. gossypii, L. grisea, L. korrae, L. longispora, L. maculans, L. maydis, L. musae, L. oryzicola, L. oryzina, L. pini, L. platanicola, L. pratensis, L. raphani, L. saccharicola, L. solani, L. solanicola, L. trifolii, L. viciae, L. woroninii, L. zeae, L. zeae-maydis), Macrophomina (e.g., M. phaseolina), Magnaporthe (e.g., M. grisea, M. oryzae, M poae), Melamspora (e.g., M. lini), Microdochium (e.g., M. nivale), Monilinia (e.g., M. fructicola), Mucor (e.g., M. piriformis), Mycosphaerella (e.g., M. fijiensis, M. graminicola, M. tassiana, M zeae-maydis), Neofabraea (e.g., N. malicorticus), Ophiostoma (e.g., O. novo-ulmi, O. ulmi), Paracoccidioides (e.g., P. braziliensis), Penicillium (e.g., P. digitatum, P. expansum, P. italicum, P. rugulosum, P. verrucosum), Phakopsora (e.g., P. gossypii, P. meibomiae, P. pachyrhizi), Phialophora (e.g., P. gregata), Phoma (e.g., P. glycinicola), Phomopsis (e.g., P. asparagi, P. coffeae, P. logicolla, P. mangiferae. P. obscurans, P. perseae, P. purnorum, P. sojae, P. sclerotioides, P. tanakae, P. theae, P. vitcola), Phymatotrichopsis (e.g., P. omnivora), Physalospora (e.g., P. obtusa), Phytomyxea, Pneumocystis (e.g., P. carinii), Podosphaera (e.g., P. oxyacanthae), Pseudocercosporella, Puccinia (e.g., P. asparagi, P. cacahata, P. coronata, P. graminis, P. kuehnii, P. melanocephala, P. porri, P. punctiformis, P. recondita, P. schedonnardii, P. sessilis, P. sorghi, P. striiformis, P. tritici, P. triticina), Pyrenophora (e.g., P. tritici-repentis), Pyricularia (e.g., P. grisea), Rhizoctonia (e.g., R. cerealis, R. solani), Rhizopus (e.g., R. nigricans, R. stolonifer), Rhynchosporium, Sclerotinia (e.g., S. borealis, S. bulborum, S. homoeocarpa, S. libertiana, S. minor, S. ricini, S. sclerotiorum, S. spermophila, S. trifoliorum), Sclerotium (e.g., S. rolfsii), Scopulariopsis (e.g., S. brevicaulis), Septoria (e.g., S. apiicola, S. aciculosa, S. ampelina, S. avenae, S. azalea, S. bataticola, S. campanulae, S. caryae, S. citri, S. cucurbitacearum, S. cytisi, S. dianthi, S. eumusae, fragariae, S. fragariaecola, S. glycines, S. helianthin, S. humuli, S. hydrangea, S. lactucae, S. lycopersici, S. malagutii, S. menthae, S. musiva, S. ostryae, S passerinii, S. pisi, S. pistaciae, S. platanifolia, S rhododendri. S. secais, S. selenophomoides), Sporisorium (e.g., S. scitamineum), Synchytrium (e.g., S. endobioticum), Taphrina (e.g., T. deformans), Thielaviopsis (e.g., T. basicola, T ceremica), Tilletia (e.g., T. barclayana, T. caries, T. controversa, T. foetida, T. indica, T. laevis, T. tritici), Typhula (e.g., T. incarnata, T. ishikariensis), Uncinula, Urocystis (e.g., U. agropyri), Uromyces (e.g., U. melanocephala), Ustilago (e.g., U. esculenta, U. maydis, U. nuda, U. scitaminea, U. striiformis, U. tritici, U. virens), Venturia, Verticillium (e.g., V. alfalfae, V. dahliae, V. isaacii, V. longisporum, V. theobromae, V. zaregamsianum), Waitea (e.g., W. circinata) and/or Zymoseptoria (e.g., Z. tritici). Additional examples of fungi that may be targeted using proteins, formulations, polynucleotides and organisms of the present disclosure may be found in Bradley, Managing Diseases, in Illinois Agronomy Handbook (2008).

In some embodiments, compositions of the present disclosure are useful for preventing, treating, suppressing, eliminating and/or reducing the severity of oomycete infestations/infections by, for example, inhibiting adhesion of an oomycete to a surface, inhibiting entry of an oomycete into a material, inhibiting habitation by an oomycete, inhibiting production of one or more amino acids, degrading one or more amino acids, inhibiting the growth of an oomycete, inhibiting the reproduction and/or proliferation of an oomycete, degrading on one or more structural components of an oomycete (e.g. cell wall components, such as celluloses and other beta-glucans), killing an oomycete, and/or reducing one or more symptoms of an oomycete infestation/infection. In some embodiments, such inhibition is complete or substantially complete, such that the oomycete fails to inhabit/feed/grow/reproduce/proliferate at a rate effective to initiate and/or sustain an appreciable infestation/infection. For example, spraying a plant with an enzyme of the present disclosure may inhibit an oomycete's ability to adhere to the surface of a plant, inhibit an oomycete's ability to enter into the plant, inhibit an oomycete's ability to inhabit the plant, inhibit production of one or more amino acids, degrade one or more amino acids, inhibit growth of an oomycete on/in the plant, inhibit the reproduction and/or proliferation of an oomycete on/in the plant, degrade one or more structural components of an oomycete (e.g. one or more beta-glucans) on/in the plant, and/or kill an oomycete, thereby reducing one or more symptoms of infestation and/or enhancing one or more characteristics of growth and/or yield in the plant, as compared to an untreated control plant.

Compositions of the present disclosure may be used to prevent and/or treat infestations/infections of myriad phytopathogenic oomycetes, including, but not limited to, phytopathogenic Albuginaceae, Peronosporaceae and Pythiaceae, such as blights, mildews, molds, root rots and rusts In some embodiments, proteins of the present disclosure are used to prevent, treat, suppress, eliminate and/or reduce the severity of an infestation/infection of a plant or plant part by one or more Achlya, Albugo (e.g., A. candida), Aphanomyces (e.g., A. cochhoides, A. euteiches, A. invadans, A. iridis, A. raphani), Bremia (e.g., B. lactucae), Hyaloperonospora (e.g., H. arabidopsidis), Peronospora (e.g., P. belbahrii, P. destructor, P. effusa, P. farinose, P. fulva, P. lotorum, P. manshurica, P. parasitica, P. potentillae, P. rubi, P. schachtii, P. sparsa, P. tabacina, P. trifolii, P. viciae), Phytophthora (e.g., P. agathidicia, P. boehmeriae, P. cactorum, P. cambivora, P. capsica, P. cinnamomi, P. citricola, P. citrophthora, P. cryptogea, P. drechsleri, P. erythroseptica, P. fragariae, P. heveae, P. infestans, P. kernoviae, P. lateralis, P. megakaryam P. megasperma, P. nicotianae, P. palmivora, P. parasitica, P. ramorum, P. sojae, P. syringae), Plasmopara (e.g., P. halstedii, P. viticola), Pseudeoperonospora (e.g., P. cubensis, P. humuli), Pseudosclerospora (e.g., P. philippinesis, P. sacchari, P. sorghi), Pythium (e.g., P. acanthicum, P. aphanidermatum, P. aristosporum, P. arrhenomanes, P. butleri, P. chondricola, P. citrinum, P. cucurbitacearum, P. debaryanum, P. delicense, P. emineosum, P. graminicola, P. heterothallicum, P. hypogynum, P. insidiosum, P. irregulare, P. iwayamae, P. middletonii, P. myriotylum, P. okanoganense, P. oopapillum, P. paddicum, P. paroecandrum, P. perniciosum, P. porphyrae, P. rostratum, P. scleroteichum, P. spinosum, P. splendens, P. sulcatum, P. tardicrescens, P. tracheiphilum, P. ultimum, P. violae, P. volutum), Saprolegnia (e.g., S. parasitica), Sclerophthora (e.g., S. macrospora, S. rayssiae) and/or Sclerospora (e.g., S. graminicola). Additional examples of fungi that may be targeted using proteins, formulations, polynucleotides and organisms of the present disclosure may be found in Bradley, Managing Diseases, in ILLINOIS AGRONOMY HANDBOOK (2008).

Compositions of the present disclosure may be particularly useful for preventing, treating, suppressing, eliminating and/or reducing the severity of infestations/infections of/by phytopathogenic fungi and oomycetes, such as Albugo (e.g., A. candida, A. occidentalis), Alternaria (e.g., A. alternata, A. alternantherae, A. arachidis, A. arborescens, A. arbusti, A. blumeae, A. brassicae, A. brassicicola, A. burnsii. A. carotiincultae, A. carthami, A. celosiae, A. cinerariae, A. citri, A. conjuncta, A. cucumerina, A. dauci, A. dianthi, A. dianthicola, A. eichhorniae, A. euphorbi/cola, A. gaisen, A. helianthi, A. hehanthicola, A. hungarica, A. infectoria, A. japonica, A. leucanthemi, A. linicola, A. longipes, A. mali, A. molesta, A. padwickii, A. panax, A. perpunctulata, A. patroselini, A. porri, A. quericola, A. radicina, A. raphani, A. saponariae, A. selini, A. senecionis, A. smyrnii, A. solani, A. sonchi, A. tenuissima, A. triticina, A. ventricosa, A. zinniae), Blumeria (e.g., B. graminis), Botrytis (e.g., B. aclada, B. allii, B. cinerea, B. elliptica, B. fabae, B. squamosa), Ceratocystis (e.g., C. fimbriata), Colletotrichum, Diplodia (e.g., D. gossypina), Erwinia (e.g., E. amylovora, E. aphidicola, E. carotovora, E. chrysanthemi, E. papayae, E. persicina, E. psidii, E. pyrifoliae, E. rhapontici, E. tracheiphila), Fusarium (e.g., F. graminearum, F. oxysporum, F. solani, F. virguliforme), Geotrichum (e.g., G. candidum), Gibberella (e.g., G. fujikuroi, G. pulicaris, G. zeae), Gilbertella (e.g., G. persicaria), Glomerella (e.g., G. cingulata), Hyaloperonospora (e.g., H. arabidopsidis), Macrophomina (e.g., M. phaseolina), Magnaporthe (e.g., M. grisea, M. oryzae), Melampsora (e.g., M. lini), Monilinia (e.g., M. fructicola), Mucor (e.g., M. piriformis), Mycosphaerella (e.g., M. graminicola), Neofabraea (e.g., N. malicorticus), Penicillium (e.g., P. digitatum, P. expansum, P. italicum, P. rugulosum, P. verrucosum), Phakopsora (e.g., P. pachyrhizi), Physalospora (e.g., P. obtusa), Phytophthora (e.g., P. capsici, P. cinnamomi, P. infestans, P. parasitica, P. ramorum, P. sojae), Plasmopara (e.g., P. viticola), Pseudoperonospora (e.g., P. cubensis), Puccinia (e.g., P. asparagi, P. cacahata, P. graminis, P. kuehnii, P. melanocephala, P. porri, P. punctiformis, P. recondita, P. schedonnardii, P. sessilis, P. sorghi, P. striiformis, P. tritici, P. triticina), Pythium (e.g., P. butleri, P. ultimum), Rhizoctonia (e.g., R. solani), Rhizopus (e.g., R. nigricans, R. stolonifer), Sclerotinia (e.g., S. borealis, S. bulborum, S. homoeocarpa, S. libertiana, S. minor, S. ricini, S. sclerotiorum, S. spermophila, S. trifoliorum), Septoria (e.g., S. cucurbitacearum, S. glycines, S. lycospersici), Ustilago (e.g., U. esculenta, U. maydis, U. nuda), Zymoseptoria (e.g., Z. tritici).

Blast infestations/infections, such as those mediated by Magnaporthe (e.g., M. grisea, M oryzae), may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.11.1 (e.g., 1.11.1.6), 3.1.1 (e.g., 3.1.1.3), 3.2.1 (e.g., 3.2.1.1, 3.2.1.4, 3.2.1.6, 3.2.1.8, 3.2.1.21), 3.4.11 and/or 3.4.21 (and corresponding formulations, polynucleotides and organisms). In some embodiments, such infestations/infections may be prevented, treated, suppressed and/or eliminated using one or more of SEQ ID NO(s): 1-8, 10-12, 14-15, 17-23, 34-40, 43-46, 48-53, 57, and enzymatically active fragments/mutants/variants thereof. In some embodiments, such infestations/infections may be prevented, treated, suppressed and/or eliminated using a combination of enzymes, such as one or more cellulases, one or more hemicellulases and one or more xylanases; one or more peptidases and one or more proteases; one or more cellulases, one or more glucosidases and one or more xylanases.

Blight infestations/infections, such as those mediated by Alternaria (e.g., A. alternata, A. carotiincultae, A. panax, A. petroselini, A. solani, A. triticina), Colletotrichum, Fusarium (e.g., F. graminearum), Gibberella (e.g., G. zeae), Phytophthora (e.g., P. capsici, P. infestans, P. ramorum), may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.10.3 (e.g., 1.10.3.2), 1.11.1 (e.g., 1.11.1.6), 3.1.1 (e.g., 3.1.1.5), 3.2.1 (e.g., 3.2.1.1, 3.2.1.3, 3.2.1.4, 3.2.1.5, 3.2.1.6, 3.2.1.8, 3.2.1.11, 3.2.1.15, 3.2.1.39, 3.2.1.41, 3.2.1.58, 3.2.1.75, 3.2.1.78, 3.2.1.111), 3.4.11 (e.g., 3.4.11.1), 3.4.21 (e.g., 3.4.21.19, 3.4.21.62), 3.4.24 (e.g., 3.4.24.28) and/or 4.2.2 (e.g., 4.2.2.2) (and corresponding formulations, polynucleotides and organisms). In some embodiments, such infestations/infections may be prevented, treated, suppressed and/or eliminated using one or more of SEQ ID NO(s): 1-15, 17-25, 34-55, 57, 61, 65, 68, 80-88, 91, 72144, and enzymatically active fragments/mutants/variants thereof. In some embodiments, such infestations/infections may be prevented, treated, suppressed and/or eliminated using a combination of enzymes, such as two or more cellulases; two or more pectinases; two or more glucanases; two or more peptidases; two or more pectinases; one or more cellulases, one or more hemicellulases and one or more xylanases; one or more glucanses and one or more xylanases; one or more amylases, one or more glucanases and one or more xylanases.

Blotch infestations/infections, such as those mediated by Mycosphaerella (e.g., M. graminicola) and Zymoseptoria (e.g., Z. tritici) may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.10.3 (e.g., 1.10.3.2), 1.11.1 (e.g., 1.11.1.6), 3.1.1 (e.g., 3.1.1.5), 3.2.1 (e.g., 3.2.1.1, 3.2.1.3, 3.2.1.6, 3.2.1.8, 3.2.1.11, 3.2.1.15, 3.2.1.39, 3.2.1.41, 3.2.1.58, 3.2.1.75, 3.2.1.78, 3.2.1.101, 3.2.1.109, 3.2.1.110, 3.2.1.112), 3.4.11 (e.g., 3.4.11.1), 3.4.21 (e.g., 3.4.21.62), 3.4.24 (e.g., 3.4.24.28) and/or 4.2.2 (e.g., 4.2.2.2) (and corresponding formulations, polynucleotides and organisms). In some embodiments, such infestations/infections may be prevented, treated, suppressed and/or eliminated using one or more of SEQ ID NO(s): 1-12, 17-24, 34-45, 48-54, 61, 80-81, 83-88, 91, and enzymatically active fragments/mutants/variants thereof. In some embodiments, such infestations/infections may be prevented, treated, suppressed and/or eliminated using a combination of enzymes, such as one or more cellulases, one or more lyases and one or more pectinases; as one or more cellulases, one or more hemicellulases and one or more xylanases; two or more cellulases; two or more glucanases; two or more pectinases; one or more glucanses and one or more xylanases; one or more peptidases and one or more proteases.

Downy mildew infestations/infections, such as those mediated by Bremia (e.g., B. lactucae), Hyaloperonospora (e.g., H. arabidopsidis, H parasitica), Peronospora (e.g., P. belbahrii, P. destructor, P. effusa, P. farinose, P. fulva, P. lotorum, P. manshurica, P. parasitica, P. potentillae, P. rubi, P. schachtii, P. sparsa, P. tabacina, P. trifolii, P. viciae), Peronosclerospora, Plasmopara (e.g., P. halstedii, P. viticola) and Pseudoperonospora (e.g., P. cubensis, P. humuli), may be prevented, treated, suppressed and/or eliminated with myriad compositions of the present disclosure, including, but not limited to, proteins exhibiting one or more activities belonging to EC 3.2.1. (e.g., 3.2.1.8, 3.2.1.78) and/or 3.4.21 (e.g., 3.4.21.19, 3.4.21.62) (and corresponding formulations, polynucleotides and organisms). In some embodiments, such infestations/infections may be prevented, treated, suppressed and/or eliminated using one or more of SEQ ID NO(s): 48-53, 80-87, and enzymatically active fragments/mutants/variants thereof.

Powdery mildew infestations/infections, such as those mediated by Blumeria (e.g., B. graminis), Erysiphe (e.g., E. cichoracearum, E. necator), Golovinomyces, Levedlula (e.g., L. taurica), Microsphaera (e.g., M. diffusa), Oidium, Phyllactinia, Podosphaera (e.g., P. aphanis, P. leucotricha, P. pannosa, P. xanthii), Sphaerotheca and Uncinula, may be prevented, treated, suppressed and/or eliminated with myriad compositions of the present disclosure, including, but not limited to, proteins exhibiting one or more activities belonging to EC 1.1.3 (e.g., 1.1.3.4), EC 1.11.1 (e.g., 1.11.1.6), 3.2.1 (e.g., 3.2.1.8, 3.2.15) and/or 3.4.21 (and corresponding formulations, polynucleotides and organisms). In some embodiments, such infestations/infections may be prevented, treated, suppressed and/or eliminated using one or more of SEQ ID NO(s): 1-5, 10-12, 48-53, 80-81, and enzymatically active fragments/mutants/variants thereof. In some embodiments, such infestations/infections may be prevented, treated, suppressed and/or eliminated using a combination of enzymes, such as two or more pectinases.

Mold infestations/infections, such as those mediated by Botrytis (e.g., B. cinerea, B. elliptica), Penicillium (e.g., P. digitatum), Phytophthora (e.g., P. capsici, P. cinnamomi, P. ramorum, P. sojae), may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.10.3 (e.g., 1.10.3.2), 1.11.1 (e.g., 1.11.1.6, 1.11.1.7), EC 1.14.99 (e.g., 1.14.99.56), EC 3.1.1 (e.g., 3.1.1.3, 3.1.1.5, 3.1.1.32), 3.2.1 (e.g., 3.2.1.1, 3.2.1.3, 3.2.1.4, 3.2.1.5, 3.2.1.6, 3.2.1.8, 3.2.1.11, 3.2.1.15, 3.2.1.17, 3.2.1.21, 3.2.1.24, 3.2.1.39, 3.2.1.41, 3.2.1.59, 3.2.1.75, 3.2.1.78, 3.2.1.101, 3.2.1.109, 3.2.1.110, 3.2.1.111, 3.2.1.112, 3.2.1.130, 3.2.1.133, 3.2.1.163, 3.2.1.198), 3.4.11 (e.g., 3.4.11.1), 3.4.21 (e.g., 3.4.21.62), 3.4.24 (e.g., 3.4.24.28) and/or 4.2.2 (e.g., 4.2.2.2) (and corresponding formulations, polynucleotides and organisms). In some embodiments, such infestations/infections may be prevented, treated, suppressed and/or eliminated using one or more of SEQ ID NO(s): 1-15, 17-24, 34-46, 48-54, 57-64, 68-69, 76-81, 83-88, 91, 45906, 72144, and enzymatically active fragments/mutants/variants thereof. In some embodiments, such infestations/infections may be prevented, treated, suppressed and/or eliminated using a combination of enzymes, such as two or more cellulases; two or more pectinases; one or more cellulases, one or more hemicellulases and one or more xylanases; one or more peptidases and one or more proteases; one or more amylases, one or more glucanases and one or more bacillolysins; one or more cellulases, one or more hemicellulases and one or more xylanases; two or more peptidases; one or more glucanses and one or more xylanases; one or more peptidases and one or more proteases; one or more cellulases, one or more glucosidases and one or more xylanases; one or more cellulases, one or more furanosidases and one or more xylanases.

Crown/fruit/root/stem rot infestations/infections, such as those mediated by Colletotrichum, Fusarium (e.g., F. solani, F. virguliforme), Phytophthora (e.g., P. capsica, P. cinnamomi, P. nicotianae, P. parasitica, P. sojae), Pythium (e.g., P. graminicola, P. ultimum), Saprolegnia (e.g., S. parasitica), may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.11.1 (e.g., 1.11.1.6), 3.2.1 (e.g., 3.2.1.6, 3.2.1.8, 3.2.1.11, 3.2.1.39, 3.2.1.58, 3.2.1.75, 3.2.1.109, 3.2.1.110, 3.2.1.111) and/or 3.4.21 (e.g., 3.4.21.19) (and corresponding formulations, polynucleotides and organisms). In some embodiments, such infestations/infections may be prevented, treated, suppressed and/or eliminated using one or more of SEQ ID NO(s): 1-13, 24, 34-41, 46, 48-54, 61, 78, 80-88, and enzymatically active fragments/mutants/variants thereof. In some embodiments, such infestations/infections may be prevented, treated, suppressed and/or eliminated using a combination of enzymes, such as two or more cellulases; two or more pectinases; two or more glucanases; two or more peptidases; one or more amylases, one or more glucanases and one or more bacillolysins; one or more cellulases, one or more lyases and one or more pectinases; one or more cellulases, one or more hemicellulases and one or more xylanases; one or more glucanses and one or more xylanases; one or more peptidases and one or more proteases.

Rust infestations/infections, such as those mediated by Albugo (e.g., A. candida, A. occidentalis), Hemileia (e.g., H. coffeicola, H. vastatrix), Melamspora (e.g., M. lini), Phakopsora (e.g., P. meibomiae, P. pachyrhizi), Puccinia (e.g., P. asparagi, P. cacahata, P. graminis, P. kuehnii, P. melanocephala, P. porri, P. punctiformis, P. recondita, P. schedonnardii, P. sessilis, P. sorghi, P. striiformis, P. tritici, P. triticina) and Uromyces (e.g., U. appendiculatus), may be prevented and/or treated with myriad compositions of the present disclosure, including, but not limited to, proteins exhibiting one or more activities belonging to EC 1.1.3 (e.g., 1.1.3.4), 1.10.3 (e.g., 1.10.3.2), 3.1.1 (e.g., 3.1.1.5), 3.2.1 (e.g., 3.2.1.1, 3.2.1.3, 3.2.1.4, 3.2.1.5, 3.2.1.6, 3.2.1.8, 3.2.1.15, 3.2.1.41, 3.2.1.58, 3.2.1.78), 3.4.11 (e.g., 3.4.11.1), 3.4.21 (e.g., 3.4.21.19, 3.4.21.62) and/or 3.4.24 (e.g., 3.4.24.28) (and corresponding formulations, polynucleotides and organisms). In some embodiments, such infestations/infections may be prevented, treated, suppressed and/or eliminated using one or more of SEQ ID NO(s): 1-5, 9, 24, 34-46, 48-53, 61, 68, 80-88, 72144, and enzymatically active fragments/mutants/variants thereof. In some embodiments, such infestations/infections may be prevented, treated, suppressed and/or eliminated using a combination of enzymes, such as two or more glucanases; one or more peptidases and one or more proteases; and one or more amylases, one or more glucanases and one or more bacillolysins

Wilt infestations/infections, such as those mediated by Fusarium (e.g., F. oxysporum,), Phytophthora (e.g., P. capsici, P. infestans, P. ramorum), may be prevented, treated, suppressed and/or eliminated with myriad compositions of the present disclosure, including, but not limited to, proteins exhibiting one or more activities belonging to EC 1.1.3 (e.g., 1.1.3.4), EC 1.1.99 (e.g., 1.1.99.18), 1.10.3 (e.g., 1.10.3.2), 1.11.1 (e.g., 1.11.1.6), 3.1.1 (e.g., 3.1.1.5), 3.2.1 (e.g., 3.2.1.1, 3.2.1.3, 3.2.1.4, 3.2.1.5, 3.2.1.6, 3.2.1.8, 3.2.1.11, 3.2.1.15, 3.2.1.39, 3.2.1.41, 3.2.1.58, 3.2.1.75, 3.2.1.78, 3.2.1.111), 3.4.11 (e.g., 3.4.11.1), 3.4.21 (e.g., 3.4.21.19, 3.4.21.62) and/or 3.4.24 (e.g., 3.4.24.28) (and corresponding formulations, polynucleotides and organisms). In some embodiments, such infestations/infections may be prevented, treated, suppressed and/or eliminated using one or more of SEQ ID NO(s): 1-15, 17-24, 34-46, 48-55, 57, 68, 80-88, 72144, and enzymatically active fragments/mutants/variants thereof. In some embodiments, such infestations/infections may be prevented, treated, suppressed and/or eliminated using a combination of enzymes, such as two or more cellulases; two or more pectinases; two or more glucanases; two or more peptidases; two or more pectinases; one or more cellulases, one or more hemicellulases and one or more xylanases; one or more glucanases and one or more xylanases; one or more amylases, one or more glucanases and one or more xylanases.

In some embodiments, compositions of the present disclosure are useful for preventing, treating, suppressing, eliminating and/or reducing the severity of gastropod infestations by, for example, reducing attraction of a gastropod to a surface, inhibiting inhabitation by a gastropod, inhibiting feeding of a gastropod, inhibiting the growth of a gastropod, inhibiting the reproduction and/or proliferation of a gastropod, degrading on one or more structural components of a gastropod, killing a gastropod, and/or reducing one or more symptoms of a gastropod infestation. In some embodiments, such inhibition is complete or substantially complete, such that the gastropod fails to inhabit/feed/grow/reproduce/proliferate at a rate effective to initiate and/or sustain an appreciable infestation. For example, spraying a plant with an enzyme of the present disclosure may reduce the attractiveness of the plant to a gastropod, reduce a gastropod's desire/ability to inhabit the plant, inhibit a gastropod's desire/ability to feed on the plant, inhibiting the growth of a gastropod after it inhabits/feeds on the plant, inhibit the reproduction and/or proliferation of a gastropod on/in the plant, degrade one or more structural components of a gastropod on/in the plant, and/or kill a gastropod, thereby reducing one or more symptoms of infestation and/or enhancing one or more characteristics of growth and/or yield in the plant, as compared to an untreated control plant.

Compositions of the present disclosure may be used to prevent, treat, suppress, eliminate and/or reduce the severity of infestations of/by myriad gastropods, including, but not limited to, slugs and snails.

In some embodiments, compositions of the present disclosure are useful for preventing, treating, suppressing, eliminating and/or reducing the severity of insect infestations by, for example, reducing attraction of an insect to a surface, inhibiting entry of an insect into a material, inhibiting inhabitation by an insect, inhibiting feeding of an insect, inhibiting production of one or more amino acids, degrading one or more amino acids, preventing an insect from utilizing one or more essential amino acids, producing ammonia in the gut of an insect, inhibiting the growth of an insect, inhibiting the reproduction and/or proliferation of an insect, degrading on one or more structural components of an insect (e.g. procuticle components, such as chitin, and epicuticle components, such as lipoproteins, hydrocarbons, polyphenols and esters of fatty acids and alcohols), killing an insect, and/or reducing one or more symptoms of an insect infestation. In some embodiments, such inhibition is complete or substantially complete, such that the insect fails to inhabit/feed/grow/reproduce/proliferate at a rate effective to initiate and/or sustain an appreciable infestation. For example, spraying a plant with an enzyme of the present disclosure may reduce the attractiveness of the plant to an insect, reduce an insect's desire/ability to inhabit the plant, inhibit an insect's desire/ability to feed on the plant, inhibit production of one or more amino acids, degrade one or more amino acids, prevent an insect from utilizing one or more essential amino acids, produce ammonia in the gut of an insect, inhibit the growth of an insect after it inhabits/feeds on the plant, inhibit the reproduction and/or proliferation of an insect on/in the plant, degrade one or more structural components of an insect (e.g. one or more chitins, one or more lipoproteins and/or one or more long chain hydrocarbons) on/in the plant, and/or kill an insect, thereby reducing one or more symptoms of infestation and/or enhancing one or more characteristics of growth and/or yield in the plant, as compared to an untreated control plant.

Compositions of the present disclosure may be used to prevent and/or treat infestations of myriad insects, including, but not limited to, Coleoptera, Dermaptera, Diptera, Hemiptera, Homoptera, Hymenoptera, Lepidoptera, Orthoptera and Thysanoptera, such as aphids, armyworms, beetles, bollworms, bugs, caterpillars, cutworms, flies, moths and thrips. In some embodiments, enzymes of the present disclosure are used to prevent, treat, suppress, eliminate and/or reduce the severity of an infestation of by one or more Acalymma, Acanthaoscelides (e.g., A. obtectus,), Anasa (e.g., A. tristis), Anastrepha (e.g., A. ludens), Anoplophora (e.g., A. glabripennis), Anthonomus (e.g., A. eugenii), Acyrthosiphon (e.g., A. pisum), Aphis (e.g., A. gossypii), Aulacorthum (e.g., A. solani), Bactrocera (e.g., B. dosalis), Bemisia (e.g., B. argentifolii, B. tabaci), Brevicoryne (e.g., B. brassicae), Bruchidius (e.g., B. atrolineatus), Bruchus (e.g., B. atomarius, B. dentipes, B. lentis, B. pisorum and/or B. rufipes), Callosobruchus (e.g., C. chinensis, C. maculatus, C. rhodesianus, C. subinnotatus, C. theobromae), Caryedon (e.g., C. serratus), Cassadinae, Ceratitis (e.g., C. capitata), Chrysomelinae, Circuhifer (e.g., C. tenellus), Criocerinae, Cryptocephalinae, Cryptolestes (e.g., C. ferrugineus, C. pusillis, C. pussilloides), Cylas (e.g., C. formicarius), Delia (e.g., D. antiqua), Diabrotica, Diaphania (e.g., D. nitidalis), Diaphorina (e.g., D. citri), Donaciinae, Ephestia (e.g, E. cautella, E. elutella, E., keuhniella), Epilachna (e.g., F. varivestris), Epiphyas (e.g., F. postvittana), Fumolpinae, Galerucinae, Helicoverpa (e.g., H. zea), Heteroligus (e.g., H. meles), Iobesia (e.g., I. botrana), Lamprosomatinae, Lasioderma (e.g., L. serricorne), Leptinotarsa (e.g., L. decemLineata), Leptoglossus, Liriomyza (e.g., L. trifolii), Macrosiphoniella (e.g., M. sanborni), Macrosiphum (e.g., M. euphorbiae), Manducca, Melanoprus (e.g., M. bivitattus, M. differntiahs, M. femurrubrum), Melittia (e.g., M. cucurbitae), Myzus (e.g., M. persicae), Nezara (e.g., N. viridula), Orzaephilus (e.g., O. merator, O. surinamensis), Ostrinia (e.g., O. nubila/is), Phthorimaea (e.g., P. operculella), Pieris (e.g., P. rapae), Plodia (e.g., P. interpunctella), Plutella (e.g., P. xylostella), Popillia (e.g., P. japonica), Prostephanus (e.g., P. truncates), Psila, Rhizopertha (e.g., R. dominica), Rhopalosiphum (e.g., R. maidis), Sagrinae, Solenopsis (e.g., S. Invicta), Spilopyrinae, Sitophilus (e.g., S. granaries, S. oryzae and/or S. zeamais), Sitotroga (e.g., S. cerealella), Spodoptera (e.g., S. frugiperda), Slegobium (e.g., S. paniceum), Synetinae, Tenebrio (e.g., T. malens and/or T. molitor), Thrips (e.g., T. tabaci), Trialeurodes (e.g., T. vaporariorum), Tribolium (e.g., T. castaneum and/or T. confusum), Trichoplusia (e.g., T. ni), Trogoderma (e.g., T. granarium) and/or Trogossitidae (e.g., T. mauritanicus). Additional species of insects that may be targeted using enzymes, formulations, polynucleotides and organisms of the present disclosure may be found in CAPINERA, HANDBOOK OF VEGETABLE PESTS (2001) and Steffey and Gray, Managing Insect Pests, in ILLINOIS AGRONOMY HANDBOOK (2008).

Compositions of the present disclosure may be particularly useful for preventing, treating, suppressing, eliminating and/or reducing the severity of infestations/infections of/by leaf-eating insects, such as aphids, beetles, grasshoppers, leaf-eating larvae, locusts, thrips and weevils.

Leaf-eating insect infestations/infections, such as those mediated by leaf-eating larvae, may be prevented, treated, suppressed and/or eliminated with myriad compositions of the present disclosure, including, but not limited to, proteins exhibiting one or more activities belonging to EC 2.3.2 (e.g., 2.3.2.2), EC 3.1.1 (e.g., EC 3.1.1.3, EC 3.1.1.74), EC 3.2.1 (e.g., EC 3.2.1.24, EC 3.2.1.130, EC 3.2.1.163, EC 3.2.1.198), EC 3.5.1 (e.g., EC 3.5.1.1, EC 3.5.1.2, EC 3.5.1.33, EC 3.5.1.35) and EC 3.5.4 (e.g., EC 3.5.4.1, EC 3.5.4.2, EC 3.5.4.3, EC 3.5.4.4) (and corresponding formulations, polynucleotides and organisms). In some embodiments, such infestations/infections may be prevented, treated, suppressed and/or eliminated using one or more of SEQ ID NO(s): 16, 17-23, 27-31, 58, 76-77, 79, 89-90, 72147, 72149 and enzymatically active fragments/mutants/variants thereof. In some embodiments, such infestations/infections may be prevented, treated, suppressed and/or eliminated using a combination of enzymes, such as two or more amidases; two or more aminoacyltransferases; two or more asparaginases; two or more cutinases; two or more glutaminases; two or more mannanases; two or more mannosidases; two or more triacylglycerol lipases; one amidase and one aminoacyltransferase.

In some embodiments, compositions of the present disclosure are useful for preventing, treating, suppressing, eliminating and/or reducing the severity of nematode infestations/infections by, for example, inhibiting adhesion of a nematode to a surface, inhibiting entry of a nematode into a material, inhibiting habitation by a nematode, inhibiting production of one or more amino acids, degrading one or more amino acids, preventing a nematode from utilizing one or more essential amino acids, inhibiting the growth of a nematode, inhibiting the reproduction and/or proliferation of a nematode, degrading on one or more structural components of a nematode (e.g. cuticle components, such as collagens, cuticlins, glycoproteins and lipids), killing a nematode, and/or reducing one or more symptoms of a nematode infestation/infection. In some embodiments, such inhibition is complete or substantially complete, such that the nematode fails to inhabit/feed/grow/reproduce/proliferate at a rate effective to initiate and/or sustain an appreciable infestation/infection. For example, spraying a plant with an enzyme of the present disclosure may inhibit a nematode's ability to adhere to the surface of a plant, inhibit a nematode's ability to enter into the plant, inhibit a nematode's ability to inhabit the plant, inhibit growth of a nematode on/in the plant, inhibit the reproduction and/or proliferation of a nematode on/in the plant, degrade one or more structural components of a nematode (e.g., one or more collagens, one or more cuticlins, one or more glycoproteins and/or one or more lipids) on/in the plant, and/or kill a nematode, thereby reducing one or more symptoms of infestation and/or enhancing one or more characteristics of growth and/or yield in the plant, as compared to an untreated control plant.

Compositions of the present disclosure may be used to prevent, treat, suppress, eliminate and/or reduce the severity of infestations/infections of/by myriad phytopathogenic nematodes, including, but not limited to, root-knot nematodes, cyst nematodes, burrowing nematodes, root lesion nematodes, wilt nematodes and reniform nematodes. In some embodiments, enzymes of the present disclosure are used to prevent, treat, suppress, eliminate and/or reduce the severity of an infestation/infection of a plant or plant part by one or more Meloidogyne (e.g., M. arenaria, M. graminicola, M hapla, M. incognita, M. javanica), Globodera (e.g., G. pallida, G. rostochiensis), Heterodera (e.g., H. avenae, H. fzlipjevi, H. glycines, H. schachtii), Pratylenchus (e.g., P. penetrans, P. thornei, P. neglectus, P. zae, P. vulnus, P. coffeae), Radopholus (e.g., R. similis), Ditylenchus (e.g., D. dipsaci, D. angustus, D. desctructor, D. africanus, D. myceliophagus, D. gigas), Bursaphelenchus (e.g., B. xylophilus, B. mucronatus), Rotylenchus (e.g., R. renformis, R. parvus), Xiphinema (e.g., X. index. X italiae, X. viuttenezi, X. diversicaudatun), and/or Nacobbus (e.g., N. aberrans). Additional examples of nematodes that may be targeted by enzymes, formulations, polynucleotides and organisms of the present disclosure may be found in CAPINERA, HANDBOOK OF VEGETABLE PESTS (2001) and Niblack, Nematodes, in ILLINOIS AGRONOMY HANDBOOK (2008)

The present disclosure thus extends to methods of using compositions of the present disclosure (e.g., proteins, formulations, polynucleotides and organisms of the present disclosure) for a) preventing, treating, suppressing and/or eliminating infestations/infections of/by various pests, including, but not limited to, phytopathogenic pests, such as acarids, bacteria, fungi, gastropods, insects, nematodes, oomycetes, protozoa, viruses and weeds; b) treating surfaces/substances that are susceptible to infestation/infection by pests; c) cleansing surfaces/substances that are infested/infected by pests; d) reducing one or more aspects of disease severity in plants and plant parts affected directly or indirectly by one or more phytopathogenic pests; e) enhancing the environments in which plants are grown; f) improving nutrient availability in plant growth media, optionally boron, calcium, carbon, copper, iron, magnesium, manganese, molybdenum, nitrogen, phosphorous, potassium, sulfur and/or zinc availability; g) improving nutrient uptake and/or accumulation by a plant or plant part, optionally boron, calcium, carbon, copper, iron, magnesium, manganese, molybdenum, nitrogen, phosphorous, potassium, sulfur and/or zinc uptake/accumulation; h) reducing the amounts of exogenous fertilizer needed to achieve a desired result (e.g., the amount of exogenous nitrogen and/or phosphorous required to produce X kilograms of harvested plant material on a given parcel of land); i) improving one or more characteristics of plant growth and/or development; j) improving one or more characteristics of plant yield; k) prolonging the shelf-life of harvested plants and plant parts; l) delaying the ripening of a plant or plant part; m) hastening the ripening of a plant or plant part; n) improving the efficacy of one or more biological pesticides; o) preventing, treating, suppressing and/or eliminating biological-pesticide-induced pest resistance; p) preventing, treating, suppressing and/or eliminating biological-pesticide-induced phytotoxicity; q) improving the efficacy of one or more chemical pesticides; r) preventing, treating, suppressing and/or eliminating chemical-pesticide-induced pest resistance; and/or s) preventing, treating, suppressing and/or eliminating chemical-pesticide-induced phytotoxicity.

In some embodiments, a protein that exhibits one or more oxidoreductase activities belonging to EC 1—for example, one or more oxidase activities belonging to EC 1.1.3 (e.g., glucose oxidase activity belonging to EC 1.1.3.4, hexose oxidase activity belonging to EC 3.1.1.5, galactose oxidase activity belonging to EC 1.1.3.9), EC 1.1.99 (e.g., cellobiose oxidase activity belonging to EC 1.1.99.18), EC 1.4.3 (e.g., D-aspartate oxidase activity belonging to EC 1.4.3.1, L-amino acid oxidase activity belonging to EC 1.4.3.2, D-amino acid oxidase activity belonging to EC 1.4.3.3, D-glutamate oxidase activity belonging to EC 1.4.3.7, L-glutamate oxidase activity belonging to EC 1.4.3.11, cyclohexylamine oxidase activity belonging to EC 1.4.3.12, protein-lysine 6-oxidase activity belonging to EC 1.4.3.13, L-lysine oxidase activity belonging to EC 1.4.3.14, D-glutamate(D-aspartate) oxidase activity belonging to EC 1.4.3.15, L-aspartate oxidase activity belonging to EC 1.4.3.16, glycine oxidase activity belonging to EC 1.4.3.19, L-lysine 6-oxidase activity belonging to EC 1.4.3.20, L-arginine oxidase activity belonging to EC 1.4.3.25), EC 1.10.3 (e.g., laccase activity belonging to EC 1.10.3.2), EC 1.11.1 (e.g., catalase activity belonging to EC 1.11.1.6, peroxidase activity belonging to EC 1.11.1.7, lignin peroxidase activity belonging to EC 1.11.1.14), EC 1.14.16 (e.g., phenylalanine 4-monooxygenase activity belonging to EC 1.14.16.1, tyrosine 3-monooxygenase activity belonging to EC 1.14.16.2, tryptophan 5-monooxygenase activity belonging to EC 1.14.16.4, phenylalanine 3-monooxygenase activity belonging to EC 1.14.16.7), EC 1.14.18 (e.g., tyrosinase activity belonging to EC 1.14.18.1) and/or EC 1.14.99 (e.g., lytic chitin monooxygenase activity belonging to EC 1.14.99.53, lytic cellulose monooxygenase activity belonging to EC 1.14.99.54, lytic starch monooxygenase activity belonging to EC 1.14.99.55, lytic cellulose monooxygenase activity belonging to EC 1.14.99.56, lytic cellulose monooxygenase activity belonging to EC 1.14.99.56)—or a corresponding formulation, polynucleotide, or organism, optionally a protein comprising, consisting essentially of, or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ TD NO(s): 1-15 or a functional fragment/mutant/variant thereof (or a corresponding formulation, polynucleotide, or organism) is applied to a plant growth medium, plant, plant part (e.g., a seed), or agricultural/floricultural//horticultural/silvicultural apparatus/facility (e.g., an apparatus/facility for planting, irrigating, fertilizing, growing, monitoring, testing, harvesting, processing, packaging and/or storing a plant or plant part, such as a cultivator, seed container, seeder, planting pot, hydroponic growth system, growth chamber, greenhouse, laboratory, broadcaster, fertilization drill, fertilizer spreader, irrigation system, harvesting apparatus, postharvest storage container, postharvest treatment chamber, or postharvest shipping container) in an amount/concentration effective to a) prevent, treat, suppress and/or eliminate infestation/infection of said plant growth medium, plant, plant part or agricultural/floricultural/horticultural/silvicultural apparatus/facility o/by one or more phytopathogenic pests; b) cleanse said plant growth medium, plant, plant part, or agricultural/floricultural/horticultural/silvicultural apparatus/facility of one or more pest(s); c) prevent, treat, suppress and/or eliminate infestation/infection of a plant grown in said plant growth medium; d) reduce one or more aspects of disease severity in a plant grown in said plant growth medium; e) reduce one or more aspects of disease severity in said plant or plant part; f) prevent, treat, suppress and/or eliminate infestation/infection of a plant or plant part grown in or contacted by said agricultural/floricultural/horticultural/silvicultural apparatus/facility; g) reduce one or more aspects of disease severity in a plant or plant part grown in or contacted by said agricultural/floricultural/horticultural/silvicultural apparatus/facility; h) enhance one or more growth and/or development characteristics of a plant grown in said plant growth medium; i) enhance one or more growth and/or development characteristics of said plant or plant part; j) enhance one or more growth and/or development characteristics of a plant grown in or contacted by said agricultural/floricultural/horticultural/silvicultural apparatus/facility; k) enhance one or more yield characteristics of a plant grown in said plant growth medium; l) enhance one or more yield characteristics in said plant or plant part; m) enhance one or more yield characteristics of a plant grown in or contacted by said agricultural/floricultural/horticultural/silvicultural apparatus/facility; n) enhance the availability of one or more nutrients in said plant growth medium for uptake and/or accumulation by a plant grown therein, optionally boron, calcium, carbon, copper, iron, magnesium, manganese, molybdenum, nitrogen, phosphorous, potassium, sulfur and/or zinc; o) enhance uptake and/or accumulation of one or more nutrients by said plant or plant part, optionally boron, calcium, carbon, copper, iron, magnesium, manganese, molybdenum, nitrogen, phosphorous, potassium, sulfur and/or zinc; p) reduce the amount(s) of exogenous fertilizer needed to achieve a desired result (e.g., the amount of exogenous nitrogen and/or phosphorous required to produce X kilograms of harvested plant material); q) prolong the shelf-life of a plant grown in said plant growth medium; r) prolong the shelf-life of said plant or plant part; s) prolong the shelf-life of a plant grown in or contacted by said agricultural/floricultural/horticultural/silvicultural apparatus/facility; t) delay the ripening of a plant grown in said plant growth medium; u) delay the ripening of said plant or plant part; v) delay the ripening of a plant grown in or contacted by said agricultural/floricultural/horticultural/silvicultural apparatus/facility; w) hasten the ripening of a plant grown in said plant growth medium; x) hastening the ripening of a plant or plant part; y) hasten the ripening of a plant grown in or contacted by said agricultural/floricultural/horticultural/silvicultural apparatus/facility; z) improve the efficacy of a biological pesticide that is applied to said plant, plant part, plant growth medium or agricultural/floricultural/horticultural/silvicultural apparatus/facility prior to, concurrently with, or after application of said protein (or corresponding formulation, polynucleotide, or organism); aa) prevent, treat, suppress and/or eliminate biological-pesticide-induced pest resistance of a plant grown in said plant growth medium; bb) prevent, treat, suppress and/or eliminate biological-pesticide-induced pest resistance of a plant grown in or contacted by said agricultural/floricultural/horticultural/silvicultural apparatus/facility; cc) improve the efficacy of a chemical pesticide that is applied to said plant, plant part, plant growth medium or agricultural/floricultural/horticultural/silvicultural apparatus/facility prior to, concurrently with, or after application of said protein (or corresponding formulation, polynucleotide, or organism); dd) prevent, treat, suppress and/or eliminate chemical-pesticide-induced pest resistance of a plant grown in said plant growth medium; and/or cc) prevent, treat, suppress and/or eliminate chemical-pesticide-induced pest resistance of a plant grown in or contacted by said agricultural/floricultural/horticultural/silvicultural apparatus/facility.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 1-15;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 1-15;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 92-106 or the cDNA sequence thereof;
d) a polypeptide derived from any one of SEQ ID NO(s): 1-15 by substitution, deletion, or insertion of one or more amino acids;
e) a polypeptide derived from a mature polypeptide of any one of SEQ ID NO(s): 1-15 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide exhibits one or more oxidoreductase activities.

In some embodiments, a protein exhibiting activity belonging to EC 1.1 for example, a protein exhibiting activity belonging to EC 1.1.3 (e.g., EC 1.1.3.4) or EC 1.1.99 (e.g., EC 1.1.99.18)—or a corresponding formulation, polynucleotide, or organism is used to prevent, treat, suppress, eliminate and/or reduce the severity of an infestation/infection of a plant or plant part of/by one or more Blumeria (e.g., B. graminis), Botrytis (e.g., B. cinerea), Fusarium (e.g., F. graminearum, F. oxysporum, F. virgulibforme), Magnaporthe (e.g., M. grisea, M. oryzae), Penicillium (e.g., P. digitatum, P. expansum, P. italicum, P. rugulosum, P. verrucosum), Phakopsora (e.g., P. pachyrhizi), Phytophthora (e.g., P. capsici, P. cinnamomi, P. infestans, P. parasitica, P. ramorum, P. sojae), Puccinia (e.g., P. striiformis) and Zymoseptoria (e.g., Z. tritici). For example, an oxidoreductase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 1-8, (or a corresponding formulation, polynucleotide, or organism) may be applied to a plant or plant part to prevent, treat, suppress, eliminate and/or reducing the severity of such infestations/infections.

In some embodiments, a protein exhibiting activity belonging to belonging to EC 1.10—for example, a protein exhibiting activity belonging to EC 1.10.3 (e.g., EC 1.10.3.2)—or a corresponding formulation, polynucleotide, or organism is used to prevent, treat, suppress, eliminate and/or reduce the severity of an infestation/infection of a plant or plant part of/by one or more Botrytis (e.g., B. cinerea), Fusarium (e.g., F. graminearum, F. oxysporum, F. virgulforme), Phakopsora (e.g., P. pachyrhizi), Phytophthora (e.g., P. capsici, P. cinnamomi, P. infestans, P. parasitica, P. ramorum, P. sojae) and Zymoseptoria (e.g., Z. tritici). For example, a oxidase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 9, (or a corresponding formulation, polynucleotide, or organism) may be applied to a plant or plant part to prevent, treat, suppress, eliminate and/or reducing the severity of such infestations/infections.

In some embodiments, a protein exhibiting activity belonging to EC 1.11—for example, a protein exhibiting activity belonging to EC 1.11.1 (e.g., EC 1.11.1.6 and EC 1.11.1.7)—or a corresponding formulation, polynucleotide, or organism is used to prevent, treat, suppress, eliminate and/or reduce the severity of an infestation/infection of a plant or plant part of/by one or more Blumeria (e.g., B. graminis), Botrytis (e.g., B. cinerea), Fusarium (e.g., F. graminearum, F. oxysporum, F. virgulforme), Magnaporthe (e.g., M. grisea, M oryzae), Penicillium (e.g., P. digitatum), Phakopsora (e.g., P. pachyrhizi), Phytophthora (e.g., P. capsici, P. cinnamomi, P. infestans, P. parasitica, P. ramorum, P. sojae), Puccinia (e.g., P. striiformis) and Zymoseptoria (e.g., Z. tritici). For example, a peroxidase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 10-13, (or a corresponding formulation, polynucleotide, or organism) maybe applied to a plant or plant part to prevent, treat, suppress, eliminate and/or reducing the severity of such infestations/infections.

In some embodiments, a protein exhibiting activity belonging to EC 1.14—for example, a protein exhibiting activity belonging to EC 1.14.99 (e.g., EC 1.14.99.56)—or a corresponding formulation, polynucleotide, or organism is used to prevent, treat, suppress, eliminate and/or reduce the severity of an infestation/infection of a plant or plant part of/by one or more Botrytis (e.g., B. cinerea), Fusarium (e.g., F. graminearum, F. oxysporum, F. virguliforme), Magnaporthe (e.g., M. grisea, M. oryzae), Penicillium (e.g., P. digitatum, P. expansum, P. italicum, P. rugulosum, P. verrucosum), Phakopsora (e.g., P. pachyrhizi), Phytophthora (e.g., P. capsici, P. cinnamomi, P. infestans, P. parasitica, P. ramorum, P. sojae) and Zymoseptoria (e.g., Z. tritici). For example, a oxygenase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 10000 identical to one or more of the amino acid sequences set forth herein as SEQ ID NO(s): 14-15, (or a corresponding formulation, polynucleotide, or organism) may be applied to a plant or plant part to prevent, treat, suppress, eliminate and/or reducing the severity of such infestations/infections.

In some embodiments, a protein that exhibits one or more transferase activities belonging to EC 2—for example, one or more aminoacyltransferase activities belonging to EC 2.3.2 (e.g., D-glutamyltransferase activity belonging to EC 2.3.2.1, gamma-glutamyltransferase activity belong to EC 2.3.2.2, aspartyltransferase activity belonging to EC 2.3.2.7, protein-glutamine gamma-glutamyltransferase activity belonging to EC 2.3.2.13, D-alanine gamma-glutamyl transferase activity belonging to EC 2.3.2.14)—or a corresponding formulation, polynucleotide, or organism, optionally a protein comprising, consisting essentially of, or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 16 or a functional fragment/mutant/variant thereof (or a corresponding formulation, polynucleotide, or organism) is applied to a plant growth medium, plant, plant part (e.g., a seed), or agricultural/floricultural/horticultural/silvicultural apparatus/facility (e.g., an apparatus/facility for planting, irrigating, fertilizing, growing, monitoring, testing, harvesting, processing, packaging and/or storing a plant or plant part, such as a cultivator, seed container, seeder, planting pot, hydroponic growth system, growth chamber, greenhouse, laboratory, broadcaster, fertilization drill, fertilizer spreader, irrigation system, harvesting apparatus, postharvest storage container, postharvest treatment chamber, or postharvest shipping container) in an amount/concentration effective to a) prevent, treat, suppress and/or eliminate infestation/infection of said plant growth medium, plant, plant part or agricultural/floricultural/horticultural/silvicultural apparatus/facility o/by one or more phytopathogenic pests; b) cleanse said plant growth medium, plant, plant part, or agricultural/floricultural/horticultural/silvicultural apparatus/facility of one or more pest(s); c) prevent, treat, suppress and/or eliminate infestation/infection of a plant grown in said plant growth medium; d) reduce one or more aspects of disease severity in a plant grown in said plant growth medium; e) reduce one or more aspects of disease severity in said plant or plant part; f) prevent, treat, suppress and/or eliminate infestation/infection of a plant or plant part grown in or contacted by said agricultural/floricultural/horticultural/silvicultural apparatus/facility; g) reduce one or more aspects of disease severity in a plant or plant part grown in or contacted by said agricultural/floricultural/horticultural/silvicultural apparatus/facility; h) enhance one or more growth and/or development characteristics of a plant grown in said plant growth medium; i) enhance one or more growth and/or development characteristics of said plant or plant part; j) enhance one or more growth and/or development characteristics of a plant grown in or contacted by said agricultural/floricultural/horticultural/silvicultural apparatus/facility; k) enhance one or more yield characteristics of a plant grown in said plant growth medium; l) enhance one or more yield characteristics in said plant or plant part; m) enhance one or more yield characteristics of a plant grown in or contacted by said agricultural/floricultural/horticultural/silvicultural apparatus/facility; n) enhance the availability of one or more nutrients in said plant growth medium for uptake and/or accumulation by a plant grown therein, optionally boron, calcium, carbon, copper, iron, magnesium, manganese, molybdenum, nitrogen, phosphorous, potassium, sulfur and/or zinc; o) enhance uptake and/or accumulation of one or more nutrients by said plant or plant part, optionally boron, calcium, carbon, copper, iron, magnesium, manganese, molybdenum, nitrogen, phosphorous, potassium, sulfur and/or zinc; p) reduce the amount(s) of exogenous fertilizer needed to achieve a desired result (e.g., the amount of exogenous nitrogen and/or phosphorous required to produce X kilograms of harvested plant material); q) prolong the shelf-life of a plant grown in said plant growth medium; r) prolong the shelf-life of said plant or plant part; s) prolong the shelf-life of a plant grown in or contacted by said agricultural/floricultural/horticultural/silvicultural apparatus/facility; t) delay the ripening of a plant grown in said plant growth medium; u) delay the ripening of said plant or plant part; v) delay the ripening of a plant grown in or contacted by said agricultural/floricultural/horticultural/silvicultural apparatus/facility; w) hasten the ripening of a plant grown in said plant growth medium; x) hastening the ripening of a plant or plant part; y) hasten the ripening of a plant grown in or contacted by said agricultural/floricultural/horticultural/silvicultural apparatus/facility; z) improve the efficacy of a biological pesticide that is applied to said plant, plant part, plant growth medium or agricultural/floricultural/horticultural/silvicultural apparatus/facility prior to, concurrently with, or after application of said protein (or corresponding formulation, polynucleotide, or organism); aa) prevent, treat, suppress and/or eliminate biological-pesticide-induced pest resistance of a plant grown in said plant growth medium; bb) prevent, treat, suppress and/or eliminate biological-pesticide-induced pest resistance of a plant grown in or contacted by said agricultural/floricultural/horticultural/silvicultural apparatus/facility; cc) improve the efficacy of a chemical pesticide that is applied to said plant, plant part, plant growth medium or agricultural/floricultural/horticultural/silvicultural apparatus/facility prior to, concurrently with, or after application of said protein (or corresponding formulation, polynucleotide, or organism); dd) prevent, treat, suppress and/or eliminate chemical-pesticide-induced pest resistance of a plant grown in said plant growth medium; and/or ee) prevent, treat, suppress and/or eliminate chemical-pesticide-induced pest resistance of a plant grown in or contacted by said agricultural/floricultural/horticultural/silvicultural apparatus/facility.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 16;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 16;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 107 or the cDNA sequence thereof;
d) a polypeptide derived from any one of SEQ ID NO(s): 16 by substitution, deletion, or insertion of one or more amino acids;
c) a polypeptide derived from a mature polypeptide of any one of SEQ ID NO(s): 16 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide exhibits one or more transferase activities.

In some embodiments, a protein exhibiting activity belonging to EC 2.3—for example, a protein exhibiting activity belonging to EC 2.3.2 (e.g., EC 2.3.2.2)—or a corresponding formulation, polynucleotide, or organism is used to prevent, treat, suppress, eliminate and/or reduce the severity of an infestation of a plant or plant part of/by one or more leaf-eating insects. For example, an aminoacyltransferase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 16 (or a corresponding formulation, polynucleotide, or organism) may be applied to a plant or plant part to prevent, treat, suppress, eliminate and/or reducing the severity of such infestations.

In some embodiments, a protein that exhibits one or more hydrolase activities belonging to EC 3—for example, one or more esterase activities belonging to EC 3.1.1 (e.g., triacylglycerol lipase activity belong to EC 3.1.1.3, phospholipase A₂ activity belonging to EC 3.1.1.4, lysophospholipase activity belonging to 3.1.1.5, pectinesterase activity belonging to 3.1.1.11, phospholipase A₁ activity belonging to 3.1.1.32, lipoprotein lipase activity belonging to EC 3.1.1.34, cutinase activity belonging to 3.1.1.74), EC 3.1.3 (e.g., alkaline phosphatase activity belonging to EC 3.1.3.1, acid phosphatase activity belonging to EC 3.1.3.2, 3-phytase activity belonging to EC 3.1.3.8, glucose-6-phosphatase activity belonging to EC 3.1.3.9, glucose-1-phosphatase activity belonging to EC 3.1.3.10, fructose-biphosphatase activity belonging to EC 3.1.3.11, sugar-phosphatase activity belonging to EC 3.1.3.23, 4-phytase activity belonging to EC 3.1.3.26, fructose-2,6-biphosphate 2-phosphatase activity belonging to EC3.1.3.46, fructose-2,6-biphosphate 6-phosphatase activity belonging to EC 3.1.3.54, 5-phytase activity belonging to EC 3.1.3.72, lipid-phosphate phosphatase activity belonging to EC 3.1.3.76), and/or EC 3.1.4 (e.g., phospholipase C activity belonging to 3.1.4.3, phospholipase D activity belonging to EC 3.1.4.4, phoshoinositide phospholipase C activity belonging to 3.1.4.11), one or more glycosylase activities belonging to EC 3.2 (e.g., alpha-amylase activity belong to EC 3.2.1.1, beta-amylase activity belong to EC 3.2.1.2, glucan 1,4-alpha-glucosidase activity belong to 3.2.1.3, cellulase activity belong to 3.2.1.4, endo-1,3(4)-beta-glucanase activity belong to 3.2.1.6, inulinase activity belong to 3.2.1.7, endo-1,4-beta-xylanase activity belong to 3.2.1.8, oligo-1,6-glucosidase activity belong to 3.2.1.10, dextranase activity belong to 3.2.1.11, chitinase activity belong to 3.2.1.14, endo-polygalacturonase (pectinase) activity belong to 3.2.1.15, lysozyme activity belong to 3.2.1.17, alpha-glucosidase activity belong to 3.2.1.20, beta-glucosidase activity belong to 3.2.1.21, alpha-galactosidase activity belong to 3.2.1.22, beta-galactosidase activity belong to 3.2.1.23, alpha-mannosidase activity belong to 3.2.1.24, beta-mannosidase activity belong to 3.2.1.25, beta-fructofuranosidase activity belong to 3.2.1.26, alpha,alpha-trehalase activity belong to 3.2.1.28, endo-1,3-beta-xylanase activity belong to 3.2.1.32, amylo-1,6-glucosidase activity belonging to EC3.2.1.33, xylan 1,4-beta-xylosidase activity belong to 3.2.1.37, glucan endo-1,3-beta-D-glucosidase activity belong to 3.2.1.39, pullulanase activity belong to 3.2.1.41, alpha-L-arabinofuranosidase activity belong to 3.2.1.55, glucan 1,3-beta-glucosidase activity belong to 3.2.1.58, glucan endo-1,3-alpha-glucosidase activity belong to 3.2.1.59, glucan 1,6-alpha-glucosidase activity belonging to EC 3.2.1.70, glucan endo-1,2-beta-glucosidase activity belonging to EC 3.2.1.71, xylan 1,3-beta-xylosidase activity belonging to EC 3.2.1.72, licheninase activity belong to 3.2.1.73, glucan 1,4-beta-glucosidase activity belonging to EC 3.2.1.74, glucan endo-1,6-beta-glucosidase activity belong to 3.2.1.75, mannan 1,2-(1,3)-alpha-mannosidase activity belonging to EC 3.2.1.77, mannan endo-1,4-p-mannosidase activity belonging to 3.2.1.78, glucan 1,3-alpha-glucosidase activity belonging to EC 3.2.1.84, cellulose 1,4-beta-cellobiosidase activity belong to 3.2.1.91, peptidoglycan beta-N-acetylmuramidase activity belonging to EC 3.2.1.92, endo-alpha-N-acetylgalactosaminidase activity belong to 3.2.1.97, mannan 1,4-mannobiosidase activity belonging to EC 3.2.1.100, mannan endo-1,6-alpha-mannosidase activity belong to 3.2.1.101, endogalactosaminidase activity belong to 3.2.1.109, 1,3-alpha-L-fucosidase activity belong to 3.2.1.111, 2-deoxyglucosidase activity belong to 3.2.1.112, glycoprotein endo-alpha-1,2-mannosidase activity belonging to EC 3.2.1.130, chitosanase activity belong to 3.2.1.132, glucan 1,4-alpha-maltohydrolase activity belong to 3.2.1.133, mannan exo-1,2-1,6-alpha-mannosidase activity belonging to EC 3.2.1.137, 1,6-alpha-D-mannosidase activity belonging to 3.2.1.163, 1,4-beta-cellobiosidase activity belonging to 3.2.1.176, galactan endo-beta-1,3-galactanase activity belonging to EC 3.2.1.181, alpha-mannan endo-1,2-alpha-mannanase activity belonging to EC 3.2.1.198, exo-chitinase activity belonging to EC 3.2.1.200, exo-chitinase activity belonging to EC 3.2.1.201), one or more aminopeptidase activities belonging to EC 3.4.11, one or more serine endopeptidase activities belonging to EC 3.4.21 (e.g., chymotrypsin activity belonging to EC 3.4.21.1, trypsin activity belonging to EC 3.4.21.4, alpha-lytic endopeptidase activity belonging to EC 3.4.21.12, glutamyl endopeptidase activity belonging to EC 3.4.21.19, cucumisin activity belonging to EC 3.4.21.25, chymase activity belonging to EC 3.4.21.39, lysyl endopeptidase activity belonging to EC 3.4.21.50, leucyl endopeptidase activity belonging to EC 3.4.21.57, subtilisin activity belonging to 3.4.21.62), one or more cysteine endopeptidase activities belonging to 3.4.22 (e.g., papain activity belonging to EC 3.4.22.2, actinidain activity belonging to EC 3.4.22.14, caricain activity belonging to EC 3.4.22.30, ananain activity belonging to EC 3.4.22.31, bromelain activity belonging to EC 3.4.22.32, bromelain activity belonging to EC 3.4.22.33, legumain activity belonging to EC 3.4.22.34, zingipain activity belonging to EC 3.4.22.67), one or more aspartic endopeptidase activities belonging to EC 3.4.23 (e.g., phytopsin activity belonging to EC 3.4.23.40), one or more metalloendopeptidase activities belonging to EC 3.4.24 (e.g., bacillolysin activity belonging to EC 3.4.24.28), one or more amidohydrolase and amidase activities belonging to EC 3.5.1 (e.g., asparaginase activity belong to EC 3.5.1.1, glutaminase activity belonging to 3.5.1.2, amidase activity belonging to 3.5.1.4, urease activity belonging to 3.5.1.5, biotinidase activity belonging to 3.5.1.12, nicotinamidase activity belonging to 3.5.1.19, N-acetylglucosamine deacetylase activity belonging to EC 3.5.1.33, D-glutaminase activity belonging to 3.5.1.35, glutamin-(asparagin-)ase activity belonging to 3.5.1.38, chitin deacetylase activity belonging to 3.5.1.41, peptidyl-glutaminase activity belonging to 3.5.1.43, protein-glutamine glutaminase activity belonging to 3.5.1.44, pentanamidase activity belonging to 3.5.1.50, peptidoglycan-N-acetylglucosamine deacetylase activity belonging to EC 3.5.1.104), one or more aminidase/deiminase activities belonging to EC 3.5.3 (e.g., arginase activity belonging to EC 3.5.3.1, arginine deiminase activity belonging to EC 3.5.3.6, D-arginase activity belonging to EC 3.5.3.10, protein-arginine deiminase activity belonging to EC 3.5.3.15), and/or one or more deaminase activities belonging to EC 3.5.4 (e.g., cytosine deaminase activity belonging to EC 3.5.4.1, adenine deaminase activity belonging to EC 3.5.4.2, guanine deaminase activity belonging to EC 3.5.4.3, adenosine deaminase activity belonging to EC 3.5.4.4)—or a corresponding formulation, polynucleotide, or organism, optionally a protein comprising, consisting essentially of, or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100/o identical to one or more of the amino acid sequences set forth herein as SEQ ID NO(s): 17-90, 45906, 72144, 72147 and 72149 or a functional fragment/mutant/variant thereof (or a corresponding formulation, polynucleotide, or organism) is applied to a plant growth medium, plant, plant part (e.g., a seed), or agricultural/floricultural/horticultural/silvicultural apparatus/facility (e.g., an apparatus/facility for planting, irrigating, fertilizing, growing, monitoring, testing, harvesting, processing, packaging and/or storing a plant or plant part, such as a cultivator, seed container, seeder, planting pot, hydroponic growth system, growth chamber, greenhouse, laboratory, broadcaster, fertilization drill, fertilizer spreader, irrigation system, harvesting apparatus, postharvest storage container, postharvest treatment chamber, or postharvest shipping container) in an amount/concentration effective to a) prevent, treat, suppress and/or eliminate infestation/infection of said plant growth medium, plant, plant part or agricultural/floricultural/horticultural/silvicultural apparatus/facility o/by one or more phytopathogenic pests; b) cleanse said plant growth medium, plant, plant part, or agricultural/floricultural/horticultural/silvicultural apparatus/facility of one or more pest(s); c) prevent, treat, suppress and/or eliminate infestation/infection of a plant grown in said plant growth medium; d) reduce one or more aspects of disease severity in a plant grown in said plant growth medium; e) reduce one or more aspects of disease severity in said plant or plant part; f) prevent, treat, suppress and/or eliminate infestation/infection of a plant or plant part grown in or contacted by said agricultural/floricultural/horticultural/silvicultural apparatus/facility; g) reduce one or more aspects of disease severity in a plant or plant part grown in or contacted by said agricultural/floricultural/horticultural/silvicultural apparatus/facility; h) enhance one or more growth and/or development characteristics of a plant grown in said plant growth medium; i) enhance one or more growth and/or development characteristics of said plant or plant part; j) enhance one or more growth and/or development characteristics of a plant grown in or contacted by said agricultural/floricultural/horticultural/silvicultural apparatus/facility; k) enhance one or more yield characteristics of a plant grown in said plant growth medium; l) enhance one or more yield characteristics in said plant or plant part; m) enhance one or more yield characteristics of a plant grown in or contacted by said agricultural/floricultural/horticultural/silvicultural apparatus/facility; n) enhance the availability of one or more nutrients in said plant growth medium for uptake and/or accumulation by a plant grown therein, optionally boron, calcium, carbon, copper, iron, magnesium, manganese, molybdenum, nitrogen, phosphorous, potassium, sulfur and/or zinc; o) enhance uptake and/or accumulation of one or more nutrients by said plant or plant part, optionally boron, calcium, carbon, copper, iron, magnesium, manganese, molybdenum, nitrogen, phosphorous, potassium, sulfur and/or zinc; p) reduce the amount(s) of exogenous fertilizer needed to achieve a desired result (e.g., the amount of exogenous nitrogen and/or phosphorous required to produce X kilograms of harvested plant material); q) prolong the shelf-life of a plant grown in said plant growth medium; r) prolong the shelf-life of said plant or plant part; s) prolong the shelf-life of a plant grown in or contacted by said agricultural/floricultural/horticultural/silvicultural apparatus/facility; t) delay the ripening of a plant grown in said plant growth medium; u) delay the ripening of said plant or plant part; v) delay the ripening of a plant grown in or contacted by said agricultural/floricultural/horticultural/silvicultural apparatus/facility; w) hasten the ripening of a plant grown in said plant growth medium; x) hastening the ripening of a plant or plant part; y) hasten the ripening of a plant grown in or contacted by said agricultural/floricultural/horticultural/silvicultural apparatus/facility; z) improve the efficacy of a biological pesticide that is applied to said plant, plant part, plant growth medium or agricultural/floricultural/horticultural/silvicultural apparatus/facility prior to, concurrently with, or after application of said protein (or corresponding formulation, polynucleotide, or organism); aa) prevent, treat, suppress and/or eliminate biological-pesticide-induced pest resistance of a plant grown in said plant growth medium; bb) prevent, treat, suppress and/or eliminate biological-pesticide-induced pest resistance of a plant grown in or contacted by said agricultural/floricultural/horticultural/silvicultural apparatus/facility; cc) improve the efficacy of a chemical pesticide that is applied to said plant, plant part, plant growth medium or agricultural/floricultural/horticultural/silvicultural apparatus/facility prior to, concurrently with, or after application of said protein (or corresponding formulation, polynucleotide, or organism); dd) prevent, treat, suppress and/or eliminate chemical-pesticide-induced pest resistance of a plant grown in said plant growth medium; and/or cc) prevent, treat, suppress and/or eliminate chemical-pesticide-induced pest resistance of a plant grown in or contacted by said agricultural/floricultural/horticultural/silvicultural apparatus/facility.

In some embodiments, the protein is selected from the group consisting of
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 17-90, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 17-90, 45906, 72144, 72147 and 72149;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 108-181, 72145-72146, 72148 and 72150 or the cDNA sequence thereof,
d) a polypeptide derived from any one of SEQ ID NO(s): 17-90, 45906, 72144, 72147 and 72149 by substitution, deletion, or insertion of one or more amino acids;
e) a polypeptide derived from a mature polypeptide of any one of SEQ ID NO(s): 17-90, 45906, 72144, 72147 and 72149 by substitution, deletion, or insertion of one or more amino acids; f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide exhibits one or more hydrolase activities.

In some embodiments, a protein exhibiting activity belonging to EC 3.1—for example, a protein exhibiting activity belonging to EC 3.1.1 (e.g., EC 3.1.1.3, EC 3.1.1.5, EC 3.1.1.11, and EC 3.1.1.74) and 3.1.4 (e.g., EC 3.1.4.3 and EC 3.1.4.11)—or a corresponding formulation, polynucleotide, or organism is used to prevent, treat, suppress, eliminate and/or reduce the severity of an infestation/infection of a plant or plant part of/by one or more Botrytis (e.g., B. cinerea), Fusarium (e.g., F. graminearum, F. oxysporum, F. virgulforme), Magnaporthe (e.g., M. grisea, M oryzae), Penicillium (e.g., P. digitatum, P. expansum, P. italicum, P. rugulosum, P. verrucosum), Phakopsora (e.g., P. pachyrhizi), Phytophthora (e.g., P. capsici, P. cinnamomi, P. infestans, P. parasitica, P. ramorum, P. sojae), Zymoseptoria (e.g., Z. tritici) and insects. For example, a hydrolase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 17-33, 72147 and 72149 (or a corresponding formulation, polynucleotide, or organism) may be applied to a plant or plant part to prevent, treat, suppress, eliminate and/or reducing the severity of such infestations/infections.

In some embodiments, a protein exhibiting activity belonging to EC 3.2 for example, a protein exhibiting activity belonging to EC 3.2.1 (e.g., EC 3.2.1.1, EC 3.2.1.3, EC 3.2.1.4, EC 3.2.1.6, EC 3.2.1.7, EC 3.2.1.8, EC 3.2.1.11, EC 3.2.1.15, EC 3.2.1.17, EC 3.2.1.21, EC 3.2.1.24, EC 3.2.1.39, EC 3.2.1.41, EC 3.2.1.55, EC 3.2.1.58, EC 3.2.1.59, EC 3.2.1.73, EC 3.2.1.75, EC 3.2.1.78, EC 3.2.1.101, EC 3.2.1.109, EC 3.2.1.130, EC 3.2.1.133, EC 3.2.1.163 and EC 3.2.1.198)—or a corresponding formulation, polynucleotide, or organism is used to prevent, treat, suppress, eliminate and/or reduce the severity of an infestation/infection of a plant or plant part of/by one or more Blumeria (e.g., B. graminis), Botrytis (e.g., B. cinerea), Fusarium (e.g., F. graminearum, F. oxysporum, F. virgulforme), Magnaporthe (e.g., M. grisea, M. oryzae), Penicillium (e.g., P. digitatum, P. expansum, P. italicum, P. rugulosum, P. verrucosum), Phakopsora (e.g., P. pachyrhizi), Phytophthora (e.g., P. capsici, P. cinnamomi, P. infestans, P. parasitica, P. ramorum, P. sojae), Pseudoperonospora (e.g., P. cubensis), Puccinia (e.g., P. striiformis), Zymoseptoria (e.g., Z. tritici) and insects. For example, a glycosylase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 34-79, 45906 and 72144, (or a corresponding formulation, polynucleotide, or organism) may be applied to a plant or plant part to prevent, treat, suppress, eliminate and/or reducing the severity of such infestations/infections.

In some embodiments, a protein exhibiting activity belonging to belonging to EC 3.4—for example, a protein exhibiting activity belonging to EC 3.4.11 (e.g., EC 3.4.11.1), EC 3.4.21 (e.g., EC 3.4.21.19, EC 3.4.21.62) and EC 3.4.24 (e.g., EC 3.4.24.28)—or a corresponding formulation, polynucleotide, or organism is used to prevent, treat, suppress, eliminate and/or reduce the severity of an infestation/infection of a plant or plant part of/by one or more Blumeria (e.g., B. graminis), Botrytis (e.g., B. cinerea), Fusarium (e.g., F. graminearum, F. oxysporum, F virguliforme), Magnaporthe (e.g., M. grisea, M. oryzae), Penicillium (e.g., P. digitatum, P. expansum, P. italicum, P. rugulosum, P. verrucosum), Phakopsora (e.g., P. pachyrhizi), Phytophthora (e.g., P. capsici, P. cinnamomi, P. infestans, P. parasitica, P. ramorum, P. sojae), Pseudoperonospora (e.g., P. cubensis), Puccinia (e.g., P. striiformis), Zymoseptoria (e.g., Z. tritici) and insects. For example, a peptidase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 80-88, (or a corresponding formulation, polynucleotide, or organism) may be applied to a plant or plant part to prevent, treat, suppress, eliminate and/or reducing the severity of such infestations/infections.

In some embodiments, a protein exhibiting activity belonging to EC 3.5—for example, a protein exhibiting activity belonging to EC 3.5.1 (e.g., EC 3.5.1.1 and EC 3.5.1.2)—or a corresponding formulation, polynucleotide, or organism is used to prevent, treat, suppress, eliminate and/or reduce the severity of an infestation/infection of a plant or plant part of/by one or more Blumeria (e.g., B. graminis), Botrytis (e.g., B. cinerea), Penicillium (e.g., P. digitatum, P. expansum, P. italicum, P. rugulosum, P. verrucosum) and insects. For example, a hydrolase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 89-90 (or a corresponding formulation, polynucleotide, or organism) may be applied to a plant or plant part to prevent, treat, suppress, eliminate and/or reducing the severity of such infestations/infections.

In some embodiments, a protein that exhibits one or more lyase activities belonging to EC 4—for example, one or more carboxy-lyase activities belonging to EC 4.1.1 (e.g., aspartate 1-decarboxylase activity belonging to EC 4.1.1.11, aspartate 4-decarboxylase activity belonging to EC 4.1.1.12, valine decarboxylase activity belonging to EC 4.1.1.14, glutamate decarboxylase activity belonging to EC 4.1.1.15, lysine decarboxylase activity belonging to EC 4.1.1.18, arginine decarboxylase activity belonging to EC 4.1.1.19, histidine decarboxylase activity belonging to EC 4.1.1.22, tyrosine decarboxylase activity belonging to EC 4.1.1.25, phenylalanine decarboxylase activity belonging to EC 4.1.1.53, methionine decarboxylase activity belonging to EC 4.1.1.57, L-tryptophan decarboxylase activity belonging to EC 4.1.1.105), one or more carbon-carbon lyase activities belonging to EC 4.1.99 (e.g., tryptophanase activity belonging to EC EC 4.1.99.1, tyrosine phenol-lyase activity belonging to EC 4.1.99.2), one or more ammonia-lyase activities belonging to EC 4.3.1 (e.g., aspartate ammonia-lyase activity belonging to EC 4.3.1.1. histidine ammonia-lyase activity belong to EC 4.3.1.3, L-serine ammonia-lyase activity belonging to EC 4.3.1.17, D-serine ammonia-lyase activity belonging to EC 4.3.1.18, threonine ammonia-lyase activity belonging to EC 4.3.1.19, tyrosine ammonia-lyase activity belonging to EC 4.3.1.23, phenylalanine ammonia-lyase activity belonging to EC 4.3.1.24, phenylalanine/tyrosine ammonia-lyase activity belonging to EC 4.3.1.25, L-lysine cyclodeaminase activity belonging to EC 4.3.1.28, L-tryptophan ammonia-lyase activity belonging to EC 4.3.1.31), and/or one or more -sulfur lyase activities belonging to EC 4.4.1 (e.g., cysteine lyase activity belonging to EC 4.4.1.10, methionine gamma-lyase activity belonging to EC 4.4.1.11, L-cysteine desulfidase activity belonging to EC 4.4.1.28, L-cysteine beta-lyase activity belonging to EC 4.4.1.35)—or a corresponding formulation, polynucleotide, or organism, optionally a protein comprising, consisting essentially of, or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 55 and 91 or a functional fragment/mutant/variant thereof (or a corresponding formulation, polynucleotide, or organism) is applied to a plant growth medium, plant, plant part (e.g., a seed), or agricultural/floricultural/horticultural/silvicultural apparatus/facility (e.g., an apparatus/facility for planting, irrigating, fertilizing, growing, monitoring, testing, harvesting, processing, packaging and/or storing a plant or plant part, such as a cultivator, seed container, seeder, planting pot, hydroponic growth system, growth chamber, greenhouse, laboratory, broadcaster, fertilization drill, fertilizer spreader, irrigation system, harvesting apparatus, postharvest storage container, postharvest treatment chamber, or postharvest shipping container) in an amount/concentration effective to a) prevent, treat, suppress and/or eliminate infestation/infection of said plant growth medium, plant, plant part or agricultural/floricultural/horticultural/silvicultural apparatus/facility o/by one or more phytopathogenic pests; b) cleanse said plant growth medium, plant, plant part, or agricultural/floricultural/horticultural/silvicultural apparatus/facility of one or more pest(s); c) prevent, treat, suppress and/or eliminate infestation/infection of a plant grown in said plant growth medium; d) reduce one or more aspects of disease severity in a plant grown in said plant growth medium; e) reduce one or more aspects of disease severity in said plant or plant part; f) prevent, treat, suppress and/or eliminate infestation/infection of a plant or plant part grown in or contacted by said agricultural/floricultural/horticultural/silvicultural apparatus/facility; g) reduce one or more aspects of disease severity in a plant or plant part grown in or contacted by said agricultural/floricultural/horticultural/silvicultural apparatus/facility; h) enhance one or more growth and/or development characteristics of a plant grown in said plant growth medium; i) enhance one or more growth and/or development characteristics of said plant or plant part; j) enhance one or more growth and/or development characteristics of a plant grown in or contacted by said agricultural/floricultural/horticultural/silvicultural apparatus/facility; k) enhance one or more yield characteristics of a plant grown in said plant growth medium; l) enhance one or more yield characteristics in said plant or plant part; m) enhance one or more yield characteristics of a plant grown in or contacted by said agricultural/floricultural/horticultural/silvicultural apparatus/facility; n) enhance the availability of one or more nutrients in said plant growth medium for uptake and/or accumulation by a plant grown therein, optionally boron, calcium, carbon, copper, iron, magnesium, manganese, molybdenum, nitrogen, phosphorous, potassium, sulfur and/or zinc; o) enhance uptake and/or accumulation of one or more nutrients by said plant or plant part, optionally boron, calcium, carbon, copper, iron, magnesium, manganese, molybdenum, nitrogen, phosphorous, potassium, sulfur and/or zinc; p) reduce the amount(s) of exogenous fertilizer needed to achieve a desired result (e.g., the amount of exogenous nitrogen and/or phosphorous required to produce X kilograms of harvested plant material); q) prolong the shelf-life of a plant grown in said plant growth medium; r) prolong the shelf-life of said plant or plant part; s) prolong the shelf-life of a plant grown in or contacted by said agricultural/floricultural/horticultural/silvicultural apparatus/facility; t) delay the ripening of a plant grown in said plant growth medium; u) delay the ripening of said plant or plant part; v) delay the ripening of a plant grown in or contacted by said agricultural/floricultural/horticultural/silvicultural apparatus/facility; w) hasten the ripening of a plant grown in said plant growth medium; x) hastening the ripening of a plant or plant part; y) hasten the ripening of a plant grown in or contacted by said agricultural/floricultural/horticultural/silvicultural apparatus/facility; z) improve the efficacy of a biological pesticide that is applied to said plant, plant part, plant growth medium or agricultural/floricultural/horticultural/silvicultural apparatus/facility prior to, concurrently with, or after application of said protein (or corresponding formulation, polynucleotide, or organism); aa) prevent, treat, suppress and/or eliminate biological-pesticide-induced pest resistance of a plant grown in said plant growth medium; bb) prevent, treat, suppress and/or eliminate biological-pesticide-induced pest resistance of a plant grown in or contacted by said agricultural/floricultural/horticultural/silvicultural apparatus/facility; cc) improve the efficacy of a chemical pesticide that is applied to said plant, plant part, plant growth medium or agricultural/floricultural/horticultural/silvicultural apparatus/facility prior to, concurrently with, or after application of said protein (or corresponding formulation, polynucleotide, or organism); dd) prevent, treat, suppress and/or eliminate chemical-pesticide-induced pest resistance of a plant grown in said plant growth medium; and/or ee) prevent, treat, suppress and/or eliminate chemical-pesticide-induced pest resistance of a plant grown in or contacted by said agricultural/floricultural/horticultural/silvicultural apparatus/facility.

In some embodiments, the protein is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 91;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one of SEQ ID NO(s): 91;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 182 or the cDNA sequence thereof,
d) a polypeptide derived from any one of SEQ ID NO(s): 91 by substitution, deletion, or insertion of one or more amino acids;
e) a polypeptide derived from a mature polypeptide of any one of SEQ ID NO(s): 91 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f)
   wherein the polypeptide exhibits one or more lyase activities.

In some embodiments, a protein exhibiting activity belonging to belonging to EC 4.2—for example, a protein exhibiting activity belonging to EC 4.2.2 (e.g., EC 4.2.2.2, 4.2.2.10)—or a corresponding formulation, polynucleotide, or organism is used to prevent, treat, suppress, eliminate and/or reduce the severity of an infestation/infection of a plant or plant part of/by one or more Botrytis (e.g., B. cinerea), Fusarium (e.g., F. graminearum, F. oxysporum, F. virguliforme), Penicillium (e.g., P. digitatum, P. expansum, P. italicum, P. rugulosum, P. verrucosum), Puccinia (e.g., P. striiformis), Phytophthora (e.g., P. capsici, P. cinnamomi, P. infestans, P. parasitica, P. ramorum, P. sojae), Puccinia (e.g., P. striiformis), Zymoseptoria (e.g., Z. tritici) and insects. For example, a carbon-oxygen lyase, optionally a protein comprising, consisting essentially of or consisting of an amino acid sequence that is about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identical to one or more of the amino acid sequences set forth herein as SEQ ID NO(s): 55 and 91, (or a corresponding formulation, polynucleotide, or organism) maybe applied to a plant or plant part to prevent, treat, suppress, eliminate and/or reducing the severity of such infestations/infections.

In some embodiments, the protein comprises, consists essentially of, or consists of a wild-type polypeptide. For example, in some embodiments, the protein comprises, consists essentially of, or consists of a wild-type polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149 or a mature polypeptide thereof.

In some embodiments, the protein comprises, consists essentially of, or consists of a variant polypeptide. For example, in some embodiments, the protein comprises, consists essentially of, or consists of a variant polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149 or a mature polypeptide thereof.

In some embodiments, the protein comprises, consists essentially of or consists of a catalytic domain, a binding module and a linker between said catalytic domain and said binder module.

In some embodiments, the protein comprises two or more catalytic domains.

In some embodiments, the protein comprises two or more binding modules.

In some embodiments, the protein is a fusion protein comprising a first polypeptide and a second polypeptide, said second polypeptide being distinct from said first polypeptide, wherein said first polypeptide is selected from the group consisting of:
a) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
b) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
c) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
d) a polypeptide derived from any one of SEQ ID NO(s): 1-91, 45906, 72144, 72147 and 72149 by substitution, deletion, or insertion of one or more amino acids;
e) a polypeptide derived from a mature polypeptide any one of SEQ ID NO(s): 1-91, 45906, 72144, 72147 and 72149 by substitution, deletion, or insertion of one or more amino acids;
f) a polypeptide derived from the polypeptide of any one of a) through e) wherein the N- and/or C-terminal end has been extended by the addition of one or more amino acids; and
g) a fragment of the polypeptide of any one of a) through f), and, optionally, wherein said second polypeptide is selected from the group consisting of:
h) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 1-91, 45906, 72144, 72147 and 72149;
i) a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to a mature polypeptide of any one or more of SEQ ID NO(s): 1-91, 45906, 72144, 72147 and 72149;
j) a polypeptide encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof;
k) a polypeptide derived from any one of SEQ ID NO(s): 1-91, 45906, 72144, 72147 and 72149 by substitution, deletion, or insertion of one or more amino acids;
l) a polypeptide derived from a mature polypeptide any one of SEQ ID NO(s): 1-91, 45906, 72144, 72147 and 72149 by substitution, deletion, or insertion of one or more amino acids;
m) a polypeptide derived from the polypeptide of any one of h) through 1) wherein the N- and/or C-terminal end has been extended by the addition of one or more amino acids; and
n) a fragment of the polypeptide of any one of h) through m).

The present disclosure encompasses methods of using one or more compositions of the present disclosure for enhancing the environments in which plants are grown by, for example, changing the pH of the environment, increasing the availability of nutrients, stimulating beneficial microorganisms and/or suppressing/eliminating harmful microorganisms.

The present disclosure encompasses methods of using one or more compositions of the present disclosure for improving plant growth, development and/or yield characteristics by, for example, increasing nutrient availability, increasing nutrient uptake, improving nutrient uptake efficiency, improving water use efficiency, stimulating the production of plant growth promoters, stimulating plant defense mechanisms, and/or preventing, treating, suppressing, eliminating and/or reducing the severity of pest infestations/infections.

The present disclosure encompasses methods of using one or more compositions of the present disclosure for increasing the availability of one or more nutrients by, for example, fixing atmospheric nitrogen, retaining nitrogen in different oxidation stages, solubilizing phosphate, releasing organic acids, and/or solubilizing micronutrients. For example, introducing an enzyme of the present disclosure into soil in the vicinity of a plant may increase the bioavailability of one or more vitamins, macrominerals, micronutrients, organic acids and/or trace minerals, as compared to a control soil lacking said enzyme.

Nitrogen bioavailability may be increased with myriad compositions of the present disclosure, including, but not limited to, proteins exhibiting one or more activities belonging to EC 1.18.6 (e.g., EC 1.18.6.1), EC 3.1.1 (e.g., EC 3.1.1.4, 3.1.1.32) and EC 3.1.4 (e.g., EC 3.1.4.3, EC 3.1.4.4, EC 3.1.4.11) (and corresponding formulations, polynucleotides and organisms). In some embodiments, nitrogen bioavailability is increased using one or more of SEQ ID NO(s): 26 and 32-33, and enzymatically active fragments/mutants/variants thereof.

Phosphate bioavailability may be increased with myriad compositions of the present disclosure, including, but not limited to, proteins exhibiting one or more activities belonging to EC 3.1.3 (e.g., EC 3.1.3.1, EC 3.1.3.2, EC 3.1.3.26), 3.1.4 (e.g., EC 3.1.4.3, EC 3.1.4.4, EC 3.1.4.11) and 3.1.21 (e.g., EC 3.1.21.1) (and corresponding formulations, polynucleotides and organisms). In some embodiments, phosphate bioavailability is increased using one or more of SEQ ID NO(s): 32-33, and enzymatically active fragments/mutants/variants thereof.

The present disclosure encompasses methods of using one or more compositions of the present disclosure for prolonging the shelf-life of harvested plants or plant parts by, for example, treating (e.g., coating) harvested plants and plant parts directly and/or treating post-harvest transport/storage containers. See generally, e.g., US 2019/159470; US 2020/022719; US 2020/352183; US 2011/244095; FR 3038495; WO 2022/049139.

The present disclosure encompasses methods of using one or more compositions of the present disclosure for reducing the need for chemical pesticides by, for example, reducing chemical pesticide-induced phytotoxicity, pest resistance, etc.

Compositions of the present disclosure may be applied to/used to treat any plant type, including, but not limited to, row crops and vegetables. In some embodiments, compositions of the present disclosure are formulated for the treatment of one or more plants selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Caricaceae (e.g., papaya), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, rye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape).

Non-limiting examples of plants that may be treated with compositions of the present disclosure include plants sold under the ACCELERON®, AGRIPRO®, AGRISURE®, AGROESTE®, AGVENTURE®, ALFOREX™, ASGROW®, AQUAMAX®, BOLLGARD II™, BOLLGARD™ 3, BREVANT™, CHANNEL™, CONFIDOR™, CORTEVA AGRISCIENCE™, CORVUS™, CREDENZ®, CROPSTAR™ DAIRYLAND™, DEKALB®, DELTAPINE™, DERUITER™, DROUGHTGARD®, ENLIST E3®, ENOGEN®, FIBERMAX®, GAUCHO™, GENUITY®, GOLDENHARVEST®, HOEGEMEYER™ INTACTA RR2 PRO™, INVIGOR®, LIBERTY LINK®, NEXGROW®, NK®, NUTECH SEED®, OPTIMUM®, PHYTOGEN®, PIONEER®, QROME®, RIB COMPLETE®, ROUNDUP READY®, ROUNDUP READY 2 YIELD®, ROUNDUP READY 2 XTEND®, SEMETES AGROCERES™, SEMINIS™, SMARTSTAX®, STONEVILLE®. SYNGENTA®, TRUFLEX™, VT DOUBLE PRO®, VT TRIPLE PRO®, YIELDGARD®, YIELDGARD VT ROOTWORM/RR2®, YIELDGARD VT TRIPLE® and/or XTENDFLEX™ tradenames.

Compositions of the present disclosure may be applied to any part/portion of a plant. In some embodiments, the compositions are applied to plant propagation materials (e.g., cuttings, rhizomes. seeds and tubers). In some embodiments, the compositions are applied to the roots of a plant. In some embodiments, the compositions are applied to the foliage of a plant. In some embodiments, the compositions are applied to both the roots and the foliage of a plant. In some embodiments, the compositions are applied to plant propagation materials and to the plants that grow from said plant propagation materials.

Compositions of the present disclosure may be applied to any plant growth medium, including, but not limited to, soil.

Compositions of the present disclosure may be applied to plants, plant parts and/or plant growth media in any suitable manner, including, but not limited to, on-seed application, in-furrow application and foliar application.

Compositions of the present disclosure may be applied using any suitable method(s), including, but not limited to, coating, dripping, dusting, encapsulating, fogging, immersing, spraying, and soaking. Batch systems, in which predetermined batch sizes of material and composition are delivered into a mixer, may be employed. Continuous treatment systems, which are calibrated to apply composition at a predefined rate in proportion to a continuous flow of material, may also be employed.

In some embodiments, compositions of the present disclosure are applied directly to plant propagation material (e.g., seeds). According to some embodiments, plant propagation materials are soaked in a composition of the present disclosure for at least 0.1, 0.2, 0.3, 0.4, 0.5, 0.6, 0.7, 0.8, 0.9, 1, 1.25, 1.5, 1.75, 2, 3, 4, 5, 6, 9, 12, 15, 18, 21, 24, 36, 48 hours. According to some embodiments, plant propagation materials are coated with the compositions. Plant propagation materials may be coated with one or more additional layers (e.g., one or more protective layers that serve to enhance the stability and/or activity of an enzyme/organism of the present disclosure and/or one or more sequestration layers comprising substances that may reduce the stability and/or activity of an enzyme/organism of the present disclosure if included in the same layer as the enzyme/organism of the present disclosure). In some embodiments, the coating comprises, consists essentially of, or consists of a composition of the present disclosure and a drying powder.

In some embodiments, compositions of the present disclosure are applied directly to a plant growth medium (e.g., a soil). According to some embodiments, the compositions are applied in the vicinity of a plant propagation material (e.g., a seed). According to some embodiments, the compositions are applied to the root zone of a plant. According to some embodiments, the compositions are applied using a drip irrigation system.

In some embodiments, compositions of the present disclosure are applied directly to plants. According to some embodiments, the compositions are fogged, misted, sprayed and/or sprinkled onto the plant(s) to be treated (e.g., foliar sprays).

In some embodiments, compositions of the present disclosure are applied to harvested plants and/or plant parts.

Individual components of the compositions (e.g., proteins of the present disclosure and chemical pesticides) may be applied separately or together. For example, in some embodiments, compositions of the present disclosure may be incorporated into integrated pest management strategies (e.g., a formulation comprising one or more proteins of the present disclosure may be applied to an orchard/vineyard as part of an integrated pest management strategy that includes separate applications of 2, 3, 4, 5 or more distinct pesticides in a rotation designed to reduce/prevent chemical pesticide-induced phytotoxicity and/or pest resistance).

In some embodiments, compositions of the present disclosure are freeze- spray- or spray-freeze-dried and then applied to plants/plant parts. For examples, in some embodiments, a formulation comprising and enzyme/organism of the present disclosure and one or more stabilizing components (e.g., one or more maltodextrins having a DEV of about 15 to about 20) is freeze- spray- or spray-freeze-dried, mixed with a drying powder (e.g., a drying powder comprising calcium stearate, attapulgite clay, montmorillonite clay, graphite, magnesium stearate, silica (e.g., fumed silica, hydrophobically-coated silica and/or precipitated silica) and/or talc), then coated on seed that was been pre-treated with one or more adhesives (e.g., an adhesive composition comprising one or more maltodextrins, one or more mono-, di- or oligosaccharides, one or more peptones, etc.), one or more pesticides and/or one or more plant signal molecules (e.g., one or more LCOs).

See, generally, e.g., U.S. Ser. No. 11/472,981; US 2020/0085065; U.S. Ser. No. 10/820,594.

Compositions of the present disclosure may be applied to plant growth media (e.g., soil), plants, plant parts and agricultural/floricultural/horticultural/silvicultural apparatuses/facilities at any time, including, but not limited to, prior to planting, at the time of planting, after planting, prior to germination, at the time of germination, after germination, prior to seedling emergence, at the time of seedling emergence, after seedling emergence, prior to the vegetative stage, during the vegetative stage, after the vegetative stage, prior to the reproductive stage, during the reproductive stage, after the reproductive stage, prior to flowering, at the time of flowering, after flowering, prior to fruiting, at the time of fruiting, after fruiting, prior to ripening, at the time of ripening, after ripening, prior to harvest, at the time of harvest, and after harvesting. Indeed, compositions of the present disclosure may be used to extend the shelf-life of harvested products by preventing, treating, suppressing, eliminating and/or reducing the severity of infestations/infections of/by acarids, bacteria, fungi, insects, oomycetes and protozoa for many weeks/months post-harvest.

In some embodiments, compositions of the present disclosure are applied to plant growth media prior to introducing a plant into the plant growth media (e.g., prior to planting seed).

In some embodiments, compositions of the present disclosure are applied to plant growth media concurrently with the introduction of a plant into the plant growth media (e.g., at the time of planting).

In some embodiments, compositions of the present disclosure are applied to plant growth media after introducing a plant into the plant growth media (e.g., by drip irrigation following planting).

In some embodiments, compositions of the present disclosure are applied to plant propagation materials (e.g., seeds) about/at least 1, 2, 3, 4, 5, 6, 7, 8, 9, 10, 11, 12, 13, 14, 15, 16, 17, 18, 19, 20, 21, 22, 23, 24, 28, 32, 36, 40, 44, 48, 52, 56, 60, 64, 68, 72, 76, 80, 84, 88, 92, 96, 100, 104 weeks prior to planting.

In some embodiments, compositions of the present disclosure are applied to plant propagation materials (e.g., seeds) at the time of planting.

In some embodiments, compositions of the present disclosure are applied to plant propagation materials (e.g., seeds) after planting but before germination.

In some embodiments, compositions of the present disclosure are applied to plants following emergence.

In some embodiments, compositions of the present disclosure are applied to a plant or plant part pre-harvest (i.e., within 1, 2, 3, 4, 5, 6, 7, 8, 9, 10, 11, 12, 13, 14, 15 or more days before the plant or plant part is (to be) harvested).

In some embodiments, compositions of the present disclosure are applied to a plant or plant part post-harvest (i.e., after the plant or plant part has been harvested).

In some embodiments, compositions of the present disclosure are applied to a harvested plant or plant part at a processing/shipping facility.

In some embodiments, compositions of the present disclosure are applied to a processed plant product.

In some embodiments, compositions of the present disclosure are applied to an agricultural, floricultural, horticultural and/or silvicultural apparatus/facility prior to contacting a plant or plant part with or introducing a plant or plant part into said apparatus/facility.

In some embodiments, compositions of the present disclosure are applied to an agricultural, floricultural, horticultural and/or silvicultural apparatus/facility concurrently with contacting a plant or plant part with or introducing a plant or plant part into said apparatus/facility.

In some embodiments, compositions of the present disclosure are applied to an agricultural, floricultural, horticultural and/or silvicultural apparatus/facility after contacting a plant or plant part with or introducing a plant or plant part into said apparatus/facility.

Compositions of the present disclosure may be applied to plants, plant parts and/or plant growth media in any suitable amount(s)/concentration(s).

In some embodiments, compositions of the present disclosure comprise one or more proteins of the present disclosure in an amount ranging from about 0.001 to about 100 milligrams per gram and/or milliliter of composition. For example, compositions of the present disclosure may comprise about/at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 milligrams of protein per gram and/or milliliter of composition. In some embodiments, compositions of the present disclosure comprise about/at least 0.05, 0.06, 0.07, 0.08, 0.09, 0.1, 0.15, 0.2, 0.25, 0.3, 0.35, 0.4, 0.45, or 0.5 milligrams of protein per gram and/or milliliter of composition.

In some embodiments, one or more proteins of the present disclosure comprise about 0.00000001 to about 95% (by weight) of the composition. In some embodiments, one or more proteins of the present disclosure comprise about/at least 1×10⁻¹⁵, 1×10⁻¹⁴, 1×10⁻¹³, 1×10⁻¹², 1×10⁻¹¹, 1×10⁻¹⁰, 1×10⁻⁹, 1×10⁻⁸, 1×10⁻⁷, 1×10⁻⁶, 1×10⁻⁵, 1×10⁻⁴, 1×10⁻³, 1×10⁻², 1×10⁻¹, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95% or more (by weight) of the composition.

In some embodiments, the composition is applied at a rate that is equivalent to about 1×10¹ to about 1×10¹⁵ enzyme units (at optimum conditions) of each protein of the present disclosure per kilogram of plant propagation material. According to some embodiments, compositions of the present disclosure are applied in an amount sufficient to ensure the plant propagation materials are coated with about/at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ or 1×10¹⁵ enzyme units (at optimum conditions) of each protein of the present disclosure per kilogram of plant propagation material. According to some embodiments, one or more compositions of the present disclosure is/are applied in an amount sufficient to ensure that an average of about/at least 1×10⁴, 1×10⁵, 1×10⁶, 1×10⁷, 1×10⁸, 1×10⁹, 1×10¹⁰, 1×10¹¹, or 1×10¹² enzyme units (at optimum conditions) of each protein of the present disclosure is applied to each seed.

In some embodiments, the composition is applied at a rate that is equivalent to about 1×10¹ to about 1×10¹⁵ enzyme units (at optimum conditions) of each protein of the present disclosure per plant. According to some embodiments, compositions of the present disclosure are applied in an amount sufficient to ensure each plant is treated with about/at least 1×10¹, 1×10², 1×10¹, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ or 1×10¹⁵ enzyme units (at optimum conditions) of each protein of the present disclosure. According to some embodiments, compositions of the present disclosure are applied in an amount sufficient to ensure that an average of about/at least 1×10⁴, 1×10⁵, 1×10⁶, 1×10⁷, 1×10⁸, 1×10⁹, 1×10¹⁰, 1×10¹¹, or 1×10¹² enzyme units (at optimum conditions) of each protein of the present disclosure is applied to each plant.

In some embodiments, the composition is applied at a rate that is equivalent to about 1×10¹ to about 1×10¹⁵ enzyme units (at optimum conditions) of each protein of the present disclosure per hectare/acre of treated crops. According to some embodiments, compositions of the present disclosure are applied in an amount sufficient to ensure each hectare/acre of treated crops is treated with about/at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ or 1×10¹⁵ enzyme units (at optimum conditions) of each protein of the present disclosure. According to some embodiments, compositions of the present disclosure are applied in an amount sufficient to ensure that an average of about/at least 1×10⁴, 1×10⁵, 1×10⁶, 1×10⁷, 1×10⁸, 1×10⁹, 1×10¹⁰, 1×10¹¹, or 1×10¹² enzyme units (at optimum conditions) of each protein of the present disclosure is applied to each hectare/acre of treated crops.

In some embodiments, the composition is applied at a rate that is equivalent to about 1×10¹ to about 1×10¹⁵ enzyme units (at optimum conditions) of each protein of the present disclosure per hectare/acre of plant growth media. According to some embodiments, compositions of the present disclosure are applied in an amount sufficient to ensure each hectare/acre of plant growth media is treated with about/at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ or 1×10¹⁵ enzyme units (at optimum conditions) of each protein of the present disclosure. According to some embodiments, compositions of the present disclosure are are applied in an amount sufficient to ensure that an average of about/at least 1×10⁴, 1×10⁵, 1×10⁶, 1×10⁷, 1×10⁸, 1×10⁹, 1×10¹⁰, 1×10¹¹, or 1×10¹² enzyme units (at optimum conditions) of each protein of the present disclosure is applied to each hectare/acre of plant growth media.

In some embodiments, the composition is applied at a rate that is equivalent to about 1×10¹ to about 1×10¹⁵ enzyme units (at optimum conditions) of each protein of the present disclosure per square inch/foot of surface area on an agricultural/floricultural/horticultural/silvicultural apparatus/facility. According to some embodiments, compositions of the present disclosure are applied in an amount sufficient to ensure each square inch/foot is treated with about/at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴ or 1×10¹⁵ enzyme units (at optimum conditions) of each protein of the present disclosure. According to some embodiments, compositions of the present disclosure are are applied in an amount sufficient to ensure that an average of about/at least 1×10⁴, 1×10⁵, 1×10⁶, 1×10⁷, 1×10⁸, 1×10⁹, 1×10¹⁰, 1×10¹¹, or 1×10¹² enzyme units (at optimum conditions) of each protein of the present disclosure is applied to each square inch/foot.

In some embodiments, the composition is applied at a rate that is equivalent to about 0.001 to about 100 milligrams of protein of the present disclosure per kilogram of plant propagation material. According to some embodiments, compositions of the present disclosure are applied in an amount sufficient to ensure the plant propagation materials are coated with about/at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 milligrams of protein of the present disclosure per kilogram of plant propagation material. According to some embodiments, one or more compositions of the present disclosure is/are applied in an amount sufficient to ensure that an average of about/at least 0.05, 0.06, 0.07, 0.08, 0.09, 0.1, 0.15, 0.2, 0.25, 0.3, 0.35, 0.4, 0.45, or 0.5 milligrams of protein of the present disclosure is applied to each seed.

In some embodiments, the composition is applied at a rate that is equivalent to about 0.001 to about 100 milligrams of protein of the present disclosure per plant. According to some embodiments, compositions of the present disclosure are applied in an amount sufficient to ensure each plant is treated with about/at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 milligrams of protein of the present disclosure. According to some embodiments, compositions of the present disclosure are applied in an amount sufficient to ensure that an average of about/at least 0.05, 0.06, 0.07, 0.08, 0.09, 0.1, 0.15, 0.2, 0.25, 0.3, 0.35, 0.4, 0.45, or 0.5 milligrams of protein of the present disclosure are applied to each plant.

In some embodiments, the composition is applied at a rate that is equivalent to about 0.001 to about 100 milligrams of protein of the present disclosure per hectare/acre of treated crops. According to some embodiments, compositions of the present disclosure are applied in an amount sufficient to ensure each hectare/acre of treated crops is treated with about/at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 milligrams of protein of the present disclosure. According to some embodiments, compositions of the present disclosure are applied in an amount sufficient to ensure that an average of about/at least 0.05, 0.06, 0.07, 0.08, 0.09, 0.1, 0.15, 0.2, 0.25, 0.3, 0.35, 0.4, 0.45, or 0.5 milligrams of protein of the present disclosure are applied to each hectare/acre of treated crops.

In some embodiments, the composition is applied at a rate that is equivalent to about 0.001 to about 100 milligrams of protein of the present disclosure per hectare/acre of plant growth media. According to some embodiments, compositions of the present disclosure are applied in an amount sufficient to ensure each hectare/acre of plant growth media is treated with about/at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 milligrams of protein of the present disclosure. According to some embodiments, compositions of the present disclosure are applied in an amount sufficient to ensure that an average of about/at least 0.05, 0.06, 0.07, 0.08, 0.09, 0.1, 0.15, 0.2, 0.25, 0.3, 0.35, 0.4, 0.45, or 0.5 milligrams of protein of the present disclosure are applied to each hectare/acre of plant growth media.

In some embodiments, compositions of the present disclosure are applied at a rate of about 0.05 to about 100 milliliters and/or grams of composition per kilogram of plant propagation material. According to some embodiments, one or more compositions of the present disclosure is/are applied in an amount sufficient to ensure the plant propagation materials are coated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of compositions per kilogram of plant propagation material. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each seed.

In some embodiments, compositions of the present disclosure are applied at a rate of about 0.5 to about 100 milliliters and/or grams of composition per plant. According to some embodiments, one or more compositions of the present disclosure is/are applied in an amount sufficient to ensure each plant is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each plant.

In some embodiments, compositions of the present disclosure are applied at a rate of about 0.5 to about 100 milliliters and/or grams of composition per hectare/acre of treated crops. According to some embodiments, one or more compositions of the present disclosure is/are applied in an amount sufficient to ensure each hectare/acre of treated crops is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each hectare/acre of treated crops.

In some embodiments, compositions of the present disclosure are applied at a rate of about 0.5 to about 100 milliliters and/or grams of composition per hectare/acre of plant growth media. According to some embodiments, one or more compositions of the present disclosure is/are applied in an amount sufficient to ensure each hectare/acre of plant growth media is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each hectare/acre of plant growth media.

In some embodiments, compositions of the present disclosure are applied at a rate of about 0.5 to about 100 milliliters and/or grams of composition per square inch/foot of surface area on an agricultural/floricultural/horticultural/silvicultural apparatus/facility. According to some embodiments, one or more compositions of the present disclosure is/are applied in an amount sufficient to ensure each square inch/foot is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each square inch/foot.

In some embodiments, compositions of the present disclosure are applied at a rate sufficient to prevent, treat, suppress and/or eliminate one or more pest infestations/infections. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to prevent, treat, suppress and/or eliminate infestation/infection by 1, 2, 3, 4, 5, 6, 7, 8, 9, 10 or more phytopathogenic pests.

In some embodiments, compositions of the present disclosure are applied at a rate sufficient to reduce one or more aspects of disease severity. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to reduce one or more aspects of disease severity by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to enhance nutrient availability, uptake and/or accumulation. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to enhance the availability, uptake and/or accumulation of one or more nutrients, optionally one or more of boron, calcium, carbon, copper, iron, magnesium, manganese, molybdenum, nitrogen, phosphorous, potassium, sulfur and/or zinc availability, uptake and/or accumulation, by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% or more, as compared to an untreated control.

In some embodiments, compositions of the present disclosure are applied at a rate sufficient to enhance one or more plant growth and/or development characteristics. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to enhance one or more plant growth and/or development characteristics, optionally biomass, carbohydrate biosynthesis, chlorophyll content, cold tolerance, drought tolerance, height, leaf length, leaf mass, leaf number, leaf surface area, leaf volume, nutrient uptake (e.g., calcium, magnesium, nitrogen, phosphorous and/or potassium uptake), rate(s) of photosynthesis, root area, root diameter, root length, root mass, root nodulation (e.g., nodule mass, nodule number, nodule volume), root number, root surface area, root volume, salt tolerance, seed germination, seedling emergence, shoot diameter, shoot length, shoot mass, shoot number, shoot surface area, shoot volume, spread, stomatal conductance and/or survival rate, by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200%6 or more, as compared to an untreated control.

In some embodiments, compositions of the present disclosure are applied at a rate sufficient to enhance one or more plant yield characteristics. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to enhance one or more plant yield characteristics, optionally biomass; bushels per acre; grain weight per plot (GWTPP); nutritional content; percentage of plants in a given area (e.g., plot) that fail to produce grain; yield at standard moisture percentage (YSMP), such as grain yield at standard moisture percentage (GYSMP); yield per plot (YPP), such as grain weight per plot (GWTPP); and yield reduction (YRED), by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200%6 or more, as compared to an untreated control.

In some embodiments, compositions of the present disclosure are applied at a rate sufficient to reduce the need for exogenous fertilizer. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to reduce the amount(s) of exogenous fertilizer, optionally exogenous boron, calcium, carbon, copper, iron, magnesium, manganese, molybdenum, nitrogen, phosphorous, potassium, sulfur and/or zinc, needed to achieve a desired result by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to enhance the efficacy of a biological pesticide. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to enhance the efficacy of a biological pesticide by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% or more, as compared to an untreated control.

In some embodiments, compositions of the present disclosure are applied at a rate sufficient to enhance the efficacy of a chemical pesticide. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to enhance the efficacy of a chemical pesticide by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% or more, as compared to an untreated control.

In some embodiments, compositions of the present disclosure are applied at a rate sufficient to reduce one or more aspects of pesticide-induced pest resistance. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to reduce one or more aspects of pesticide-induced pest resistance by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to reduce one or more aspects of pesticide-induced phytotoxicity. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to reduce one or more aspects of pesticide-induced phytotoxicity by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to delay ripening of a harvest plant or plant part. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to delay ripening of a harvest plant or plant part by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to hasten ripening of a harvest plant or plant part. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to hasten ripening of a harvest plant or plant part by about/at least 5, 10, 15, 20, 25, 30, 35, 40, 45, 50, 55, 60, 65, 70, 75, 80, 85, 90, 95, or 100%, as compared to an untreated control.

In some embodiments, compositions of the present disclosure are applied at a rate sufficient to extend the shelf-life of a harvested plant or plant part. According to some embodiments, one or more compositions of the present disclosure is/are applied in an amount sufficient to ensure the plant, plant part, plant growth medium and/or agricultural/floricultural/horticultural/silvicultural apparatus/facility is treated with an amount sufficient to extend the shelf-life of a harvested plant or plant part by about/at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200% or more, as compared to an untreated control.

The present disclosure extends to plant growth media, plants, plant parts and agricultural/floricultural/horticultural/silvicultural apparatuses/facilities that have been treated with a composition of the present disclosure (e.g., plant propagation materials coated with a formulation comprising one or more enzymes of the present disclosure, plants sprayed with a formulation comprising one or more enzymes of the present disclosure, harvested plant parts coated with a formulation comprising one or more enzymes of the present disclosure), to plants grown from plant propagation materials that were treated with a composition of the present disclosure, to plant parts harvested from plants that have been treated with a composition of the present disclosure, to plant parts harvested from plants grown from plant propagation materials that were treated with a composition of the present disclosure, to crops comprising a plurality of plants that were treated with a composition of the present disclosure, to crops comprising a plurality of plants grown from plant propagation materials that were treated with a composition of the present disclosure, to crops treated with a composition of the present disclosure, to processed products derived from plants that were treated with a composition of the present disclosure, to processed products derived from plants grown from plant parts that were treated with a composition of the present disclosure, and to processed products treated with a composition of the present disclosure.

The present disclosure encompasses coated plant propagation materials comprising, consisting essentially of, or consisting of a plant propagation material and a coating that covers at least a portion of the outer surface of the plant propagation material, said coating comprising, consisting essentially of, or consisting of one or more compositions of the present disclosure.

In some embodiments, the coating comprises two, three, four, five or more layers. According to some embodiments, the coating comprises an inner layer that contains one or more proteins of the present disclosure and one or more outer layers free or substantially free of proteins of the present disclosure. In some embodiments, the coating comprises an inner layer that is a composition of the present disclosure and an outer layer that is equivalent to a composition of the present disclosure except that it does not contain proteins of the present disclosure.

In some embodiments, the coating comprises, consists essentially of, or consists of a composition of the present disclosure and a drying powder. Drying powders may be applied in any suitable amount(s)/concentration(s). The absolute value of the amount/concentration that is/are sufficient to cause the desired effect(s) may be affected by factors such as the type, size and volume of material to which the composition will be applied, the type(s) of proteins in the composition, the number of proteins in the composition, the stability of the proteins in the composition and storage conditions (e.g., temperature, relative humidity, duration). Those skilled in the art will understand how to select an effective amount/concentration using routine dose-response experiments. Guidance for the selection of appropriate amounts/concentrations can be found, for example, in International Patent Application Nos. PCT/US2016/050529 and PCT/US2016/050647 and U.S. Provisional Patent Application Nos. 62/296,798; 62/271,857; 62/347,773; 62/343,217; 62/296,784; 62/271,873; 62/347,785; 62/347,794; and 62/347,805. In some embodiments, the drying powder is applied in an amount ranging from about 0.5 to about 10 grams of drying powder per kilogram of plant propagation material. For example, in some embodiments, about 0.5, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10 grams or more of drying powder (e.g., drying powder comprising magnesium stearate, magnesium sulfate, powdered milk, silica, soy lecithin and/or talc) is applied per kilogram of seed. In some embodiments, a drying powder comprising calcium stearate, attapulgite clay, montmorillonite clay, graphite, magnesium stearate, silica (e.g., fumed silica, hydrophobically-coated silica and/or precipitated silica) and/or talc is applied to seeds coated with a composition of the present disclosure at a rate of about 1, 1.25, 1.5, 1.75, 2, 2.25, 2.5, 2.75, or 3 grams per kilogram of seed.

In some embodiments, the coating completely covers the outer surface of the plant propagation material.

In some embodiments, the average thickness of the coating is at least 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.5, 5 μm or more. In some embodiments, the average thickness of the coating is about 1.5 to about 3.0 μm.

The present disclosure extends to kits comprising, consisting essentially of, or consisting of one or more plants and/or plant parts (e.g., coated plant propagation materials) that have been treated with the compositions of the present disclosure and a container housing the treated plant(s) and/or plant part(s). In some embodiments, the kit further comprises one or more oxygen scavengers, such as activated carbon, ascorbic acid, iron powder, mixtures of ferrous carbonate and metal halide catalysts, sodium chloride and/or sodium hydrogen carbonate.

The container may comprise any suitable material(s), including, but not limited to, materials that reduce the amount of light, moisture and/or oxygen that contact the coated plant propagation material when the container is sealed. In some embodiments, the container comprises, consists essentially of, or consists of a material having light permeability of less than about 1, 2, 3, 4, 5, 6, 7, 8, 9, 10, 15, 20, 25, 30, 35, 40, 45, 50, 55, 60, 65, 70 or 75%. In some embodiments, the container comprises, consists essentially of, or consists of a material having an oxygen transmission rate of less than about 5, 10, 15, 20, 25, 30, 35, 40, 45, 50, 60, 70, 80, 90, 100, 125, 150, 175, 200, 225, 250, 275, 300, 325, 350, 375, 400, 425, 450, 475, or 500 cm³/m²·day (as measured in accordance with ASTM D3985).

In some embodiments, the container reduces the amount of ambient light that reaches said coated plant propagation material by about 5, 10, 15, 20, 25, 30, 35, 40, 45, 50, 55, 60, 65, 70, 75, 80, 85, 90, 95 or 100% when sealed.

In some embodiments, the container reduces the amount of ambient moisture that reaches said plant propagation material by about 5, 10, 15, 20, 25, 30, 35, 40, 45, 50, 55, 60, 65, 70, 75, 80, 85, 90, 95 or 100% when sealed.

In some embodiments, the container reduces the amount of ambient oxygen that reaches said plant propagation material by about 5, 10, 15, 20, 25, 30, 35, 40, 45, 50, 55, 60, 65, 70, 75, 80, 85, 90, 95 or 100% when sealed.

In some embodiments, kits of the present disclosure comprise 1, 2, 3, 4, 5 or more additional containers. The additional containers may comprise any suitable component(s) or composition(s), including, but not limited to, agriculturally beneficial microorganisms, biostimulants, drying agents, nutrients, oxidation control components and pesticides. Examples of agriculturally beneficial microorganisms, biostimulants, drying agents, nutrients, oxidation control components and pesticides that may be included in the additional containers are described above.

As noted above, proteins of the present disclosure may be formulated into compositions comprising a variety of components, such as adhesives (stickers), chemical actives, dispersants (spreaders), drying agents, emulsifiers, microbes, nutrients, pest attractants and feeding stimulants, pH control components, postharvest treatments, rain fasteners, rhcalogical agents, safeners, stabilizers, UV protectants and wetting agents. It is to be understood that compositions and methods of the present disclosure may likewise be used in combination with such components as separate and distinct compositions (as part of an integrated pest management strategy, for example).

It is to be understood that compositions and methods of the present disclosure may be combined with known compositions and methods, such as fertilization compositions/methods, inoculant compositions/methods, pesticide compositions/methods, and post-harvest compositions/methods.

In some instances, compositions and methods of the present disclosure are used as part of an Integrated Pest Management program/strategy. According to some embodiments, one or more proteins, organisms and/or formulations of the present disclosure is/are applied to a plant, plant part or plant growth medium as part of an Integrated Pest Management program/strategy comprising one or more biological pesticides and/or one or more chemical pesticides.

It is to be understood that compositions and methods of the present disclosure are not limited to agricultural/floricultural/horticultural/silvicultural uses. The same activities that make enzymes, formulations, nucleic acids and organisms of the present disclosure useful for preventing, treating, suppressing and/or eliminating infestations/infections of/by phytopathogenic pests likewise render them useful for preventing/treating/suppressing/eliminating/reducing the detrimental effects of infestations/infections of/by arachnids, bacteria, fungi, insects, oomycetes, protozoa and viruses in and on myriad surfaces/substances, such as food storage containers, animal bedding/feed, clothing, hard surfaces, medical instruments, etc. Thus, it is to be understood that compositions and methods of the present disclosure may be modified for use in any other industry or endeavor in which such prevention/treatment/suppression/elimination/reduction may be of benefit.

For example, in some embodiments, the present disclosure extends to animal feed compositions comprising, consisting essentially of or consisting of a food component and an enzyme component, said enzyme component comprising, consisting essentially of, or consisting of one or more compositions of the present disclosure.

Animal feed compositions of the present disclosure may comprise any suitable food component, including, but not limited to, fodder (e.g., grains, hay, legumes, silage and/or straw) and forage (e.g., grass).

Animal feed compositions of the present disclosure may be fed to any suitable animal, including, but not limited to, farm animals, zoo animals, laboratory animals and/or companion animals. In some embodiments, the animal feed composition is formulated to meet the dietary needs of birds (e.g., chickens, ducks, quails and/or turkeys), bovids (e.g., antelopes, bison, cattle, gazelles, goats, impala, oxen, sheep and/or wildebeasts), canines, cervids (e.g., caribou, deer, elk and/or moose), equines (e.g., donkeys, horses and/or zebras), felines, fish, pigs, rabbits, rodents (e.g., guinea pigs, hamsters, mice and/or rats) and the like.

The following is a non-exhaustive listing of concepts and embodiments encompassed by the present disclosure: Use of a composition of the present disclosure (e.g., a protein or formulation of the present disclosure) for any one, two, three, four, five, six, seven, eight, nine, ten or more of the following:
1) preventing, treating, suppressing and/or eliminating infestation/infection of a surface/substance of/by one or more pests, optionally one or more acarids, bacteria, fungi, gastropods, insects, nematodes, oomycetes, protozoa and/or viruses;
2) preventing, treating, suppressing and/or eliminating infestation/infection of a plant, plant part, plant growth medium or agricultural/floricultural/horticultural/silvicultural apparatus/facility (e.g., an apparatus/facility for planting, irrigating, fertilizing, growing, monitoring, testing, harvesting, processing, packaging and/or storing a plant or plant part) of/by one or more phytopathogenic pests, optionally one or more arachnids, bacteria, fungi, gastropods, insects, nematodes, oomycetes protozoa, viruses and/or weeds;
3) reducing one or more aspects of disease severity in a plant or plant part affected by one or more phytopathogenic pests, optionally one or more arachnids, bacteria, fungi, gastropods, insects, nematodes, oomycetes protozoa, viruses and/or weeds;
4) reducing one or more aspects of disease severity in a plant or plant part grown in a plant growth medium that is infested/infected by/with one or more phytopathogenic pests, optionally one or more arachnids, bacteria, fungi, gastropods, insects, nematodes, oomycetes protozoa, viruses and/or weeds;
5) reducing one or more aspects of disease severity in a plant or plant part grown in or contacted by agricultural/floricultural/horticultural/silvicultural apparatus/facility that is infested/infected by/with one or more phytopathogenic pests, optionally one or more arachnids, bacteria, fungi, gastropods, insects, nematodes, oomycetes protozoa, viruses and/or weeds;
6) treating a surface/substance that is susceptible to infestation/infection by one or more pests, optionally one or more acarids, bacteria, fungi, gastropods, insects, nematodes, oomycetes, protozoa and/or viruses;
7) cleaning a surface/substance that is infested/infected by one or more pests, optionally one or more acarids, bacteria, fungi, gastropods, insects, nematodes, oomycetes, protozoa and/or viruses;
8) treating (e.g., coating, dipping, drenching, fogging, misting, soaking, spraying) a plant or plant part;
9) treating (e.g., drenching, fogging, misting, spraying) a plant growth medium;
10) treating (e.g., coating, dipping, drenching, fogging, irrigating, misting, soaking, spraying) an agricultural/floricultural/horticultural/silvicultural apparatus/facility, optionally an apparatus/facility for planting, irrigating, fertilizing, growing, monitoring, testing, harvesting, processing, packaging and/or storing a plant or plant part;
11) improving one or more soil characteristics;
12) improving nutrient availability/uptake/accumulation, optionally boron, calcium, carbon, copper, iron, magnesium, manganese, molybdenum, nitrogen, phosphorous, potassium, sulfur and/or zinc availability/uptake/accumulation;
13) improving one or more characteristics of plant growth and/or development;
14) reducing the need for exogenous fertilize;
15) reducing the amount(s) of exogenous fertilizer needed to achieve desired result;
16) improving one or more characteristics of plant yield;
17) prolonging the shelf-life of a harvested plant or plant part;
18) delaying the ripening of a harvested plant or plant part;
19) hastening the ripening of a harvested plant or plant part;
20) improving the efficacy of a chemical pesticide, optionally an acaricide, bactericide, fungicide, gastropodicide, herbicide, insecticide, nematicide, oomyceticide, protozoacide or viricide;
21) improving the efficacy of a biological pesticide, optionally an acaricide, bactericide, fungicide, gastropodicide, herbicide, insecticide, nematicide, oomyceticide, protozoacide or viricide;
22) improving the efficacy of a preharvest treatment;
23) improving the efficacy of a postharvest treatment;
24) preventing, treating, suppressing and/or eliminating chemical pesticide-induced pest resistance and/or phytotoxicity;
25) preventing, treating, suppressing and/or eliminating biological pesticide-induced pest resistance and/or phytotoxicity;
26) inclusion as part of an Integrated Pest Management program/strategy, optionally an Integrated Pest Management program/strategy comprising one or more chemical pesticides.

Any of the foregoing uses in which any one, two, three, four, five, six, seven, eight, nine, ten or more of the following is true:
1) the composition comprises, consists essentially of or consists of an enzyme
2) the composition comprises, consists essentially of or consists of an enzyme belonging to EC 1
3) the composition comprises, consists essentially of or consists of an enzyme belonging to EC 1.1
4) the composition comprises, consists essentially of or consists of an enzyme belonging to EC 1.1.3
5) the composition comprises, consists essentially of or consists of an enzyme belonging to EC 1.1.3.4
6) the composition comprises, consists essentially of or consists of an enzyme belonging to EC 1.1.99
7) the composition comprises, consists essentially of or consists of an enzyme belonging to EC 1.1.99.18
8) the composition comprises, consists essentially of or consists of an enzyme belonging to EC 1.4
9) the composition comprises, consists essentially of or consists of an enzyme belonging to EC 1.4.3
10) the composition comprises, consists essentially of or consists of an enzyme belonging to EC 1.4.3.1
11) the composition comprises, consists essentially of or consists of an enzyme belonging to EC 1.4.3.2
12) the composition comprises, consists essentially of or consists of an enzyme belonging to EC 1.4.3.3
13) the composition comprises, consists essentially of or consists of an enzyme belonging to EC 1.4.3.7
14) the composition comprises, consists essentially of or consists of an enzyme belonging to EC 1.4.3.11
15) the composition comprises, consists essentially of or consists of an enzyme belonging to EC 1.4.3.12
16) the composition comprises, consists essentially of or consists of an enzyme belonging to EC 1.4.3.13
17) the composition comprises, consists essentially of or consists of an enzyme belonging to EC 1.4.3.14
18) the composition comprises, consists essentially of or consists of an enzyme belonging to EC 1.4.3.15
19) the composition comprises, consists essentially of or consists of an enzyme belonging to EC 1.4.3.16
20) the composition comprises, consists essentially of or consists of an enzyme belonging to EC 1.4.3.19
21) the composition comprises, consists essentially of or consists of an enzyme belonging to EC 1.4.3.20
22) the composition comprises, consists essentially of or consists of an enzyme belonging to EC 1.4.3.25
23) the composition comprises, consists essentially of or consists of an enzyme belonging to EC 1.10
24) the composition comprises, consists essentially of or consists of an enzyme belonging to EC 1.10.3
25) the composition comprises, consists essentially of or consists of an enzyme belonging to EC 1.10.3.2
26) the composition comprises, consists essentially of or consists of an enzyme belonging to EC 1.11
27) the composition comprises, consists essentially of or consists of an enzyme belonging to EC 1.11.1
28) the composition comprises, consists essentially of or consists of an enzyme belonging to EC 1.11.1.6
29) the composition comprises, consists essentially of or consists of an enzyme belonging to EC 1.11.1.7
30) the composition comprises, consists essentially of or consists of an enzyme belonging to EC 1.14
31) the composition comprises, consists essentially of or consists of an enzyme belonging to EC 1.14.18
32) the composition comprises, consists essentially of or consists of an enzyme belonging to EC 1.14.18.1
33) the composition comprises, consists essentially of or consists of an enzyme belonging to EC 1.14.99
34) the composition comprises, consists essentially of or consists of an enzyme belonging to EC 1.14.99.56
35) the composition comprises, consists essentially of or consists of an enzyme belonging to EC 2
36) the composition comprises, consists essentially of or consists of an enzyme belonging to EC 2.3
37) the composition comprises, consists essentially of or consists of an enzyme belonging to EC 2.3.2
38) the composition comprises, consists essentially of or consists of an enzyme belonging to EC 2.3.2.2
39) the composition comprises, consists essentially of or consists of an enzyme belonging to EC 3
40) the composition comprises, consists essentially of or consists of an enzyme belonging to EC 3.1
41) the composition comprises, consists essentially of or consists of an enzyme belonging to EC 3.1.1
42) the composition comprises, consists essentially of or consists of an enzyme belonging to EC 3.1.1.3
43) the composition comprises, consists essentially of or consists of an enzyme belonging to EC 3.1.1.5
44) the composition comprises, consists essentially of or consists of an enzyme belonging to EC 3.1.1.11
45) the composition comprises, consists essentially of or consists of an enzyme belonging to EC 3.1.1.32
46) the composition comprises, consists essentially of or consists of an enzyme belonging to EC 3.1.1.74
47) the composition comprises, consists essentially of or consists of an enzyme belonging to EC 3.1.4
48) the composition comprises, consists essentially of or consists of an enzyme belonging to EC 3.1.4.3
49) the composition comprises, consists essentially of or consists of an enzyme belonging to EC 3.1.4.11
50) the composition comprises, consists essentially of or consists of an enzyme belonging to EC 3.2
51) the composition comprises, consists essentially of or consists of an enzyme belonging to EC 3.2.1
52) the composition comprises, consists essentially of or consists of an enzyme belonging to EC 3.2.1.1
53) the composition comprises, consists essentially of or consists of an enzyme belonging to EC 3.2.1.2
54) the composition comprises, consists essentially of or consists of an enzyme belonging to EC 3.2.1.3
55) the composition comprises, consists essentially of or consists of an enzyme belonging to EC 3.2.1.4
56) the composition comprises, consists essentially of or consists of an enzyme belonging to EC 3.2.1.5
57) the composition comprises, consists essentially of or consists of an enzyme belonging to EC 3.2.1.6
58) the composition comprises, consists essentially of or consists of an enzyme belonging to EC 3.2.1.7
59) the composition comprises, consists essentially of or consists of an enzyme belonging to EC 3.2.1.8
60) the composition comprises, consists essentially of or consists of an enzyme belonging to EC 3.2.1.10
61) the composition comprises, consists essentially of or consists of an enzyme belonging to EC 3.2.1.11
62) the composition comprises, consists essentially of or consists of an enzyme belonging to EC 3.2.1.14
63) the composition comprises, consists essentially of or consists of an enzyme belonging to EC 3.2.1.15
64) the composition comprises, consists essentially of or consists of an enzyme belonging to EC 3.2.1.17
65) the composition comprises, consists essentially of or consists of an enzyme belonging to EC 3.2.1.20
66) the composition comprises, consists essentially of or consists of an enzyme belonging to EC 3.2.1.21
67) the composition comprises, consists essentially of or consists of an enzyme belonging to EC 3.2.1.22
68) the composition comprises, consists essentially of or consists of an enzyme belonging to EC 3.2.1.23
69) the composition comprises, consists essentially of or consists of an enzyme belonging to EC 3.2.1.24
70) the composition comprises, consists essentially of or consists of an enzyme belonging to EC 3.2.1.25
71) the composition comprises, consists essentially of or consists of an enzyme belonging to EC 3.2.1.26
72) the composition comprises, consists essentially of or consists of an enzyme belonging to EC 3.2.1.28
73) the composition comprises, consists essentially of or consists of an enzyme belonging to EC 3.2.1.32
74) the composition comprises, consists essentially of or consists of an enzyme belonging to EC 3.2.1.37
75) the composition comprises, consists essentially of or consists of an enzyme belonging to EC 3.2.1.39
76) the composition comprises, consists essentially of or consists of an enzyme belonging to EC 3.2.1.41
77) the composition comprises, consists essentially of or consists of an enzyme belonging to EC 3.2.1.55
78) the composition comprises, consists essentially of or consists of an enzyme belonging to EC 3.2.1.58
79) the composition comprises, consists essentially of or consists of an enzyme belonging to EC 3.2.1.59
80) the composition comprises, consists essentially of or consists of an enzyme belonging to EC 3.2.1.73
81) the composition comprises, consists essentially of or consists of an enzyme belonging to EC 3.2.1.75
82) the composition comprises, consists essentially of or consists of an enzyme belonging to EC 3.2.1.78
83) the composition comprises, consists essentially of or consists of an enzyme belonging to EC 3.2.1.91
84) the composition comprises, consists essentially of or consists of an enzyme belonging to EC 3.2.1.92
85) the composition comprises, consists essentially of or consists of an enzyme belonging to EC 3.2.1.97
86) the composition comprises, consists essentially of or consists of an enzyme belonging to EC 3.2.1.101
87) the composition comprises, consists essentially of or consists of an enzyme belonging to EC 3.2.1.109
88) the composition comprises, consists essentially of or consists of an enzyme belonging to EC 3.2.1.111
89) the composition comprises, consists essentially of or consists of an enzyme belonging to EC 3.2.1.112
90) the composition comprises, consists essentially of or consists of an enzyme belonging to EC 3.2.1.125
91) the composition comprises, consists essentially of or consists of an enzyme belonging to EC 3.2.1.130
92) the composition comprises, consists essentially of or consists of an enzyme belonging to EC 3.2.1.132
93) the composition comprises, consists essentially of or consists of an enzyme belonging to EC 3.2.1.133
94) the composition comprises, consists essentially of or consists of an enzyme belonging to EC 3.2.1.163
95) the composition comprises, consists essentially of or consists of an enzyme belonging to EC 3.2.1.176
96) the composition comprises, consists essentially of or consists of an enzyme belonging to EC 3.2.1.198
97) the composition comprises, consists essentially of or consists of an enzyme belonging to EC 3.4
98) the composition comprises, consists essentially of or consists of an enzyme belonging to EC 3.4.11
99) the composition comprises, consists essentially of or consists of an enzyme belonging to EC 3.4.11.1
100) the composition comprises, consists essentially of or consists of an enzyme belonging to EC 3.4.21
101) the composition comprises, consists essentially of or consists of an enzyme belonging to EC 3.4.21.19
102) the composition comprises, consists essentially of or consists of an enzyme belonging to EC 3.4.21.62
103) the composition comprises, consists essentially of or consists of an enzyme belonging to EC 3.4.24
104) the composition comprises, consists essentially of or consists of an enzyme belonging to EC 3.4.24.28
105) the composition comprises, consists essentially of or consists of an enzyme belonging to EC 3.5
106) the composition comprises, consists essentially of or consists of an enzyme belonging to EC 3.5.1
107) the composition comprises, consists essentially of or consists of an enzyme belonging to EC 3.5.1.1
108) the composition comprises, consists essentially of or consists of an enzyme belonging to EC 3.5.1.2
109) the composition comprises, consists essentially of or consists of an enzyme belonging to EC 3.5.1.4
110) the composition comprises, consists essentially of or consists of an enzyme belonging to EC 3.5.1.5
111) the composition comprises, consists essentially of or consists of an enzyme belonging to EC 3.5.1.12
112) the composition comprises, consists essentially of or consists of an enzyme belonging to EC 3.5.1.19
113) the composition comprises, consists essentially of or consists of an enzyme belonging to EC 3.5.1.33
114) the composition comprises, consists essentially of or consists of an enzyme belonging to EC 3.5.1.35
115) the composition comprises, consists essentially of or consists of an enzyme belonging to EC 3.5.1.38
116) the composition comprises, consists essentially of or consists of an enzyme belonging to EC 3.5.1.41
117) the composition comprises, consists essentially of or consists of an enzyme belonging to EC 3.5.1.43
118) the composition comprises, consists essentially of or consists of an enzyme belonging to EC 3.5.1.44
119) the composition comprises, consists essentially of or consists of an enzyme belonging to EC 3.5.1.50
120) the composition comprises, consists essentially of or consists of an enzyme belonging to EC 3.5.1.104
121) the composition comprises, consists essentially of or consists of an enzyme belonging to EC 4
122) the composition comprises, consists essentially of or consists of an enzyme belonging to EC 4.2
123) the composition comprises, consists essentially of or consists of an enzyme belonging to EC 4.2.2
124) the composition comprises, consists essentially of or consists of an enzyme belonging to EC 4.2.2.2
125) the composition comprises, consists essentially of or consists of an enzyme belonging to EC 4.2.2.10
126) the composition comprises, consists essentially of or consists of an enzyme belonging to EC 4.2.2.13
127) the composition comprises, consists essentially of or consists of an enzyme belonging to EC 4.2.2.25
128) the composition comprises, consists essentially of or consists of an enzyme belonging to EC 4.2.2.26
129) the composition comprises, consists essentially of or consists of an enzyme belonging to EC 4.2.2.27
130) the composition comprises, consists essentially of or consists of an enzyme belonging to EC 4.3
131) the composition comprises, consists essentially of or consists of an enzyme belonging to EC 4.3.1
132) the composition comprises, consists essentially of or consists of an enzyme belonging to EC 4.3.1.1
133) the composition comprises, consists essentially of or consists of an enzyme belonging to EC 4.3.1.3
134) the composition comprises, consists essentially of or consists of an enzyme belonging to EC 4.3.1.17
135) the composition comprises, consists essentially of or consists of an enzyme belonging to EC 4.3.1.18
136) the composition comprises, consists essentially of or consists of an enzyme belonging to EC 4.3.1.19
137) the composition comprises, consists essentially of or consists of an enzyme belonging to EC 4.3.1.23
138) the composition comprises, consists essentially of or consists of an enzyme belonging to EC 4.3.1.24
139) the composition comprises, consists essentially of or consists of an enzyme belonging to EC 4.3.1.25
140) the composition comprises, consists essentially of or consists of an enzyme belonging to EC 4.3.1.31
141) the composition comprises, consists essentially of or consists of one or more polypeptides having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 1-91, 45906, 72144, 72147 and 72149 or a mature polypeptide thereof
142) the composition comprises, consists essentially of or consists of one or more polypeptides encoded by a polynucleotide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof
143) the composition comprises, consists essentially of or consists of one or more polypeptides derived from any one of SEQ ID NO(s): 1-91, 45906, 72144, 72147 and 72149 by substitution, deletion, or insertion of one or more amino acids
144) the composition comprises, consists essentially of or consists of one or more polypeptides derived from a mature polypeptide of any one of SEQ ID NO(s): 1-91, 45906, 72144, 72147 and 72149 by substitution, deletion, or insertion of one or more amino acids
145) the composition comprises, consists essentially of or consists of one or more polypeptides derived from any one of 71) through 74) wherein the N- and/or C-terminal end has been extended by the addition of one or more amino acids
146) the composition comprises, consists essentially of or consists of a fragment of any one of 71) through 75)
147) the composition comprises, consists essentially of or consists of an enzymatically active fragment/mutant/variant of any one of SEQ ID NO(s): 1-91, 45906, 72144, 72147 and 72149 or a mature polypeptide thereof
148) the composition comprises a fusion protein, optionally a fusion protein comprising a first polypeptide and a second polypeptide wherein at least one of said first and second polypeptides is a protein of the present disclosure
149) the composition comprises 2, 3, 4, 5, 6, 7, 8, 9, 10 or more proteins of the present disclosure or enzymatically active fragments/mutations/variants thereof
150) the composition is an aqueous enzyme solution, optionally an aqueous enzyme solution comprising, consisting essentially of or consisting of water, one or more proteins of the present disclosure and one or more preservatives (e.g., potassium sorbate, sodium benzoate, 1,2-benzisothiazolin-3-one) and/or stabilizers (e.g., glycerol, potassium chloride, propylene glycol, sodium chloride, sorbitol)
151) the composition comprises an agriculturally acceptable carrier
152) the composition comprises a seed-compatible carrier
153) the composition comprises a foliar-compatible carrier
154) the composition comprises a soil-compatible carrier
155) the composition comprises one or more adhesives (stickers), optionally one or more disaccharides (e.g., sucrose), gums and/or maltodextrins
156) the composition comprises one or more beneficial agents
157) the composition comprises one or more microorganisms
158) the composition comprises one or more diazotrophs
159) the composition comprises one or more phosphate-solubilizing microorganisms
160) the composition comprises one or more bacteria, optionally one or more Alkalihalobacillus, Azospirillum, Bacillus, Bradyrhizobium, Campylobacter, Chryseobacterium, Clostridium, Dicytoglomus, Effusibacillus, Enterococcus, Erwinia, Escherichia, Flavobacterium, Fusobacterium, Geobacillus, Helicobacter, Ilyobacter, Lactobacillus, Lactococcus, Lederbergia, Lysinibacillus, Lysobacter, Neisseria, Neobacillus, Nocardiopsis, Oceanobacillus, Paenibacillus, Pseudomonas, Rhizobium, Salmonella, Sinorhizobium, Staphylococcus, Streptococcus, Streptomyces, Sutcliffiella, Ureaplasma and/or Yersinia
161) the composition comprises one or more fungi, optionally one or more Acremonium, Acrophialophora, Aspergillus, Aureobasidium, Bjerkandera, Ceriporiopsis, Chaetomium, Chrysosporium, Colletotrichum, Coprinopsis, Coprinus, Coriolus, Cryphonectria, Cryptococcus, Evansstolkia, Filibasidium, Fusarium, Gliocladium, Glomus, Humicola, Magnaporthe, Metarhizium, Microdochium, Mucor, Myceliophthora, Neocallimastix, Neurospora, Ostropa, Paecilomyces, Penicilhum, Phlebia, Piromyces, Pleurotus, Pseudoplectania, Schizophyllum, Sodiomyces, Stenocarpella, Talaromyces, Thermoascus, Themochaetoides, Thermomyces, Thermothielavioides, Thermothelomyces, Thielavia, Tolypocladium, Trametes, Trichoderma, Trichophaea and/or Urnula
162) the composition comprises one or more yeast, optionally one or more Candida, Hansenula, Komagataella, Kluyveromyces, Pichia, Saccharomyces, Schizosaccharomyces and/or Yarrowia
163) the composition comprises one or more plant cells, optionally one or more Amaranthaceae, Asteraceae, Brassicaceae, Caricaceae, Cucurbitaceae, Fabaceae, Malvaceac, Poaceae, Polygonaceae, Rosaceae, Solanaceae and/or Vitaceae cells
164) the composition comprises one or more biological pesticides, optionally one ormore biological acaricides, biological bactericides, biological fungicides, biological gastropodicides, biological herbicides, biological insecticides, biological miticides, biological nematicides, biological oomyceticides and/or biological protozoacides
165) the composition comprises one or more chemical pesticides, optionally one or more chemical acaricides, chemical bactericides, chemical fungicides, chemical gastropodicides, chemical herbicides, chemical insecticides, chemical miticides, chemical nematicides, chemical oomyceticides and/or chemical protozoacides
166) the composition comprises one or more microbial pesticides, optionally one or more microbial acaricides, microbial bactericides, one or more microbial fungicides, microbial gastropodicides, microbial herbicides, microbial insecticides, microbial miticides, microbial nematicides, microbial oomyceticides and/or microbial protozoacides
167) the composition comprises one or more dispersants, optionally one or more alcohol ethoxylates and/or polyvinylpyrrolidones
168) the composition comprises one or more drying agents
169) the composition comprises one or more nutrients, optionally one or more organic acids (e.g., acetic acid, citric acid, lactic acid, malic acid, taurine, etc.), macrominerals (e.g., phosphorous, calcium, magnesium, potassium, sodium, iron, etc.), trace minerals (e.g., boron, cobalt, chloride, chromium, copper, fluoride, iodine, manganese, molybdenum, selenium, zinc, etc.), and/or vitamins (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.)
170) the composition comprises one or more pest attractants
171) the composition comprises one or more pest feeding stimulants
172) the composition comprises one or more pH control components
173) the composition has a pH within the operable range for each enzyme therein
174) the composition has a pH within +/−1 pH-unit of the optimal pH for at least one enzyme therein
175) the composition has a pH within +/−1 pH-unit of the optimal pH for each enzyme therein
176) the composition has a pH of about 2.5 to about 7
177) the composition has a pH of about 2.5 to about 6.5
178) the composition has a pH of about 2.5 to about 6
179) the composition has a pH of about 2.5 to about 5.5
180) the composition has a pH of about 2.5 to about 5
181) the composition has a pH of about 2.5 to about 4.5
182) the composition has a pH of about 2.5 to about 4
183) the composition has a pH of about 2.5 to about 3.5
184) the composition has a pH of about 2.5 to about 3
185) the composition has a pH of about 3 to about 7
186) the composition has a pH of about 3 to about 6.5
187) the composition has a pH of about 3 to about 6
188) the composition has a pH of about 3 to about 5.5
189) the composition has a pH of about 3 to about 5
190) the composition has a pH of about 3 to about 4.5
191) the composition has a pH of about 3 to about 4
192) the composition has a pH of about 3 to about 3.5
193) the composition has a pH of about 3.5 to about 7
194) the composition has a pH of about 3.5 to about 6.5
195) the composition has a pH of about 3.5 to about 6
196) the composition has a pH of about 3.5 to about 5.5
197) the composition has a pH of about 3.5 to about 5
198) the composition has a pH of about 3.5 to about 4.5
199) the composition has a pH of about 3.5 to about 4
200) the composition has a pH of about 4 to about 7
201) the composition has a pH of about 4 to about 6.5
202) the composition has a pH of about 4 to about 6
203) the composition has a pH of about 4 to about 5.5
204) the composition has a pH of about 4 to about 5
205) the composition has a pH of about 4 to about 4.5
206) the composition has a pH of about 4.5 to about 8.5
207) the composition has a pH of about 4.5 to about 8
208) the composition has a pH of about 4.5 to about 7.5
209) the composition has a pH of about 4.5 to about 7
210) the composition has a pH of about 4.5 to about 6.5
211) the composition has a pH of about 4.5 to about 6
212) the composition has a pH of about 4.5 to about 5.5
213) the composition has a pH of about 4.5 to about 5
214) the composition has a pH of about 5 to about 9
215) the composition has a pH of about 5 to about 8.5
216) the composition has a pH of about 5 to about 8
217) the composition has a pH of about 5 to about 7.5
218) the composition has a pH of about 5 to about 7
219) the composition has a pH of about 5 to about 6.5
220) the composition has a pH of about 5 to about 6
221) the composition has a pH of about 5 to about 5.5
222) the composition has a pH of about 5.5 to about 8.5
223) the composition has a pH of about 5.5 to about 8
224) the composition has a pH of about 5.5 to about 7.5
225) the composition has a pH of about 5.5 to about 7
226) the composition has a pH of about 5.5 to about 6.5
227) the composition has a pH of about 5.5 to about 6
228) the composition has a pH of about 6 to about 11
229) the composition has a pH of about 6 to about 10.5
230) the composition has a pH of about 6 to about 10
231) the composition has a pH of about 6 to about 9.5
232) the composition has a pH of about 6 to about 9
233) the composition has a pH of about 6 to about 8.5
234) the composition has a pH of about 6 to about 8
235) the composition has a pH of about 6 to about 7.5
236) the composition has a pH of about 6 to about 7
237) the composition has a pH of about 6 to about 6.5
238) the composition has a pH of about 6.5 to about 11
239) the composition has a pH of about 6.5 to about 10.5
240) the composition has a pH of about 6.5 to about 10
241) the composition has a pH of about 6.5 to about 9.5
242) the composition has a pH of about 6.5 to about 9
243) the composition has a pH of about 6.5 to about 8.5
244) the composition has a pH of about 6.5 to about 8
245) the composition has a pH of about 6.5 to about 7.5
246) the composition has a pH of about 6.5 to about 7
247) the composition has a pH of about 7 to about 11
248) the composition has a pH of about 7 to about 10.5
249) the composition has a pH of about 7 to about 10
250) the composition has a pH of about 7 to about 9.5
251) the composition has a pH of about 7 to about 9
252) the composition has a pH of about 7 to about 8.5
253) the composition has a pH of about 7 to about 8
254) the composition has a pH of about 7 to about 7.5
255) the composition has a pH of about 7.5 to about 11
256) the composition has a pH of about 7.5 to about 10.5
257) the composition has a pH of about 7.5 to about 10
258) the composition has a pH of about 7.5 to about 9.5
259) the composition has a pH of about 7.5 to about 9
260) the composition has a pH of about 7.5 to about 8.5
261) the composition has a pH of about 7.5 to about 8
262) the composition has a pH of about 8 to about 11
263) the composition has a pH of about 8 to about 10.5
264) the composition has a pH of about 8 to about 10
265) the composition has a pH of about 8 to about 9.5
266) the composition has a pH of about 8 to about 9
267) the composition has a pH of about 8 to about 8.5
268) the composition has a pH of about 8.5 to about 11
269) the composition has a pH of about 8.5 to about 10.5
270) the composition has a pH of about 8.5 to about 10
271) the composition has a pH of about 8.5 to about 9.5
272) the composition has a pH of about 8.5 to about 9
273) the composition has a pH of less than 4
274) the composition has a pH of more than 4
275) the composition has a pH of less than 5
276) the composition has a pH of more than 5
277) the composition has a pH of less than 6
278) the composition has a pH of more than 6
279) the composition has a pH of less than 6.5
280) the composition has a pH of more than 6.5
281) the composition has a pH of less than 7
282) the composition has a pH of more than 7
283) the composition has a pH of less than 7.5
284) the composition has a pH of more than 7.5
285) the composition has a pH of less than 8
286) the composition has a pH of more than 8
287) the composition has a pH of less than 9
288) the composition has a pH of more than 9
289) the composition comprises one or more postharvest treatments
290) the composition comprises one or more essential oils
291) the composition comprises one or more ethylene biosynthesis inhibitors
292) the composition comprises one or more cyclopropenes
293) the composition comprises one or more waxes
294) the composition comprises one or more preservatives
295) the composition comprises one or more benzoates (e.g., sodium benzoate)
296) the composition comprises benzoic acid
297) the composition comprises methyl paraben
298) the composition comprises phenoxy ethanol
299) the composition comprises one or more proprionates (e.g., ammonium proprionate, calcium proprionate, sodium proprionate)
300) the composition comprises proprionic acid
301) the composition comprises one or more sorbates (e.g., potassium sorbate, sodium sorbate)
302) the composition comprises 1,2-benzisothiazolin-3-one (PROXEL®; Basel, Switzerland)
303) the composition comprises one or more rain fasteners
304) the composition comprises one or more organo-modified siloxanes
305) the composition comprises one or more trisiloxanes
306) the composition comprises one or more polysiloxanes
307) the composition comprises one or more rhealogical agents
308) the composition comprises one or more safeners
309) the composition comprises one or more stabilizers, optionally one or more boric acid derivatives, inorganic salts, lactic acid, polyols, sugars, sugar alcohols and/or reversible protease inhibitors
310) the composition comprises boric acid
311) the composition comprises glycerol
312) the composition comprises one or more UV protectants, optionally one or more aromatic amino acids, carotenoids, cinnamates, lignosulfonates, melanins, mycosporines, polyphenols and/or salicylates
313) the composition comprises one or more wetting agents, optionally one or more naphthalene sulfonates
314) the composition is applied in a pesticidally effective amount
315) the composition is applied in an acaricidally effective amount
316) the composition is applied in a bactericidally effective amount
317) the composition is applied in a fungicidally effective amount
318) the composition is applied in a gastropodicidally effective amount
319) the composition is applied in an herbicidally effective amount
320) the composition is applied in an insecticidally effective amount
321) the composition is applied in an miticidally effective amount
322) the composition is applied in a nematicidally effective amount
323) the composition is applied in an oomyceticidally effective amount
324) the composition is applied in a protozoacidally effective amount
325) the composition is applied in a pesticidally ineffective amount (e.g., as an adjuvant)
326) the composition is applied in an acaricidally ineffective amount (e.g., as an adjuvant)
327) the composition is applied in a bactericidally ineffective amount (e.g., as an adjuvant)
328) the composition is applied in a fungicidally ineffective amount (e.g., as an adjuvant)
329) the composition is applied in a gastropodicidally ineffective amount (e.g., as an adjuvant)
330) the composition is applied in a herbicidially ineffective amount (e.g., as an adjuvant)
331) the composition is applied in an insecticidally ineffective amount (e.g., as an adjuvant)
332) the composition is applied in an miticidally ineffective amount (e.g., as an adjuvant)
333) the composition is applied in a nematicidally ineffective amount (e.g., as an adjuvant)
334) the composition is applied in an oomyceticidally ineffective amount (e.g., as an adjuvant)
335) the composition is applied in a protozoacidally ineffective amount (e.g., as an adjuvant)
336) the composition is applied prior to planting
337) the composition is applied at the time of planting
338) the composition is applied after planting
339) the composition is applied prior to germination
340) the composition is applied at the time of germination
341) the composition is applied after germination
342) the composition is applied prior to seedling emergence
343) the composition is applied at the time of seedling emergence
344) the composition is applied after seeding emergence
345) the composition is applied prior to vegetative stage
346) the composition is applied during vegetative stage
347) the composition is applied after vegetative stage
348) the composition is applied prior to reproductive stage
349) the composition is applied during reproductive stage
350) the composition is applied after reproductive stage
351) the composition is applied prior to flowering
352) the composition is applied at the time of flowering
353) the composition is applied after flowering
354) the composition is applied prior to fruiting
355) the composition is applied at the time of fruiting
356) the composition is applied after fruiting
357) the composition is applied prior to ripening
358) the composition is applied at the time of ripening
359) the composition is applied after ripening
360) the composition is applied prior to pruning
361) the composition is applied at the time of pruning
362) the composition is applied after pruning
363) the composition is applied prior to harvest
364) the composition is applied at the time of harvest
365) the composition is applied after harvest
366) the composition is applied prior to storage and/or transport
367) the composition is applied at the time of storage and/or transport
368) the composition is applied after storage and/or transport
369) the composition is used in combination with one or more adhesives
370) the composition is used in combination with one or more beneficial agents
371) the composition is used in combination with one or more microorganisms
372) the composition is used in combination with one or more diazotrophs
373) the composition is used in combination with one or more phosphate-solubilizing microorganisms
374) the composition is used in combination with one or more bacteria, optionally one or more Alkalihalobacillus, Azospirillum, Bacillus, Bradyrhizobium, Campylobacter, Chryseobacterium, Clostridium, Dicytoglomus, Effusibacillus, Enterococcus, Erwinia, Escherichia, Flavobacterium, Fusobacterium, Geobacillus, Helicobacter, Ivobacter, Lactobacillus, Lactococcus, Lederbergia, Lysinibacillus, Lysobacter, Neisseria, Neobacillus, Nocardiopsis, Oceanobacillus, Paenibacillus, Pseudomonas, Rhizobium, Salmonella, Sinorhizobium, Staphylococcus, Streptococcus, Streptomyces, Sutclffiella, Ureaplasma and/or Yersinia 375) the composition is used in combination with one or more fungi, optionally one or more Acremonium, Acrophialophora, Aspergillus, Aureobasidium, Bjerkandera, Ceriporiopsis, Chaetomium, Chrysosporium, Colletotrichum, Coprinopsis, Coprinus, Coriolus, Cryphonectria, Cryptococcus, Fvansstolkia, Filibasidium, Fusarium, Gliocladium, Glomus, Humicola, Magnaporthe, Metarhizium, Microdochium, Mucor, Mycelhophthora, Neocallimastix, Neurospora, Ostropa, Paecilomyces, Penicilhum, Phlebia, Piromyces, Pleurotus, Pseudoplectania, Schizophyllum, Sodiomyces, Stenocarpella, Talaromyces, Thermoascus, Themochaetoides, Thermomyces, Thermothielavioides, Thermothelomyces, Thielavia, Tolypocladium, Trametes, Trichoderma, Trichophaea and/or Urnula 376) the composition is used in combination with one or more yeast, optionally one or more Candida, Hansenula, Komagataella, Kluyveromyces, Pichia, Saccharomyces, Schizosaccharomyces and/or Yarrowia
377) the composition is used in combination with one or more biological pesticides, optionally one or more biological acaricides, biological bactericides, biological fungicides, biological gastropodicides, biological herbicides, biological insecticides, biological miticides, biological nematicides, biological oomyceticides and/or biological protozoacides
378) the composition is used in combination with one or more chemical pesticides, optionally one or more chemical acaricides, chemical bactericides, chemical fungicides, chemical gastropodicides, chemical herbicides, chemical insecticides, chemical miticides, chemical nematicides, chemical oomyceticides and/or chemical protozoacides
379) the composition is used in combination with one or more microbial pesticides, optionally one or more microbial acaricides, microbial bactericides, one or more microbial fungicides, microbial gastropodicides, microbial herbicides, microbial insecticides, microbial miticides, microbial nematicides, microbial oomyceticides and/or microbial protozoacides
380) the composition is used in combination with one or more pest attractants
381) the composition is used in combination with one or more pest feeding stimulants
382) the composition is used in combination with one or more plant growth regulators
383) the composition is used in combination with one or more rain fasteners
384) the composition is used in combination with one or more organo-modified siloxanes
385) the composition is used in combination with one or more trisiloxanes
386) the composition is used in combination with one or more polysiloxanes
387) the composition is used in combination with one or more preharvest treatments
388) the composition is used in combination with one or more postharvest treatments
389) the composition is used in combination with one or more essential oils
390) the composition is used in combination with one or more ethylene biosynthesis inhibitors
391) the composition is used in combination with one or more cyclopropenes
392) the composition is used in combination with one or more waxes
393) the composition is used in combination with one or more stabilizers, optionally one or more boric acid derivatives, inorganic salts, lactic acid, polyols, sugars, sugar alcohols and/or reversible protease inhibitors
394) the composition is used in combination with one or more UV protectants, optionally one ormore aromatic amino acids, carotenoids, cinnamates, lignosulfonates, melanins, mycosporines, polyphenols and/or salicylates
395) the composition is used in combination with one or more wetting agents, optionally one or more naphthalene sulfonates

Transgenic microorganisms, plants or plant parts for which any one, two, three, four, five, six, seven, eight, nine, ten or more of the following are true:
1) comprises one or more polynucleotides encoding a polypeptide having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 1-91, 45906, 72144, 72147 and 72149 or a mature polypeptide thereof
2) comprises one or more polynucleotides having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to any one or more of SEQ ID NO(s): 92-182, 72145-72146, 72148 and 72150 or the cDNA sequence thereof
3) expresses one or more polypeptides having about/at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to one or more of SEQ ID NO(s): 1-91, 45906, 72144, 72147 and 72149 or a mature polypeptide thereof
4) comprises one or more polynucleotides encoding a polypeptide derived from any one of SEQ ID NO(s): 1-91, 45906, 72144, 72147 and 72149 by substitution, deletion, or insertion of one or more amino acids
5) expresses one or more polypeptides derived from any one of SEQ ID NO(s): 1-91, 45906, 72144, 72147 and 72149 by substitution, deletion, or insertion of one or more amino acids
6) comprises one or more polynucleotides encoding a polypeptide derived from a mature polypeptide of any one of SEQ ID NO(s): 1-91, 45906, 72144, 72147 and 72149 by substitution, deletion, or insertion of one or more amino acids
7) expresses one or more polypeptides derived from a mature polypeptide of any one of SEQ ID NO(s): 1-91, 45906, 72144, 72147 and 72149 by substitution, deletion, or insertion of one or more amino acids
8) comprises one or more polynucleotides encoding a polypeptide derived from any one of any one of SEQ ID NO(s): 1-91, 45906, 72144, 72147 and 72149 above wherein the N- and/or C-terminal end has been extended by the addition of one or more amino acids
9) expresses one or more polypeptides derived from any one of SEQ ID NO(s): 1-91, 45906, 72144, 72147 and 72149 wherein the N- and/or C-terminal end has been extended by the addition of one or more amino acids
10) comprises one or more polynucleotides encoding an enzymatically active fragment of any one of SEQ ID NO(s): 1-91, 45906, 72144, 72147 and 72149
11) expresses an enzymatically active fragment of any one SEQ ID NO(s): 1-91, 45906, 72144, 72147 and 72149
12) expresses an enzymatically active a fusion protein comprising a first polypeptide and a second polypeptide wherein at least one of said first and second polypeptides is a protein of the present disclosure.

### EXAMPLES

The following examples are provided to illustrate certain embodiments and are not to be construed as limiting the inventive concepts described in the present disclosure. Enzyme treatments are described in the corresponding Figures with reference to the predominant enzyme(s) comprised therein. It is to be understood that enzyme treatments may possess other enzymatic activities not expressly disclosed in the corresponding Figures. For example, an enzyme treatment having a predominant beta-glucanase activity may exhibit lesser side activities, such as cellulase activity and/or xylanase activity.

### Example 1

### Effective Control of Powdery Mildew

### Principle

Tested the efficacy of enzyme solutions for control of powdery mildew on wheat plants. Plants were sprayed with buffered enzyme solutions and subsequently inoculated with Blumeria graminis. Percent leaf area attacked was assessed nine days post-inoculation and again with two-day intervals until the full effects were seen. For scoring the standard EPPO scales (PP2/.10 (1)) were used. Experiments were conducted by Arhus University, Flakkebjerg, Denmark.

### Procedures

1) Powdery mildew-susceptible wheat plants (var. Anja) were grown in a greenhouse for approximately two weeks until reaching the two-leaf crop growth stage (BBCH12).
2) Plants were then sprayed with an enzyme solution comprising 0.1% TWEEN® 20, or a control solution comprising 0.1% TWEEN® 20 with no enzyme added, using a cabin sprayer (volume 600 mL; 150 L/ha, 3.6 km/hour, yellow nozzles 0.2). Six replicates were made for each condition.
3) 24 hours after the initial spray application, the plants were sprayed with a Blumeria graminis spore suspension.
4) After Blumeria graminis inoculation, the plants were kept under high relative humidity for 24 hours to ensure infection before being placed under normal greenhouse conditions.
5) Percent leaf area attacked by powdery mildew was determined nine days after inoculation and again every two days until the full effects were found. For scoring, the standard EPPO scale was used (EPPO standards: Guidelines on good plant protection practice. Wheat. PP 2/10(1)).

### Results

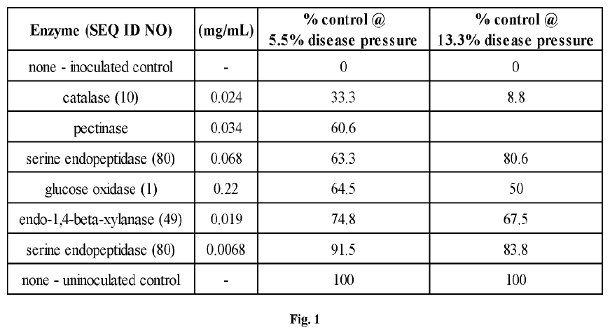

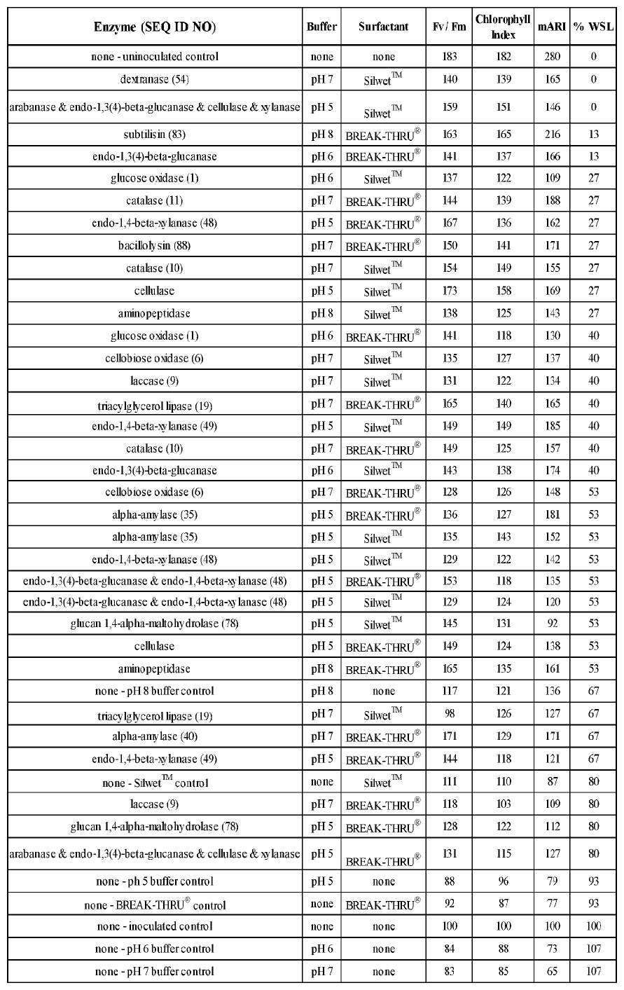

Enzymes were evaluated for efficacy against Blumeria graminis as described above. Results are presented in FIG. 1 as percent disease control relative to an untreated, uninoculated control and an untreated, inoculated control at two timepoints/disease pressures. 100% control means no disease was detected in the treatment and 0% control means disease was at the same level as the untreated, inoculated control comparator.

### Example 2

### Effective Control of Gray Mold

### Principle

Evaluated the efficacy of enzyme solutions for control of gray mold on tomato leaf discs. Leaf discs were painted with buffered enzyme solutions and subsequently inoculated with Botrytis cinerea. After incubation for 24-96 hours, selected plant health parameters were evaluated using multispectral analysis. Testing was conducted at Ghent University (Ghent, Belgium).

### Procedures

1) Tomato plants were grown in potting soil for 28 days. Leaf discs (diameter 20 mm) were cut from the 4^{th} composite leaves using a cork bore. Only one disc was punched from each leaf. All leaf discs were kept in a water bath until use.
2) One leaf disc was placed in each well of a 24-well microtiter plate.
3) Leaf discs were painted with enzyme solutions comprising 1 mg enzyme protein per mL in a 10 mM buffer solution (sodium acetate pH 5, potassium phosphate pH 6, potassium phosphate pH 7, or potassium phosphate pH 8), with or without 0.1% w/v surfactant (BREAK-THRU® S 301 or SILWET™ L-77), or with a corresponding control solution comprising no enzyme, and then air dried. Six replicates were made for each condition.
4) After drying, discs were inoculated with two 5 μL drops of Botrytis cinerea spore suspension (106 spores per mL in 10 mM glucose, 0.067 mM K-phosphate buffer, pH 5) or with a corresponding solution comprising no Botrytis cinerea spores.
5) Disease progression was monitored through multispectral imaging and visual inspection for water-soaked lesions. Multispectral images were taken at the start of the experiment (To) and after incubation for 24, 48, 72 and 96 hours. Fv/Fm, ChlIdx and mARI were evaluated from the spectral images. See Table 2. Mean Fv/Fm, ChlIdx and mARI values for leaves at T₀ were used as a reference (100%) to evaluate the Fv/Fm, ChlIdx and mARI values for treated leaves.

TABLE 2
Multispectral imaging parameters

| Parameter | Description | Reference |
|---|---|---|
| Fv/Fm | Efficiency of photosystem II in a dark-adapted state. Fv/Fm is deemed | Baker, ANN. REV. |
| | to be a proxy for potential phytotoxic effects of the enzyme on the | PLANT BIOL. |
| | photosynthesis for early time points. At later time points, the ratio is a | 59: 89-113 (2008). |
| | proxy for damage due to progressing infection by B. cinerea. The | |
| | higher values preferred. | |
| ChlIdx | Chlorophyll index, vegetation index for estimation of chlorophyll | Gitelson et al., J. |
| | content in leaves. Higher the chlorophyll indices indicate healthier leaf | PLANT PHYSIOL. |
| | tissue. | 160: 271-82 (2003). |
| mARI | Modified anthocyanin reflectance index; index for estimation of | Gitelson et al., AM. |
| | anthocyanin content in leaves. Anthocyanins play a dual role in plants | J. BOT. |
| | and can be associated with plant defense as well as with phytotoxicity | 96(10): 1861-68 |
| | and pathogenicity. Higher mARI values coincide with a lower | (2009). |
| | percentage of water-soaked lesions. | |
| cGFP | GFP fluorescence, corrected for autofluorescence due to leaf | |
| | senescence. Accumulation of GPF-tagged Fusarium graminearum PH- | |
| | 1 actively growing biomass was evaluated by means of its (corrected) | |
| | GFP-signal (cGFP). | |

### Results

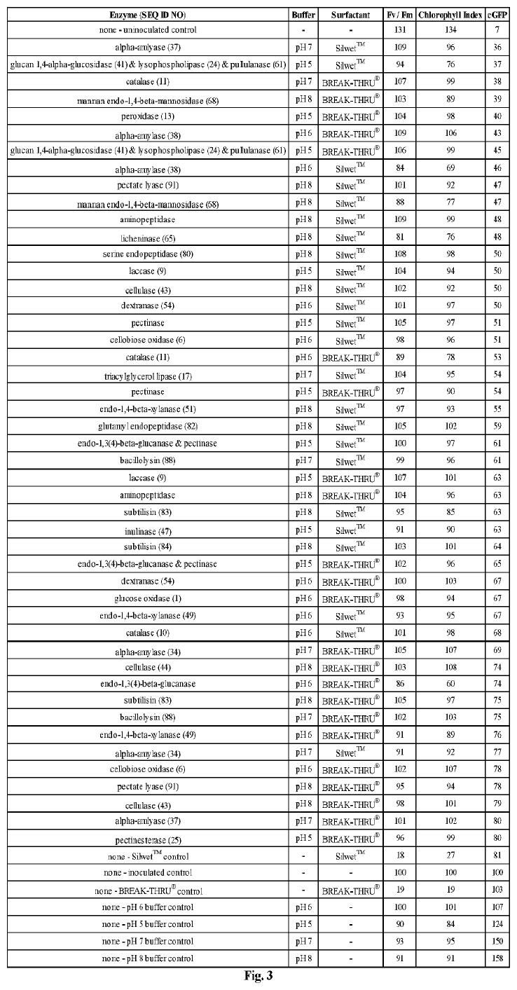

Enzymes were evaluated for efficacy against Botrytis cinerea as described above. Results are presented in FIG. 2 as percentage increase of the spectral parameters (Fv/Fm, ChlIdx and mARI) and the percentage of water-soaked lesions (% WSL) as compared to the untreated, inoculated control.

### Example 3

### Effective Control of Head Blight

### Principle

Evaluated the efficacy of enzyme solutions for control of Fusarium head blight on detached wheat leaves. Detached leaves were treated with enzyme solution and subsequently inoculated with GFP-transformed Fusarium graminearum. Pathogen biomass was quantified via GFP, and plant health parameters were collected up to 96 hours post-inoculation. Testing was conducted at Ghent University (Ghent, Belgium).

### Procedures

1) GFP-transformed Fusarium graminearum strain PH-1 was grown on potato dextrose agar for seven days at 21° C. under a regime of 12 h dark and 12 h combined UVA and UVC light to induce sporulation.
2) A working spore suspension was created by adding approx. 20 mL of phosphate buffered saline containing 0.01% TWEEN® 80, pH 7.2-7.4, to the petri dish, agitating the mycelium with a spatula, separating spores from mycelium using a sterile filter, and then diluting to a concentration of 0.5×10⁶ spores per mL.
3) Wheat plants were grown in potting soil for 10 days, after which leaves (approx. 6 cm) were detached and used in the bioassays described below.
4) Detached leaves (leaf tops) were placed on their abaxial surface in rectangular petri dishes containing 0.5% (w/v) water agar supplemented with 40 mg benzimidazole per mL. The centers of the leaves were wounded with a scalpel by gently scraping the epidermal layer of the leaves.
5) Wounded leaves were painted with enzyme solutions comprising 1 mg enzyme protein per mL in a 10 mM buffer solution (sodium acetate pH 5, potassium phosphate pH 6, potassium phosphate pH 7, or potassium phosphate pH 8), with or without 0.1% w/v surfactant (BREAK-THRU© S 301 or SILWET™ L-77), or with a corresponding control solution comprising no enzyme, and then air dried.

Six replicates were made for each condition.
6) After drying, each leaf was inoculated with a 10 μl droplet of the Fusarium graminearum PH-1 spore suspension or with a corresponding solution comprising no Fusarium graminearum spores.
7) Plates were sealed with parafilm and incubated at 18° C. under a 16-hour light/8-hour dark cycle.
8) Disease progression was monitored through multispectral imaging and visual inspection. Multispectral images were taken at start of experiment (To) and after incubation for 24, 48, 72 and 96 hours. Fv/Fm, ChlIdx and cGFP were evaluated from the spectral images. See Table 2. Mean Fv/Fm, ChlIdx and cGFP values for leaves at T₀ were used as a reference (100%) to evaluate the Fv/Fm, ChlIdx and cGFP values for treated leaves.

### Results

Enzymes were evaluated for efficacy against Fusarium graminearum as described above. Results are presented in FIG. 3A as percentage increase of the spectral parameters (Fv/Fm, ChlIdx and cGFP) as compared to the untreated, inoculated control after 72 hours.

### Example 4

### Effective Control of Rice Blast

### Principle

Evaluated the efficacy of enzyme solutions for control of Magnaporthe grisea using high-throughput imaging. Serial dilutions were used to determine the half maximal effective concentration (EC₅₀) for two distinct indicators of fungal inhibition: absorbance at 612 nm (indicator of mycelia growth inhibition) and fluorescence after reaction with Resazurin (ex 570 nm-em 585 nm; indicator of cell viability). Experiments were conducted by Fundación Medina, Centro de Exelencia en Investigación de Medicamentos Innovadores en Andalucia (Granada, Spain).

### Procedures

1) Starting with 10 mg/mL enzyme stock solutions, a 1:2 dilution series of each enzyme was prepared (1×, 0.5×, 0.25×, 0.05×, 0.025×, 0.0125×, 0.00625× and 0.00325×) in 1/10 PBS buffer in 96 well plates, obtaining eight doses ranging from 10 mg/mL to 0.03125 mg/mL.
2) Final enzyme test concentrations ranging from 3.91 to 500 μg/mL were obtained by adding 5 μL of each enzyme dose to 95 μL of Magnaporthe grisea CF-105765 conidia suspension in triplicate.
3) 10 μL of 500 μg/mL Amphotericin B in 90 μL conidia suspension was used as a positive control (assay final concentration of 50 μg/mL) and 10 μL 20% DMSO in 90 μL conidia suspension was used as a negative control (assay final concentration of 2%). All assay plates included quadruplicates of both the positive and the negative control. Furthermore, all assay plates also included an Amphotericin B standard curve consisting of a 1:2 serial dilution ranging from 50 μg/mL to 0.390625 μg/mL.
4) Assay plates were incubated for 24 h at _° C.
5) Absorbance at 612 nm was measured before (T₀) and after the 24 h incubation period (T_{f}) using a spectrophotometer.
6) Resazurin was then added to each reaction well (0.002% per well) and fluorescence at ex. 570 nm-em. 585 nm measured using a spectrophotometer after 2 h incubation at _° C. The percent of inhibition (% NH) based on absorbance (Abs) was calculated following the formula:

$$\% INH ( X ) = 100 - \frac{X ( Tf - T 0 ) - I nhibitor ( Tf - T 0 )}{Neutral ( Tf - T 0 ) - I nhibitor ( Tf - T 0 )} \times 100$$

where:
X=Abs value of enzyme candidate
Inhibitor=100% of inhibition (Amphotericin B at 50 μg/mL)
Neutral=0% of inhibition (DMSO at 2%)
T₀: Abs at time zero
T_{F}: Abs after incubation time-final Abs
7) The % of inhibition based on fluorescence was calculated following the formula:

$$\% de Reduction = \frac{Inhibitor - Neutral}{X - Neutral} \cdot 100$$

$$\% of Inhibition = 100 - \% of Reduction$$

where:
Inhibitor=Fluorescence intensity of 100% of Amphotericin B at 50 μg/mL
Neutral=Fluorescence intensity of vehicle
X=Fluorescence intensity of enzyme candidate

### Results

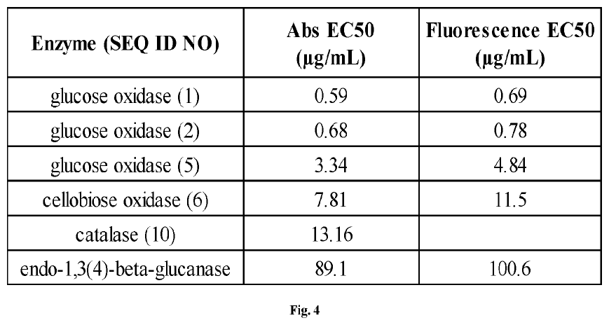

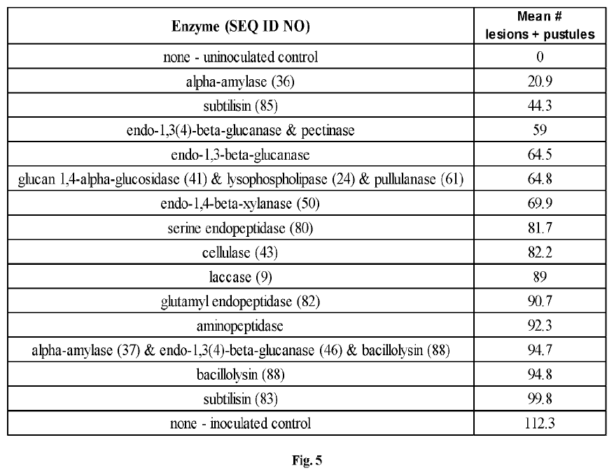

Enzymes were evaluated for efficacy against Magnaporthe grisea as described above. Data were normalized to two controls, a 100% inhibition control (50 μg/m Lamphotericin B) and a 0% inhibition control (2% dimethylsulfoxide). Results are presented in FIG. 4 as the half maximal effective concentration (EC₅₀) for absorbance at 612 nm (indicator of mycelia growth inhibition) and fluorescence after reaction with Resazurin (ex 570 nm-em 585 nm; indicator of cell viability). Data are presented as averages of triplicate samples.

### Example 5

### Effective Control of Asian Soybean Rust

### Principle

Evaluated the efficacy of enzyme solutions for control of Asian soybean rust on detached soybean leaves. Detached leaves were treated with buffered enzyme solutions and subsequently inoculated with Phakopsora pachyrhizi. The leaves were incubated in clear plastic clamshell containers for two weeks and evaluated for severity of ASR (lesion and pustule counts). Efficacy was evaluated in two separate experiments conducted at the University of Florida.

### Procedures

1) ASR-susceptible soybean plants (Williams 82) were grown in a greenhouse to growth stage V2.5, at which point fully expanded trifoliate leaves were excised and used in the detach leaf assays described below.
2) Detached leaves were placed, top (adaxial) side up, into clamshell incubation containers lined with moistened paper towels. Two leaves (six leaflets) were placed in each container. Each DLA experiment was repeated as two blocks (six leaflets per treatment in each block) for a total of four trifoliate leaves (12 leaflets) per treatment. Incubation containers were kept at room temperature, 21° C.±1°, under 12-24 hours of natural and fluorescent light per day.
3) On Day 1 of the assays, leaves were sprayed with enzyme solutions comprising 0.5 mg enzyme protein per mL in a 0.1×PBS buffer (pH 7.3), with or without 0.1% TWEEN® 20, or with a corresponding control solution comprising no enzyme. The treatments were sprayed on the top side of the soybean leaves up to the point just before runoff, which was approximately 0.6 mL per leaf. Separate, 2 fl. oz hand-pump sprayers (Hydior™) were used to make each application.
4) On Day 2 of the assays, leaves were inoculated with a suspension of soybean rust spores collected on the day of the inoculation. The top side of each leaf was sprayed with 0.6 mL of a Phakopsora pachyrhizi spore suspension (in water with 0.1% TWEEN). For the first experimental replicate, naturally occurring Phakopsora pachyrhizi spores were isolated directly from wild kudzu and suspended (1.62×10⁵ spores per mL). For the second experimental replicate, spores derived from the Phakopsorapachyrhizi strain were cultured on Williams 82 soybeans, then isolated and suspended (1.75×10⁶ spores per mL).
5) Leaves were maintained at room temperature and moisture levels were maintained as needed.
6) Leaves were examined using a dissecting microscope. The underside (abaxial side) of each leaflet was examined, where pustules (uredinia) develop. A 1.0 or 0.4 sq cm circle was placed on the leaflet and the number of ASR pustules and or lesions found within the circle were counted. Both sides of the leaflet (divided by the midvein) were counted and recorded for a total of two counts/leaflet. The counting circle was placed to avoid any areas of the leaflet with decaying tissue. A rating of leaflet decay (as % affected area) was also performed for each leaflet. For the first experimental replicate, sampling and rating of leaves occurred from days 15 to 18 days post-infection. For the second experimental replicate, sampling and rating was conducted from days 12 to 16 days post-infection.

### Results

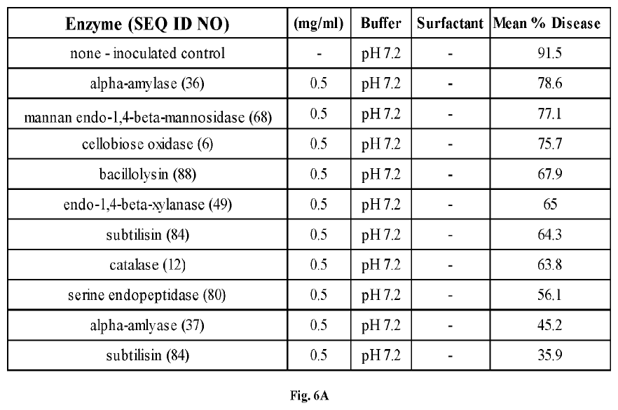

Enzymes were evaluated for efficacy against Phakopsora pachyrhizi as described above. Results are presented in FIG. 5 as the mean number of combined lesions and pustules from experimental replicates 1 and 2.

### Example 6

### Effective Control of Late Blight

### Principle

Evaluated the efficacy of enzyme solutions for control of late blight on tomato and potato leaf discs. Leaf discs were treated with enzyme and then inoculated with Phytophthora infestans US 23. Disease was assessed up to six days post-inoculation. Experiments were conducted by Novozymes Biologicals, Salem, VA.

### Procedures

1) Blight-susceptible tomato and potato plants were grown in a greenhouse for approximately three weeks. Leaves were detached from the mid-region of each plant, and leaf discs were excised using a cork bore (diameter 14 mm) and randomized amongst treatments.
2) One leaf disc was placed, adaxial side up, in each well of a 24-well microtiter plate comprising solidified 0.5% Butterfield's Buffer agar (5 g agar per liter of Butterfield's Buffer).
3) Leaf discs were treated with enzymes in Butterfield's Buffer (8.3 mM phosphate, pH 7.2) or potassium phosphate buffer (0.05 M, pH 7.88 or pH 8), with or without 0.1% w/v surfactant (BREAK-THRU® SP 133 or SILWET™ L-77), or with a corresponding control solution comprising no enzyme, and then air dried. Enzyme solutions were applied using two different methods: A) 50 μL was pipetted onto the adaxial surface of leaf disc, spreading with sterile inoculation loop as necessary; B) enzyme solution was sprayed onto the adaxial surface of leaf disc using an atomizer spray bottle, two pumps each, which adequately covered the surface without pooling.
4) After drying, discs were inoculated with 10 μL of Phytophthora infestans isolate US 23 spore suspension (1×10 spores per mL in distilled water) or sterile water comprising no Phytophthora infestans spores.
5) Plates were sealed with parafilm and incubated at 18° C. under a 16-hour light/8-hour dark cycle.
6) Images of the leaf disk plates were captured and analyzed for disease at 6 days post-infection.

### Results

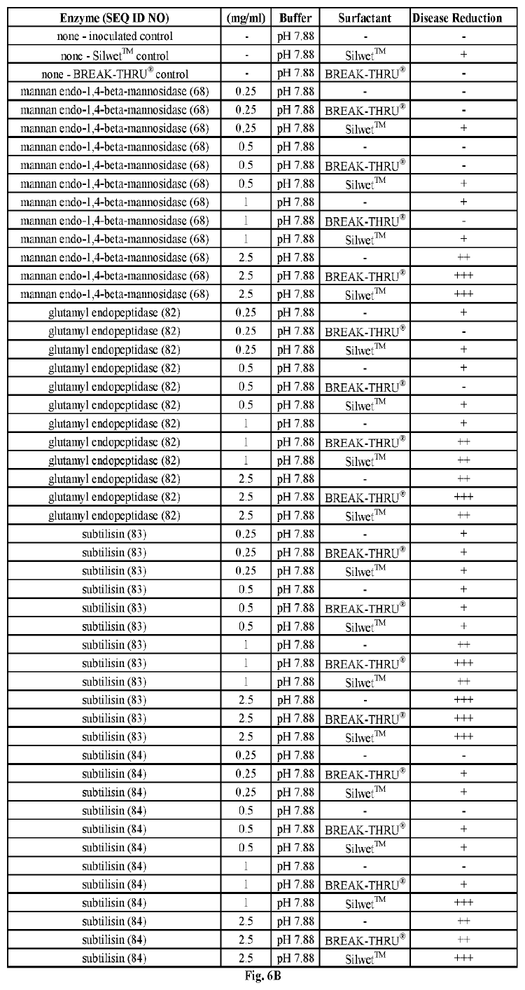

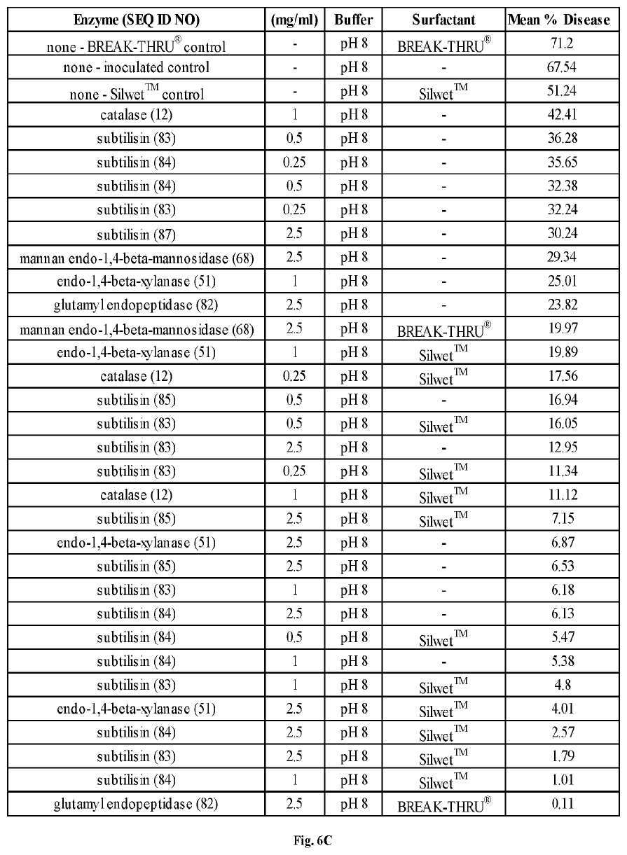

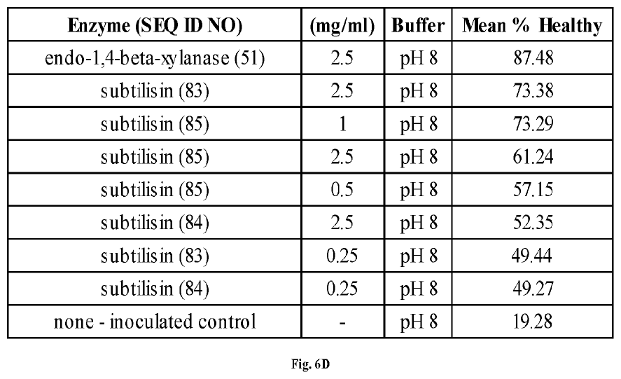

Enzymes were evaluated for efficacy against Phytophthora infestans as described above. A minimum two experimental repeats with four replicates were completed for each treatment. Results for pipette-treated tomato leaf discs treated are presented in FIG. 6A as mean percent disease. Results for spray-treated tomato leaf discs are presented in FIG. 6B as (−) no disease reduction, (+) mild disease reduction, (++) moderate disease reduction or (+++) extreme disease reduction and in FIG. 6C as mean percent disease. Results for spray-treated potato leaf discs are presented in FIG. 6D as mean percent healthy.

### Example 7

### Effective Control of Downy Mildew

### Principle

Evaluated the efficacy of enzyme solutions for control of downy mildew on cucumber leaf discs. Leaf discs were treated with buffered enzyme solutions and subsequently inoculated with Pseudoperonospora cubensis. Plates of inoculated leaf discs were incubated and assessed for disease severity after five days.

### Procedures

1) Downy mildew-susceptible cucumber plants were grown in a greenhouse for approximately four weeks. Leaves were detached from the mid-region of each plant, and leaf discs were excised using a cork bore (diameter 8 mm).
2) One leaf disc was placed, abaxial side up, in each well of a 48-well microtiter plate comprising solidified 1% water agar.
3) Leaf discs were treated with enzymes in a buffer solution-20 mM sodium acetate, pH 5; 1M potassium phosphate, pH 7; or 20 mM TRIS, pH 8), with or without surfactant (SAFER® insecticidal soap or TWEEN® 20), or with a corresponding control solution comprising no enzyme, and then air dried. Six replicates were made for each condition. Enzyme treatments were applied by pipetting 15 μL of the treatment solution onto center of the abaxial surface of the leaf disc.
4) After drying, discs were inoculated with 10 μL of Pseudoperonospora cubensis spore suspension (1-3×10⁵ spores per mL in distilled water) or sterile water comprising no Pseudoperonospora cubensis spores.
5) Plates were sealed with parafilm and incubated at 18° C. under a 16-hour light/8-hour dark cycle.
6) Images of the leaf disk plates were captured and analyzed for disease five days post-infection.

### Results

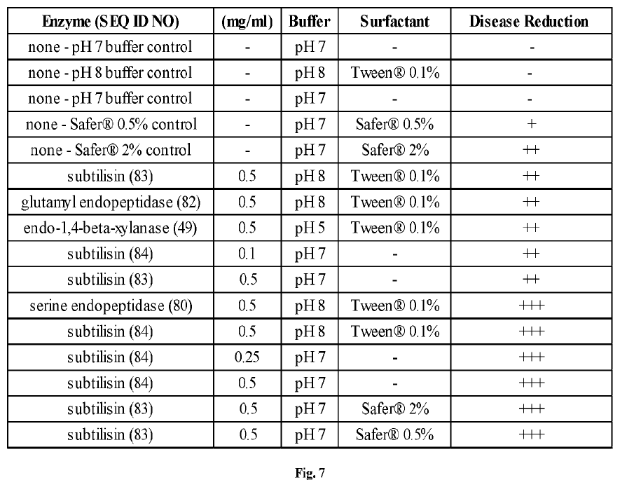

Enzymes were evaluated for efficacy against Pseudoperonospora cubensis as described above. Results are presented in FIG. 7 as (−) no disease reduction, (+) mild disease reduction, (++) moderate disease reduction or (+++) extreme disease reduction. A minimum six replicates were completed for each treatment.

### Example 8

### Effective Control of Septoria Tritici Blotch

### Principle

Evaluated the efficacy of enzyme solutions for control of Septoria tritici blotch (STB) on wheat plants. A winter wheat cultivar (var. Hereford) growing in 1 L pots was first sprayed with enzyme and after 24 hours sprayed with a spore suspension of Zymoseptoria tritici. Percent leaf area attacked was assessed 14 days after inoculation and again with 3 days interval until the full effects were found. For scoring the standard EPPO scales were used (EPPO standards: Guidelines on good plant protection practice. Wheat. PP 2/10(1)). Experiments were conducted by Aarhus University, Flakkebjerg, Denmark.

### Procedures

1) STB-susceptible wheat plants (var. Hereford) were grown in a greenhouse for approximately two weeks until reaching the two-leaf crop growth stage (BBCH12).
2) Plants were sprayed with an enzyme solution comprising 0.1% w/v surfactant (BREAK-THRU© S 301, SILWET™ L-77, or TWEEN® 20) in a 10 mM buffer (sodium acetate, pH 5; potassium phosphate, pH 6; potassium phosphate, pH 7; or potassium phosphate, pH 8), or with a corresponding control solution comprising no enzyme and/or buffer, using a cabin sprayer (volume—600 mL; 150 L/ha, 3.6 km/hour, yellow nozzles 0.2). Six replicates were made for each condition.
3) 24 hours after the initial spray application, the plants were sprayed with a Zymoseptoria tritici spore suspension (2×10⁶ spores per mL in 0.1% TWEEN® 20). Two distinct Zymoseptoria tritici cultures were used: A) a culture comprising five isolates collected from naturally infected plant in Denmark in 2020 and B) a culture comprising five isolates collected from naturally infected plant in Denmark in 2021.
4) After Zymoseptoria tritici inoculation, the plants were kept under high relative humidity for 24 hours to ensure infection before being placed under normal greenhouse conditions.
5) Percent leaf area attacked by STB was determined 14 days after inoculation and again every three days until the full effects were found. For scoring, the standard EPPO scale was used (EPPO standards: Guidelines on good plant protection practice. Wheat. PP 2/10(1)).

### Results

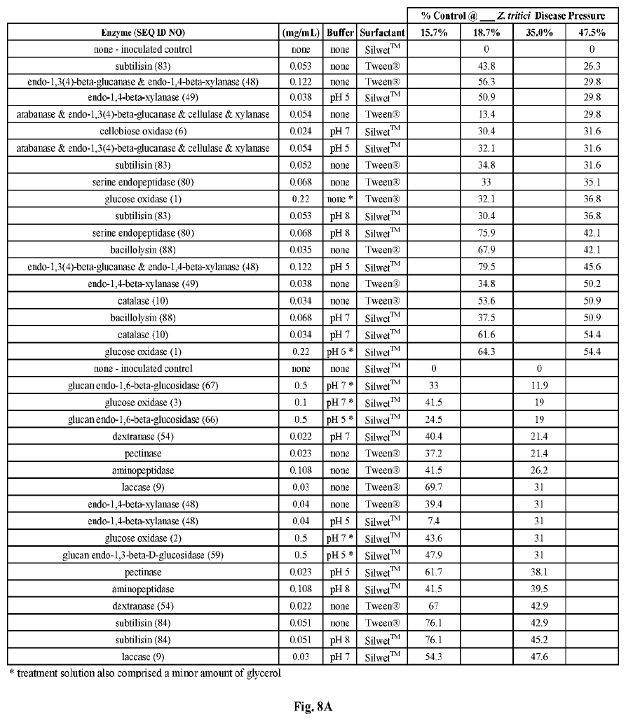

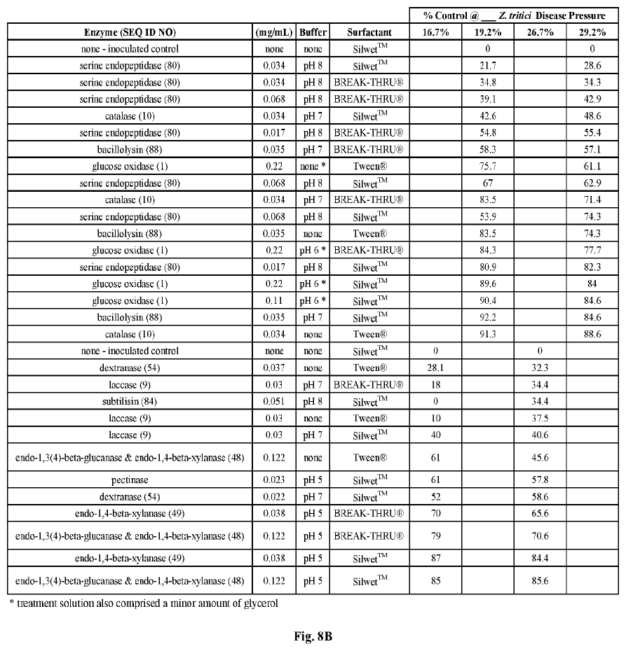

Enzymes were evaluated for efficacy on Zymoseptoria tritici infected winter wheat as described above using Inoculum A or Inoculum B. Results are presented in FIG. 8A (2020 inoculum) and FIG. 8B (2021 inoculum) as percent disease control relative to untreated, inoculated control at various timepoints/disease pressures. 100% control means no disease was detected in the treatment and 0% control means disease was at the same level as the untreated, inoculated control comparator.

### Example 9

### Effective Fungal Growth Inhibition

### Principle

Evaluated the efficacy of enzyme solutions for inhibiting fungal growth using high-throughput imaging. Serial dilutions were used to determine the minimum inhibitory concentration (MIC₅₀) for each enzyme against four fungal phytopathogens: Botrytis cinerea, Fusarium graminearum, Fusarium virguliforme, and Zymoseptoria tritici.

### Procedures

1) Enzyme dilution series ranging from 0.0078 to 0.5 mg/mL were added in quadruplicate to 384-well imaging plates. Plates were centrifuged for two minutes at 2500 rpm to ensure all enzyme samples were at the bottom of the wells.
2) Fungal spore suspensions comprising 2-2.5×10⁴ spores per mL of sterile growth medium were added to the wells. Four fungal phytopathogens were analyzed: Botrytis cinerea, Fusarium graminearum, Fusarium virguliforme, and Zymoseptoria tritici. Sterile growth medium comprised one part Potato Dexrtrose Broth and nine parts M9 minimal medium (200 mL M9 salt solution (64.0 g Na₂HPO₄ 2H₂O, 15 g KH₂PO₄, 2.5 g NaCl, 5.0 g NH₄Cl ad 1 L distilled water), 2 mL 1 M MgSO₄, 20 mL 20% glucose, 100 μl 1 M CaCl₂ in 1 L distilled water).
3) Plates were sealed with BREATHE-EASY® sealing membrane (SIGMA-ALDRICH® catalog no. Z380059) and placed in a lidded plastic container with a wet paper towel.
4) Following incubation at room temperature, 21° C.±1°, for 48 hours, plates were imaged with a high-throughput microscope using a 10× objective, capturing four non-overlapping fields per well.
5) GE Developer software and proprietary, in-house scripts were used to determine total mycelium area, total counts, total end nodes, total hyphae length, average hyphal branch length, intensity levels, and total ungerminated spores.

### Results

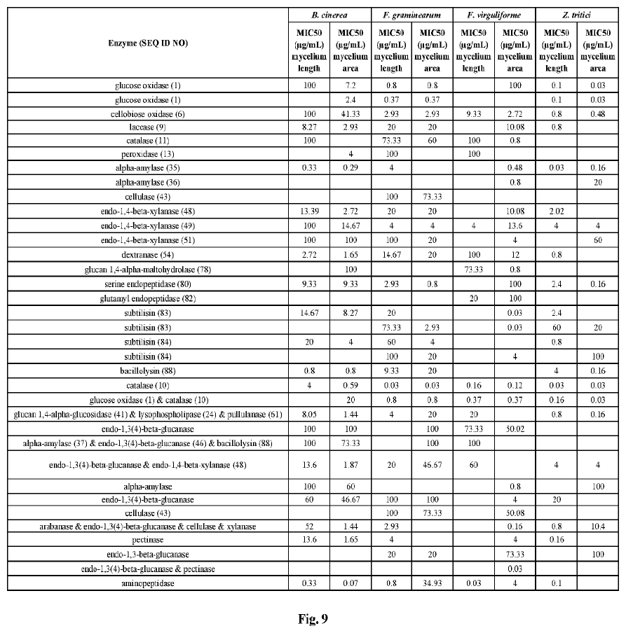

Enzymes were evaluated for efficacy against Botrytis cinerea, Fusarium graminearum, Fusarium virgulforme and Zymoseptoria tritici as described above. Data were normalized to two controls, a 100% inhibition control (cycloheximide) and a 0% inhibition control (1:9 Potato Dextrose Broth:M9; MILLIPORE® catalog no. P6685). Results are presented in FIG. 9 as the minimum inhibitory concentration (MIC₅₀) for average hyphal branch length and total mycelium area (i.e., the enzyme concentration required to inhibit average hyphal branch length/total mycelium area to 50% or less as compared to that of corresponding untreated control). Data are presented as averages of quadruplicate samples.

### Example 10

### Effective Fungal Growth Inhibition

### Principle

Evaluated the efficacy of enzyme solutions for inhibiting fungal growth using Radial Diffusion Assays (RDA), which consisted of plating a fungal pathogen in potato dextrose agar (PDA) in sufficient amount to obtain a homogeneous mat of mycelium, allowing the mycelium mat to dry, and then adding a 5 μL drop of enzyme solution. Growth inhibition was scored as the presence/absence of an inhibition- or stress-zone created by the enzyme after incubation of the assay plates at 26° C. up to 15 days.

### Procedures

1) Plates comprising potato dextrose agar were inoculated with a fungal phytopathogen-Botrytis cinerea ATCC56594, Fusarium graminearum Schwabe ATCC 36885, Magnaporthe grisea, Penicillium digitatum CBS658.68, Penicillium expansum DSM1282, Penicillium italicum. Phytophthora infestans, or Zymoseptoria tritici GPD-GFPB3 (Rohel et al., MOL. PLANT MICROBE INTERACT. 14(2):156-63 (February 2001))—by adding a droplet of fungal suspension and spreading the droplet “pprox.”y across the surface of the medium using glass beads.
2) After the plates were dry, test plates were treated with an enzyme solution, with or without buffer (potassium phosphate, pH 6; potassium phosphate, pH 7; or potassium phosphate, pH 8), and with or without surfactant (SILWET™ L-77). Tests were conducted in duplicate. An untreated control plate was kept for each fungal phytopathogen.
3) Plates were incubated at 26° C. and assessed daily for 15 days.

### Results

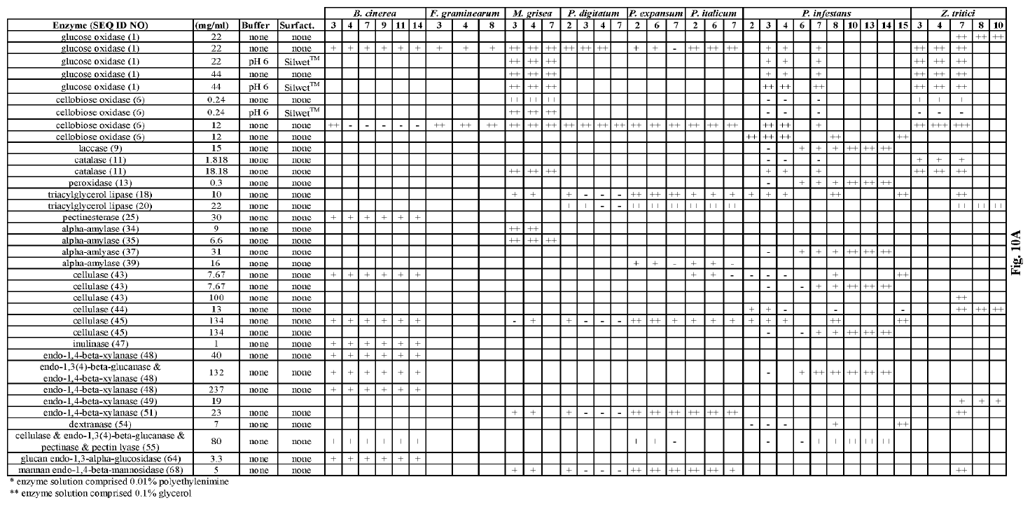

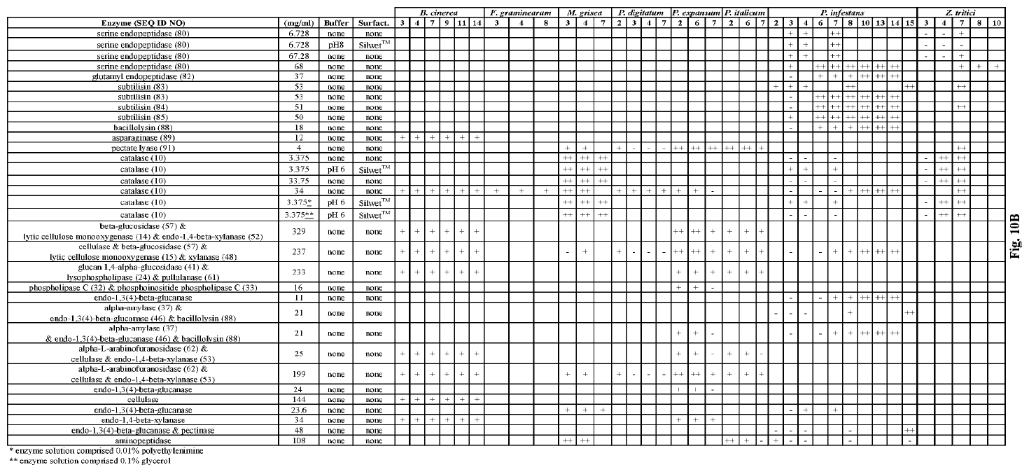

Enzymes were evaluated for efficacy against Botrytis cinerea, Fusarium graminerum, Magnaporthe grisea, Penicilium digitatum, Penicillium expansum, Penicillium italicum, Phytophthora infestans and Zymoseptoria tritici as described above. Results are presented in FIG. 10A and FIG. 10B as (−) no growth inhibition, (+) visible stress reaction and/or mild growth inhibition, (++) moderate growth inhibition or (+++) extreme growth inhibition.

### Example 11

### Effective Control of Leaf-Eating Insects

### Principle

Evaluated the efficacy of enzyme solutions for inhibiting growth/development of leaf-eating insects. Leaf discs were treated with buffered enzyme solutions and subsequently exposed to 2^{nd} instar cabbage looper larvae. The relative growth rate of each larvae was determined after two days of feeding.

### Procedures

1) Leaves were detached from Chinese cabbage plants (var. Minuet) and leaf discs were excised using a #12 cork borer. Only one disc was punched from each leaf. All leaf discs were kept in a water bath until use.
2) One leaf disc was placed in each well of a 12-well plate comprising solidified 1% water agar.
3) Leaf discs were painted with enzyme solutions comprising 1 mg enzyme protein per mL in a phosphate buffer solution (0.163 g K₂HPO₄ and 0.09 g of KH₂PO₄ in 100 mL distilled water) comprising 0.05% SILWET™ L-77, or with a corresponding control solution comprising no enzyme, and then air dried. Twelve replicates were made for each condition.
4) After drying, one 2ⁿ instar cabbage looper larva that had been weighed to the nearest tenth of a milligram was added to each leaf disc.
5) Cabbage loopers were reweighed after two days.
6) The growth rate of the cabbage loopers were determined using the following equation from Bouvaine et al., PLOS ONE 9(1):e86256 (2014):

$$Relative Growth Rate ( RGR ) = [ ln ( final weight / initial weight ) ] / days$$

### Results

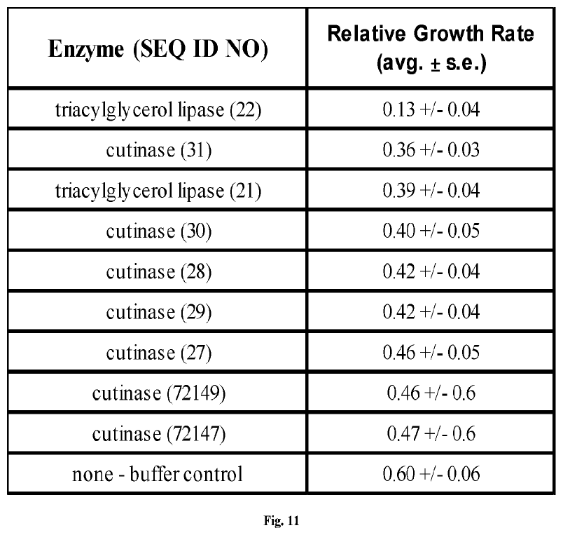

Enzymes were evaluated for efficacy against leaf-eating insects as described above. Results are presented in FIG. 11 as relative growth rates (RGR) after two days of feeding on treated leaf discs.

### Example 12

### Effective Control of Leaf-Eating Insects

### Principle

Evaluated the efficacy of enzyme solutions for inhibiting growth/development of leaf-eating insects was evaluated. Leaf discs were treated with buffered enzyme solutions and subsequently exposed to 2^{nd} instar cabbage looper larvae. The relative growth rate of each larvae was determined after two days of feeding.

### Procedures

1) Leaves were detached from Chinese cabbage plants (var. Minuet) and leaf discs were excised using a #12 cork borer. Only one disc was punched from each leaf. All leaf discs were kept in a water bath until use.
2) One leaf disc was placed in each well of a 12-well plate comprising solidified 1% water agar.
3) Leaf discs were painted with enzyme solutions comprising 1 mg enzyme protein per mL in a phosphate buffer solution (0.163 g K₂HPO₄ and 0.09 g of KH₂PO₄ in 100 mL distilled water) comprising 0.05% SILWET™ L-77, or with a corresponding control solution comprising no enzyme, and then air dried. Twelve replicates were made for each condition.
4) After drying, one 2^{nd} instar cabbage looper larva that had been weighed to the nearest tenth of a milligram was added to each leaf disc.
5) Cabbage loopers were reweighed after two days.
6) The growth rate of the cabbage loopers were determined using the following equation from Bouvaine et al., PLOS ONE 9(1):e86256 (2014):

$$Relative Growth Rate ( RGR ) = [ ln ( final weight / initial weight ) ] / days$$

### Results

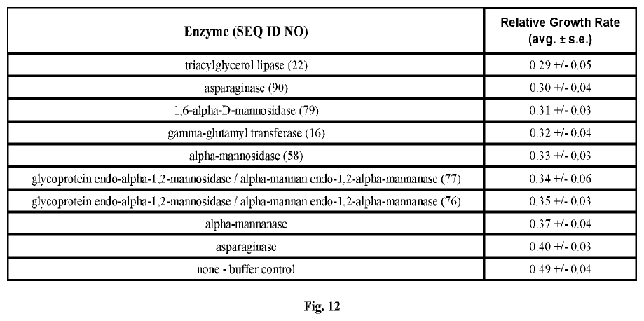

Enzymes were evaluated for efficacy against leaf-eating insects as described above. Results are presented in FIG. 12 as relative growth rates (RGR) after two days of feeding on treated leaf discs.

### Example 13

### Effective Control of Gray Mold

### Principle

Evaluated the efficacy of enzyme solutions for control of gray mold on grapes. Harvested grapes were surface sterilized, inoculated with Botrytis cinerea, wounded, and then treated with buffered enzyme solution. Disease severity was assessed after 14 days. Experiments were conducted by Novozymes Biologicals (Salem, VA).

### Procedures

1. Table grapes were cut into bunches containing 6 grapes. Bunches were washed for five minutes in tap water, two minutes in a 10% bleach solution, and one minute in DI water. Grapes were then allowed to dry for approximately 1-hour.
2. A 10 μl droplet of Botrytis cinerea conidia suspension (1.5×10⁶ conidia per ml water) was pipetted onto the surface of each grape. The inoculum was dried for approximately 1-hour.
3. The skin of each grape was then gently punctured at the site of dried inoculum using a sterile dissecting needle.
4. Grapes were treated with enzyme solutions comprising 2.5 mg enzyme protein per mL in a 0.05 M buffer solution (sodium acetate pH 5, potassium phosphate pH 7, or potassium phosphate pH 8). Enzyme treatments were applied via atomizer sprayer, adequately covering the fruit surface with no pooling.
5. Enzyme treatments were dried onto the fruit for approximately three hours. Each grape bunch was stored in an individual plastic container with lid.
6. Grapes were incubated at 20° C. in the dark for a 14-day period. Disease progression was monitored for 14-days and expressed as a percentage of lesion area to total grape area on a 2D plane.

### Results

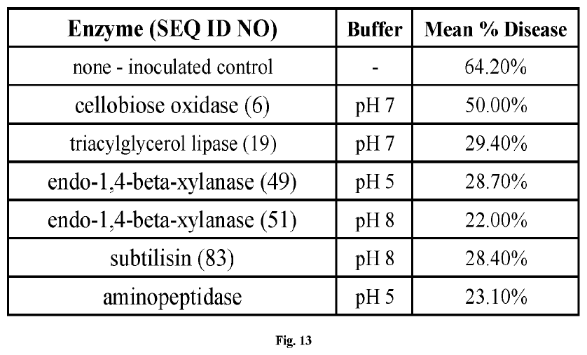

Enzymes were evaluated for efficacy against Botrytis cinerea as described above. Results are presented in FIG. 13 as mean percent disease. A minimum three experimental repeats with six replicates were completed for each treatment.

### Example 14

### Effective Control of Head Blight

### Principle

Evaluated the efficacy of enzyme solutions for control of Fusarium head blight on wheat plants. Plants were treated with enzyme solution and subsequently inoculated with GFP-transformed Fusarium graminearum.

Pathogen biomass was quantified via GFP, and plant health parameters were collected up to 14 days post-inoculation. Testing was conducted at Ghent University (Ghent, Belgium).

### Procedures

1) GFP-transformed Fusarium graminearum strain PH-1 was grown on potato dextrose agar for seven days at 21° C. under a regime of 12 h dark and 12 h combined UVA and UVC light to induce sporulation. 2) A working spore suspension was created by adding “pprox. 20 mL of phosphate buffered saline containing 0.01% TWEEN® 80, pH 7.2-7.4, to the petri dish, agitating the mycelium with a spatula, separating spores from mycelium using a sterile filter, and then diluting to a concentration of 0.5×10⁶ spores per mL.
3) Wheat plants were grown in potting soil for three months under controlled greenhouse conditions (T_{day} 21° C., T_{night} 16° C., 60% relative humidity). Two separate pots, each containing approximately 10 wheat ears in anthesis, were selected for each treatment in the bioassays described below.
4) Plants were sprayed until runoff with enzyme solutions comprising 0.5 or 1 mg enzyme protein per mL in a 10 mM buffer solution (sodium acetate pH 5, potassium phosphate pH 6, potassium phosphate pH 7, or potassium phosphate pH 8), with or without 0.05% w/v surfactant (SILWET™ L-77), or with a corresponding control solution comprising no enzyme, one day prior to inoculation with Fusarium graminearum PH-1 spore suspension.
5) Plants were double-inoculated with Fusarium graminearum PH-1: (i) six ears per plant were point-inoculated on one spikelet with a 10 μl droplet of the Fusarium graminearum PH-1 spore suspension or with a corresponding solution comprising no Fusarium graminearum spores, and (ii) plants were sprayed until runoff with the Fusarium graminearum PH-1 spore suspension or with a corresponding solution comprising no Fusarium graminearum spores.
6) Infected plants and uninoculated control plants were grown under controlled greenhouse conditions (T_{day} 21° C., T_{night} 16° C., 60% relative humidity), except that infected plants were kept at 100% relative humidity during the first 48 hours post-inoculation. Infected plants were grown in a quarantine compartment to prevent infection of uninoculated control plants.
7) Disease progression was monitored through visual inspection and multispectral imaging. Fv/Fm, ChlIdx and cGFP were evaluated from the multispectral images. See Table 2. Mean Fv/Fm, ChlIdx and cGFP values for untreated, inoculated control plants were used as a reference (100%) to evaluate the Fv/Fm, ChlIdx and cGFP values for treated leaves.

### Results

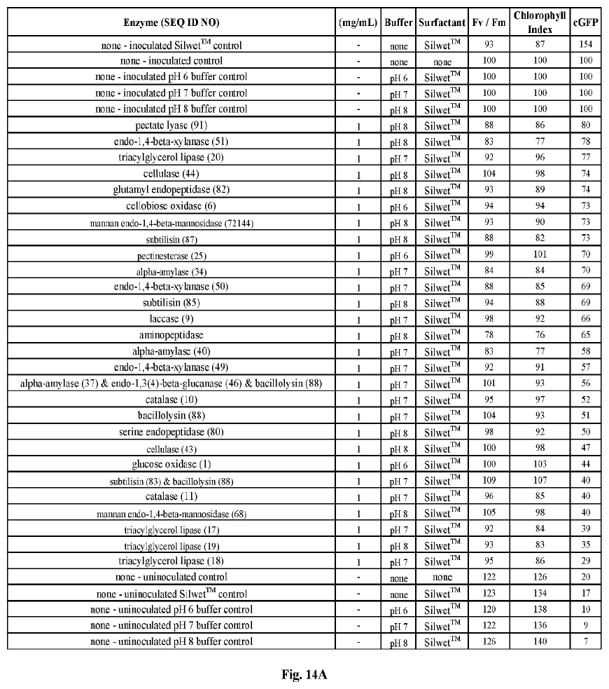

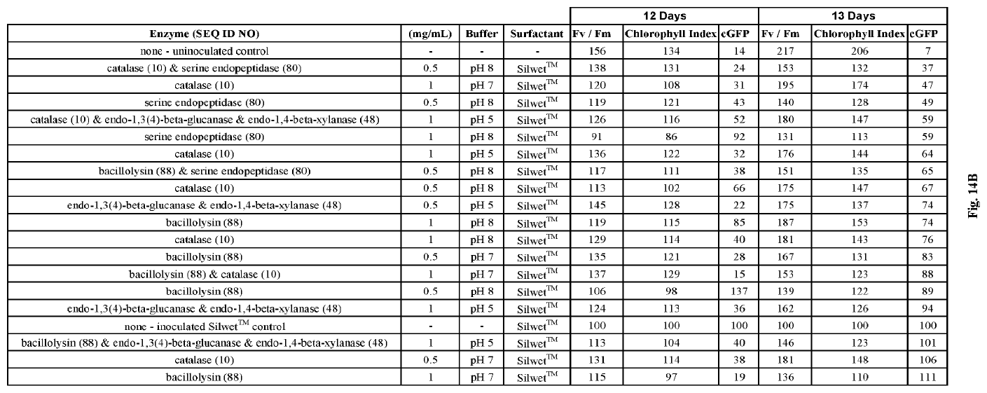

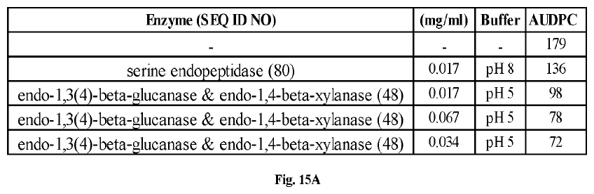

Enzymes were evaluated for efficacy against Fusarium graminearum as described above. Results are presented in FIG. 14A (four days post-inoculation) and FIG. 14B (twelve and thirteen days post-inoculation) as percentage increase of the spectral parameters (Fv/Fm, ChlIdx and cGFP) as compared to a corresponding untreated, inoculated controls.

### Example 15

### Effective Control of Yellow Rust

### Principle

Evaluated the efficacy of enzyme solutions for control of yellow rust on winter wheat plants. Plants were treated with buffered enzyme solution and subsequently inoculated with Puccinia striiformis. Percent leaf area attacked was assessed fifteen days post-inoculation and again with two- or three-day intervals until the full effects were seen. For scoring the standard EPPO scales were used. Experiments were conducted by Arhus University, Flakkebjerg, Denmark.

### Procedures

1) Winter wheat kernels (var. Morocco) were sown 10 seeds/plants per pot in 0.5 L pots and allowed to germinate and grow in a greenhouse for approximately two weeks until reaching the two-leaf crop growth stage (BBCH12).
2) Plants were then sprayed with enzyme solution comprising 0.1 to 0.132 mg enzyme protein per mL in a 10 mM buffer solution (sodium acetate pH 5, potassium phosphate pH 6, potassium phosphate pH 7, or potassium phosphate pH 8) with 0.1% w/v surfactant (SILWET™ L-77), or with a corresponding control solution comprising no enzyme, using a cabin sprayer (volume 600 mL; 150 L/ha, 5.5 km/hour, yellow nozzles 0.2; 2.24 bar). Six replicates were made for each condition.
3) 24 hours after the initial spray application, the plants were sprayed with a Puccinia striiformis spore suspension (Puccinia striiformis f. sp. tritici Dk229/19).
4) After Puccinia striiformis inoculation, the plants were kept under high relative humidity for 24 hours at 10-12° C. to ensure infection before being placed under normal greenhouse conditions.
5) Percent leaf area attacked by yellow rust was determined fifteen days after inoculation and again every two to three days until the full effects were found. For scoring, the standard EPPO scale (EPPO standards: Guidelines on good plant protection practice. Wheat. PP/26 (4)) was used. The area under the disease progress curve was calculated to summarize effects across the performed assessments.

### Results

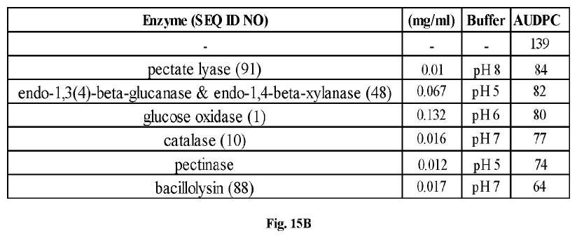

Enzymes were evaluated for efficacy against Puccinia striiformis as described above. Results are presented in FIG. 15 as area under the disease progress curve.

### Example 16

### Degradation of Fungal Cell Wall Components

### Principle

Evaluated the efficacy of enzyme solutions for degradation of fungal cell wall components. Fungal mycelium were isolated and treated with buffered enzyme solution. A reducing end assay was used to analyze cell wall degradation.

### Procedures

1) Shake flasks comprising sterile YP media with 2% glucose were inoculated with a fungal phytopathogen—Botrytis cinerea ATCC56594, Fusarium graminerum Schwabe ATCC 36885, Penicilhum digitatum CBS658.68, Penicillium expansum DSM1282, Penicillium italicum, Phytophthora infestans, Pyricularia grisea, or Zymoseptoria tritici—and incubated for seven days at 26° C. with shaking.
2) After incubation, mycelia were harvested by centrifugation (10 minutes, 3000 rpm). Supernatant was removed by decanting, and pellet was washed 3 times in 3×50 mL demineralized water.
3) After final wash, pellet was suspended in 5 mL demineralized water and homogenized in ball mill with 2 glass beads for 150 seconds at room temperature.
4) Homogenate was washed thoroughly in 50 mL centrifuge tube with 2×20 mL demineralized water
5) The washed pellet was freeze dried and the dried product homogenized in mortar and kept frozen until use. This material is the crude cell wall preparation.
6) For the hydrolysis, 250 L of 20 mg/mL crude cell wall preparation suspended in phosphate buffer was mixed with 250 μL buffered enzyme solution (sodium acetate pH 5, potassium phosphate pH 6, potassium phosphate pH 7, or potassium phosphate pH 8), or with a corresponding solution comprising no enzyme, in 1.5 mL Eppendorf tube and incubated overnight at 40° C. with shaking. Enzymes were evaluated as duplicates.
7) After hydrolysis, the supernatant was isolated by centrifugation (5 minutes at 14000 rpm) and the amount of solubilized oligosaccharides were determined by measuring the reducing ends using the PAHBAH assay:
   a. 20 μL supernatant from hydrolysis or 20 μL standard plus 80 μL demineralized water is mixed with 75 μL freshly prepared PAHBAH solution (15 mg/mL 4-hydroxybenzhydrazide in PAHBAH buffer (50 g/L K—Na-tartrate tetrahydrate+20 g/L NaOH)) in a 96 well PCR plate.
   b. The plate is incubated for 10 minutes at 95° C. in PCR machine and then cooled down to room temperature.
   c. 100 μL is then transferred to new 96 well transparent microtiter plate and the absorbance is measured at 405 nm.
   d. A standard curve is prepared by plotting the absorbance of glucose standards (0.0125 to 0.25 mg/mL) against the glucose concentration and the number of reducing ends are calculated as glucose equivalents from the standard curve. The results are given as mg glucose equivalents per gram of crude cell wall material (mg Glc/g CW).

### Results

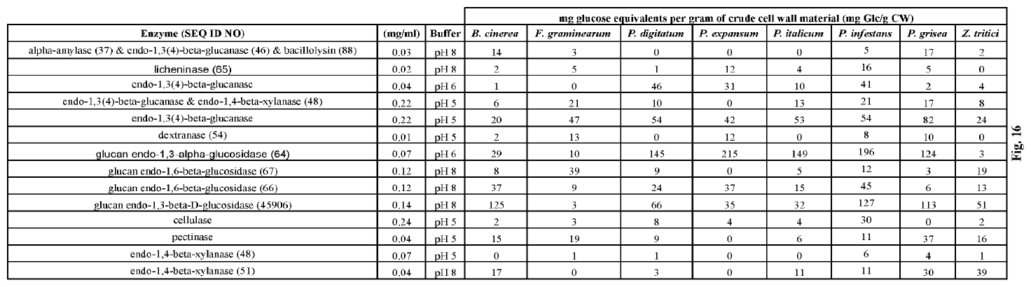

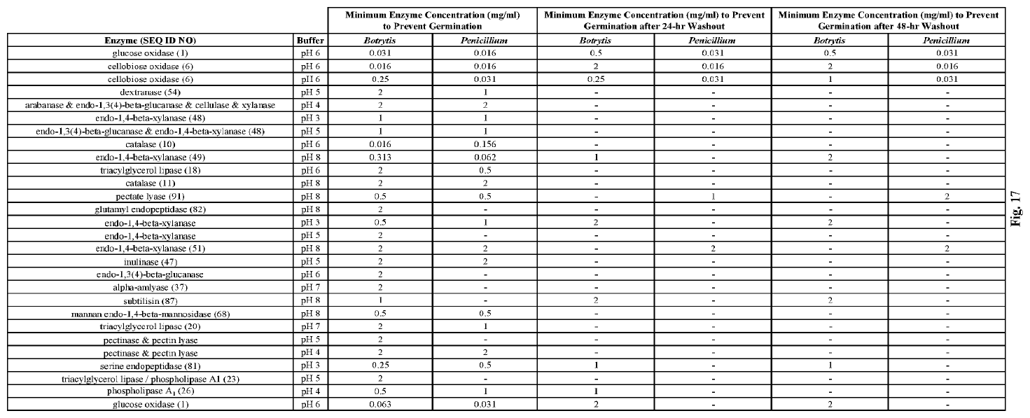

Enzymes were evaluated for efficacy against Botrytis cinerea, Fusarium graminerum, Penicillium digitatum, Penicillium expansum, Penicillium italicum, Phytophthora infestans, Pyricularia grisea and Zymoseptoria tritici as described above. Results are presented in FIG. 16 as mg glucose equivalents per gram of crude cell wall material (mg Glc/g CW).

### Example 17

### Effective Fungal Germination Inhibition

Principle Evaluated the efficacy of enzyme solutions for inhibiting fungal germination. Serial dilutions were used to determine the minimum enzyme concentration required to prevent germination of Botrytis cinerea and Penicillium expansum spores.

### Procedures

1) Enzyme dilution series ranging from 0.002 to 2 mg/mL in 100 μL 10 mM buffer solution (sodium citrate pH 3, sodium citrate pH 4, sodium citrate pH 5, potassium phosphate pH 6, potassium phosphate pH 7, or potassium phosphate pH 8) were added in triplicate to 96-well imaging plates, alongside corresponding control solutions comprising no enzyme.
2) Fungal spore suspensions comprising 5×10⁴ spores per mL of sterile growth medium (Potato Dextrose Broth) were added to the wells. Two fungal phytopathogens were analyzed: Botrytis cinerea and Penicillium expansum.
3) Plates were sealed with BREATHE-EASY® sealing membrane (SIGMA-ALDRICH® catalog no. Z380059) and placed in a 25° C. incubator until ready for imaging.
4) Following incubation at 25° C. for 24 hours, plates were imaged using a high-throughput inverted microscope using a 10× objective. Images were visually analyzed to determine which enzyme treatments allowed/prevented spore germination. Untreated control solutions exhibiting (substantially) complete germination were used as positive controls.
5) After imaging, enzyme solutions were replaced with sterile PBS. Plates were centrifuged at 3000 rpm for ten minutes, supernatant was removed, and sterile PBS was added. Steps were repeated twice more to ensure thorough enzyme washout.
6) Washed fungal spore suspensions were replated in fresh 96-well imaging plates containing sterile growth medium (Potato Dextrose Broth) and incubated for up to 48 hours at 25° C.
7) Washed and replated fungal spore suspensions were reimaged after 24 hours and 48 hours at 25° C. to determine which enzyme treatments allowed/prevented germination even after washout.

### Results

Enzymes were evaluated for efficacy against Botrytis cinerea and Penicillium expansum as described above. Results are presented in FIG. 17 as the minimum enzyme concentration that completely prevent fungal spore germination after 24 hours with enzyme, 24 hours after enzyme washout, and 48 hours after enzyme washout. Data are presented as averages of triplicate samples.

### Example 18

### Effective Fungal Growth Inhibition

Principle Evaluated the efficacy of enzyme solutions for inhibiting fungal growth using high-throughput imaging. Serial dilutions were used to determine the minimum inhibitory concentration (MIC₅₀) of enzyme needed to inhibit growth of Botrytis cinerea.

### Procedures

1) Enzyme dilution series ranging from 0.0078 to 0.5 mg/mL were added in quadruplicate to 384-well imaging plates. A serial dilution of fludioxonil (SCHOLAR® SC; Syngenta Crop Protection, LLC (Greensboro, NC)) was included as a positive control. Plates were centrifuged for two minutes at 2500 rpm to ensure all enzyme samples were at the bottom of the wells.
2) Fungal spore suspensions comprising 2-2.5×10⁴ spores of Botrytis cinerea per mL of sterile growth medium were added to the wells. Sterile growth medium comprised one part Potato Dexrtrose Broth and nine parts M9 minimal medium (200 mL M9 salt solution (64.0 g Na₂HPO₄ 2H₂O, 15 g KH₂PO₄, 2.5 g NaCl, 5.0 g NH₄Cl ad 1 L distilled water), 2 mL 1 M MgSO₄, 20 mL 20% glucose, 100 μl 1 M CaCl₂ in 1 L distilled water).
3) Plates were sealed with BREATHE-EASY® sealing membrane (SIGMA-ALDRICH® catalog no. Z380059) and placed in a lidded plastic container with a wet paper towel.
4) Following incubation at room temperature, 21° C.±1°, for 48 hours, plates were imaged with a high-throughput microscope using a 10× objective, capturing four non-overlapping fields per well.
5) Proprietary, in-house scripts were used to quantify total mycelium area, average hyphal branch length, and total ungerminated spores.

### Results

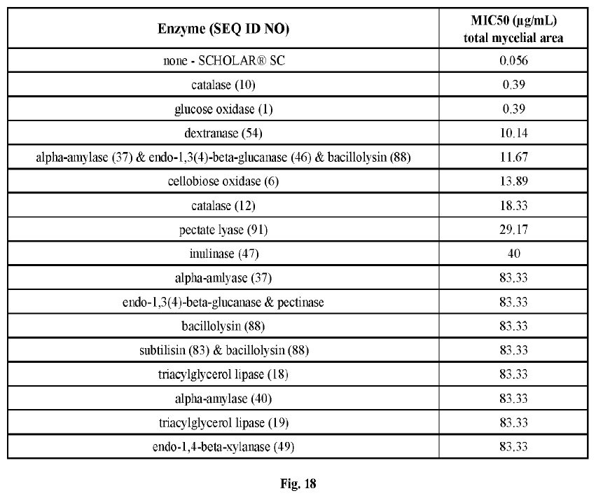

Enzymes were evaluated for efficacy against Botrytis cinerea as described above. Data were normalized to two controls, a 100% inhibition control (cycloheximide) and a 0% inhibition control (1:9 Potato Dextrose Broth:M9; MILLIPORE® catalog no. P6685). Results are presented in FIG. 18 as the minimum inhibitory concentration (MIC₅₀) for average total mycelium area (i.e., the enzyme concentration required to inhibit mycelial area to 50% or less as compared to that of corresponding untreated control). Data are presented as averages of three biological replicates with quadruplicate technical replicates.

### Example 19

### Effective Fungal Growth Inhibition

### Principle

Evaluated the efficacy of enzyme solutions for inhibiting germ tube elongation in Botrytis cinerea and Penicillium expansum using high-throughput imaging.

### Procedures

1) Fungal spore suspensions comprising 5×10⁴ spores per mL of sterile growth medium (Potato Dextrose Broth) were added to 384-well imaging plates.
2) Plates were sealed with BREATHE-EASY® sealing membrane (SIGMA-ALDRICH® catalog no. Z380059), centrifuged for one minute at 1000 rpm, and incubated at 18° C. for six hours.
3) Each well was treated with an enzyme solution comprising enzyme protein in 10 mM buffer solution (sodium citrate pH 3, sodium citrate pH 4, sodium citrate pH 5, potassium phosphate pH 6, potassium phosphate pH 7, or potassium phosphate pH 8) to a final concentration of 2 mg per mL, or with a corresponding control solution comprising no enzyme. All treatments were performed in triplicate.
4) Plates were sealed with BREATHE-EASY® sealing membrane (SIGMA-ALDRICH® catalog no. Z380059), centrifuged for one minute at 1000 rpm, and incubated at 18° C. under ready for imaging.
5) Plates were imaged with a high-throughput microscope using a 20× objective, capturing four non-overlapping fields per well.
6) Proprietary, in-house scripts were used to quantify total hyphal area and germ tube length.

### Results

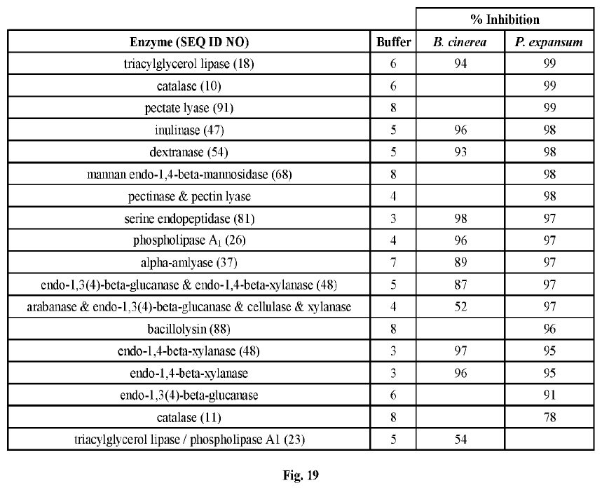

Enzymes were evaluated for efficacy against Botrytis cinerea and Penicillium expansum as described above. Results are presented in FIG. 19 as percent inhibition of germ tube elongation as compared to the corresponding buffer control solution. Data are presented as averages of triplicate samples.

### Example 20

### Exemplary Polypeptide Sequences of the Present Disclosure

<AA; Aspergillus niger>
SEQ ID NO: 1
MQTLLVSSLVVSLAAALPHYIRSNGIEASLLTDPKDVSGRTVDYIIAGGGLTGLTTAARLTENPNISVL
VIESGSYESDRGPIIEDLNAYGDIFGSSVDHAYETVELATNNQTALIRSGNGLGGSTLVNGGTWTRPHK
AQVDSWETVFGNEGWNWDNVAAYSLQAERARAPNAKQIAAGHYFNASCHGTNGTVHAGPRDTGDDYSPI
VKALMSAVEDRGVPTKKDFGCGDPHGVSMFPNTLHEDQVRSDAAREWLLPNYQRPNLQVLTGQYVGKVL
LSQNGTTPRAVGVEFGTHKGNTHNVYAEHEVLLAAGSAVSPTILEYSGIGMKSILEPLGIDTVVDLPVG
LNLQDQTTATVRSRITSAGAGQGQAAWFATFNETFGDYSEKAHELLNTKLEQWAEEAVARGGFHNTTAL
LIQYENYRDWIVNHNVAYSELFLDTAGVASFDVWDLLPFTRGYVHILDKDPYLHHFAYDPQYFLNELDL
LGQAAATQLARNISNSGAMQTYFAGETIPGDNLAYDADLSAWTEYIPYHFRPNYHGVGTCSMMPKEMGG
VVDNAARVYGVQGLRVIDGSIPPTQMSSHVMTVFYAMALKISDAILEDYASMQ;
<AA; Aspergillus chevalieri>
SEQ ID NO: 2
MKPLLWSLFLSVATALPKFPREHLGVEPQLLTDPTVLANTTVDYIIAGGGLTGLTIAARLTEDPNIKVL
VIESGYFESNRGPIIEDLNRYGEIFGTEVDHAFETVQLAVNNRTEIIRSGNGLGGSTLINGGTWTRPHK
VQVDSWETVFGNQGWNWDDLLPYMLKIEKARPPNQRQIEAGHYFNPQCHGFNGSVHAGPRDTGEPYSPI
MRALMDTVSAEGVPVRKDLCCGDPHGVSMFLNTLYPSQIRADAAREYLVPNYYRPNFQVLTGQRVGKVL
LDKTVPGSPKAIGVEFGTHRTRKYEAYARREVLLAAGSTISPTILEYSGIGMKSVLDSVGIEQVVELPV
GVNLQDQTTVHVESRITPAGAGQGQAAYFATFNETFGDFAPQAHELLNTKLDQWAEEVVARGGFNNATA
LRIQYENYRNWLVNDNVAFSELFLDTAGKISFDVWDLIPFTRGYVHIADKDPYLRRLYNNPQYLLNELD
VLGEAAASKLARELSSKGAMAQYYAGETVPGFDHLPADASLRDWAKYVKDRFRPNYHAVSTCAMMSKEL
GGVVDSAARVYDVERLRVVDGSIPPTQVSSHVMTVFYGMAEKIAEAILQDYHARK;
<AA; Penicillium viridicatum>
SEQ ID NO: 3
MKLLGVLSGLGLVVVATALPLQELDLQSTLLTDPRKVAGETFDYVIAGGGLTGLTVAARLTENPDINVL
VIESGFYESNIGPIIENLNHYGDIFTTSVDQAFETVPLAIHNRTEIVRSGKGLGGSTLVNGGSWTRPHK
AQINSWEKVFGMEGWNWDNLLPYMNKVEASRPPNAAQIAAGHYFDPACHGVNGTVQVGPRDTGESYSPM
IKSLMETAKKSGVPVQKDFSCGVPHGISMFPNDVHEDQTRSDAAREWLLPNYKRKNLKVLTGQMVGRVL
FDTTTSTPKAVGVNFGTHNKVNFDVHAKHEVLLAAGSTVSPQILEHSGVGLKTVLDKVGIKQVVELPVG
LNLQDQTTTTVRSAINPIGAGQGQAAYFATFNETFGDQAPRAHQLLNTKLEEWAKDAVSRGGFHNETAL
LIQYQNYRDWLVNSDVSYAEIFIDTAGKLSLDLWDLIPFTRGYVHILDSDPYLRRFAYDPQYFLNELDL
LGQAAASKLAREISNKGEMTKYFNSETVPGNNLAYNATLDQWVDYVKQNFRPNYHGVGTCSMMSKELGG
VVDAAARVYDVEGLRVIDGSIPPTQVSSHVMTVFYGMAEKISEAVLSDYHASN;
<AA; Aspergillus niveoglaucus>
SEQ ID NO: 4
MRPLIWPLFLSVATALPKFPREHFDVQPQLLTDPTVLANTTVDYIIAGGGLTGLTIAARLTEDPKIKVL
VIESGFFESNRGPIIEDLNSYGEIFGTEVDHAFETVSLAVNNRTENIRSGNGLGGSTLINGGTWTRPHK
VQIDSWERVFGNEGWNWDDLLPYMLGIEKARHPNQRQIEAGHYFNPQCHGSNGTVHAGPRDTGESYSPI
MKALMDTVSADGVPVRKDLGCGDPHGVSMFPNSLYESQIRADAAREWLVPNYYRPNLQVLTGQSVGKVL
LDKTVPGAPKAIGVEFGTHRTRKYETFASREVLLAAGSTISPTILEYSGIGIKSVLDSVGIEQVVDLPV
GLNLQDQTTVNLQSRITPDGAGQGQAAYFATFNETFGDFAPQAHELLNTKLDQWAEEVVARGGFHNATA
LRIQYENYRDWLVNDNVAYSELFLDTAGKISFDVWDLIPFTRGYVHIADKDPYLRRLSNDPQYFLNELD
VLGQAAASKLARELSSRGAMAQYFASETVPGYDRLPADASLQDWAKYVKDRFRANYHAVSTCSMMPKEM
GGVVDSTARVYGVERLRVVDGSIPPTQVSSHVMTVFYGMAVKIAEAILQDHHTRK;
<AA; Talaromyces bacillisporus>
SEQ ID NO: 5
MKSILIVSALASLAGAQGYTPAEQIDVQSSLISDPKNVSGRTFDYIIAGGGLTGLTVAAKLSEDPNISV
LVIEKGFYESNDGPIIENPNDYGLIFGSSVDQNYLTVPMAINNRTLDIKSGKGLGGSTLINGDSWTGPD
KVQIDSWETVLGNPGWNYHALKEYMNKAERARYPTTTQIAAGMYFNSTCHGFNGTVNSGPRDDGRPYSP
LMKALMNTTSAMGVPTQADLLCGHPRGVSMIYNNLLPDQTRADAAREWLLPNYKRPNLSILTGQIVGKV
LFDKTPTGPRAVGVNFGTNKAVNFNVSAKHEVLLAAGSNVSPLILEHSGIGLKSVLDQFNISQLVELPV
GLNMQDQTTTTVRARAKASAAGQGQAVYFANFTEVFGNHTAQATNLLNTKLEQWANETVARGGFNNATA
LLIQYENYRKWLLDEDVAYVELFFDTNGKMNFDLWDLIPFTRGSVHIAHADPYLQSFSNNPMFLLNELD
LLGQAAGSMLARELQNSGELSTYFDGEDIPGAELLPYDATLDGWVGYVKDNFRANWHAVSTCSMMPKEL
GGVVDSAARVYGTQGLRVIDGSIPPTQVSSHVMTVFYAMALKIADAILADYTA;
<AA; Microdochium nivale>
SEQ ID NO: 6
LVTRGAIEACLSAAGVPIDIPGTADYERDVEPFNIRLPYIPTAIAQTQTTAHIQSAVQCAKKLNLKVSA
KSGGHSYASFGFGGENGHLMVQLDRMIDVISYNDKTGIAHVEPGARLGHLATVLNDKYGRAISHGTCPG
VGISGHFAHGGFGFSSHMHGLAVDSVVGVTVVLADGRIVEASATENADLFWGIKGAGSNFGIVAVWKLA
TFPAPKVLTRFGVTLNWKNKTSALKGIEAVEDYARWVAPREVNFRIGDYGAGNPGIEGLYYGTPEQWRA
AFQPLLDTLPAGYVVNPTTSLNWIESVLSYSNFDHVDFITPQPVENFYAKSLTLKSIKGDAVKNFVDYY
FDVSNKVKDRFWFYQLDVHGGKNSQVTKVTNAETAYPHRDKLWLIQFYDRYDNNQTYPETSFKFLDGWV
NSVTKALPKSDWGMYINYADPRMDRDYATKVYYGENLARLQKLKAKFDPTDRFYYPQAVRPVK;
<AA; Chaetomium globosum>
SEQ ID NO: 7
MQTLAPLLALLGLAATSHATSFARFTNETGTLTDCLTKAGVPIGESGTPEYKIDVASFNLRLNYTPAAV
VAASTAQHVQDAVVCANKFGIKPTAKCGGHSYASFGLGGEDGHLVIEMSRMNKVVLDNVTGIATAEGGT
RLGHLAMELYTQGKRAISHGTCPGVGVGGHLLHGGYGMSSHTHGLALDYVIGATVVLANGTVVECSETQ
HGELFWALRGAGASMGIVTEFRLKTFEAPGQLTYFVAPVQWPTEARALVGVTAVQEFAKTMPAELNMRL
FIARRFVNLEGLFYGDKEDLKAALAPLVAKTNATLALATTGDWMEQIKHFGGGSNIDQGHGYEEHETFY
STSLYTKELNDEQLQNFVGYWFNQAKNNTRDWYVQIDLHGGENSAVSAPAADSTAYAHRDYLLMYLLYD
RVDKGEYPAEGHTVMENFAGNITQGMERSDWGMYINYPNSRLDQKDAQLNYWGDNLPKLQAIKKDVDPE
DVFHFPQGVMPAVRHQHQHQH;
<AA; Neurospora crassa>
SEQ ID NO: 8
MWLPLILLTATAAPILANPLSSPLNKRAAIDDCLKSAGVPADAQGSSEWRTDVNPFNQRLPYTPVAIAV
PTTIEHIQGAVSCATKLGIKVTPKSGGHSYASFGLGGENGHLVVELDRMSKVTLDKTTNIADVQSGARL
GHVATELPYFLAWPGLTKEGNRVGVGGHSLHGGFGFSSHTYGLAVDWIAAATVVLANSTVVTASPTENP
DLFWALRGAGSNFGIVASFKFNTFAAPSQVTAFQINLPWNSASSIASGWEKLQDWLAAGNMPKEMNMRV
FGSPSQTQLQGLYHGSSSALRTAVQPLLSTLGASLSNAQQYDWMGAFTYYTYGGTVDVTHPYNTVETFY
SKSLVTTALPSAALNSVANYWINTAKRVSRDWFIIIDMHGGPKSAITSSTTNSANYTSSYAYRAPEYLF
LYELYDRVMFGSYPSNGFSFLDGWVKSFTDNMKQEQWGMYINYADPTMKRAEAVGNYYRSSLSRLQKVK
AQYDPNEVFYYPQSVEPARHQHQHQH;
<AA; Thermothelomyces thermophilus>
SEQ ID NO: 9
MKSFISAATLLVGILTPSVAAAPPSTPEQRDLLVPITEREEAAVKARQQSCNTPSNRACWTDGYDINTD
YEVDSPDTGVVRPYTLTLTEVDNWTGPDGVVKEKVMLVNNSIIGPTIFADWGDTIQVTVINNLETNGTS
IHWHGLHQKGTNLHDGANGITECPIPPKGGRKVYRFKAQQYGTSWYHSHFSAQYGNGVVGAIQINGPAS
LPYDTDLGVFPISDYYYSSADELVELTKNSGAPFSDNVLFNGTAKHPETGEGEYANVTLTPGRRHRLRL
INTSVENHFQVSLVNHTMTIIAADMVPVNAMTVDSLFLGVGQRYDVVIEASRTPGNYWFNVTFGGGLLC
GGSRNPYPAAIFHYAGAPGGPPTDEGKAPVDHNCLDLPNLKPVVARDVPLSGFAKRPDNTLDVTLDTTG
TPLFVWKVNGSAINIDWGRPVVDYVLTQNTSFPPGYNIVEVNGADQWSYWLIENDPGAPFTLPHPMHLH
GHDFYVLGRSPDESPASNERHVFDPARDAGLLSGANPVRRDVTMLPAFGWVVLAFRADNPGAWLFHCHI
AWHVSGGLGVVYLERADDLRGAVSDADADDLDRLCADWRRYWPTNPYPKSDSGLKHRWVEEGEWLVKA;
<AA; unidentified>
SEQ ID NO: 10
QCPYLSGEMSFTQEQDNAGDTIEVTEQTIDNTLYVNDTGSYMTTDFGTPISDQYSLKAGRRGPTLLEDF
IFRQKLQRFDHERVPERVVHARGAGAYGTFKSYADWSNVTAADFLSANEKETPMFCRFSTVVGFRGSVD
TARDVHGHACRFYTDEGNYDIVGINFAPFFIQDAIQFPDLVHAIKPMPNNEIPQAATAHTSAWDFFSQQ
STALHSALWLMSGNGIPRSFRHMNGYGVHSFRFVAANGTSKVVRYRWKSQQGVASLVWEEAQAAAGKNS
DYHRQDLYNAIANGHYPKYELQAQIMDEADMLRFGFDLLDPTKLVPEEVVPYTPLGMMELNANPTNYFA
EVEQAGFQPGHVVPGIDFTDDPLLQGRLFSYLDTQLTRHGGPNFEQIPVNRPRKPVHNNNRDGFGQQQI
PTNNWAYTPNTMSNGYPMQANQTHGHGFFTSPYRYASGHLIREPSPTFHDHWSQPAMFWNSLIPAEQQM
VVNAIVFENSKVNSPHVRKNVVNQLNMVNNNLAVRVARGLGLDEPSPNPTYYTSNKTSNVGTFGKPLLS
IEGLQVGFLASNSHPESIKQGQAMAAQFSAAGVDLNIVTEAYADGVNTTYALSDAIDFDALIIADGVQS
LFASPSLANQMNSTATSTLYPPARPYQILVDSFRYGKPVAAVGSGSAALKNAGIDTSRSGVYTGSSETT
ENIAKEVLKGLYTFRFVDRFALDE;
<AA; Thermoascus aurantiacus>
SEQ ID NO: 11
MRAIGLLPGIIGIAGAACPYMTGELPRSFAENPHAINRRAEGGGGAAAETEKFLSQFYLNDNDTFMTTD
VGGPIEDQNSLSAGDRGPTLLEDFILRQKIQRFDHERVPERAVHARGAGAHGVFTSYADWSNITAASFL
SAAGKETPVFVRFSTVAGSRGSADTARDVHGFATRFYTDEGNFDIVGNNIPVFFIQDAIQFPDLIHAVK
PSPNNEIPQAATAHDSAWDFFSQQPSSLHTLFWAMAGHGIPRSYRNMDGFGIHTFRFVTDDGASKLVKF
HWTSLQGKASLVWEEAQAVAGKNADYHRQDLWDAIEAGRYPEWELGVQIMDEEDQLRFGFDLLDPTKIV
PEEYVPITKLGKMQLNRNPLNYFAETEQIMFQPGHVVRGIDFTEDPLLQGRLFSYLDTQLNRHGGPNFE
QIPINRPRTPIHNNNRDGAAQMYIPLNKAAYTPNTLNNGSPKQANQTVGKGFFTTPGRTASGRLVRAVS
STFADVWSQPRLFYNSLVPAEQQFLINAIRFETAHITSDVVKNNVIIQLNRVSNNLAKRVARAIGVAEP
EPDPTLYHNNKTANVGVFGKPLARLDGLQVGVLATVNKPDSIKQAASLKASFAADNVDVKVVAERLADG
VDETYSAADAVNFDAILVANGAEGLFARDSFTARPANSTTATLYPAGRPLQILVDGFRYGKPVGALGSG
AKALDAAEISTTRAGVYVANSTTDSFINGVRDGLRTFKFLDRFAIDEDAE;
<AA; Thermoascus aurantiacus>
SEQ ID NO: 12
ACPYMTGELPRSFAENPHAINRRAEGGGGAAAETEKFLSQFYLNDNDTFMTTDVGGPIEDQNSLSAGDR
GPTLLEDFILRQKIQRFDHERVPERAVHARGAGAHGVFTSYADWSNITAASFLSAAGKETPVFVRFSTV
AGSRGSADTARDVHGFATRFYTDEGNFDIVGNNIPVFFIQDAIQFPDLIHAVKPSPNNEIPQAATAHDS
AWDFFSQQPSSLHTLFWAMAGHGIPRSYRNMDGFGIHTFRFVTDDGASKLVKFHWTSLQGKASLVWEEA
QAVAGKNADYHRQDLWDAIEAGRYPEWELGVQIMDEEDQLRFGFDLLDPTKIVPEEYVPITKLGKMQLN
RNPLNYFAETEQIMFQPGHVVRGIDFTEDPLLQGRLFSYLDTQLNRHGGPNFEQIPINRPRTPIHNNNR
DGAAQMYIPLNKAAYTPNTLNNGSPKQANQTVGKGFFTTPGRTASGRLVRAVSSTFADVWSQPRLFYNS
LVPAEQQFLINAIRFETAHITSDVVKNNVIIQLNRVSNNLAKRVARAIGVAEPEPDPTLYHNNKTANVG
VFGKPLARLDGLQVGVLATVNKPDSIKQAASLKASFAADNVDVKVVAERLADGVDETYSAADAVNFDAI
LVANGAEGLFARDSFTARPANSTTATLYPAGRPLQILVDGFRYGKPVGALGSGAKALDAAEISTTRAGV
YVANSTTDSFINGVRDGLRTFKFLDRFAIDEDAE;
<AA; Coprinopsis cinerea>
SEQ ID NO: 13
MKLSLLSTFAAVIIGALALPQGPGGGGSVTCPGGQSTSNSQCCVWFDVLDDLQTNFYQGSKCESPVRKI
LRIVFHDAIGFSPALTAAGQFGGGGADGSIIAHSNIELAFPANGGLTDTVEALRAVGINHGVSFGDLIQ
FATAVGMSNCPGSPRLEFLTGRSNSSQPSPPSLIPGPGNTVTAILDRMGDAGFSPDEVVDLLAAHSLAS
QEGLNSAIFRSPLDSTPQVFDTQFYIETLLKGTTQPGPSLGFAEELSPFPGEFRMRSDALLARDSRTAC
RWQSMTSSNEVMGQRYRAAMAKMSVLGFDRNALTDCSDVIPSAVSNNAAPVIPGGLTVDDIEVSCPSEP
FPEIATASGPLPSLAPAP;
<AA; Penicillium emersonii>
SEQ ID NO: 14
MLSSTTRTLAFTGLAGLLSAPLVKAHGFVQGIVIGDQFYSGYIVNSFPYESNPPPVIGWATTATDLGFV
DGTGYQGPDIICHRNATPAPLTAPVAAGGTVELQWTPWPDSHHGPVITYLAPCNGNCSTVDKTTLEFFK
IDQQGLIDDTSPPGTWASDNLIANNNSWTVTIPNSVAPGNYVLRHEIIALHSANNKDGAQNYPQCINIE
VTGGGSDAPEGTLGEDLYHDTDPGILVDIYEPIATYTIPGPPEPTF;
<AA; Thermoascus aurantiacus>
SEQ ID NO: 15
MSFSKIIATAGVLASASLVAGHGFVQNIVIDGKNYGGYLVNQYPYMSNPPEVIAWSTTATDLGFVDGTG
YQTPDIICHRGAKPGALTAPVSPGGTVELQWTPWPDSHHGPVINYLAPCNGDCSTVDKTQLEFFKIAES
GLINDDNPPGIWASDNLIAANNSWTVTIPTTIAPGNYVLRHEIIALHSAQNQDGAQNYPQCINLQVTGG
GSDNPAGTLGTALYHDTDPGILINIYQKLSSYIIPGPPLYTG;
<AA; Bacillus licheniformis>
SEQ ID NO: 16
EGVMSGGGGDKVAVGKDGMVATAHPLASKIGAEVLKKGGNAIDAAIAIQYALNVTEPMMSGIGGGGFMM
VYDGETKETSIINSRERAPEGAKPDMFLDGDGKVIPFSERSRHGNAVGVPGTLKGLEAAHKKWGTKKME
DLISPSIKLAEEGFPIDSVLADAIKDHQDKLSKTAAKDIFLPDGEPLKEGDILVQKDLAKTFKLIRKEG
SKAFYDGEIGRAIADVVQDFGGSMTPDDLSRYEVTTDKPIWGEYHGYDIASMPPPSSGGVFMLQMLKLI
DDFHLSQYDPKSFEKYHLLAETMHLSYADRAAYAGDPEFVDVPLRGLLDPDYIKERQKLISLDSMNRDV
KEGDPWKYEEGEPNYEIVPQPEDKTIGETTHFTVTDQWGNVVSYTTTIEQLFGTGILVPGYGLFLNNEL
TDFDAVPGGANEVQPNKRPLSSMTPTIVFKDEKPVLTVGSPGGTTIIASVFQTILNYFEYGMSLQDAIE
EPRIYTNSLTSYRYESGMPEDVRRKLNDFGHKFGANPVDIGNVQSIFIDRENKTFMGVADSSRNGTAVG
VNIKTSAK;
<AA; Thermomyces lanuginosus>
SEQ ID NO: 17
MRSSLVLFFVSAWTALASPIRREVSQDLFNQFNLFAQYSAAAYCGKNNDAPAGTNITCTGNACPEVEKA
DATFLYSFEDSGVGDVTGFLALDNTNKLIVLSFRGSRSIENWIGNLNFDLKEINDICSGCRGHDGFTSS
WRSVADTLRQKVEDAVREHPDYRVVFTGHSLGGALATVAGADLRGNGYDIDVFSYGAPRVGNRAFAEFL
TVQTGGTLYRITHTNDIVPRLPPREFGYSHSSPEYWIKSGTLVPVTRNDIVKIEGIDATGGNNQPNIPD
IPAHLWYFGLIGTCL;
<AA; Thermomyces lanuginosus>
SEQ ID NO: 18
EVSQDLFNQFNLFAQYSAAAYCGKNNRAPAGTNITCTGNACPEVEKADATFLYSFEDSGVGDVTGFLAL
DNTNKLIVLSFRGSRSIENWINNLRFDLKEINDICSGCRGHAGFTSSWRSVADTLRQKVEDAVREHPDY
RVVFTGHSLGGALATVAGADLRGNGYDIDVFSYGAPRVGNRAFAEFLTVQTGGTLYRITHTNDIVPRLP
PREFGYSHPSPEYWIKSGTGVPVTRNDIVKIEGIDATGGNNQPNIPDITAHLWYFGLIGTCL;
<AA; Humicola insolens>
SEQ ID NO: 19
QLGAIQNDLESGSPDACPDAILIFARGSTEPGNMGITVGPALANGLKEHIPNIWIQGVGGPYDAALATN
FLPRGTSQANIDEGKRLFHLAHQKCPNTPVVAGGYSQGAALIAAAVSELSGAVKEQVKGVVLFGYTQNL
QNRGGIPNYPRERTKVFCNVGDAVCTGTLIITPAHLSYTIQARGEAARFLVDRIRA;
<AA; Thermomyces lanuginosus>
SEQ ID NO: 20
SPIRREVSQDLFNQFNLFAQYSAAAYCGKNNDAPAGTNITCTGNACPEVEKADATFLYSFEDSGVGDVT
GFLALDNTNKLIVLSFRGSRSIENWIGNLNFDLKEINDICSGCRGHDGFTSSWRSVADTLRQKVEDAVR
EHPDYRVVFTGHSLGGALATVAGADLRGNGYDIDVFSYGAPRVGNRAFAEFLTVQTGGTLYRITHTNDI
VPRLPPREFGYSHSSPEYWIKSGTLVPVRRRDIVKIEGIDATGGNNQPNIPDIPAHLWYFGLIGTCL;
<AA; Penicillium thomii>
SEQ ID NO: 21
FPAIQERATADTAAWSFLQRAADLSSAAYTGCLGTAFDVTITKQIYDAATNAKGFVGYSTINKKISVVM
KGSTTETDIMNDVDTTLVTPSLSGVTFPSGAQIMHGISSPWSAVHDTIISEVKTLVEKYPDYTLESTGH
SLGGSLTYISYVALSANFPDKEVTSNAMAAFPIGNQAWATFAESFNGTLNRGNNVDDGVPNMYIELPYD
FVHYGTEYYSYGTEATVLKCSGERDTSCSAGNGQYGVTAGHFSSFGVEMGYAGCSSL;
<AA; Cryphonectria parasitica>
SEQ ID NO: 22
SPLQARDSAVLDETSYENMKYYVQYAASAYCDSEDAVGTLVSCGDSGCPNVTANGATIVGTMPTTTTFD
LEGYVAVDPTREEIVVAFRGSSDLRNWIADFDFIEVAYSDCTGCYVHDGFYESWKEIQTYAVGYVEAAY
ETYPDYTLVITGHSLGAAVATLAGVQFRIDGYPCDIYTVGSPRIGNLAWAEFVTAQDGAEYRATHYDDP
VPRLPPIVLGYYHTSPEFWLAAGPATNIDYTIDGIDVCVGNANTSCNAGTTGFDADAHEYYFQYMGCGD
ESNGIDMRKRNITDAELAQKLTNYTMQDIAFSEKLASSS;
<AA; Thermomyces lanuginosus>
SEQ ID NO: 23
EVSQDLFNQFNLFAQYSAAAYCGKNNDAPAGTNITCTGNACPEVEKADATFLYSFEDSGVGDVTGFLAL
DNTNKLIVLSFRGSRSIENWIANLNFWLKKINDICSGCRGHDGFTSSWRSVADTLRQKVEDAVREHPDY
RVVFTGHSLGGALATVAGADLRGNGYDIDVFSYGAPRVGNRAFAEFLTVQTGGTLYRITHTNDIVPRLP
PREFGYSHSSPEYWIKSGTLVPVTRNDIVKIEGIDATGGNNQPNIPDIPAHLWYFQATDACNAGGFS;
<AA; Aspergillus niger>
SEQ ID NO: 24
MKLPLFAAAAAGLANAASLPVERAEAEVASVAADLIVRALPNAPDGYTPSNVTCPSTRPSIRDASGIST
NETEWLKVRRNATLTPMKNLLSRLNLTGFDTTSYINEHSSNISNIPNIAIAASGGGYRALTNGAGALKA
FDSRSDNATNSGQLGGLLQAATYVSGLSGGSWLVGSMFVNNFSSIGELQASEKVWRFDKSLLEGPNFDH
IQIVSTVEYWKDITEEVDGKANAGENTSFTDYWGRALSYQLVNASDDKGGPDYTWSSIALMDDFKNGQY
PMPIVVADGRNPGEIIVETNATVYEVNPWEFGSFDPSVYAFAPLQYLGSRFENGSIPDNGTCVSGFDNA
GFIMGSSSTLFNQFLLQINSTSIPTILKDAFTDILEDLGERNDDIAVYSPNPFSGYRDSSEDYATAKDL
DVVDGGEDGENIPLHPLIQPERAVDVIFAIDSSADTDYYWPNGTSLVATYERSLEPSIANGTAFPAVPD
QNTFVNLGLNSRPTFFGCDPKNISGTAPLVIYLPNSPYTYDSNFSTFKLTYSDEERDSVITNGWNVVTR
GNGTVDDNFPSCVACAILQRSTYRTNTSLPDICTTCFNDYCWNGTTNSTTPGAYEPSV;
<AA; Aspergillus aculeatus>
SEQ ID NO: 25
MVKSVLASALFAVSALAASRTTAPSGAIVVAKSGGDYTTIGDAIDALSTSTTDTQTIFIEEGTYDEQVY
LPAMTGKVIIYGQTENTDSYADNLVTITHAISYEDAGESDDLTATFRNKAVGSQVYNLNIANTCGQACH
QALALSAWADQQGYYGCNFTGYQDTLLAQTGNQLYINSYIEGAVDFIFGQHARAWFQNVDIRVVEGPTS
ASITANGRSSETDTSYYVINKSTVAAKEGDDVAEGTYYLGRPWSEYARVVFQQTSMTNVINSLGWTEWS
TSTPNTEYVTFGEYANTGAGSEGTRASFAEKLDAKLTITDILGSDYTSWVDTSYF;
<AA; Evansstolkia leycettana>
SEQ ID NO: 26
MHRPLQLWALAALTSLVTAAPAPVLRRDVSSSVLSELDLFAQYSAAAYCSSNIGSPGTKLTCSVGNCPR
VEAADTETLIEFNESSSFGDVTGYIAVDRTNSLLVLAFRGSSTVSNWEADLDFPLTDASSLCSGCEIHS
GFWAAWQTVQASITSTLESAIASYPGYTLVFTGHSYGAALAAIAATTLRNAGYTIQLYDYGQPRLGNLA
LAQYITAQTQGANYRVTHTDDIVPKLPPELFGYHHFSPEYWITSGDNVTVTTSDVQVVTGIDSTAGNDG
TLLDSTSAHDWYIVYIDGCD;
<AA; Nemania serpens>
SEQ ID NO: 27
IPMTLDLSEEMIDKLKAGKASGVSEATATGTTANEFLDGGCRPVIFLFARGSRQEGNVGGTPGPQTIDQ
LKARLGDGAVAAQGLEYPADLLDNLRSGGCDPADVDSFGALIARAAAECPGSRLVLSGYSQGAALVHAA
ARKLAADVVVRIAAAVTYGDTRKEQDGGVIPGIDPARTLILCHDGDLVCDGTLIVTDNHYDYDDLAPTA
VDFIASKV;
<AA; Trichophaea saccata>
SEQ ID NO: 28
TPFTGLDTPIDHFKRDENGTVPTRTLDISTSIYEKWDALFSAAATPSASSAASVKIRTSSDVREDLNNG
MCGNVVLIYARGTYSSGNIGIGVGVAFVDALAAALPNQVIVQGVEPYDNDIAGYLAGGDETGANSMVAL
TQKAASQCPSAKIVWGGYSQGAQVTHKAGERLPSALYPRIAGIMLFGDPDNGDPFPGSLNNHVKTYCHS
DDPICDGIPLPIAGHLTYDENATEAAAVVATWV;
<AA; Thermochaetoides thermophila>
SEQ ID NO: 29
LPTAPVEETASHLEARQSTTRNDLENGNSNNCPGVIFIFARASTEPGNMGISAGPNVANGLAAHYGSNL
WVQGVGGPYTAALADNFLPAGTSRAAIDEAKRLFNLASTKCPNAAIVAGGYSQGTAVMSNAISELSSSV
RDKVKGVVLFGYTKNLQNGGQIPNYPSNRLEVYCNASDAVCWGTLFITPAHFLYTTESTIDAPRFLISK
VGF;
<AA; Pyrenophora tritici-repentis>
SEQ ID NO: 30
APTPETGVSFPITDLLDSDLTINEYAAQLEAKNVEERDLTKRQYNSDTYNQLTDGTACRPITVIWARGT
TQSGNVGEPNSEGPVFFNAIAARVGGISRLAIQGVTYPANVFGFLAGGDAAGATTMFNLINQAISQCPS
TKIVVTGYSQGAQLVHTATQRLSAAAAARVTAVVTFGDADRDESFGSVAASKVLIICHEGDNICDNGII
ITPQHRNYEIDAPTAAAFVAARV;
<AA; Acrophialophora fusispora>
SEQ ID NO: 31
FPTAPVELESTAEIEARQLSSTRTDLESGSSSNCPRVIFIFARASTETGNMGLSAGPNVANGLAARYGS
NLWVQGVGGPYTAGLAENFLPAGTSRAAIDEAKRLFTMANQKCPNAAVVAGGYSQGTAVMSNAISELST
TIKNQIKGVVLFGYTKNLQNGGRIPNFDTAKTEVYCNVSDAVCWGTLFIAPAHFLYTTDSTINAPAFLI
SKIGA;
<AA; Fusarium longipes>
SEQ ID NO: 72147
MKPTQVLLALAAIAYAAPVAEDTKVEAVEFDDDFPLAELEAYFENHLNGQASESGSLQARQFSSSTYNQ
LTDGTPCRPVTMIYARGTTQAGNVGDPAAVGPVLFNNLASRIGLNNLAVQGVTYPANVAGFLAGGDAAG
SRTMATLISRAASQCPSTKIIISGYSQGAQLVHNAAGMLSASVANRVTAAVTFGDPKQNEAFGTIPSSR
TRVYCRSGDNICTGGIIITPAHSQYQQDAPAAAQWIAARV;
<AA; Colletotrichum graminicola>
SEQ ID NO: 72149
MHRSTPALLLAVLGMASASPFAIVPRQADFSGNQQDGLSGPCQPTMVIFARGTTERGNVGTLAGPPFFQ
ALSAQVGGALAVQGVEYPASVQGFLAGGDAKGSQTMATLTEQAITKCPNSSIIMSGYSQGGQLVHNAAA
MMSQSMVASVAGAVIFGDPLNGQPVQGVPTQRTMVICHNGDNICEGGSQIRRAHLTYGDNAAEAAQFAA
SMTPPSAAAA;
<AA; Pseudomonas sp.>
SEQ ID NO: 32
AQESPAFIDPASWNTPFNGIAQVACHNCYEKQYANTFSSVLDSVRTLELDFWDQRDAVSGGSPHHWFVR
HNPGTLFQSGNDNNCTGDGTGKNDLEACLNDVKNWSDKHPGHFPITLILDKKQGWSKESSGRTPKDFDE
LVARVFQGKLFTPQDLATHIGSGAGALQGNLKGKSWPTANDLQGKVLLVLNHSENQKLSQYAEARTSKA
KVFISPVTNGQNDISGKVSGMSSQSSGYVAMNNMGKGDKSWAKQAFAYSHIGRVWGDDEVSFAQHINQK
INLSAYYRFAAQSAGGYRIRPF;
<AA; Bacillus thuringiensis>
SEQ ID NO: 33
WSAEDKHKEGVNSHLWIVNRAIDIMSRNTTLVKQDRVAQLNEWRTELENGIYAADYENPYYDNSTFASH
FYDPDNGKTYIPFAKQAKETGAKYFKLAGESYKNKDMKQAFFYLGLSLHYLGDVNQPMHAANFTNLSYP
QGFHSKYENFVDTIKDNYKVTDGNGYWNWKGTNPEDWIHGAAVVAKQDYSGIVNDNTKDWFVKAAVSQE
YADKWRAEVTPMTGKRLMDAQRVTAGYIQLWFDTYGDR;
<AA; Geobacillus stearothermophilus>
SEQ ID NO: 34
APFNGTMMQYFEWYLPDDGTLWTKVANEANNLSSLGITALWLPPAYKGTSRSDVGYGVYDLYDLGEFNQ
KGTVRTKYGTKAQYLQAIQAAHAAGMQVYADVVFDHKGGADGTEWVDAVEVNPSDRNQEISGTYQIQAW
TKFDFPGRGNTYSSFKWRWYHFDGVDWDESRKLSRIYKFRGKAWDWEVDTEFGNYDYLMYADLDMDHPE
VVTELKNWGKWYVNTTNIDGFRLDAVKHIKFSFFPDWLSYVRSQTGKPLFTVGEYWSYDINKLHNYITK
TDGTMSLFDAPLHNKFYTASKSGGAFDMRTLMTNTLMKDQPTLAVTFVDNHDTEPGQALQSWVDPWFKP
LAYAFILTRQEGYPCVFYGDYYGIPQYNIPSLKSKIDPLLIARRDYAYGTQHDYLDHSDIIGWTREGGT
EKPGSGLAALITDGPGGSKWMYVGKQHAGKVFYDLTGNRSDTVTINSDGWGEFKVNGGSVSVWVPRKTT
VS;
<AA; Geobacillus stearothermophilus>
SEQ ID NO: 35
MKQQKRLYARLLTLLFALIFLLPHSAAAAPFNGTMMQYFEWYLPDDGTLWTKVANEANNLSSLGITALW
LPPAYKGTSRSDVGYGVYDLYDLGEFNQKGTVRTKYGTKAQYLQAIQAAHAAGMQVYADVVFDHKGGAD
GTEWVDAVEVNPSDRNQEISGTYQIQAWTKFDFPGRGNTYSSFKWRWYHFDGVDWDESRKLSRIYKFRG
KAWDWEVDTEFGNYDYLMYADLDMDHPEVVTELKNWGKWYVNTTNIDGFRLDAVKHIKFSFFPDWLSYV
RSQTGKPLFTVGEYWSYDINKLHNYITKTDGTMSLFDAPLHNKFYTASKSGGAFDMRTLMTNTLMKDQP
TLAVTFVDNHDTEPGQALQSWVDPWFKPLAYAFILTRQEGYPCVFYGDYYGIPQYNIPSLKSKIDPLLI
ARRDYAYGTQHDYLDHSDIIGWTREGGTEKPGSGLAALITDGPGGSKWMYVGKQHAGKVFYDLTGNRSD
TVTINSDGWGEFKVNGGSVSVWVPRKTTVSTIARPITTRPWTGEFVRWTEPRLVAWP;
<AA; Sutcliffiella halmapala>
SEQ ID NO: 36
HHNGTNGTMMQYFEWHLPNDGNHWNRLRDDASNLRNRGITAIWIPPAWKGTSQNDVGYGAYDLYDLGEF
NQKGTVRTKYGTRSQLESAIHALKNNGVQVYGDVVMNHKGGADATENVLAVEVNPNNRNQEISGDYTIE
AWTKFDFPGRGNTYSDFKWRWYHFDGVDWDQSRQFQNRIYKFRGKAWDWEVDSENGNYDYLMYADVDMD
HPEVVNELRRWGEWYTNTLNLDGFRIDAVKHIKYSFTRDWLTHVRNATGKEMFAVAEFWKNDLGALENY
LNKTNWNHSVFDVPLHYNLYNASNSGGNYDMAKLLNGTVVQKHPMHAVTFVDNHDSQPGESLESFVQEW
FKPLAYALILTREQGYPSVFYGDYYGIPTHSVPAMKAKIDPILEARQNFAYGTQHDYFDHHNIIGWTRE
GNTTHPNSGLATIMSDGPGGEKWMYVGQNKAGQVWHDITGNKPGTVTINADGWANFSVNGGSVSIWVK
R;
<AA; Bacillus amyloliquefaciens>
SEQ ID NO: 37
VNGTLMQYFEWYTPNDGQHWKRLQNDAEHLSDIGITAVWIPPAYKGLSQSDNGYGPYDLYDLGEFQQKG
TVRTKYGTKSELQDAIGSLHSRNVQVYGDVVLNHKAGADATEDVTAVEVNPANRNQETSEEYQIKAWTD
FRFPGRGNTYSDFKWHWYHFDGADWDESRKISRIFKFRGEGKAWDWEVSSENGNYDYLMYADVDYDHPD
VVAETKKWGIWYANELSLDGFRIDAAKHIKFSFLRDWVQAVRQATGKEMFTVAEYWQNNAGKLENYLNK
TSFNQSVFDVPLHFNLQAASSQGGGYDMRRLLDGTVVSRHPEKAVTFVENHDTQPGQSLESTVQTWFKP
LAYAFILTRESGYPQVFYGDMYGTKGTSPKEIPSLKDNIEPILKARKEYAYGPQHDYIDHPDVIGWTRE
GDSSAAKSGLAALITDGPGGSKRMYAGLKNAGETWYDITGNRSDTVKIGSDGWGEFHVNDGSVSIYVQ
K;
<AA; Aspergillus oryzae>
SEQ ID NO: 38
MVAWWSLFLYGLQVAAPALAATPADWRSQSIYFLLTDRFARTDGSTTATCNTADQKYCGGTWQGIIDKL
DYIQGMGFTAIWITPVTAQLPQTTAYGDAYHGYWQQDIYSLNENYGTADDLKALSSALHERGMYLMVDV
VANHMGYDGAGSSVDYSVFKPFSSQDYFHPFCFIQNYEDQTQVEDCWLGDNTVSLPDLDTTKDVVKNEW
YDWVGSLVSNYSIDGLRIDTVKHVQKDFWPGYNKAAGVYCIGEVLDGDPAYTCPYQNVMDGVLNYPIYY
PLLNAFKSTSGSMDDLYNMINTVKSDCPDSTLLGTFVENHDNPRFASYTNDIALAKNVAAFIILNDGIP
IIYAGQEQHYAGGNDPANREATWLSGYPTDSELYKLIASANAIRNYAISKDTGFVTYKNWPIYKDDTTI
AMRKGTDGSQIVTILSNKGASGDSYTLSLSGAGYTAGQQLTEVIGCTTVTVGSDGNVPVPMAGGLPRVL
YPTEKLAGSKICSSS;
<AA; Geobacillus stearothermophilus>
SEQ ID NO: 39
MKQQKRLYARLLTLLFALIFLLPHSAAAAAPFNGTMMQYFEWYLPDDGTLWTKVANEANNLSSLGITAL
WLPPAYKGTSRSDVGYGAYDLYDLGEFNQKGTVRTKYGTKAQYLQAIQAAHAAGMQVYADVVFDHKGGA
DGTEWVDAVEVNPSDRNQVISGTYQIQAWTKFDFPGRGNTYSSFKWRWYHFDGVDWDESRKLSRIYLFS
GKQWDWPVDNEFGNYDYLMYADLDMDHPEVTTELKNWGKWYVNTTNIDGFRLDAVKHIKFYFFPDWLSY
VRSSTGKPLFTVGEYWSGDINKLHNYITITDGTVSLFDAPLHYKFYNASKSGGAFDMRTLMTNTLMKDQ
PTLAVTFVDNHDTEPGQALQSWVDPWFKPLAYAFILTRQEGYPCVFYGDYYGIPQYNIPSLKSKIDPLL
IARRDYAYGTQHDYLDHSDIIGWTREGGTEKPGSGLAALITDGPGGSKWMYVGKQHAGKVFYDLTGNRS
DTVTINSDGWGEFKVNGGSVSVWVPRKTTVSTIARP;
<AA; Alkalihalobacillus akibai>
SEQ ID NO: 40
HHNGTNGTMMQYFEWYLPNDGNHWNRLRSDASNLKDKGISAVWIPPAWKGASQNDVGYGAYDLYDLGEF
NQKGTIRTKYGTRNQLQAAVNALKSNGIQVYGDVVMNHKGGADATEMVKAVEVNPNNRNQEVSGEYTIE
AWTKFDFPGRGNTHSNFKWRWYHFDGVDWDQSRKLNNRIYKFRGKGWDWEVDTEFGNYDYLMYADIDMD
HPEVVNELRNWGVWYTNTLGLDGFRIDAVKHIKYSFTRDWINHVRSATGKNMFAVAEFWKNDLGAIENY
LNKTNWNHSVFDVPLHYNLYNASKSGGNYDMRQIFNGTVVQKHPMHAVTFVDNHDSQPEEALESFVEEW
FKPLAYALTLTREQGYPSVFYGDYYGIPTHGVPAMKSKIDPILEARQKYAYGRONDYLDHHNIIGWTRE
GNTAHPNSGLATIMSDGAGGNKWMFVGRNKAGQVWTDITGNKAGTVTINADGWGNFSVNGGSVSIWVN
K;
<AA; Aspergillus niger>
SEQ ID NO: 41
MSFRSLLALSGLVCTGLANVISKRATLDSWLSNEATVARTAILNNIGADGAWVSGADSGIVVASPSTDN
PDYFYTWTRDSGLVLKTLVDLFRNGDTSLLSTIENYISAQAIVQGISNPSGDLSSGAGLGEPKFNVDET
AYTGSWGRPQRDGPALRATAMIGFGQWLLDNGYTSTATDIVWPLVRNDLSYVAQYWNQTGYDLWEEVNG
SSFFTIAVQHRALVEGSAFATAVGSSCSWCDSQAPEILCYLQSFWTGSFILANFDSSRSGKDANTLLGS
IHTFDPEAACDDSTFQPCSPRALANHKEVVDSFRSIYTLNDGLSDSEAVAVGRYPEDTYYNGNPWFLCT
LAAAEQLYDALYQWDKQGSLEVTDVSLDFFKALYSDAATGTYSSSSSTYSSIVDAVKTFADGFVSIVET
HAASNGSMSEQYDKSDGEQLSARDLTWSYAALLTANNRRNSVVPASWGETSASSVPGTCAATSAIGTYS
SVTVTSWPSIVATGGTTTTATPTGSGSVTSTSKTTATASKTSTSTSSTSCTTPTAVAVTFDLTATTTYG
ENIYLVGSISQLGDWETSDGIALSADKYTSSDPLWYVTVTLPAGESFEYKFIRIESDDSVEWESDPNRE
YTVPQACGTSTATVTDTWR;
<AA; Aspergillus niger>
SEQ ID NO: 42
ATLDSWLSNEATVARTAILNNIGADGAWVSGADSGIVVASPSTDNPDYFYTWTRDSGLVLKTLVDLFRN
GDTSLLSTIENYISAQAIVQGISNPSGDLSSGAGLGEPKFNVDETAYTGSWGRPQRDGPALRATAMIGF
GQWLLDNGYTSTATDIVWPLVRNDLSYVAQYWNQTGYDLWEEVNGSSFFTIAVQHRALVEGSAFATAVG
SSCSWCDSQAPEILCYLQSFWTGSFILANFDSSRSGKDANTLLGSIHTFDPEAACDDSTFQPCSPRALA
NHKEVVDSFRSIYTLNDGLSDSEAVAVGRYPEDTYYNGNPWFLCTLAAAEQLYDALYQWDKQGSLEVTD
VSLDFFKALYSDAATGTYSSSSSTYSSIVDAVKTFADGFVSIVETHAASNGSMSEQYDKSDGEQLSARD
LTWSYAALLTANNRRNSVVPASWGETSASSVPGTCAATSAIGTYSSVTVTSWPSIVATGGTTTTATPTG
SGSVTSTSKTTATASKTSTSTSSTSCTTPTAVAVTFDLTATTTYGENIYLVGSISQLGDWETSDGIALS
ADKYTSSDPLWYVTVTLPAGESFEYKFIRIESDDSVEWESDPNREYTVPQACGTSTATVTDTWR;
<AA; Humicola insolens>
SEQ ID NO: 43
MARGTALLGLTALLLGLVNGQKPGETKEVHPQLTTFRCTKRGGCKPATNFIVLDSLSHPIHRAEGLGPG
GCGDWGNPPPKDVCPDVESCAKNCIMEGIPDYSQYGVTTNGTSLRLQHILPDGRVPSPRVYLLDKTKRR
YEMLHLTGFEFTFDVDATKLPCGMNSALYLSEMHPTGAKSKYNPGGAYYGTGYCDAQCFVTPFINGLGN
IEGKGSCCNEMDIWEANSRASHVAPHTCNKKGLYLCEGEECAFEGVCDKNGCGWNNYRVNVTDYYGRGE
EFKVNTLKPFTVVTQFLANRRGKLEKIHRFYVQDGKVIESFYTNKEGVPYTNMIDDEFCEATGSRKYME
LGATQGMGEALTRGMVLAMSIWWDQGGNMEWLDHGEAGPCAKGEGAPSNIVQVEPFPEVTYTNLRWGEI
GSTYQEVQKPKPKPGHGPRSD;
<AA; Humicola insolens>
SEQ ID NO: 44
MRSSPLLRSAVVAALPVLALAADGRSTRYWDCCKPSCGWAKKAPVNQPVFSCNANFQRITDFDAKSGCE
PGGVAYSCADQTPWAVNDDFALGFAATSIAGSNEAGWCCACYELTFTSGPVAGKKMVVQSTSTGGDLGS
NHFDLNIPGGGVGIFDGCTPQFGGLPGQRYGGISSRNECDRFPDALKPGCYWRFDWFKNADNPSFSFRQ
VQCPAELVARTGCRRNDDGNFPAVQIPSSSTSSPVNQPTSTSTTSTSTTSSPPVQPTTPSGCTAERWAQ
CGGNGWSGCTTCVAGSTCTKINDWYHQCL;
<AA; Thermothielavioides terrestris>
SEQ ID NO: 45
MRSTPVLRTTLAAALPLVASAASGSGQSTRYWDCCKPSCAWPGKAAVSQPVYACDANFQRLSDENVQSG
CNGGSAYSCADQTPWAVNDNLAYGFAATSIAGGSESSWCCACYALTFTSGPVAGKTMVVQSTSTGGDLG
SNHFDIAMPGGGVGIFNGCSSQFGGLPGAQYGGISSRDQCDSFPAPLKPGCQWRFDWFQNADNPTFTFQ
QVQCPAEIVARSGCKRNDDSSFPVFTPPSGGNGGTGTPTSTAPGSGQTSPGGGSGCTSQKWAQCGGIGF
SGCTTCVSGTTCQKLNDYYSQCL;
<AA; Bacillus amyloliquefaciens>
SEQ ID NO: 46
FNSYNSGLWQKADGYSNGDMFNCTWRANNVSMTSSGEMRLALTSPSYNKFDCGENRSVQTYGYGLYEVR
MKPAKNTGIVSSFFTYTGPTEGTPWDEIDIEFLGKDTTKVQFNYYTNGAGNHEKLADLGFDAANAYHTY
AFDWQPNSIKWYVDGQLKHTATTQIPAAPGKIMMNLWNGTGVDDWLGSYNGVNPLYAHYDWVRYT;
<AA; Aspergillus niger>
SEQ ID NO: 47
MLNPKVAYMVWMTCLGLTLPSQAQSNDYRPSYHFTPDQYWMNEPNGLIKIGSTWHLFFQHNPTANVWGN
ICWGHATSTDLMHWAHKPTAIADENGVEAFTGTAYYDPNNTSGLGDSANPPYLAWFTGYTTSSQTQDQR
LAFSVDNGATWTKFQGNPIISTSQEAPHDITGGLESRDPKVFFHRQSGNWIMVLAHGGQDKLSFWTSAD
TINWTWQSDLKSTSINGLSSDITGWEVPDMFELPVEGTEETTWVVMMTPAEGSPAGGNGVLAITGSFDG
KSFTADPVDASTMWLDNGRDFDGALSWVNVPASDGRRIIAAVMNSYGSNPPTTTWKGMLSFPRTLSLKK
VGTQQHFVQQPITELDTISTSLQILANQTITPGQTLLSSIRGTALDVRVAFYPDAGSVLSLAVRKGASE
QTVIKYTQSDATLSVDRTESGDISYDPAAGGVHTAKLEEDGTGLVSIRVLVDTCSVEVFGGQGEAVISD
LIFPSDSSDGLALEVTGGNAVLQSVDVRSVSLE;
<AA; Aspergillus niger>
SEQ ID NO: 48
MVGLLSITAALAATVLPNIVSAVGLDQAAVAKGLQYFGTATDNPELTDIPYVTQLNNTADFGQITPGNS
MKWDATEPSQGTFTFTKGDVIADLAEGNGQYLRCHTLVWYNQLPSWVTSGTWTNATLTAALKNHITNVV
SHYKGKCLHWDVVNEALNDDGTYRTNIFYTTIGEAYIPIAFAAAAAADPDAKLFYNDYNLEYGGAKAAS
ARAIVQLVKNAGAKIDGVGLQAHFSVGTVPSTSSLVSVLQSFTALGVEVAYTEADVRILLPTTATTLAQ
QSSDFQALVQSCVQTTGCVGFTIWDWTDKYSWVPSTFSGYGAALPWDENLVKKPAYNGLLAGMGVTVTT
TTTTTTATATGKTTTTTTGATSTGTTAAHWGQCGGLNWSGPTACATGYTCTYVNDYYSQCL;
<AA; Dictyoglomus thermophilum>
SEQ ID NO: 49
MQTSITLTSNASGTFDGYYYELWKDTGNTTMTVYTQGRFSCQWSNINNALFRTGKKYNQNWQSLGTIRI
TYSATYNPNGNSYLCIYGWSTNPLVEFYIVESWGNWRPPGATSLGQVTIDGGTYDIYRTTRVNQPSIVG
TATFDQYWSVRTSKRTSGTVTVTDHFRAWANRGLNLGTIDQITLCVEGYQSSGSANITQNTFSQGS;
<AA; Thermomyces lanuginosus>
SEQ ID NO: 50
MVGFTPVALAALAATGALAFPAGNATELEKRQTTPNSEGWHDGYYYSWWSDGGAQATYTNLEGGTYEIS
WGDGGNLVGGKGWNPGLNARAIHFEGVYQPNGNSYLAVYGWTRNPLVEYYIVENFGTYDPSSGATDLGT
VECDGSIYRLGKTTRVNAPSIDGTQTFDQYWSVRQDKRTSGTVQTGCHFDAWARAGLNVNGDHYYQIVA
TEGYFSSGYARITVADVG;
<AA; Bacillus agaradhaerens>
SEQ ID NO: 51
QIVTDNSIGNHDGYDYEFWKDSGGSGTMILNHGGTFSAQWNNVNNILFRKGKKFNETQTHQQVGNMSIN
YGANFQPNGNAYLCVYGWTVDPLVEYYIVDSWGNWRPPGATPKGTITVDGGTYDIYETLRVNQPSIKGI
ATFKQYWSVRRSKRTSGTISVSNHFRAWENLGMNMGKMYEVALTVEGYQSSGSANVYSNTLRINGNPL;
<AA; Aspergillus fumigatus>
SEQ ID NO: 52
MVHLSSLAAALAALPLVYGAGLNTAAKAKGLKYFGSATDNPELTDSAYVAQLSNTDDFGQITPGNSMKW
DATEPSQNSFSFANGDAVVNLANKNGQLMRCHTLVWHSQLPNWVSSGSWTNATLLAAMKNHITNVVTHY
KGKCYAWDVVNEALNEDGTFRNSVFYQIIGPAYIPIAFATAAAADPDVKLYYNDYNIEYSGAKATAAQN
IVKMIKAYGAKIDGVGLQAHFIVGSTPSQSDLTTVLKGYTALGVEVAYTELDIRMQLPSTAAKLAQQST
DFQGVAAACVSTTGCVGVTIWDWTDKYSWVPSVFQGYGAPLPWDENYVKKPAYDGLMAGLGASGSGTTT
TTTTTSTTTGGTDPTGVAQKWGQCGGIGWTGPTTCVSGTTCQKLNDWYSQCL;
<AA; Evansstolkia leycettana>
SEQ ID NO: 53
MVHLSSLALALAAGSQLAQAAGLNTAAKAIGKLYFGTATDNPELSDSTYMQETDNTDDFGQLTPANSMK
WDATEPSQNTFTFTNGDQIANLAKSNGQMLRCHNLVWYNQLPSWVTSGSWTNATLLAAMKNHITNVVTH
YKGQCYAWDVVNEALNDDGTYRSNVFYQYIGEAYIPIAFATAAAADPNAKLYYNDYNIEYPGAKATAAQ
NIVKMVKAYGAKIDGVGLQSHFIVGSTPSQSSQQSNMAAFTALGVEVAITELDIRMTLPSTSALLAQQS
TDYQSTVSACVNTPKCIGITLWDWTDKYSWVPNTFSGQGDACPWDSNYQKKPAYYGILTALGGSASTST
TTTLVTSTRTSTTTSTSATSTSTGVAQHWGQCGGIGWTGPTTCASPYTCQELNPYYYQCL;
<AA; unidentified>
SEQ ID NO: 54
IRQRAGNHTVCNSQLCTWWHDNGEINTASMVQLGNVRQSHKYLVQVSIAGVNDFYDSFAYESIPRNGRG
RIYSPWDPPNSDTLGSDVDDGITIETSAGINMAWSQFEYSTGVDVKILTRDGSRLPDPSGVKIRPTAIS
YDIRSSSDGGIVIRVPHDPNGRRFSVEFDNDLYTYRSDGSRYVSSGGSIVGVEPRNALVIFASPFLPDN
MVPRIDGPDTKVMTPGPINQGDWGSSGILYFPPGVYWMNSNQQGQTPKIGENHIRLHPNTYWAYLAPGA
YVKGAIEYSTKSDFYATGHGVLSGEHYVYQANPATYYQALKSDATSLRMWWHNNLGGGQTWYCQGPTIN
APPFNTMDFHGSSDITTRISDYKQVGAFFFQTDGPQMYPNSQVHDVFYHVNDDAIKTYYSGVTVTRATI
WKAHNDPIIQMGWDTRDVTGVTLQDLYIIHTRYIKSETYVPSAIIGASPFYMPGRSVDPAKSISMTISN
LVCEGLCPALMRITPLQNYRDFRIQNVAFPDGLQANSIGTGKSIVPASSGLKFGVAISNWTVGGEQVTM
SNFQSDSLGQLDIDVSYWGQWVIR;
<AA; Sodiomyces alcalophilus>
SEQ ID NO: 56
RIPGFDISGWQPTTDFARAYANGDRFVYIKATEGTTFKSSAFSRQYTGATQNGFIRGAYHFAQPAASSG
AAQARYFASNGGGWSKDGITLPGALDIEYNPNGATCYGLSQSAMVNWIEDFVTTYHGITSRWPVIYTTT
DWWTQCTGNSNRFANRCPLWIARYASSVGTLPNGWGFYTFWQYNDKYPQGGDSNWFNGDASRLRALANG
D;
<AA; Aspergillus fumigatus>
SEQ ID NO: 57
MRFGWLEVAALTAASVANAQELAFSPPFYPSPWADGQGEWADAHRRAVEIVSQMTLAEKVNLTTGTGWE
MDRCVGQTGSVPRLGINWGLCGQDSPLGIRDSDLNSAFPAGTNVAATWDKTLAYLRGKAMGEEFNDKGV
DILLGPAAGPLGKYPDGGRIWEGFSPDPVLTGVLFAETIKGIQDAGVIATAKHYILNEQEHFRQVGEAQ
GYGYNITETISSNVDDKTMHELYLWPFADAVRAGVGAVMCSYNQINNSYGCQNSQTLNKLLKAELGFQG
FVMSDWGAHHSGVGAALAGLDMSMPGDISFDDGLSFWGTNLTVSVLNGTVPAWRVDDMAVRIMTAYYKV
GRDRLRIPPNFSSWTRDEYGWEHSAVSEGAWTKVNDFVNVQRSHSQIIREIGAASTVLLKNTGALPLTG
KEVKVGVLGEDAGSNPWGANGCPDRGCDNGTLAMAWGSGTAEFPYLVTPEQAIQREVISNGGNVFAVTD
NGALSQMADVASQSSVSLVFVNADSGEGYISVDGNEGDRKNLTLWKNGEAVIDTVVSHCNNTIVVIHSV
GPVLIDRWYDNPNVTAIIWAGLPGQESGNSLVDVLYGRVNPSAKTPFTWGKTRESYGAPLLTEPNNGNG
APQDDFNEGVFIDYRHFDKRNETPIYEFGHGLSYTTFGYSHLRVQALNSSSSAYVPTSGETKPAPTYGE
IGSAADYLYPEGLKRITKFIYPWLNSTDLEDSSDDPNYGWEDSEYIPEGARDGSPQPLLKAGGAPGGNP
TLYQDLVRVSATITNTGNVAGYEVPQLYVSLGGPNEPRVVLRKFDRIFLAPGEQKVWTTTLNRRDLANW
DVEAQDWVITKYPKKVHVGSSSRKLPLRAPLPRVY;
<AA; Neobacillus novalis>
SEQ ID NO: 58
SSDSTSASKTDFFSSFEKSDLQLTWTNTVETDANGKKMSSGIDGNVKRDLILGDITDKVVQVTASANNP
PNEIDSKLIDGDPTTKWLAFEPTANIVLKLAEPVAVVKYALTSANDAKGRDPKNWTLYGSLDGTNWTAV
DTREGEDFKDRFQRNMYDLKNTTKYLYYKLDITKNAGDSITQLAEISLSDGIEVPAPPPGDMKSLIGKG
PTSSYTAKTNVGWTGLGALNYSGTHLSDGRAYSYNKLYDVDILVTPATELSYFIAPEFTDKNHNDYSST
YVSVDLAFSDGTYLHDLKAVDQYGVGLNPKDQGDSKYLYVNQWNTIKSTIGSVAAGKTIKRILVAYDNP
KGPGAFRGSIDDIKIDGKPVQKAFGSPIDYVNILRGTQSNGSFSRGNNFPAVAIPHGFNFWTPTTNAGS
SWIYQYHESNSVNNLPQIQAFSVSHEPSPWMGDRQTFQVMPSASTAATPNANRDSRALEFNHANEIAQP
HYYSVKFENGIRTEMTPTDHAAMFKFTFTGATSNLIFDNVNNNGGLTIDAKSGEITGYSDVKSGLSTGA
TRLFVYAAFDKPVIKSGKLTGESRNNVTGYVRFDTSKDEDKVVTMKIATSLISVEQAKKNLEQEIGLND
TFEGLKEKAKTEWNKKLGIIEVEGASEDQLVTLYSNLYRLFLYPNSAFENVGTTTDPVYKYASPYSAAT
GQDTATTTGAKIVDGKTYVNNGFWDTYRTAWPAYSLLTPTFAGELIDGFVQQYRDGGWIARWSSPGFAN
LMPGTSSDVAFADAYLKGVTNFDVQSFYQSAIRNAEAVSPNAGTGRKGLTTSIFDGYTNTSTGEGLAWA
MDGYINDFGIANLAKALKEKGDKSDPYYANYAADYQYFLNRAQNYVHMFNPSIEFFNGRTANGAWRSTP
DNFNPAVWGSDYTETNGWNMAFHVPQDGQGLANLYGGKEGLATKLDQFFSTSETGLFPGSYGGTIHEMR
EARDVRMGMYGHSNQPSHHIAYMYDYAGQPWKTQEKVREALNRLYIGSAIGQGYSGDEDNGEMSAWYIL
SAMGFYPLKMGTPEYAIGAPLFKKATIHLENGKSIVINAPNNSKENKYVQSMKVNGKAYAKTSILHADI
ANGAVIDFEMGSKPSKWGSGDQDILQSITPGSTDGTSLSPLPLRDVTDRLIAAEKGAVTVSDEGNGQLL
FDNTSNTQLSMKSKTPSIVYQFKEGKQNVKMYTLTSSKASQNEDPKSWVLKGSNDGKSWSVLDQRKNET
FQWRQYTRAFTIQHPGKYSQYKLEITENAGAEVTTLAELELLGYDDVTNSYQAVYELMEQFKQSKDLTG
PMAVQLNNSLTTSLDHFKKDHKDQAIKHLEDFLKHLNNKGLQDRISSKAKGVLSADANQLIVLLARD;
<AA; Trichoderma atroviride>
SEQ ID NO: 59
MKSLTAVVALLGAVAASPAVVRHHGRSALQPRANSSFWYAAMDHTGQFKGSAPYVDASYNVFVAVSPGD
AGSLQNAIDSAGSGNRQNEWLASQPRVVYIPSGTYELSSTLNMRTDTILFGDATNPPVIKAAAGFSDNY
LVNGQDPSTGDAGELSFAVGLKNVVLDTTAVDGSSSISALYWGVAQACQLQNVKITLAPSVNGKGHTGV
QLGRGSTLALADIRIENGQTGIWHNGHQQALYKSIYFYKNTVGMLISGGNTISLLNPTFDSVGTGVSNT
GGSPFIGIVDATSINSGVTFTTSVYPSIVIDNLTKDTDSDVAVIRGTTTVGASKNVVNYSYGNTVGANP
VYGGVNGNTTRPSGVAPGGRIPAVAVPNYANNPVTDFVNVKDPSQNGGQTVKGDGSTDDSAALNKVLQF
AATNNKIAYFPFGDYRVESTLLVPVGSQLVGEAWATISGGGDFFKDASSPKPVVQVGNEGDVGVAQIQD
FRFTVSDVLPGAIIVQFNAAGSKPGDVALFNSLVTVGGTHGADALTNACTDASNECQAAFLGLHFTEGS
SAYVENTWNWVADHITEGFSGGSNIAAKGGALVESTKGTWLNGLGSEHWWLYQLNLKAASNVAVTLLQS
ETNYDQGDNTKQVPPAPWTADVQGWGDPDFSWCDSTARCHMGLANYVNGGSDIYYYGSASWAFFSGPGY
QGCAGSYQCQDYMHFISATPTNLQMYGMCSKDTSVALRLGDGTKINAQPDFTGGWSPGADIGRYTS;
<AA; Trichoderma harzianum>
SEQ ID NO: 60
MKLLPSLIGLASLASLAVAAPSLIPRVSPFSIAKPGGVDDIVITANNTLNGTYHSSTTVSKNVIQTRAQ
AGHLPIQLVNNFSGSQVNAYISGLDTDNRVVFVRGDGSLVYPSSGGSSVPVAISTPINIALPAQGQSIT
VNVPIVISSARIYFSVGDLQFFMVKIPNGDGLVQPSQFNLQDPSAGLLWGFVELTYTTDLAVYANISYV
DFVGMILSMSLSATDGSATQTTKGLGSSALTQICQGLVQQASVDGFPWSSMCIANSAGTLVRALSPGDY
SVINAAAFQNYWSAYVDQVWSQYTNTPLTINTQTSAGSVNCQVSGDTLNCNGDNRGYAKPAAGDIWGCN
SGPFAIQSGDNAVHSAVVPRLCAAFVRSTLLLAGGNVQPGLGQSSYYTVSPTNHYSRLVHQFEIDGRGY
AFPYDDVNPDGNENASGTLASGAPNVLTVYVGAPPS;
<AA; Trichoderma harzianum>
SEQ ID NO: 45906
APSLIPRVSPFSIAKPGGVDDIVITANNTLNGTYHSSTTVSKNVIQTRAQAGHLPIQLVNNFSGSQVNA
YISGLDTDNRVVFVRGDGSLVYPSSGGSSVPVAISTPINIALPAQGQSITVNVPIVISSARIYFSVGDL
QFFMVKIPNGDGLVQPSQFNLQDPSAGLLWGFVELTYTTDLAVYANISYVDFVGMILSMSLSATDGSAT
QTTKGLGSSALTQICQGLVQQASVDGFPWSSMCIANSAGTLVRALSPGDYSVINAAAFQNYWSAYVDQV
WSQYTNTPLTINTQTSAGSVNCQVSGDTLNCNGDNRGYAKPAAGDIWGCNSGPFAIQSGDNAVHSAVVP
RLCAAFVRSTLLLAGGNVQPGLGQSSYYTVSPTNHYSRLVHQFEIDGRGYAFPYDDVNPDGNENASGTL
ASGAPNVLTVYVGAPPS;
<AA; Bacillus deramificans>
SEQ ID NO: 61
MAKKLIYVCLSVCLVLTWAFNVKGQSAHADGSTTTIIVHYFRPAGDYQPWSLWMWPEGGSGAEYDFNGT
DSYGEVANVSIPGNPSQVGIIVRTQDWTKDVSADRYIDLSKGHEVWLVQGNSQIFYNEKDAEDAAKPAV
SNAYLDASNQVLVKLSQPFTLGEGASGFTVHDDTVNKDIPVTSVTDASLGQNVTAVLAGTFQHIFGGSD
WAPDNHSTLLKKVNNNLYQFSGDLPEGNYQYKVALNDSWNNPSYPSNNIDLTVPTGGAHVTFSYVPSTH
AVYDSINNPGADLPVNGSGVKTDLVTVTLGEDPDVSHTLSIQTDGYQAKQVISRNVLDSSQYYYSGDDL
GNTYTHKATTFKVWAPTSTQVNVLLYNSATGSVTKTVPMTASGHGVWEATVNONLENWYYMYEVTGQGS
TRTAVDPYATAIAPNGTRGMIVDLAKTDPAGWNSDKHITPKNIEDEVIYEMDVRDFSIDPNSGMKNKGK
YLALTEKGTKGPDNVKTGIDSLKQLGITHVQLMPVFAFNSVDETDPTQDNWGYDPRNYDVPEGQYATNA
NGTARIKEFKEMVLSLHREHIGVNMDVVYNHTFATQISDFDKIVPEYYYRTDDAGNYTNGSGTGNEIAA
ERPMVQKFIIDSLKYWVNEYHIDGFRFDLMALLGKDTMSKAASELHAINPGIALYGEPWTGGTSALPED
QLLTKGAQKGMGVAVFNDNLRNALDGNVFDSSAQGFATGATGLTDAIKNGVEGSINDFTSSPGETINYV
TSHDNYTLWDKIALSNPNDSEADRIKMDELAQAVVMTSQGVPFMQGGEEMLRTKGGNDNSYNAGDTVNE
FDWSRKAQYPDVFNYYSGLIHLRLDHPAFRMTTANEINSHLQFLNSPENTVAYELTDHVNKDKWGNIIV
VYNPNKTAATINLPSGKWAINATSGKVGESTLGQAEGSVQVPGISMMILHQEVSPDHGKK;
<AA; Talaromyces pinophilus>
SEQ ID NO: 62
MHFLAALLAVLPLVSGSPVPEKRSGCALPSTYKWTSTGPLASPKSGLVALRDYSHVIYNGQHLVYGSTA
NTAGSYGSMNFGLFSDWSEMSSASQNTMSTGAVAPTIFYFAPKSVWILAYQWGPYAFSYRTSTDPSNAN
GWSSPQPLFTGTISGSSTGVIDQTVIGDSENMYLFFAGDNGHIYRASMPIGDFPGSFGSASTIVLSDST
NNLFEAVEVYTVEGQNQYLMIVEAIGANGRYFRSFTASSLGGTWTAQASTESNPFAGKANSGATWTNDI
SSGDLVRTNPDQTQTIDACNLQFLYQGRSTSSGGDYNLLPYQPGLLTLA;
<AA; Clonostachys rosea>
SEQ ID NO: 63
MLFKTAFLGLIATVNFVHGAAIGHAAEQTSLEKRAEAADRLVFCHFMIGIVGNRNSASQYDDDMRRAKA
AGIDAFALNIGVDGYTDAQLDLAYKSAADNDMKVFISFDFNWFKTGEAATVGQKIAKYANQAAQLKVDN
RVFASSFAGDGLDVAAMKNAAGVDVFFVPNFHPEQTANPDSIDGGFNWMAWPNDGNNKAPKPGSSISVE
DGDNKYLSWLGTKPYMAPISPWFFTHFGPEVSFSKNWVFPGGALIFDRWNQILDKGFPLVEMITWNDYG
ESHHFGPLSSPHYDDGNSKWTNDLPHNGWLELSKPFIAAYKAKEKAVDKYITGDQIVYWYRRTLKGLDC
DATDTTAGRPANNDSGNYFMGRPDGWELMDDVVYVATLLKEAGTLTVNSGGQSVEKEVPAGANLIQVPA
AVGSQTFSLSRNGKVVLEDKSLMDISNICPCGLYNFNPYVGTVPPGESDPLQPDGLASLTIGLHVSTCQ
ATPSLGTNPPISTTGGSPPPSSTGSSPPPSTTAGPTSTGGSVPSQTSSVPAPTSTGSSQPCNGGTNADG
ESGNYSGLCSFACSYGYCPPGPCKCTSNGTPGNPPSGDGRNGCPADGLGDGYKGLCSFACSHGYCPDTA
CKYC;
<AA; Trichoderma harazianum>
SEQ ID NO: 64
MLGVVRRLGLGALAAAALSSLGSAAPANVAIRSLEERASSADRLVFCHFMIGIVGDRGSSADYDDDMQR
AKAAGIDAFALNIGVDGYTDQQLGYAYDSADRNGMKVFISFDFNWWSPGNAVGVGQKIAQYASRPAQLY
VDNRPFASSFAGDGLDVNALRSAAGSNVYFVPNFHPGQSSPSNIDGALNWMAWDNDGNNKAPKPGQTVT
VADGDNAYKNWLGGKPYLAPVSPWFFTHFGPEVSYSKNWVFPGGPLIYNRWQQVLQQGFPMVEIVTWND
YGESHYVGPLKSKHFDDGNSKWVNDMPHDGFLDLSKPFIAAYKNRDTDISKYVQNEQLVYWYRRNLKAL
DCDATDTTSNRPANNGSGNYFMGRPDGWQTMDDTVYVAALLKTAGSVTVTSGGTTQTFQANAGANLFQI
PASIGQQKFALTRNGQTVFSGTSLMDITNVCSCGIYNFNPYVGTIPAGFDDPLQADGLFSLTIGLHVTT
CQAKPSLGTNPPVTSGPVSSLPASSTTRASSPPVSSTRVSSPPVSSPPVSRTSSPPPPPASSTPPSGQV
CVAGTVADGESGNYIGLCQFSCNYGYCPPGPCKCTAFGAPISPPASNGRNGCPLPGEGDGYLGLCSFSC
NHNYCPPTACQYC;
<AA; Salipaludibacillus agaradhaerens>
SEQ ID NO: 65
MIKINKTIKILMLTLLMMSFAGAAYAHNPVTDEEVYHSFNSHDWQNWNMSDGWKNDDYFFGCHWSQNRV
NFYGGQMELSLRTNYSYAPPYNYECAEYTTNNFYGYGLYEVSMKPAKVSGVISSFFTYTGPSYNGAPWD
EIDIEFLGNDTTKVQFNYYTDGVGGNEILYDLGFDAADSYNTYAFDWQENYINWYVNGQLVATATENIP
SNPSKIMMNIWNTYGIDEWAGRYYGEDANASYNWVRYTPNR;
<AA; Trichoderma atroviride>
SEQ ID NO: 66
MLSAYAFSLLVASASAWTPQSNAKVFSRDTEGVMRWLPGNDKFRGVNLGSQFIIEPWMASDEFSGMGCG
GLNDEWSCVQSLGQDAADAAFQKHWDSWITQDDITQIKNLGLNTVRIPVGFWIREDLVQQGEFFPRGGI
QYLDRLVGWCNDAGIYVIMDLHGGPGAQFPNQQYTGHGVSQPGFYTEANYERAANFLEWMTERIHTNAT
YASVGMLEVINEPVHSGDFPSQAADMVNTYYPLAWNRIRDTESKLGVSDDKRLHIQFMASAWGSGDPTS
ALPSTDFAAFDDHRYLKWDTSVTATKDGYLNAACSDKRDDNVIVGEWSISVADNVQDNDELGIKNRSDQ
ADWYQKFWAAQVLAFEKSAGWVFWTWKCNWITGYDDWRWCYQSAVAAGAIPKDAGSAASINPC;
<AA; unidentified>
SEQ ID NO: 67
ATSFYYPNMDHVNAPRGFAPDLDGDFNYPIYQTVNAGDGNALQNAITTDGKGGSRHPQWFASQPRVVYI
PPGTYTISKTLRFNTDTILMGDPTNPPIIKAAAGFSGDQTLISAQDPSTNEKGELSFAVAIKNVVLDTT
AIPGGNSFTALWWGVAQAAHLQNVRITMSSSSGGNGHTGIRMGRGSTLGLADVRVERGQNGIWIDGHQQ
ASFHNIYFFQNTIGMLISGGNTFSIFSSTFDTCGTGISNTGGSPWIALIDAKSINSGVTFTTNQFPSFM
IENLTKDNGTPVVVVRGSTLVGASSHVNTYSYGNTVGRNPTYGDVTSSNTRPGALAPGGRYPYVAPPTY
GDLPISSFLNVKDPAQNGGRTVKGDNTIDEAATLNAILELAASQNKVAYFPFGKYRVDSTLFIPKGSRI
VGEAWATITGNGNFFKNENSPQPVVSVGRAGDVGIAQIQDMRFTVNDVLSGAIVVQFNMAGNNPGDVAL
WNSLVTVGGTRGASALANACTNNSNECKGAFIGIHVAKGSSPYIQNVWNWVADHIAENFSGGTSIAGKG
GILVQSTKATWLYAIGSEHWWLYQLNLHNAANVVVSLLQAETNYHQGANTQQIPPAPWVANIGTWGDPD
FAWCNGGDKRCRMGPANFINGGSNIYTYASAAWAFFSGPGQGCAQFECQQTMHWIASTPSNLQAFGLCS
KDSVNTLRLGDGTFINTQNGYTGGWTPGGGDVGRYTT;
<AA; Alkalihalobacillus bogoriensis>
SEQ ID NO: 68
MKNLLIAFLLILFVTGCAEPEQQQTEEATDNIEETQEELEVNEEPIVIADNLDIPWSIEKAGDTFYITE
RGGHIVKVENGAMERQTVHLEQELSSVPEAGLLGFVLNPNFPDTNLAYAYYTYEKNADPYNRIVTLRYE
NDQWQEENILLDNIPSGTYHHGGRLKIGPDGFLYATTGDASIPEIAQDIDSLGGKIIRVELDGAIPEDN
PFANSSIYSFGHRNPQGLTWAIDGSFYSSEHGSSANDEINEIEPGLNYGWPIIQGDEEREGMVSPLFTS
GNESTWAPSGMDYYNNRLYVAALRGAAIIEFNLETGEHQEIITDLGRIRDIKIEDDTLYFISNNTDGRG
NPEEDDDKLYRLSLQE;
<AA; Alkalihalobacillus bogoriensis>
SEQ ID NO: 72144
MKQQKRLYARLLTLLFALIFLLPHSAAAANSGFYVSGTTLYDANGNPFVMRGINHGHTWYKDQATTAIE
GIANTGANTVRIVLSDGGQWTKDDIHTVRNLISLAEDNHLVAVLEVHDATGQDDIASLNRAVDYWIEMR
SALIGKEKTVIINIANEWFGSWEGDPWARGYKQAIPRLRNAGLNHTLMVDAAGWGQFPQSIHDYGREVF
NADPQRNTMFSIHMYEYAGGTARQVRTNIDGVLNQDLALVIGEFGHRHTNGDVDEATIMSYSEQRGVGW
LAWSWKGNGPEFEYLDLSNDWAGNNLTAWGNTIVHGPYGIRETSRPVTVFTGGGSDGGTSP;
<AA; Talaromyces pinophilus>
SEQ ID NO: 69
MTFKTSYLASALLLLTARASAQTANAEAAFATLQEWYDPVTGLWNTAGWWNGANAMTVIAELAAVDASI
VQDAISIFETTFSVAPSVNPSNGVEKSVGTNGLIQTTYPAGWPNNTISKRAIQDSTDPTVWLDGANDDA
EWWGLAWVAAYDVTGNETYLNLAEGIFNEIAAGWGTNCGNGGIYWETTNHYVNAIASELFISLAAHLAN
RVPANAATYTAWAEEAWNWFASTGMINANGTINDGLTTDCVNNGQPVWSYNQGVILGGLTELNKISPNE
SYIESANSIAQAAIAALADSNYVIHDYACEPSDCEPNGTQFKGIFMRNLLLLQRASPNDLYSKVIEACA
ASIWANDRDTTTGELGVNWAGPFVGGDATTQSSAMGALVAAISVE;
<AA; Fusarium solani>
SEQ ID NO: 70
MVAFSRIAVTTLALGGSTAQAKPATAARATGLADFKPGVQWEICIHHPIKHDSAADLIPTKAKVWDIDM
GHAQEFPNMIPMLKSAGKFVICYFNAGALQDWDDDKSKFPKEVIGHSLSYPYDSEEWYLDIRDSRVLEL
QTARLDIAAKIGCDAVDPDNVDAWQQDDEDPTGFKLKSSDYTNYLKNLAKYAHSIKTKDGQPLLVGQKN
APEIAEDLVSTLDFAVLESCRGNSDPNEESWPFCEDFQTYIDAGKPVLQIEYPPSVEKTGKVSASDNKY
YCTAEDEDKGFSKIIKWASAQLDGWGQYCGEEPFRTPAAKY;
<AA; Ostropa barbara>
SEQ ID NO: 71
MVLPSSILLPLYIYPSASTPSPWQPLYDSLGTHSNIHFDIIINPGSGPLLVPPTDIQLSDYMPAIAKLR
TYKNVDLLGYVHTSWGLASQSDLLANISRYATWETVNGQDVHLDGLFFDEVVTAYDETDYDLLSTATTL
ARTTPNFKKIVFNPGQPADERYYALANYLTVFEGTAAAFDSSTVAALDVATRANSSVVIHDFTGTEAEQ
AILVNELVGLGLGSVDVTTLADYNDWSALWADFTGEVGDAV;
<AA; Stenocarpella maydis>
SEQ ID NO: 72
MHSFYLFPAVLAATAASATKILLPLYVYPTTGAGWTSIYNAIETHPSIDFQIVLNPSSGPGGSTPGYNT
HWISAVSKLHSYDNVDTLGYVSAGYGSRDLDEITTDIKDWNQWNHYTGADITIDGIFFDETPNWNGTRG
ANDVSFMQQVTKLADRGGYTKVFNLGQACSHDEYFQIADTVVMFEGEAESYNSTVLDDVVESGVADRSA
ILIHHFEGSGLTSGFAVRWVIDLVNRGLGSFAIINTDWTQANSDVAPMGISILADMLESVLDHQY;
<AA; Urnula criterium>
SEQ ID NO: 73
MTSKILSLALATLNVAYAATTICSTKCPYTNLGSSDAVTKSDWWKPALGATWQVVLTPADDDAGPQLTD
DYEVYDLDVFDVDASVFKSLQAKDKKVICYFSAGSYEDWREDCGCFKSSDLGSDLDGWEGEAWLNTDSQ
NVRNIMAARLDVAVEKGCNGVDPDNVDGYDNDNGLGLSESTAKSYLQFLSDEAHSRGLAIGLKNAGSIA
ADVVDSMEWEVNEECIAQENCEDYKTFVDAGKPVFNIEYPPEDHIDDAWTEAEIEERCAQKADASNGAT
KFSTVLKNHENVDEWIRTCSE;
<AA; Bjerkandera fumosa>
SEQ ID NO: 74
MFTFAKIFTTACSLAGLFGSTFGTGIIVPLYVWPGDPNVCSGWTPLFNVISTTPSLTFYVVINPNSGPG
DPSTQPVDSYSTCIQQLKGAKSGSNLRVLGYVATGGGSSTTVVRDITTYSGWNAAYRPDGIFFDQGSTA
SSQVSTYAGWANQARQILGSSTYTIVNPGQPPQDNAYFNSFDQIVTREDFLAQFSPSQLSITPASPAAK
QAVILTDAFTFPSSVVSQLVSGNHIGSLWVTDDTQANQQNPYDSLPSYLTTFVNAVVADN;
<AA; Pseudoplectania vogesiaca>
SEQ ID NO: 75
MSLSLSPVATTGVLLPLYIYPAPSTAWDPLYSAITNNPSVNFYVIINPDSGPGGDEFPASDYISAIQKL
KTYDTVTPLGYVHTTFGDQTTSAVEANITKYKNWDTYSTTSPISVDGIFFDEAPSSAADVSYMTTLSSS
AKTTFGTGRDFVVFNPGSIVDPGYFNAADAIVIFENTEATYSESVFSSQSGGTSRQKAAIIHDFTGSES
EQTSVVEGMVNQQAGLVYITDQSSYNSFSGLWSSFVSALQAAIA;
<AA; Lysobacter gummosus>
SEQ ID NO: 76
PRADARQALNPRAIAFYYNWYGSPKLDGAPLHWAHDVLRENDRDRTTKRLPGNGDISANFYPQLGEYSS
ADPAVIERHMAMIASARIGVIAVTWLGVDDPSFKSLRPLFEAAQRHGVRICFQIEPVARKTAADARASI
RYLVETFGAHPAFYRDPVSKRPLFFVYDSYVIDAKDWAQVLGRDGADSIRGTAFDADVLGLWVGADEHA
FFEHSGFDGFYTYFASRGFSYGATPEHWPELQRWAKQHGKLFIPSVGPGYIDTRVRPWNAKNTKDRDGG
RYYDAMFQAAIDSGAPFIGITSFNEWHEGTQIEPAVAFKHEAFEYLDYAPRPPDYYLERTAHWLQRYKP
AAAQ;
<AA; Chryseobacterium viscerum>
SEQ ID NO: 77
PRADARQALNPRAIAFYYNWYGSPKLDGAPLHWAHDVLRENDRDRTTKRLPGNGDISANFYPQLGEYSS
ADPAVIERHMAMIASARIGVIAVTWLGVDDPSFKSLRPLFEAAQRHGVRICFQIEPVARKTAADARASI
RYLVETFGAHPAFYRDPVSKRPLFFVYDSYVIDAKDWAQVLGRDGADSIRGTAFDADVLGLWVGADEHA
FFEHSGFDGFYTYFASRGFSYGATPEHWPELQRWAKQHGKLFIPSVGPGYIDTRVRPWNAKNTKDRDGG
RYYDAMFQAAIDSGAPFIGITSFNEWHEGTQIEPAVAFKHEAFEYLDYAPRPPDYYLERTAHWLQRYKP
AKK;
<AA; Effusibacillus pohliae>
SEQ ID NO: 78
SSSASVKGDVIYQIIIDRFYDGDTTNNNPAKSYGLYDPTKSKWKMYWGGDLEGVRQKLPYLKQLGVTTI
WLSPVLDNLDTLAGTDNTGYHGYWTRDFKQIEEHFGNWTTFDTLVNDAHQNGIKVIVDFVPNHSTPFKA
NDSTFAEGGALYNNGTYMGNYFDDATKGYFHHNGDISNWDDRYEAQWKNFTDPAGFSLADLSQENGTIA
QYLTDAAVQLVAHGADGLRIDAVKHFNSGFSKSLADKLYQKKDIFLVGEWYGDDPGTANHLEKVRYANN
SGVNVLDFDLNTVIRNVFGTFTQTMYDLNNMVNQTGNEYKYKENLITFIDNHDMSRFLSVNSNKANLHQ
ALAFILTSRGTPSIYYGTEQYMAGGNDPYNRGMMPAFDTTTTAFKEVSTLAGLRRNNAAIQYGTTTQRW
INNDVYIYERKFFNDVVLVAINRNTQSSYSISGLQTALPNGSYADYLSGLLGGNGISVSNGSVASFTLA
PGAVSVWQYSTSASAPQIGSVAPNMGIPGNVVTIDGKGFGTTQGTVTFGGVTATVKSWTSNRIEVYVPN
MAAGLTDVKVTAGGVSSNLYSYNILSGTQTSVVFTVKSAPPTNLGDKIYLTGNIPELGNWSTDTSGAVN
NAQGPLLAPNYPDWFYVFSVPAGKTIQFKFFIKRADGTIQWENGSNHVATTPTGATGNITVTWQN;
<AA; Evansstolkia leycettana>
SEQ ID NO: 79
TSGSQDDIAVLRRACPDYTSYSTVPHAPYSGGPLNLPFQRPAEACRTFTSPAVEQVIQDVTSRMVDKDM
AQLFRNAFPNTLDTTIRWHTNGSTTTSTRRKSKKDAAQWQGAQTFVVTGDINAEWLRDSTNQLAGYQAL
AKKDKSLYTLILGAINTQVEYVIQSPYCNAFQPPPPSGIRPTSNGQGDTVHPAYEPSFVFECKYELDSL
AHFLTLGTQFYENTGSTEFLTSRWYTALNTLLDVLDAQSQPTFNANNQYVTNQYTFQRQTTAGTETLSL
RGIGNPLNSGTGLIRSAFRPSDDATILGFFIPPNAMMSVQLKKIAEVIKAAGGDSSLVKKLQQRGHDLE
QSVWEHGVVDHPKYGKVFAFEVDGYGSRILMDDANIPSLLSLPLLGFVDKNDETYQNTRKMILDKSGNP
YYLTGSDFHGIGGPHVGLENAWPMSLLVQAQTTDSDSEIAECINLVRNSSRLGLVHESINVNNIRDYTR
PWFAWANSLFAQTILKIAAEKPHLVFGSDASPYVVE;
<AA; Nocardiopsis sp.>
SEQ ID NO: 80
ADIIGGLAYTMGGRCSVGFAATNAAGQPGFVTAGHCGRVGTQVTIGNGRGVFEQSVFPGNDAAFVRGTS
NFTLTNLVSRYNTGGYATVAGHNQAPIGSSVCRSGSTTGWHCGTIQARGQSVSYPEGTVTNMTRTTVCA
EPGDSGGSYISGTQAQGVTSGGSGNCRTGGTTFYQEVTPMVNSWGVRLRT;
<AA; Trametes hirsuta>
SEQ ID NO: 81
MVATSLLVASLFTLALGTPTGRNLKLHEAREDLPAGFSLRGAASPDTTLKLRIALVQNNFAELEDKLYD
VSTPSSANYGNHLSKEEVEQYIAPAPESVKAVNAWLTENGLDAHTISPAGDWLAFEVPVSKANELFDAD
FSVFTHDESGLEAIRTLAYSIPAELQGHLDLVHPTVTFPNPNAHLPVVRSTQPIRNLTGRAIPASCAST
ITPACLQAIYGIPTTKATQSSNKLAVSGFIDQFANKADLKSFLAQFRKDISSSTTFSLQTLDGGENDQS
PSEAGIEANLDIQYTVGLATGVPTTFISVGDDFQDGNLEGFLDIINFLLGESNPPQVLTTSYGQNENTI
SAKLANQLCNAYAQLGARGTSILFASGDGGVSGSQSAHCSNFVPTFPSGCPFMTSVGATQGVSPETAAA
FSSGGFSNVFGIPSYQASAVSGYLSALGSTNSGKFNRSGRGFPDVSTQGVDFQIVSGGQTIGVDGTSCA
SPTFASVISLVNDRLIAAGKSPLGFLNPFLYSSAGKAALNDVTSGSNPGCSTNGFPAKAGWDPVTGLGT
PNFAKLLTAVGL;
<AA; Bacillus licheniformis>
SEQ ID NO: 82
LVSKKSVKRGLITGLIGISIYSLGMHPAQAAPSPHTPVSSDPSYKAETSVTYDPNIKSDQYGLYSKAFT
GTGKVNETKEKAEKKSPAKAPYSIKSVIGSDDRTRVTNTTAYPYRAIVHISSSIGSCTGWMIGPKTVAT
AGHCIYDTSSGSFAGTATVSPGRNGTSYPYGSVKSTRYFIPSGWRSGNTNYDYGAIELSEPIGNTVGYF
GYSYTTSSLVGTTVTISGYPGDKTAGTQWQHSGPIAISETYKLQYAMDTYGGQSGSPVFEQSSSRTNCS
GPCSLAVHTNGVYGGSSYNRGTRITKEVFDNLTNWKNSAQ;
<AA; Bacillus sp.>
SEQ ID NO: 83
AQTVPYGIPLIKADKVQAQGFKGANVKVAVLDTGIQASHPDLNVVGGASFVAGEAYNTDGNGHGTHVAG
TVAALDNTTGVLGVAPSVSLYAVKVLNSSGSGSYSGIVSGIEWATTNGMDVINMSLGGASGSTAMKQAV
DNAYARGVVVVAAAGNSGSSGNTNTIGYPAKYDSVIAVGAVDSNSNRASFSSVGAELEVMAPGAGVYST
YPTNTYATLNGTSMASPHVAGAAALILSKHPNLSASQVRNRLSSTATYLGSSFYYGKGLINVEAAAQ;
<AA; Lederbergia lenta>
SEQ ID NO: 84
AQSVPWGISRVQAPAAHNRGLTGSGVKVAVLDTGISTHPDLNIRGGASFVPGEPSTQDGNGHGTHVAGT
IAALNNSIGVLGVAPSAELYAVKVLGASGSGSVSSIAQGLEWAGNNGMHVANLSLGSPSPSATLEQAVN
SATSRGVLVVAASGNSGAGSISYPARYANAMAVGATDQNNNRASFSQYGAGLDIVAPGVNVQSTYPGST
YASLNGTSSATPHVAGAAALVKQKNPSWSNVQIRNHLKNTATSLGSTNLYGSGLVNAEAATR;
<AA; Alkalihalobacillus clausii>
SEQ ID NO: 85
MMRKKSFWLGMLTAFMLVFTMAFSDSASAAEEAKEKYLIGFNEQEAVSEFVEQVEANDEVAILSEEEEV
EIELLHEFETIPVLSVELSPEDVDALELDPAISYIEEDAEVTTMAQSVPWGISRVQAPAAHNRGLTGSG
VKVAVLDTGISTHPDLNIRGGASFVPGEPSTQDGNGHGTHVAGTIAALNNSIGVLGVAPSAELYAVKVL
GASGSGSVSSIAQGLEWAGNNGMHVANLSLGSPSPSATLEQAVNSATSRGVLVVAASGNSGAGSISYPA
RYANAMAVGATDQNNNRASFSQYGAGLDIVAPGVNVQSTYPGSTYASLNGTSSATPHVAGAAALVKQKN
PSWSNVQIRNHLKNTATSLGSTNLYGSGLVNAEAATR;
<AA; Alkalihalobacillus clausii>
SEQ ID NO: 86
AQSVPWGISRVQAPAAHNRGLTGSGVKVAVLDTGISTHPDLNIRGGASFVPGEPSTQDGNGHGTHAAGT
IAALNNSIGVLGVAPSAELYAVKVLGASGSGSVSAIAQGLEWAGNNGMHVANLSLGSPSPSATLEQAVN
SATSRGVLVVAASGNSGAGSISYPARYANAMAVGATDQNNNRASFSQYGAGLDIVAPGVNVQSTYPGST
YASLNGTSMATPHVAGAAALVKQKNPSWSNVQIRNHLKNTATSLGSTNLYGSGLVNAEAATR;
<AA; Bacillus sp.>
SEQ ID NO: 87
AVPSTQTPWGIKSIYNDQSITKTTGGKGIKVAVLDTGVYTSHLDLAGSAEQCKDFTQSNPLVDGSCTDR
QGHGTHVAGTVLAHGGSNGQGVYGVAPQAKLWAYKVLGDKGEGYSDDIAAAIRHVADEASRTGSKVVIN
MSLGSSAKDSLIASAVDYAYGKGVLIVAAAGNEGPKPNTIGYPAGFVNAVAVAALENVQEKGTYRVADF
SSRGNPATAGDYIIQERDIEVSAPGASVESTWYTGGYNTISGTSMATPHVAGLAAKIWSANTSLSHSQL
RTELQNRAKVYDIKGGIGAGPGDDYASGFGYPRVK;
<AA; Bacillus sp.>
SEQ ID NO: 88
AATTGTGTTLKGKTVSLNISSESGKYVLRDLSKPTGTQIITYDLQNREYNLPGTLVSSTINQFTTSSQR
AAVDAHYNLGKVYDYFYQKFNRNSYDNKGGKIVSSVHYGSRYNNAAWIGDQMIYGDGDGILFSPLSGSL
DVTAHEMTHGVTQETANLNYENQPGALNESFSDVFGYFNDTEDWDIGEDITISQPALRSLSNPTKYGQP
DNFKNYKNLPNTPAGDYGGVHTNSGIPNKAAYNTITKIGVNKAEQIYYRALTVYLTPSSTFKDAKAALI
QSARDLYGSQDAASVEAAWNAVGL;
<AA; Aspergillus oryzae>
SEQ ID NO: 89
MGVNFKVLALSALATISHASPLLYPRATDSNVTYVFTNPNGLNFTQMNTTLPNVTIFATGGTIAGSSAD
NTATTGYKAGAVGIQTLIDAVPEMLNVANVAGVQVTNVGSPDITSDILLRLSKQINEVVCNDPTMAGAV
VTHGTDTLEESAFFLDATVNCRKPVVIVGAMRPSTAISADGPLNLLQSVTVAASPKARDRGALIVMNDR
IVSAFYASKTNANTVDTFKAIEMGNLGEVVSNKPYFFYPPVKPTGKTEVDIRNITSIPRVDILYSYEDM
HNDTLYSAIDNGAKGIVIAGSGSGSVSTPFSAAMEDITTKHNIPIVASTRTGNGEVPSSAESSQIASGY
LNPAKSRVLLGLLLAQGKSIEEMRAVFERIGVA;
<AA; Aspergillus niger>
SEQ ID NO: 90
SPLLYSRTTNETFVFTNANGLNFTQMNTTLPNVTIFATGGTIAGSDSSSTATTGYTSGAVGVLSLIDAV
PSMLDVANVAGVQVANVGSEDITSDILISMSKKLNRVVCEDPTMAGAVITHGTDTLEETAFFLDATVNC
GKPIVIVGAMRPSTAISADGPFNLLEAVTVAASTSARDRGAMVVMNDRIASAYYVTKTNANTMDTFKAM
EMGYLGEMISNTPFFFYPPVKPTGKVAFDITNVTEIPRVDILFSYEDMHNDTLYNAISSGAQGIVIAGA
GAGGVTTSFNEAIEDVINRLEIPVVQSMRTVNGEVPLSDVSSDTATHIASGYLNPQKSRILLGLLLSQG
KNITEIADVFALGTDA;
<AA; Bacillus licheniformis>
SEQ ID NO: 91
MKLIKNASFIISFLAAAGIYFLLGTVAASAANEDYPEQMIRLESSSGLNITPAGNQDNAPLTAKQTSGE
KEERWRLDTSDGKQFKIRNMDSGKIIIPAHYALSDNNPAVVYYDNSRKEELWNIIGADKDGNGDFITYK
IVSAQNSSLALTLDGSGVKLAKYTGSSVQKWKLPSDGLEGFAGYARETNGKQKTGTTGGLLGKVVYVNN
LGELKANIEDSTPRTIVVSSNIGASAKTVLTVGANKTIIGSYEKHKLNNIYFKTKADSGNVIFKNLVIA
HDASINENNDIPVYITDSRNYWIDHVTFQGHSYTANGHDLDKLLYVGAKADYVTLSHSTFTDHRYGLIL
GWPQDDKQYHSIYNGYPRMTISHNRFENLYVRAPGLMRYGYYHVKSNYINNYHLGFTITTLAKIYSEAN
YFGTGNEKGILDDYGDGAFKDVGSYPAIKGQKSPETSWTPSSNYSYRTMKAGNAKAFAKRYAGAQRTAL
YYANYSQFKKD;
<AA; Aspergillus tubingensis>
SEQ ID NO: 55
MKYSTIFSAAAAVFAGSAAAVGVSGSAEGFAKGVTGGGSATPVYPDTIDELVSYLGDDEARVIVLTKTF
DFTDSEGTTTGTGCAPWGTASACQVAIDQDDWCENYEPDAPSVSVEYYNAGTLGITVTSNKSLIGEGSS
GAIKGKGLRIVSGAENIIIQNIAVTDINAKYVWGGDAITLDDCDLVWIDHVTTARIGRQHYVLGTSADN
RVSLTNNYIDGVSDYSATCDGYHYWAIYLDGDADLVTMKGNYIYHTSGRSPKVQDNTLLHAVNNYWYDI
SGHAFEIGEGGYVLAEGNVFQNVDTVLETYEGEAFTVPSTTAGEVCSTYLGRDCVINGFGSSGTFSEDS
TSFLSDFEGKNIASASAYTSVASSVVANAGQGNL;

1. (canceled)

2. A method comprising applying an enzymatically active protein, to a plant, plant part, plant growth medium and/or agricultural/floricultural/horticultural/silvicultural apparatus/facility, said enzymatically active protein selected from the group consisting of:
i. polypeptides having at least 70% sequence identity to one or more of SEQ ID NO(s): 1-91, 45906, 72144, 72147 and 72149;
ii. polypeptides comprising any one of SEQ ID NO(s): 1-91, 183-72144, 72147 and 72149;
iii. enzymatically active fragments/mutants/variants of any one of SEQ ID NO(s): 1-91, 183-72144, 72147 and 72149;
iv. enzymatically active polypeptides derived from any one of SEQ ID NO(s): 1-91, 183-72144, 72147 and 72149 by substitution, deletion, or insertion of one or more amino acids;
v. enzymatically active polypeptides derived from the polypeptide of any one of i) through iv) wherein the N- and/or C-terminal end has been extended by the addition of one or more amino acids;
vi. enzymatically active fragments of the polypeptide of any one of i) through v); and
vii. fusion proteins comprising a first polypeptide having a first enzymatic activity and a second polypeptide having a second enzymatic activity, wherein at least one of said first polypeptide and said second polypeptide comprises the polypeptide of any one of i) through vi).

3. A formulation comprising an enzymatically active protein in an agriculturally acceptable carrier, said enzymatically active protein selected from the group consisting of:
i. polypeptides having at least 70% sequence identity to one or more of SEQ ID NO(s): 1-91, 45906, 72144, 72147 and 72149;
ii. polypeptides comprising any one of SEQ ID NO(s): 1-91, 183-72144, 72147 and 72149;
iii. enzymatically active fragments/mutants/variants of any one of SEQ ID NO(s): 1-91, 183-72144, 72147 and 72149;
iv. enzymatically active polypeptides derived from any one of SEQ ID NO(s): 1-91, 183-72144, 72147 and 72149 by substitution, deletion, or insertion of one or more amino acids;
v. enzymatically active polypeptides derived from the polypeptide of any one of i) through iv) wherein the N- and/or C-terminal end has been extended by the addition of one or more amino acids;
vi. enzymatically active fragments of the polypeptide of any one of i) through v); and
vii. fusion proteins comprising a first polypeptide having a first enzymatic activity and a second polypeptide having a second enzymatic activity, wherein at least one of said first polypeptide and said second polypeptide comprises the polypeptide of any one of i) through vi).

4. A method comprising applying the formulation of claim 3 to a plant, plant part, plant growth medium and/or agricultural/floricultural/horticultural/silvicultural apparatus/facility.

5-9. (canceled)

10. A method comprising introducing an enzymatically active protein into an agriculturally acceptable carrier that comprises one or more pesticides, pest attractants, pest feeding stimulants, plant growth regulators, rain fasteners and/or UV protectants, said enzymatically active protein selected from the group consisting of:
i. polypeptides having at least 70% sequence identity to one or more of SEQ ID NO(s): 1-91, 45906, 72144, 72147 and 72149;
ii. polypeptides comprising any one of SEQ ID NO(s): 1-91, 183-72144, 72147 and 72149;
iii. enzymatically active fragments/mutants/variants of any one of SEQ ID NO(s): 1-91, 183-72144, 72147 and 72149;
iv. enzymatically active polypeptides derived from any one of SEQ ID NO(s): 1-91, 183-72144, 72147 and 72149 by substitution, deletion, or insertion of one or more amino acids;
v. enzymatically active polypeptides derived from the polypeptide of any one of i) through iv) wherein the N- and/or C-terminal end has been extended by the addition of one or more amino acids;
vi. enzymatically active fragments of the polypeptide of any one of i) through v); and
vii. fusion proteins comprising a first polypeptide having a first enzymatic activity and a second polypeptide having a second enzymatic activity, wherein at least one of said first polypeptide and said second polypeptide comprises the polypeptide of any one of i) through vi).

11. The method of claim 1, wherein application of said enzymatically active protein comprises applying a microorganism that expresses said enzymatically active protein to said plant, plant part, plant growth medium and/or agricultural/floricultural/horticultural/silvicultural apparatus/facility.

12. The method of claim 1, wherein application of said enzymatically active protein comprises introducing a heterologous polynucleotide that encodes said enzymatically active protein into said plant or plant part.
